(19) United States
(12) Patent Application Publication
(10) Pub. No.: US 2024/0006023 A1
(43) Pub. Date: Jan. 4, 2024

(54) SYSTEMS AND METHODS FOR IDENTIFYING NOVEL CRISPR ASSOCIATED PROTEINS

(71) Applicant: The Regents of the University of California, Oakland, CA (US)

(72) Inventor: Omar Akbari, La Jolla, CA (US)

(21) Appl. No.: 18/038,316

(22) PCT Filed: Nov. 23, 2021
(86) PCT No.: PCT/US2021/060547

§ 371 Date: May 23, 2023

Related U.S. Application Data
(60) Provisional application No. 63/117,441, filed on Nov. 23, 2020.
(60) Provisional application No. 63/118,307, filed on Nov. 25, 2020.

(51) Int. Cl. G16B 20/30; C12N 15/10; G16B 35/00

(52) U.S. Cl. CPC G16B 20/30; C12N 15/1089; G16B 35/00

(57) ABSTRACT

Provided herein are systems and methods for identifying Clustered Regularly Interspaced Short Palindromic Repeat (CRISPR)-associated proteins. For example, a method of identifying Clustered Regularly Interspaced Short Palindromic Repeat (CRISPR)-associated proteins can include: (a) obtaining a plurality of genomic sequences, wherein a genomic sequence of the plurality of genomic sequences comprises a CRISPR-associated array; (b) determining a subset of the plurality of genomic sequences comprising a plurality of coding sequences within a 20 kilobase (kb) sequence flanking region either at the 3′ or 5′ end of the CRISPR-associated array; and (c) analyzing a coding sequence of the plurality of coding sequences and thereby identifying the CRISPR-associated protein based on the coding sequence.

### CROSS-REFERENCE TO RELATED APPLICATIONS

This application claims priority to U.S. Provisional Patent Application No. 63/117,441, filed on Nov. 23, 2020, and U.S. Provisional Patent Application No. 63/118,307, filed on Nov. 25, 2020. The disclosure of these prior applications are considered part of the disclosure of this application, and are incorporated in their entireties into this application.

### TECHNICAL FIELD

The present disclosure relates to systems, methods, and materials for identifying candidate CRISPR associated proteins.

### SEQUENCE LISTING

This application contains a Sequence Listing that has been submitted electronically as an ASCII text file named SequenceListing.txt. The ASCII text file, created on Nov. 22, 2021, is 531 kilobytes in size. The material in the ASCII text file is hereby incorporated by reference in its entirety.

### BACKGROUND

The systematic interrogation of genomes and genetic reprogramming of cells involves targeting sets of genes for expression or repression. Currently the most common approach for targeting arbitrary genes for regulation is to use RNA interference (RNAi). This approach has limitations. For example, RNAi can exhibit significant off-target effects and toxicity.

Clustered Regularly interspaced Short Palindromic Repeats (CRISPR) and the CRISPR-associated (Cas) genes, collectively known as the CRISPR-Cas or CRISPR/Cas systems, are currently understood to provide immunity to bacteria and archaea against phage infection. The CRISPR-Cas systems of prokaryotic adaptive immunity are an extremely-diverse group of proteins effectors, non-coding elements, as well as loci architectures, some examples of which have been engineered and adapted to produce important biotechnologies. The components of the systems involved in host defense include one or more effector proteins capable of modifying DNA or RNA and a RNA guide element that is responsible for targeting these protein activities to a specific sequence on the phage DNA or RNA. CRISPR-Cas systems can be broadly classified into two classes: Class 1 systems are composed of multiple effector proteins that together form a complex around a crRNA, and Class 2 systems that consist of a single effector protein that complexes with the crRNA to target DNA or RNA substrates. The single-subunit effector compositions of the Class 2 systems provide a simpler component set for engineering and application translation, and has thus far been important sources of programmable effectors. The discovery, engineering, and optimization of novel Class 2 systems may lead to widespread and powerful programmable technologies for genome engineering and beyond.

There is need in the field for a technology that allows precise targeting of nuclease activity (or other protein activities) to distinct locations within a target DNA in a manner that does not require the design of a new protein for each new target sequence. In addition, there is a need in the art for methods of controlling gene expression with minimal off-target effects.

### SUMMARY

This document provides compositions, methods, and material for identifying Clustered Regularly Interspaced Short Palindromic Repeat (CRISPR)-associated proteins. For example, provided herein are methods including (a) obtaining a set of genomic sequences, wherein a genomic sequence of the set of genomic sequences comprises a CRISPR-associated array; (b) determining coding sequences within a 20 kilobase (kb) sequence flanking either 3′ or 5′ of the CRISPR-associated array; and (c) filtering the coding sequences and using the filtered coding sequences to identify CRISPR-associated proteins. The present disclosure is based on the discovery that methods, including computational methods, can be used to mine prokaryotic genomes and metagenomes for novel CRISPR-associated proteins.

Provided herein are methods of identifying a Clustered Regularly Interspaced Short Palindromic Repeat (CRISPR)-associated protein comprising: (a) obtaining a plurality of genomic sequences, wherein a genomic sequence of the plurality of genomic sequences comprises a CRISPR-associated array; (b) determining a subset of the plurality of genomic sequences comprising a plurality of coding sequences within a 20 kilobase (kb) sequence flanking region either at the 3′ or 5′ end of the CRISPR-associated array; and (c) analyzing a coding sequence of the plurality of coding sequences and thereby identifying the CRISPR-associated protein based on the coding sequence.

In some embodiments, the obtaining step comprises selecting, within the plurality of genomic sequences, a genomic sequence comprising a CRISPR-associated array.

Also provided herein are methods of identifying a CRISPR-associated protein comprising: (a) obtaining a plurality of genomic sequences; (b) selecting, within the plurality of genomic sequences, a genomic sequence comprising a CRISPR-associated array; (c) determining a subset of the plurality of genomic sequences comprising a plurality of coding sequences within a 20 kilobase (kb) sequence flanking region either at the 3′ or 5′ end of the CRISPR-associated array; and (d) analyzing a coding sequence of the plurality of coding sequences and thereby identifying the CRISPR-associated protein based on the coding sequence.

In some embodiments, the plurality of genomic sequences comprise one or more of genomes, wherein the one or more of genomes are selected from: a prokaryotic genome and metagenome. In some embodiments, the selecting step comprises using an algorithm selected from the group consisting of PILER-CR, CRISPR Recognition Tool (CRT), and combinations thereof. In some embodiments, the determining step comprises using an algorithm selected from the group consisting of MetaGeneMark, Prodigal, and combinations thereof.

In some embodiments, the analyzing step comprises filtering the coding sequence that comprises more than 500 amino acids. In some embodiments, the analyzing step comprises filtering a coding sequence that comprises more than 800 amino acids. In some embodiments, the analyzing step further comprises classifying the CRISPR-associated array based on having three or more coding sequences present in the 20 kb flanking region. In some embodiments, the analyzing step further comprises determining a relative position of the coding sequence in the 20 kb flanking region relative to the CRISPR-associated array.

In some embodiments, the analyzing of the coding sequence further comprises removing known CRISPR-associated proteins from the identified CRISPR-associated proteins. In some embodiments, the analyzing of the coding sequence comprises using an algorithm selected from the group consisting of HHMSCAN and RPS-BLAST. In some embodiments, the analyzing of the coding sequence further comprises determining the presence of a structural domain. In some embodiments, the analyzing of the coding sequence comprises determining the presence of a functional domain. In some embodiments, the functional domain comprises a DNA binding domain, a RNA binding domain, a nuclease, a helicase, a restriction domain, or a structural maintenance of chromosomes (SMC) domain.

Also provided herein are computer implemented methods comprising: (a) obtaining a plurality of genomic sequences; (b) selecting, within the plurality of genomic sequences, a genomic sequence comprising a CRISPR-associated array; (c) determining a subset of the plurality of genomic sequences comprising a plurality of coding sequences within a 20 kilobase (kb) sequence flanking region either at the 3′ or 5′ end of the CRISPR-associated array; and (d) analyzing a coding sequence of the plurality of coding sequences and thereby identifying a CRISPR-associated protein based on the coding sequence.

In some embodiments, the plurality of genomic sequences comprises one or more of genomes, wherein the one or more of genomes are selected from: a prokaryotic genome and metagenome. In some embodiments, the selecting step comprises using an algorithm selected from the group consisting of PILER-CR, CRISPR Recognition Tool (CRT), and combinations thereof. In some embodiments, the determining step comprises using an algorithm selected from the group consisting of MetaGeneMark, Prodigal, and combinations thereof.

In some embodiments, the analyzing step comprises filtering the coding sequence that comprises more than 500 amino acids. In some embodiments, the analyzing step comprises filtering a coding sequence that comprises more than 800 amino acids. In some embodiments, the analyzing step further comprises classifying the CRISPR-associated array based on having three or more coding sequences present in the 20 kb flanking region. In some embodiments, the analyzing step further comprises determining a relative position of the coding sequence in the 20 kb flanking region relative to the CRISPR-associated array.

In some embodiments, the analyzing of the coding sequence further comprises removing known CRISPR-associated proteins from the identified CRISPR-associated proteins. In some embodiments, the analyzing of the coding sequence comprises using an algorithm selected from the group consisting of HHMSCAN and RPS-BLAST. In some embodiments, the analyzing of the coding sequence further comprises determining the presence of a structural domain. In some embodiments, the analyzing of the coding sequence comprises determining the presence of a functional domain. In some embodiments, the functional domain comprises a DNA binding domain, a RNA binding domain, a nuclease, a helicase, a restriction domain, or a structural maintenance of chromosomes (SMC) domain.

Also provided herein are non-naturally occurring CRISPR/Cas systems comprising: (a) a guide RNA, wherein the guide RNA comprises a repeat sequence and a spacer sequence capable of hybridizing to a target nucleic acid; and (b) a CRISPR-associated protein or a nucleic acid encoding the CRISPR-associated protein, wherein the CRISPR-associated protein comprises an amino acid sequence that is at least 80% identical to a sequence selected from SEQ ID NOs: 1-50.

In some embodiments, the CRISPR-associated protein is capable of binding to the guide RNA. In some embodiments, the CRISPR-associated protein comprises an amino acid sequence that is at least 85% identical to a sequence selected from SEQ ID NOs: 1-50. In some embodiments, the CRISPR-associated protein comprises an amino acid sequence that is at least 90% identical to a sequence selected from SEQ ID NOs: 1-50. In some embodiments, the CRISPR-associated protein comprises an amino acid sequence that is at least 95% identical to a sequence selected from SEQ ID NOs: 1-50. In some embodiments, the CRISPR-associated protein comprises an amino acid sequence selected from SEQ ID NO: 1-50.

In some embodiments, the target nucleic acid is an RNA or DNA. In some embodiments, the targeting of the target nucleic acid results in a modification of the target nucleic acid. In some embodiments, the modification of the target nucleic acid is a cleavage event.

In some embodiments, the guide RNA further comprises a trans-activating CRISPR RNA (tracrRNA). In some embodiments, the system is present in a delivery system. In some embodiments, the delivery system comprises a delivery vehicle selected from the group consisting of an adeno-associated virus, a nanoparticle, and a liposome.

Also provided herein are methods of treating a condition or disease in a subject in need thereof, the method comprising administering to the subject any one of the systems provided herein, wherein the spacer sequence is substantially complementary to a target nucleic acid associated with the condition or disease; wherein the CRISPR-associated protein associates with the guide RNA to form a complex; wherein the complex binds to the target nucleic acid sequence; and wherein upon binding of the complex to the target nucleic acid sequence the CRISPR-associated protein cleaves the target nucleic acid, thereby treating the condition or disease in the subject.

Unless otherwise defined, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the disclosure will be apparent from the following detailed description, and from the claims.

### BRIEF DESCRIPTION OF DRAWINGS

FIG. 1 is a schematic diagram showing an exemplary method for identifying CRISPR-associated proteins.

FIG. 2 is a schematic diagram showing exemplary step 1 and exemplary step 2 of a method for identifying CRISPR-associated proteins.

FIG. 3 is a schematic diagram showing exemplary step 3 of a method for identifying CRISPR-associated proteins.

FIGS. 4A-413 show the Cas9 size distribution by member and cluster count.

FIGS. 5A-5C are histograms showing number of CRISPR-associated proteins typically associated with the different types of Cas Type II effectors.

FIGS. 6A and 6B are schematic diagrams showing further annotation and filtering done on the 10,913 candidate clusters.

FIG. 7 shows a summary of the method as described herein.

FIG. 8 is a schematic diagram showing an exemplary workflow.

### DETAILED DESCRIPTION

This document provides methods of identifying Clustered Regularly Interspaced Short Palindromic Repeat (CRISPR)-associated proteins where the method includes computation identification. In some embodiments, these computational methods are directed to identifying CRISRP-associated proteins that co-occur in close proximity to CRISPR arrays. It should be understood that the methods and calculations described herein may be performed on one or more computing devices.

Various non-limiting aspects of these methods and systems are described herein, and can be used in any combination without limitation. Additional aspects of various components of systems and methods for identifying CRISPR associated proteins are known in the art.

It must be noted that, as used in the specification and the appended claims, the singular forms “a,” “an” and “the” include plural referents unless the context clearly dictates otherwise.

As used herein, the terms “about” and “approximately,” when used to modify an amount specified in a numeric value or range, indicate that the numeric value as well as reasonable deviations from the value known to the skilled person in the art, for example ±20%, ±10%, or ±5%, are within the intended meaning of the recited value.

As used herein, a “cell” can refer to either a prokaryotic or eukaryotic cell, optionally obtained from a subject or a commercially available source.

As used herein, “delivering”, “gene delivery”, “gene transfer”, “transducing” can refer to the introduction of an exogenous polynucleotide into a host cell, irrespective of the method used for the introduction. Such methods include a variety of well-known techniques such as vector-mediated gene transfer (e.g., viral infection/transfection, or various other protein-based or lipid-based gene delivery complexes) as well as techniques facilitating the delivery of “naked” polynucleotides (e.g., electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

In some embodiments, a polynucleotide can be inserted into a host cell by a gene delivery molecule. Examples of gene delivery molecules can include, but are not limited to, liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

As used herein, the term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The term “exogenous” refers to any material introduced from or originating from outside a cell, a tissue or an organism that is not produced by or does not originate from the same cell, tissue, or organism in which it is being introduced.

As used herein, “nucleic acid” is used to include any compound and/or substance that comprise a polymer of nucleotides. In some embodiments, a polymer of nucleotides are referred to as polynucleotides. Exemplary nucleic acids or polynucleotides can include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a (3-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A deoxyribonucleic acid (DNA) can have one or more bases selected from the group consisting of adenine (A), thymine (T), cytosine (C), or guanine (G), and a ribonucleic acid (RNA) can have one or more bases selected from the group consisting of uracil (U), adenine (A), cytosine (C), or guanine (G).

In some embodiments, the term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the isolated nucleic acids described herein, the isolated nucleic acid is DNA. In some embodiments of any of the isolated nucleic acids described herein, the isolated nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

Unless otherwise specified, a “nucleotide sequence encoding a protein” includes all nucleotide sequences that are degenerate versions of each other and thus encode the same amino acid sequence.

The term “plurality” can refer to a state of having a plural (e.g., more than one) number of different types of things (e.g., a cell, a genomic sequence, a subject, a system, or a protein). In some embodiments, a plurality of genomic sequences can be more than one genomic sequence wherein each genomic sequence is different from each other.

The term “subject” is intended to include any mammal. In some embodiments, the subject is cat, a dog, a goat, a human, a non-human primate, a rodent (e.g., a mouse or a rat), a pig, or a sheep.

The term “transduced”, “transfected”, or “transformed” refers to a process by which exogenous nucleic acid is introduced or transferred into a cell. A “transduced,” “transfected,” or “transformed” mammalian cell is one that has been transduced, transfected or transformed with exogenous nucleic acid (e.g., a gene delivery vector) that includes an exogenous nucleic acid encoding RNA-binding zinc finger domain).

The term “treating” means a reduction in the number, frequency, severity, or duration of one or more (e.g., two, three, four, five, or six) symptoms of a disease or disorder in a subject (e.g., any of the subjects described herein), and/or results in a decrease in the development and/or worsening of one or more symptoms of a disease or disorder in a subject.

The term “promoter” means a DNA sequence recognized by enzymes/proteins in a mammalian cell required to initiate the transcription of an operably linked coding sequence (e.g., a nucleic acid encoding a fusion protein (e.g., a RNA-binding zinc finger domain and a fusion partner)). A promoter typically refers, to e.g. a nucleotide sequence to which an RNA polymerase and/or any associated factor binds and at which transcription is initiated. The promoter can be constitutive, inducible, or tissue-specific (e.g., a brain-specific promoter).

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 (Genentech, South San Francisco, Calif) or Megalign (DNASTAR). The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

### Clustered Regularly Interspaced Short Palindromic Repeats (CRISPR)

As used herein, the term “CRISPR” refers to a technique of sequence specific genetic manipulation relying on the clustered regularly interspaced short palindromic repeats pathway, which unlike RNA interference regulates gene expression at a transcriptional level. The term “gRNA” or “guide RNA” refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7 and Graham, D., et al. Genome Biol. 2015; 16: 260. The term “Single guide RNA” or “sgRNA” is a specific type of gRNA that combines tracrRNA (transactivating RNA), which binds to Cas9 to activate the complex to create the necessary strand breaks, and crRNA (CRISPR RNA), comprising complimentary nucleotides to the tracrRNA, into a single RNA construct. Exemplary methods of employing the CRISPR technique are described in WO 2017/091630, which is incorporated by reference in its entirety.

In some embodiments, the single guide RNA can recognize a target RNA, for example, by hybridizing to the target RNA. In some embodiments, the single guide RNA comprises a sequence that is complementary to the target RNA. In some embodiments, the sgRNA can include one or more modified nucleotides. In some embodiments, the sgRNA has a length that is about 10 nt (e.g., about 20 nt, about 30 nt, about 40 nt, about 50 nt, about 60 nt, about 70 nt, about 80 nt, about 90 nt, about 100 nt, about 120 nt, about 140 nt, about 160 nt, about 180 nt, about 200 nt, about 300 nt, about 400 nt, about 500 nt, about 600 nt, about 700 nt, about 800 nt, about 900 nt, about 1000 nt, or about 2000 nt).

In some embodiments, a single guide RNA can recognize a variety of RNA targets. For example, a target RNA can be messenger RNA (mRNA), ribosomal RNA (rRNA), signal recognition particle RNA (SRP RNA), transfer RNA (tRNA), small nuclear RNA (snRNA), small nucleolar RNA (snoRNA), antisense RNA (aRNA), long noncoding RNA (lncRNA), microRNA (miRNA), piwi-interacting RNA (piRNA), small interfering RNA (siRNA), short hairpin RNA (shRNA), retrotransposon RNA, viral genome RNA, or viral noncoding RNA. In some embodiments, a target RNA can be an RNA involved in pathogenesis of conditions such as cancers, neurodegeneration, cutaneous conditions, endocrine conditions, intestinal diseases, infectious conditions, neurological conditions, liver diseases, heart disorders, or autoimmune diseases. In some embodiments, a target RNA can be a therapeutic target for conditions such as cancers, neurodegeneration, cutaneous conditions, endocrine conditions, intestinal diseases, infectious conditions, neurological conditions, liver diseases, heart disorders, or autoimmune diseases.

As used herein, a “CRISPR-associated protein” can refer to an enzyme that uses CRISPR sequences as a guide to recognize and cleave specific nucleic acid strands that are complementary to the CRISPR sequence. A CRISPR-associated protein can associate with a CRISPR RNA sequence to bind to, and alter DNA or RNA target sequences. In some embodiments, a CRISPR-associated protein can be a Cas9 endonuclease that makes a double-stranded break in a target DNA sequence. In some embodiments, a CRISPR-associated protein can be a Cas12a nuclease that also makes a double-stranded break in a target DNA sequence. In some embodiments, a CRISPR-associated protein can be a Cas13 nuclease which targets RNA. Additional CRISPR-associated proteins within the scope of the disclosure as identified by the novel method presented herein also include SEQ ID NOs: 1-50.

As used herein, a “CRISPR-associated array” can refer to a component of a CRISPR-Cas system, wherein a CRISPR-associated array can include alternating conserved repeats and spacers that are transcribed into a precursor CRISPR RNA and processed into individual CRISPR RNAs. In some embodiments, a CRISPR-associated array includes between two and several hundred repeating sequences separated by unique spacers. Both the repeats and spacers in an array have interesting features, wherein each DNA repeat is a partial palindrome while spacers all share a common sequence called a Proto-spacer Adjacent Motif (PAM) that Cas9 requires to recognize its DNA target. In some embodiments, a CRISPR-associated array has a 20 kb flanking region either at the 3′ or 5′ end of the CRISPR-associated array. In some embodiments, the CRISPR-associated array has a 20 kb flanking region at both the 3′ and 5′ end of the CRISPR-associated array. In some embodiments, a flanking region can include a coding sequence. In some embodiments, a flanking region can include a plurality of coding sequences. In some embodiments, a flanking region can include three or more coding sequences.

### CRISPR/Cas System

Provided herein are non-naturally occurring CRISPR/Cas systems including (a) a guide RNA, wherein the guide RNA comprises a repeat sequence and a spacer sequence capable of hybridizing to a target nucleic acid; and (b) a CRISPR-associated protein or a nucleic acid encoding the CRISPR-associated protein, wherein the CRISPR-associated protein comprises an amino acid sequence that is at least 80%, at least 85%, at least 86%, at least 87%, at least 88%, or at least 89% identical to a sequence selected from SEQ ID NOs: 1-50.

In some embodiments, the CRISPR-associated protein comprises an amino acid sequence that is at least 90%, at least 91%, at least 92%, at least 93%, at least 94%, at least 95%, at least 96%, at least 97%, at least 98%, or at least 99% identical to a sequence selected from SEQ ID NOs: 1-50. In some embodiments, the CRISPR-associated protein comprises an amino acid sequence selected from SEQ ID NO: 1-50.

In some embodiments, the CRISPR-associated protein is capable of binding to the guide RNA and of targeting the nucleic acid sequence complementary to the guide RNA spacer sequence. In some embodiments, the target nucleic acid is an RNA or DNA. In some embodiments, the targeting of the target nucleic acid results in a modification of the target nucleic acid. In some embodiments, the modification of the target nucleic acid is a cleavage event. In some embodiments, the guide RNA further comprises a trans-activating CRISPR RNA (tracrRNA).

In some embodiments, the system is present in a delivery system. In some embodiments, the delivery system comprises a delivery vehicle selected from the group consisting of an adeno-associated virus, a nanoparticle, and a liposome.

TABLE 1

| SEQ ID | | |
|---|---|---|
| NO: | Protein ID | Amino acid Sequences |
| SEQ ID | gene_5155455 | MTKPYSIGLDIGTNSVGWAVITDNYKVPSKKMKVLGNTSKKYIKKNL |
| NO: 1 | | LGVLLFDSGITAEGRRLKRTARRRYTRRRNRILYLQEIFSTEMATLDD |
| | | AFFQRLDDSFLVPDDKRDSKYPIFGNLVEEKAYHDEFPTIYHLRKYLA |
| | | DSTKKADLRLVYLALAHMIKYRGHFLIEGEFNSKNNDIQKNFQDFLD |
| | | TYNAIFESDLSLENSKQLEEIVKDKISKLEKKDRILKLFPGEKNSGIFSE |
| | | FLKLIVGNQADFRKCFNLDEKASLHFSKESYDEDLETLLGYIGDDYSD |
| | | VFLKAKKLYDAILLSGFLTVTDNETEAPLSSAMIKRYNEHKEDLALLK |
| | | EYIRNISLKTYNEVFKDDTKNGYAGYIDGKTNQEDFYVYLKNLLAEF |
| | | EGADYFLEKIDREDFLRKQRTFDNGSIPYQIHLQEMRAILDKQAKFYP |
| | | FLAKNKERIEKILTFRIPYYVGPLARGNSDFAWSIRKRNEKITPWNFED |
| | | VIDKESSAEAFINRMTSFDLYLPEEKVLPKHSLLYETFNVYNELTKVRF |
| | | IAESMRDYQFLDSKQKKDIVRLYFKDKRKVTDKDIIEYLHAIYGYDGI |
| | | ELKGIEKQFNSSLSTYHDLLNIINDKEFLDDSSNEAIIEEIIHTLTIFEDRE |
| | | MIKQRLSKFENIFDKSVLKKLSRRHYTGWGKLSAKLINGIRDEKSGNT |
| | | ILDYLIDDGISNRNFMQLIHDDALSFKKKIQKAQIIGDEDKGNIKEVVK |
| | | SLPGSPAIKKGILQSIKIVDELVKVMGGRKPESIVVEMARENQYTNQG |
| | | KSNSQQRLKRLEKSLKELGSKILKENIPAKLSKIDNNALQNDRLYLYY |
| | | LQNGKDMYTGDDLDIDRLSNYDIDHIIPQAFLKDNSIDNKVLVSSASN |
| | | RGKSDDFPSLEVVKKRKTFWYQLLKSKLISQRKFDNLTKAERGGLLP |
| | | EDKAGFIQRQLVETRQITKHVARLLDEKFNSNKKDENNRAVRTVKIIT |
| | | LKSTLVSQFRKDFELYKVREINDFHHAHDAYLNAVIASALLKKYPKL |
| | | EPEFVYGDYPKYNSFRERKSATEKVYFYSNIMNIFKKSISLADGRVIER |
| | | PLIEVNEETGESVWNKESDLATVRRVLSYPQVNVVKKVEEQNHGLDR |
| | | GKPKGLFNANLSSKPKPNSNENLVGAKEYLDPKKYGGYAGISNSFAV |
| | | LVKGTIEKGAKKKITNVLEFQGISILDRINYRKDKLNFLLEKGYKDIELI |
| | | IELPKYSLFELSDGSRRMLASILSTNNKRGEIHKGNQIFLSQKFVKLLY |
| | | HAKRISNTINENHRKYVENHKKEFEELFYYILEFNENYVGAKKNGKL |
| | | LNSAFQSWQNHSIDELCSSFIGPTGSERKGLFELTSRGSAADFEFLGVK |
| | | IPRYRDYTPSSLLKDATLIHQSVTGLYETRIDLAKLGEG |
| SEQ ID | gene_3815793 | MSIRSFKLKIKTKSGVNAEELRRGLWRTHQLINDGIAYYMNWLVLLR |
| NO: 2 | | QEDLFIRNEETNEIEKRSKEEIQGELLERVHKQQQRNQWSGEVDDQTL |
| | | LQTLRHLYEEIVPSVIGKSGNASLKARFFLGPLVDPNNKTTKDVSKSG |
| | | PTPKWKKMKDAGDPNWVQEYEKYMAERQTLVRLEEMGLIPLFPMY |
| | | TDEVGDIHWLPQASGYTRTWDRDMFQQAIERLLSWESWNRRVRERR |
| | | AQFEKKTHDFASRFSESDVQWMNKLREYEAQQEKSLEENAFAPNEPY |
| | | ALTKKALRGWERVYHSWMRLDSAASEEAYWQEVATCQTAMRGEFG |
| | | DPAIYQFLAQKENHDIWRGYPERVIDFAELNHLQRELRRAKEDATFTL |
| | | PDSVDHPLWVRYEAPGGTNIHGYDLVQDTKRNLTLILDKFILPDENGS |
| | | WHEVKKVPFSLAKSKQFHRQVWLQEEQKQKKREVVFYDYSTNLPHL |
| | | GTLAGAKLQWDRNFLNKRTQQQIEETGEIGKVFFNISVDVRPAVEVK |
| | | NGRLQNGLGKALTVLTHPDGTKIVTGWKAEQLEKWVGESGRVSSLG |
| | | LDSLSEGLRVMSIDLGQRTSATVSVFEITKEAPDNPYKFFYQLEGTELF |
| | | AVHQRSFLLALPGENPPQKIKQMREIRWKERNRIKQQVDQLSAILRLH |
| | | KKVNEDERIQAIDKLLQKVASWQLNEEIATAWNQALSQLYSKAKEN |
| | | DLQWNQAIKNAHHQLEPVVGKQISLWRKDLSTGRQGIAGLSLWSIEE |
| | | LEATKKLLTRWSKRSREPGVVKRIERFETFAKQIQHHINQVKENRLKQ |
| | | LANLIVMTALGYKYDQEQKKWIEVYPACQVVLFENLRSYRFSYERSR |
| | | RENKKLMEWSHRSIPKLVQMQGELFGLQVADVYAAYSSRYHGRTGA |
| | | PGIRCHALTEADLRNETNIIHELIEAGFIKEEHRPYLQQGDLVPWSGGE |
| | | LFATLQKPYDNPRILTLHADINAAQNIQKRFWHPSMWFRVNCESVME |
| | | GEIVTYVPKNKTVHKKQGKTFRFVKVEGSDVYEWAKWSKNRNKNT |
| | | FSSITERKPPSSMILFRDPSGTFFKEQEWVEQKTFWGKVQSMIQAYMK |
| | | KTIVQRMEE |
| SEQ ID | gene_2964877 | MNKAADNYTGGNYDEFIALSKVQKTLRNELKPTPFTAEHIKQRGIISE |
| NO: 3 | | DEYRAQQSLELKKIADEYYRNYITHKLNDINNLDFYNLFDAIEEKYKK |
| | | NDKDNRDKLDLVEKSKRGEIAKMLSADDNFKSMFEAKLITKLLPDYV |
| | | ERNYTGEDKEKALETLALFKGFTTYFKGYFKTRKNMFSGEGGASSIC |
| | | HRIVNVNASIFYDNLKTFMRIQEKAGDEIALIEEELTEKLDGWRLEHIF |
| | | SRDYYNEVLAQKGIDYYNQICGDINKHMNLYCQQNKFKANIFKMMK |
| | | LQKQIMGISEKVFEIPPMYQNDEEVYASFNEFISRLEEVKLTDRLRNIL |
| | | QNINIYNTAKIYINARYYTNVSTYVYGGWGVIESAIERYLCNTIAGKG |
| | | QSKVKKIENAKKDNKFMSVKELDSIVAEYEPDYFNAPYIDDDDNAVK |
| | | VFGGQGVLGYFNKMSELLADVSLYTIDYNSDDSLIENKESALRIKKQL |
| | | DDIMSLYHWLQTFIIDEVVEKDNAFYAELEDICCELENVVTLYDRIRN |
| | | YVTKKPYSTQKFKLNFASPTLAAGWSRSKEFDNNAIILLRNNKYYIAI |
| | | FNVNNKPDKQIIKGSEEQRLSTDYKKMVYNLLPGPNKMLPKVFIKSD |
| | | TGKRDYNPSSYILEGYEKNRHIKSSGNFDINYCHDLIDYYKACINKHP |
| | | EWKNYGFKFEETTQYNDIGQFYKDVEKQGYSISWVYISEADINRLDE |
| | | EGKIYLFEIYNKDLSSHSTGKDNLHTMYLKNIFSEDNLKNICIELNGNA |
| | | ELFYRKSSMKRNITHKKDTVLVNKTYINEAGVRVSLTDEDYIKVYNY |
| | | YNNDYVIDVEKDKKLVEILERIGHRKNPIDIIKDKRYTEDKYFLHLPITI |
| | | NYGVDDENINAKMIEYIAKHNNMNVIGIDRGERNLIYISVINNKGNIIE |
| | | QKSFNLVNNYDYKNKLKNMEKTRDNARKNWQEIGKIKDVKSGYLS |
| | | GVISEIARMVIDYNAIIVMEDLNKGFKRGRFKVERQVYQKFENMLISK |
| | | LNYLVFKERKADENGGILRGYQLTYIPKSIKNVGKQCGCIFYVPAAYT |
| | | SKIDPATGFINIFDFKKYSGSGINAKVKDKKEFLMSMNSIRYINEGSEE |
| | | YEKIGHRELFAFSFDYNNFKTYNVSSPVNEWTAYTYGERIKKLYKDG |
| | | RWLRSEVLNLTENLIKLMEQYNIEYKDGHDIREDISHMDETRNADFIC |
| | | SLFEELKYTVQLRNSKSEAEDENYDRLVSPILNSSNGFYDSSDYMENE |
| | | NNTTHIMPKDADANGAYCIALKGLYEINKIKQNWSDDKKFKENELYI |
| | | NVVEWLDYIQNRRFE |
| SEQ ID | gene_4147644 | MKLSKEKHTRSAVANNGDIKSAEVNNGNTKSEEVNNGDIRSAVANE |
| NO: 4 | | EQNIGGILYRFPGKSIDGVKDQMLRRDKEVKKLYNVFNQIQVGTKPK |
| | | KWNNDEKLSPEENERRAQQKNIKMKNYKWREACSKYVESSQRIIND |
| | | VIFYSYRKAENKLRYMRKNEDILKKMQEAEKLSKFSGGKLEDFVAYT |
| | | LRKSLVVSKYDTQEFDSVAAMVVFLECIGKNNISDHEREIVCKLLELI |
| | | RKDFSKLDPNVKGSQGANIVRSVRNQNMIVQPQGDRFLFPQVYAKEN |
| | | ETVTNKNVEKEGLNEFLLNYANLDDEKRAESLRKLRRILDVYFSAPN |
| | | HYEKDMDITLSDNIEKEKFNVWEKHECGKKETGLFVDIPDVLMEAEA |
| | | ENIKLDAVVEKRERKVLNDRVRKQNIICYRYTRAVVEKYNSNEPLFFE |
| | | NNAINQYWIHHIENAVERILKNCKAGKLFKLRKGYLAEKVWKDAINL |
| | | ISIKYIALGKAVYNFALDDIWKDKKNKELGIVDERIRNGITSFDYEMIK |
| | | AHENLQRELAVDIAFSVNNLARAVCDMSNLGNKESDFLLWKRNDIA |
| | | DKLKNKDDMASVSAVLQFFGGKSSWDINIFKEAYKGKKKYNYEVRFI |
| | | DDLRKAIYCARNENFHFKTALVNDEKWNTELFGKIFERETEFCLNVE |
| | | KDRFYSNNLYMFYQVSELRNMLDHLYSRSVSRAAQVPSYNSVIVRTA |
| | | FPEYITNVLGYQKPGYDADTLGKWYSACYYLLKEIYYNSFLQSDRAL |
| | | QLFEKSVKTLSWDDKKQQRAVDNFKDHFSDIKSACTSLAQVCQIYMT |
| | | EYNQQNNQIKKVRSSNDSIFDQPVYQHYKVLLKKAIANAFADYLKNN |
| | | KDLFGFIGKPFKANEIREIDKEQFLPDWTSRKYEALCIEVSGSQELQK |
| | | WYIVGKFLNAMSLNLMVGSMRSYIQYVTDIKRRAASIGNELHVSVQD |
| | | VEKVEKWVQVIEVCSLLASRTSNQFEDYFNDKDDYARYLKSYVDFS |
| | | NVDMPSEYSALVDFSNEEQSDLYVDPKNPKVNRNIVHSKLFAADHIL |
| | | RDIVEPVSKDNIEEFYSQKAEIAYCKIKGKEITAEEQKAVLKYQKLKN |
| | | RVELRDIVEYGEIINELLGQLINWSFMRERDLLYFQLGFHYDCLRNDS |
| | | KKPEGYKNIKVDENSIKDAILYQIIGMYVNGVTVYAPEKDGDKLKEQ |
| | | CVKGGVGVKVSAFHRYSKYLGLNEKTLYNAGLEIFEVVAEHEDIINL |
| | | RNGIDHFKYYLGDYRSMLSIYSEVFDRFFTYDIKYQKNVLNLLQNILL |
| | | RHNVIVEPILESGFKTIGEQTKPGAKLSIRSIKSDTFQYKVKGGTLITDA |
| | | KDERYLETIRKILYYAENEEDNLKKSVVVTNADKYEKNKESDDQNK |
| | | QKEKKNKDNKGKKNEETKSDAEKNNNERLSYNPFANFDFKLLN |
| SEQ ID | meta_gene_ | MAKKNKMKPRELREAQKKARQLKAAEINNNAAPAIAAMPVAEAAA |
| NO: 5 | 174274 | PAAEKKKSSVKAAGMKSILVSENKMYITSFGKGNSAVLEYEVDNND |
| | | YNKTQLSSKDNSNIELGDVNEVNITFSSKHGFESGVEINTSNPTHRSGE |
| | | SSPVRGDMLGLKSELEKRFFGKTFDDNIHIQLIYNILDIEKILAVYVINI |
| | | VYALNNMLGEGDESNYDFMGYLSTFNTYKVFTNPNGSTLSDDKKENI |
| | | RKSLSKFNALLKTKRLGYFGLEEPKTKDTRVLEAYKKRVYYMLAIVG |
| | | QIRQCVFHDLSEHSEYDLYSFIDNSKKVYRECRETLDYLVDERFDSIN |
| | | KGFIQGNKVNISLLIDMMKGYEPDDIIRLYYDFIVLKSQKNLGFSIKKL |
| | | REKMLDEYGFRFKDKQYDSVRSKMYKLMDFLLFCNYYRNDVAAGE |
| | | ALVRKLRFSMTDDEKEGIYADEAAKLWGKFRNDFENIADHMNGDVI |
| | | KELGKADMNFDEKILDSEKKNASDLLYFSKMIYMLTYFLDGKEINDL |
| | | LTTLISKFDNIKEFLKIMKSSAVDVECELTAGYKLFNDSQRITNELFIVK |
| | | NIASMRKPAASAKLTMFRDALTILGIDDKITDDRISEILKLKEKGKGIH |
| | | GLRNFITNNVIESSRFVYLIKYANAQKIREVAKNEKVVMFVLGGIPDT |
| | | QIERYYKSCVEFPDMNSSLEAKRSELARMIKNISFDDFKNVKQQAKG |
| | | RENVAKERAKAVIGLYLTVMYLLVKNLVNVNARYVIAIHCLERDFGL |
| | | YKEIIPELASKNLKNDYRILSQTLCELCDKSPNLFLKKNERLRKCVEVD |
| | | INNADSSMTRKYRNRIAHLTVVRELKEYIGDIRTVDSYFSIYHYVMQR |
| | | CITKREDDTKQGEKIKYEDDLLKNHGYTKDFVKALNSPFGYNIPRFKN |
| | | LSIEQLFDRNEYLTEK |
| SEQ ID | gene_4200106 | MPAAEVIAPAAEKKKSSVKAAGMKSILVSENKMYITSFGKGNSAVLE |
| NO: 6 | | YEVDNNDYNQTQLSSEDSSNIELCGVTKVNITFSSKHGLESGVEINTSN |
| | | PTHRSGESSPVRWDMLGLKSELEKRFFGKTFDDNIHIQLIYNILDIEKIL |
| | | AVYVTNIVYALNNMLGIKKSESYDDFMGYLSARNTYEVFTHPDKSNL |
| | | SDKAKGNIKKSFSTFNDLLKTKRLGYFGLEEPKTKDTRVSQAYKKRV |
| | | YHMLAIVGQIRQCVFHDKSGAKKFDLYSFINNIDSEYRETLDYLVDER |
| | | FDSINKGFIQGNKVNISLLIDMMKGYKADDIIRLYYDFIVLKSQKNLGF |
| | | SIKKLREKMLDEYGFRFKDKQYDSVRSKMYKLMDFLLFCNYYRNDV |
| | | IAGEDLVRKLRFSMTDDEKEGIYADEAEKLWGKFRNDFENIADHMN |
| | | GDVIKELGQADMDFDEKILDSEKKNASDLLYFSKMIYMLTYFLDGKE |
| | | INDLLTTLISKFDNIKEFLKIMKSSAVDVECELTAGYKLFNDSQRITNE |
| | | LFIVKNIASMRKPAASAKLTMFRDALTILGIDDKITDDRISEI |
| SEQ ID | meta_gene_ | MLQQPYTIDYGSRKTGSKAAAVGDNYYPTFFLDLLIIIGRPLQPITQSN |
| NO: 7 | 524079 | DRFFDNVTVTFKNWRASYSSKHVHGLPFDLDHRTFRLATAATREAW |
| | | YIVMHPTASTITDLPSSRRERRKRLEKSSQSSALQLHHAHFLAGYIKW |
| | | VFLIDDLLGEGVEPSWTINGPHLTKITFNKWTAFQNRFMEEWDSYVQ |
| | | EYSCDNFWMENQPAFHAYDYGANIEIEIREESELSKQLKSLPKETRLR |
| | | RNNEESESEEEDTNILEDGTQLMSSRSNSREVSEAPEEINYQSLYTEGL |
| | | RQLRTELERKYILNNISSISYALAVDIGCQDSNSPDPEDKQVYCLLADR |
| | | NKVLGDFRGPRDFTFYPLAFHPAYGNFSSPGPPSFLIDNVLAVMRDN |
| | | MSYQNDGADTLSYGYFQAYSNIKRSIRHKPEDLLATKGIATAALALPE |
| | | SEANASSHIKAKRQRLLQRLQGQATPEDPDSSKPFERERQLIEAAIVAE |
| | | KFDFRMEQVLTIQVSRLIDSRRNFSTVLNPIFQLVRFYLMESHRYTHLL |
| | | RWFPPSVFPGILGSFARIFGLAIDEIYARFKAGGSKGLSIALAEGVSALD |
| | | RLGSYCFTGFPKSLMGSVLSPLGTIDGIEQGAWPYINPRMLDLQDGGG |
| | | SLCLSQWPRGENKRPLLMHVASIGFYYGPEVAASRHSNVWFKEFGG |
| | | MSIKGPSGAAKFLEDLFQDLWIPQTVAFVDHQLNRGLRQGSGSADKT |
| | | KEELLLLEHQQALIRQWLQSEHPFSWAYVNDRRAVKCCS |
| SEQ ID | meta_crt_ | MRIPVTQARNNLLGGERSWRDMVSPAQRFLQPRSARAPRSLAGSKM |
| NO: 8 | array_ | PNSPRETRLTHNNFRGSRLLAEHRHDCAGAGMARIRGIARVGLDDYD |
| | WNGG01011662.1 | VKIIPGDDGPGLRDLARHDHGHVGRDSGRRWRAVARVCGPVHATGG |
| | | GSRTVLEALERGWSRTRAQRVVLRPLRAPEVHQLPDRSRDGANRLVS |
| | | NDKGAARAEGEEQDAARGVSATAAHVTDVDFDGVGAKRRVRVPAA |
| | | HGEPAAAGGNGSRRGGTAVTPVDGRCVVGHRAVRVSIGEAGHHRIG |
| | | RDGVGAAQGLAGCRQGGIGDGRRARSGGAVADVVDVGDRRCDREG |
| | | PLLSVGVRATHREGSTGRTGDGARGGNAAVAPVDRRRVFARRCLGV |
| | | GICDGGDCAANGYSLSCRDRGSRGLDGRVCGGHPRLGSGGLLQCSSV |
| | | INKGRRDGICPHSATVGVGERDLAVDVRRARDRAAGAHGAHVGPAV |
| | | DAEGDGLPRLRAATLEDGRGHGVAAADWVSGRCRSQADVGLDSDA |
| | | AAAEQDVWRARDGSPSVDVRRRVGLGHGRVCSQCQHCPVTREVVE |
| | | EGMHRAAPVGQVGVVEPGRRPGRGDDGDAAGTGGAPRCELMRSVG |
| | | AQSVHDWHRGSRWSSAIPGVRGAELATRVDVGAGRACHCTREGNAF |
| | | SLEEIASPGAARARVVEGRVGADQNLVASTGHHHRLPDGGVGLSGG |
| | | VGLVALAGRIADDERLIGGELACRHSVPLGVSRQGDGEAVGLSLLLL |
| | | QALPAAIGSGARGYAASQDEHLGGGSMRVSDGA* |
| SEQ ID | meta_gene_ | MEIIDKVNANSFYKMRDKFLYSSDVRENIALRDNVFAPIFILCEVNEIR |
| NO: 9 | 336895 | NFDGERNDKLSYFEMKLNYETKEIDLGSNYRSLVDRIKVIKKEMKIFY |
| | | EEMLKNERDVDVSFINSNKEIKEKLIDFIKEKKEFFEMSDDDFIDLYKR |
| | | FYRLLYCINDEQSKLIIGENIKREINFYRNIVNTKDKTRLLEKKYNVED |
| | | DPYLVLFSVFYNGISDYEYSIFNIGMLKREVNKLFMLNKLNNEFYNLL |
| | | IDMHVMSMEEILFSENNLGIKYSELETMVKYSLHDRIGIVENADRLLII |
| | | KEDEKTKEKNTETNIYKNFNLEYYDKVKTLDLINIEFKISEDIDENIKK |
| | | LLDIKTQENLKFFVVTYLIEHGYINYYKGNKLKNITISTKQEKNEEERT |
| | | INRGFIYLKLSEKDFQFEGTLELEFEIRLGLKIIQKREKLSFTEEDIKSDK |
| | | VESVSKILLNSNDSNKNYFDGNTTFGLPRNQVDFNVFFRKFKEYFSSN |
| | | RSRKFTGYDIKINRDRTREKNIPITLKRINKSAYCEKIRVNPLTGEIISNN |
| | | KYNKSLEDVMLHTYLYIYMQSLFLMVKTRLKNNGEFKTLDLFNFESF |
| | | MGLINNINVPDTRHGLFRYINYYYFEKYEPFIENKIKYKPITKDGKIIKN |
| | | EIENIINNMEDYILLAKISEFIYLQILHNFSLENIIDIEIATNEEIKSILNLN |
| | | YNDVELKKESKYIKTIMNYYAEFLETFNNEIKIKEEN |
| SEQ ID | meta_gene_ | MVGEYLGLTTFEKAIEQKPITLTKVDIDKSIINDYKEINDFILAKKSTVS |
| NO: 10 | 321445 | LLNEDNKKMVEYAKIHGIDTKEILKEIKSLHKAENKELEKDMKSSELD |
| | | KNYAWYLENKENKAIKDVLETKKNTFLSEWSKEIGNLESDEKLAYLK |
| | | GTNDVNFKNEIYNFSDEKIKKVLDIYTNFKEEYTANQKEYFNNNGVEI |
| | | EKEEEKEKNFHSINEINQNYLSDKEKINDILSVLKITVEDIEKTEEQIKK |
| | | RNPDLNDKTIADMITNHIYDNMIKENVALIDFSKKEFFNFGNENITKE |
| | | MELERYFNLKEKNSIESFDEKDLVSFDKVDYLIKDRENIINNERQYLLS |
| | | NELKEHQDINYQAKKEIALILTNTNALDREIKAEMLEFKDDKLIVITPE |
| | | YNAEIKTEKLTENNIDKIRNIILNGIENKTSLNDMEKLNKNNEILFNLG |
| | | QRDKLREFGNSFYKDMIFDKENEIIKEIKVEIVKEKYLELSDREKVLER |
| | | AKELGITLEKEPVFTTKSITKDMEDAEPNIDKDNEVTINYFENRNDIYQ |
| | | FFENSLYLKNALRIYEDTLDFDYKEIYPYNNLEENAKFIVANILELIPEI |
| | | KNNKEEFGIDSINLHEYLKEMSFDDLELWINNIDETIKEVIENKVEKDD |
| | | DYVPEVTDKTEDISNNNEDENKRIENDKEKNKEKDDEYNF |
| SEQ ID | gene_3820393 | MIEAPGDPVERQFDEWLTRWSRWAEPEAARRSRETLRRELAAAARQ |
| NO: 11 | | LDLHSDTQELILGVGLLCWRSPRGDEVFRHLLTAPVQIVVDKQTGRV |
| | | GVHLRDEGELALEDQYFLTEQDGYVASRVEPLRGALSEVSDPLDDQA |
| | | KALLHKWASHGLETPCKFEPVWSTPETGGPHALVSLSPALVLRHRSS |
| | | NRLAEFYQGIHASLSDPEGVAPLGWAQLMFPMEPEERLAWHRATRG |
| | | TAGTSRLLSEEPLFPLAMNDEQRLAFDKLSKDTVLVIEGPPGTGKTHTI |
| | | ANLMSALLAEGKRVLVTSARDKALNVLFDDGMLPKPLQRLCVRLDD |
| | | QRGNRGKELTRSVTALSDASAERSKEEILERARMLTDRRSELKREISL |
| | | VHRQLWELIEAETTDLGEVAPGYRGRRADIAERVADTASTHSWIGIM |
| | | PDSAAPVPLNSQEAQELAQLLRTPAQNDQPLPTLRAGNPPTPDEFTAL |
| | | VSAAHQTLPASGVGARLAERLSTLDEGAFRTVSAFWELACNALQGLR |
| | | LPGDTASWSSIDWQGTAALSILQGGDVSAWKHLWEATRTAAPHAQE |
| | | LARLTGRYLQIPALHGAGAAEAASAAEAYSRFLKAGGRPGKIKKSPE |
| | | QRMAERSLAECFVDGRRPSTVADFDMLTTALRAVAVLSGLSNRWRR |
| | | SGVKTNTPDNVSQNLEALVGREADLAHLIRFAEALESLHQHLPDRSAI |
| | | HASGSWDWPALVEGFTAAPAHMKSARARRNLDSLRARIADADHPLF |
| | | REMTTAVERRDLAAYTTAFEIWKTQAHSQRLAERRSELVDRVAAVH |
| | | PALAHRLATATMDDDWTSRLETLDEAWAWSAAAAAVSSRSVESTAE |
| | | LQRELDRLEDALMKTTAELASEQAWWHCLQRMSVREASALRSFARE |
| | | MKRVGRGKGRYAGRHRQGAREAMRLARDAVPAWVMPVRQVAETI |
| | | DPRPDAFDVIIIDEASQLPVESAFLLWLAPQVIVVGDDKQCSPPMRVS |
| | | GELEPIYERIEEYLPDVPRAFRHDLTPKSNLYELMNVRFPGGQRLTDH |
| | | HRSMPEIIAWSSRMFYDGSLTPLRQYGTDRLPPLRVVDVPDGYREGR |
| | | DQNVRNPPEAEKLVTELKAMIEDPAYSGKTFGIISLQGGERSGHIRLIE |
| | | QLLDEHLPDQALRERLKIRVGTPPDFQGDQRDVILLSMVATGTPRIQG |
| | | GADFEQQRWNVAATRARDQMVLFASTTLTQLKSDDLRASLLKHML |
| | | DTPMRETTPQHLLHVEPQTKHPEFDSLFEQKVFLKIRERGYEVVPQYP |
| | | AGRNMRIDLVIVGEKGRLAVECDGRYWHSGAKQVQDDLLRERILRR |
| | | AGWTFWRLRESDFLLDPDVSLRPLWALLDRIGIHPAKGQ |
| SEQ ID | meta_gene_ | MAQFNFTKKLDIDETQIEQTDVMTGDNNRNRYLYYQLKLSMLHAKK |
| NO: 12 | 180752 | IDIIVSFLMESGVRLILNDLKTALDRGVQIRILTGNYLGITQPSALYLLK |
| | | NELGNRVDMRFYNDKHRSFHPKAYIFHYENYEDIYIGSSNISRSALTS |
| | | GIEWNYRLNSQDNHKDFVLFYDTFQDLFENHSIIIDDNELKRYSKNW |
| | | HKPAVSKDLARYDAVEDNSDTPVRKLFQPRGPQIEALYALADSRSEG |
| | | ATKGLVHTATGIGKTYLAAFDSAKYQKVLFVAHREEILKQAAISFRN |
| | | VRQSNDYGFFYGKQKDKDKSVIFASVATLGRSEYLTENYFAPDYFDY |
| | | LIIDEFHHAVNDQYQRIINYFKPKFLLGLTATPERLDGKDIYEICDYNV |
| | | PYEISLKEAINKGVLVPFHYYGIYDTVDYSSIHLVRGHYDEKQLDKAY |
| | | IGNKDRYDLIYKYYKKYPSKRALGFCCSRKHANEMAKEFCARGIDAV |
| | | AVYSNTNGEPSEERNIAIQKLKSQEIKVIFSVDMFNEGVDIPDLDMVM |
| | | FLRPTESPVVFLQQLGRGLRISKGKTYLNVLDFIGNYEKAGRVPLLLT |
| | | GGGDSNKNAPTDLSSIEYPDDCIVDFDMRLIDLFKKLDQKSLTAKERI |
| | | THEFYRVKEKLDGKIPTRMQLFTYMDDDVYRYCITHAKENPFRHYLE |
| | | FLEKLHELSETEETLCSGLGKDFLTLIETTDMQKVYKMPILYSFFNHG |
| | | NVRLAVKDDEVLAAWKDFFNTGKNWKDFAADITYDEYKSITDKQHL |
| | | RKAKSMPIKYLKASGKGFFVEKDGFALAIRDDLKDIVKNDAFIKHMH |
| | | DILEYRTMEYYRRRYLEKI |
| SEQ ID | gene_771418 | MRRNPEFTFFSHKNVPEVSGYEGGLVNSTIMNSLHTSPTLGIDIGSTTV |
| NO: 13 | | KVALLDAEHNILFSDYERHYANIQETLAELLRKAREKAGPMEVVSVIT |
| | | GSGGLALSHHLQVPFVQEVVAVASALQDYAPKTDVAIELGGEDAKII |
| | | YFSGGIDQRMNGICAGGTGSFIDQMASLLQTDAAGLNDYARHYKAIY |
| | | PIAARCGVFAKSDIQPLINEGATREDLSASIFQAVVNQTISGLACGKPIR |
| | | GNVAFLGGPLHFLPELRNAFIRTLHLTGSQIIAPDNSHLFAAIGAALNP |
| | | QEGQTSSSLLSMIERLSSGIKMDFEVKRMEPLFRDQADYDEFDRRHA |
| | | GHQVKTGDLARYSGNCYLGIDAGSTTTKVALVGEGGELLYRFYDNN |
| | | NGSPLATAIRAMSEIREILPPTAHIAWSCSTGYGEALLKSALMLDEGEV |
| | | ETISHYYAAAFFEPDVDCILDIGGQDMKCIKIKDGTVDSVQLNEACSS |
| | | GCGSFIETFAKSLNYSVEDFAKEALFAENPTDLGTRCTVFMNSNVKQ |
| | | AQKEGATVADISAGLAYSVIKNALFKVIKITRPSDLGRHVVVQGGTFY |
| | | NDAVLRSFEKISGCEAVRPDIAGIMGAFGAALIARERWHMQPADSGR |
| | | ETSMLPLDKITSLKYTTSMTRCKGCNNHCVLTINQFGSGRRFISGNRC |
| | | ERGLGIEKSKKEIPNLFDYKYHRMFGYTPLPLDKAHRGVVGIPRVLN |
| | | MYENFPFWAVFFERLGYHVTLSPQSTRQLYELGIESIPSESECYPAKLV |
| | | HGHISWLIKQGVKFIFYPCIPYERNETPDAGNHYNCPMVTSYAENIKN |
| | | NVEELAEEHVNFMNPFMAFTNEEILTKALVAEFANAFDIPAAEVRMA |
| | | AHAGWEELLQSRRDMEAKGEEVLDWLKQTGKRGIVLAGRPYHVDP |
| | | EIHHGIPELITSYGFAVLTEDSVSHLGKVERPLVVTDQWMYHSRLYAA |
| | | ASFVKTQENLDLIQLNSFGCGLDAVTTDQVSDILTRSGKIYTVLKIDEV |
| | | NNLGAARIRIRSLIAALRVRDQRNFERKVVSSAYHRAVFTKEMKKDY |
| | | TLLCPQMSPIHFDLIEPAIRSFGYKIEVLQNHNRSAVDVGLQYVNNDA |
| | | CYPSLIVIGQIMDALLSGRYDLNHTAVFMSQTGGGCRASNYIGFIRRA |
| | | LEKAGMPQIPVISVNANGMETNPGFTITLPLLTKAMQGVVYGDIFMR |
| | | VLYATRPYEAEPGSANALHEKWKKRCVASLSKRSSSMMEFGRNIRGI |
| | | IRDFDALPLRDVRKPRVGIVGEILVKFSPLANNHIVELLESEGAEAVMP |
| | | DLMDFLLYCFYNSNFKSKHLGTKKSTTYLCNAGIALLEYFRRTARKE |
| | | LEASKHFTPPAAIDELARMAQGFVSLGNQTGEGWFLTGEMLELIHSG |
| | | VENIICTQPFGCLPNHIVGKGVIKELRRHYPQSNIIAVDYDPGASEVNQ |
| | | LNRIKLMLATAQKNLKKGTN |
| SEQ ID | gene_1433645 | MGSGSEYGIKSLKNLDGIEHIRLRPRMYTDIGSEIGCHHIAQEVLDNCG |
| NO: 14 | | DEAIGGFCSRITVEIESDHVICISDNGRGIPVETDEASGMSGVEMVLTQ |
| | | DKAGGKFDHDSYQVSGGLHGVGVTVTNALSSFLEATVKRDGGEWF |
| | | MRLEKGRVIEKLRRVADCGPRTRGTSIRFSPDPEIYEQAKFRVQQIRQ |
| | | QAMDKAILIPGLEVIFKAPGLEAERFCFKRGLAEYMEANMADSPVFEF |
| | | SGALGDVEKVHWFFAAFDEPVDSFIRSYANTVPTPRGGTHEKGFADG |
| | | MLKAVREYLDLRPELKKTLGKNTRIAPSDVMANSQMGLSVYIKDISF |
| | | EGQTKQKLGSREATKFVGGVIHDAASLLLHRDVELSDAWVKMVIDR |
| | | ASARTALENGKKKKVERKSYTGRTPLPGKLQDCRFNGIEGTEIYIVEG |
| | | DSAGGSAKQACNRDTQAVIPIKGKILNCEGINQEDAIASEAVADLVTA |
| | | VGSGVGDVCDPANRRYGKVIIMTDADVDGLHIQNLLGTFFYRLMKPL |
| | | IDAGCVYIVQPPLYGVTIGKQKHYAQDQEELDGLKAMALAEKKKISY |
| | | TRYKGLGEMDPPELAETCMDAENRVLVKVLPRSDKRMDALMTKLM |
| | | GDDADQRKNLLMGVEIEDAVHLEPVEEPCDVTEDVKELCQPNSYDS |
| | | GNNKVAPFETVFREMYRGYGLQVVGGRAIPDVRDGLKPVHRRILYA |
| | | MEMLKLRSDGPTKKAARVVGDVIGKYHPHGDSSVYDAMVRMSQPW |
| | | KMRYPYIHPQGNWGSIDGDSAAAMRYTEARLTPIAEAMLSTDLKEGI |
| | | SEYQPNYDDEDIEPLLLPAPFPSVLMNGTTGNPGVGFKSEIPPHNLTEL |
| | | MGACIALADKRIRTGEAESPQDFASVRKHITAPDFPGGGIIAGSHDDLE |
| | | KMYASGRGKMLLRSKWHVEKLERGAWQIVITEIPYGIEKSPLLISMG |
| | | QCISDPTLPERKRLPMLEDIRDESEGTDIRIILYPKSKGLDPHDIMLHLF |
| | | SVTNLQVTIEYASYALEDWVLAPNGDRYRLPRLFALDQMIRSFLNNR |
| | | EQIVTARSTVRLAEIEKRLHILDGLLLAYPNIRDIVEIILENDEPKPIIMK |
| | | KYALSDPQVVAILAIRLSQLRKLEEMKLQGEHNQLSAEAVELRQTIDD |
| | | YTHRWKKIKKELQHVRKTFGDERRTEVDPDAARARIMSKEQLVARE |
| | | PVTAVLSKAGWLKGMRGSNIDVENVKFREGDTILDHAAGHTTSRVV |
| | | LIGRTGRAFNMLAADLPSGRGNGEPISKNFIFSIDEAPTRLFMINPDAE |
| | | YMVVTTLGHAFRAKGEDMLTANKKGKAFINFPTGSKLLCIREIDPGH |
| | | DAIAFITDDGCLGIVKLDEFPLLAKGKGLTAVTMKKGVKLLRDAAPV |
| | | NTSAAVRVGTEKRSTAFEPDEQAETYIIERGRPARPLPKACVNGMLII |
| SEQ ID | gene_4426209 | MKEMNKSETKSSKLLGIVLFHSFIPGKLFKVKAIGHSNNTGDNGAGKS |
| NO: 15 | | TLLSLLPAFYGADPSKLVDRQADKVSFVDYYLPTPKSVIVFEYEKLGE |
| | | RKCSVMYRNGSSVAYRFLTGTAEQLFSQHLYDELIKQGSETRTWLKN |
| | | LVSQSMSVSSQIETSVDYRSVILNNKKRLAQRRSTGKNLVAIAHEYSL |
| | | CSASHNMNHIDILTATMMRHKKMLSRFKTMIVDCFLNNTSMDDVPY |
| | | KKEYSELINSLDVFVQLETKKSKFDEALANKDSLEEYIKQLNSYRAQI |
| | | ASYLHQLALSDTQLSDKIRSQKEQHEILVNERKGKLHTFNSELNNQRI |
| | | EFERKSKIIDAIYNKRDKYENEDDILGKITLYNSLSDMLREVESARKHY |
| | | DNLLEDVRTEETELKSQVQKLELECSDFRFRKQQEINSVLKAKEEIVE |
| | | QKSERLEAMQSDLNFEKKKLQDAFDEQSERIKQEQLRLATLEGQSLD |
| | | FTSEQKAELRILENDLDKKRREFNASQNTVIYLNEQLRQATKTHEGSL |
| | | SAYHACRDELKEISDEIISVSRALLPTKGSLNEFLEQKVPGWRCNIGKV |
| | | IDPNLLNSKNLKPFFDLDTTESMFGLHLDLDSISLPDFCLSEEKLSERLS |
| | | TLKIKELETETREEKAKSRAKSDEQETEKLQKEVKIQSQRSKVLEDEL |
| | | SKLNLLKDQKTAQFESDAESRTYEVKKQKSVLESEFFAIKSELKAKLE |
| | | NEEQRHQQERVQVKANFDYRLSEEDAKKSAIEALIKDKEKVTSDRISD |
| | | CKLAFNQALMNKGVDPVSIESAKLKWELLERQCEEIKAFQALIIDYHT |
| | | WLEAEWKYIDTYNSEKLDLERQIARGVAKRDDYEKSVGRKIDDVAT |
| | | SIKLDEQELITVKEAIGQLTTCTNNLEKAVDESDLASLEDVSVDLEFHS |
| | | VEHAVSLVTDKITAINTLKKEIVSKVKDVSNTILGLDDNNEIKMMWE |
| | | QMRSATMTKLSDKYDYAINYDSPQFSLACLGDLEGLVLNVIPDVRDV |
| | | KIETLRSISTQISNYHQTLKQVNSKVDSVSSTLDKSIETGNPFPAIDSIHI |
| | | KLSSKIHTFDLWKDLNLFSVELDRWSGETSRGLPSKAFLASFKQLLAS |
| | | FKEAQISKNLESLVEMEITIVENGRPAVVRNDEDLEKVGSEGISKLAII |
| | | VVFCGMTRFLCQDEDVAIHWPLDELGKISISNLAILFDMMAQKGICLF |
| | | TAQPDLHPATYKYFATKNHIVKNVGVKSFIGGRRSRVNPLLSESKLNQ |
| | | STEVVE |
| SEQ ID | gene_5411831 | MNKSNLKKFAIEARQELREKTKAQLKRLGIEEKKIEEGKDMGSQVEIY |
| NO: 16 | | GKLYSKSSYQHLLVKYHSLGYEELVEESAYLWFNRLTALAYMELHD |
| | | CFTEHMIFSKGNKGEPDILDEYFQADFFQKMPLEKQEELHQLRDKNTS |
| | | DSLETLYSILMEEKCEELSKIMPFLFSKKGKYADILFPSGLLMQDSVLK |
| | | KLQVILLEIQEEDQSIPVEILGWLYQYYNSERREVVYDGSMKKSKIKR |
| | | EFIPAATQLFTPDWIVRYIVDNTVGRLAEEQFSISKDIIKKWQYYIAPEI |
| | | VSKNEKMQIESLKILDPAMGSGHMLTYAFDILFDVYQELGWSKKESV |
| | | LSILQNNLYGLEIDDRAGQLAAFALLMKGKEKFPRLFQVLEREENFE |
| | | MPVISLQESNAISKRMYTMLEECPTLQDLLKGFEDTKEYGSILKIDSFE |
| | | ESILQEEYHKLQEKIQNQGQFSLLNNNEFLEGDLEEDLERLEHIIRQYK |
| | | IMIQKYDVVITNPPYMGNARMNPKLKTYIEKYYPNVKTDLFSVFFIKC |
| | | CEMTTEKGYLGFMSPFVWMFIKSYEELRTLFIHSKTIISLVQLEYSGFE |
| | | DATVPICTFILQNTVIKKIGEYIKLSDFKGVKNQPIKTLEAIQNENCTW |
| | | RYQANQKDFTKIPGSPIAYWVSDRIREIFEKEKKLGEVGDAKVGLQTG |
| | | DNNKFVRLWHEINFNKIGFGMQNSEEALKSKKKWFPYNKGGEKRKW |
| | | YGNQEYVVNWERDGYEIKHFCDTNGKLRSRPQNTEYYFKKSISWGLI |
| | | TSSGSSFRFYPEGFIYDVSGMSYFIEDKFLTYLGILNTKIYSKLTKLINP |
| | | TINLQIGDILNLPVANIQNPLFEQLVSLILWISFEEWASRETSWDFERLT |
| | | LLNGENLSKAYKKYCTYWESKFFSVHSSEEDLNRILLESYSLQEEMDE |
| | | KVDFSDITLLKKEASIVENTDSAASCGYLENRGVRLEFHSLELVKQFL |
| | | SYAIGCIMGRYSLDKPGLIMANSDDVLTMSSNKITVSGVNGAIRHEIL |
| | | NPSFFPEEFGILSVTTEERFENDVVSRVIAFISAAYGKEHLAENLEFITE |
| | | VLGKKAGESHEEVLRNYFIKDFYTDHCQRYQKRPIYWMLHSGKKNG |
| | | FSALIYLHRYEKDTIARMRSDYLLPYQEFMEQQEAHYSKIASDEISTPK |
| | | EKKDAQKKVKELHDILKELKDYANKVKHIAEQRISLDLDDGVKVNY |
| | | EKLGSILKKI |
| SEQ ID | gene_941761 | MALKGDKLLCTNFEFLKVKKEFTSFSDACIEAEKSILVSPATTAILSRR |
| NO: 17 | | ALELAVKWVYSFDEELGIPYRDNISSLIHNGSFIELIDSEMLPLLKFVIN |
| | | LGNVAVHTNKTVTREEAILSLHNLYQFINWIDYCYGDDYKEKKFNEN |
| | | SLLQGEEKRVRPEELKDLYDKLSSKDKKLEEIIKENEELRKVITQKRKE |
| | | NIENYDFNIEEISEFDTRKIYIDVELKLAGWDFNKDIGEEIELFGMPNN |
| | | AEKGYADYVLYGDNGKPLAVVEAKRTSRDAKAGQQQAKLYADCLE |
| | | KQYNVRPVIFFTNGLETYIWDDYNGYSERRIYGFFKKDELQLMIDRRT |
| | | QKKTLRNIDIKDEISNRYYQKEAITACCEELERRKRKLLLVMATGTGK |
| | | TRTAISLVDVLTRHTWVKNILFLADRTALVKQAKKNFSNLLPDLSLCN |
| | | LLDSKDNPEESRMIFSTYPTMMNAIDDTKAKDGKKLFTCGHFDLIIVD |
| | | ESHRSIYKKYKAVFDYFDAYLIGLTATPKDEVDKNTYGIFDMENGVP |
| | | TYAYEFDKAVEDEFLVEYETIEVKSKIMEDGIKYDELSDEDKEEYEEK |
| | | FDKDENIGEEIQSSAINQWLFNANTIDLVLNKLMEKGLRIEGNEKLGK |
| | | TIIFAKNHKHAEAIKERFDILYPELGSNYAKVIDNQINYVDSLIDDFSG |
| | | KDKLPQIAISVDMLDTGIDIPEILNLVFFKKIRSKTKFWQMIGRGTRLC |
| | | EDLLGIGQHKDKFLIFDFCNNFEFFRMNPKGFKGNLGQTLSERIFNLKL |
| | | DLVKELQDLRYSDEEYVSHRNELLKYLIEDVNNLNEDSFMVKMNLK |
| | | YVQKYKNKNEWQSLGAVNAKDIKEHIAPLISKLNDDEFAKRFDILMY |
| | | TIELANLQGNNATRPIKSVIETAESLSKLGTIPQIQQQKYIIDKVRTTEF |
| | | WEDVDLFELDEVRSALRELLKYLGKTTQKTYYTHFEDMIINEESHGA |
| | | MYNVNDLKNYRKKVEYYLKEHENELAIYKLKNNKQLTKQDLETLES |
| | | IMWQELGTKADYEKEFGDMPVNKLVRKMVGLNRNTTNELFSEFLNN |
| | | ENLNIKQIHFVKLIIDYVVKNGFIDDNRILMEDPFRTVGNLSVLFKDN |
| | | MKEAKSIMGKISQIKENAEKIV |
| SEQ ID | gene_1546948 | MRLIALELENFRQYAHAQVAFESGVTAIVGANGAGKTTLLEAILWAL |
| NO: 18 | | YGARVLRDDTHTLRFLWSQGGAKVRVLLEFALGSRRYRVRRTPTDA |
| | | ELAQLNPDGAWLSLARGANAVNRLVEQLLGMNHLQFQTSFCARQKE |
| | | LEFLGYTPQKRREEISRMLGYERVGAAVEAIGRAERELKASVEGLRQ |
| | | GVGDPRALEAQLDAVEQALQATETALHAEQVALQRAVAARDAARA |
| | | HYDAQAALREQYLQLHQQRTLLQNDRQHAERRIDELRAQWEQLKA |
| | | ACDRYKVIKPDAERYRQLARELEAMEQLAQAAQQRAQLQARLDALG |
| | | ERRAQLHAERDALLQKQAHLDALQPQRARAEQLARELQTLRHIARQ |
| | | AAQRAQLEAQLQAIAEQRQRLHALATERDALAQQAQRAEADLHARH |
| | | TACAQTEAELQQTLQAWSQQRADLDAQLRAVQTTLQQQRARVQQL |
| | | EALGESSECPTCGQPLGDAYQRVLTAAQQEAQATERELRALRQQRRA |
| | | LEQEPDAIRTLRQQLAQQQQARDDAQRQLAELQARLRQLDAELRQT |
| | | AALERQQRDLEQRLAQIPPYDPEAEQRAQAELDALQPALQQAHALEG |
| | | ELRRLPAIERELSQTEREAQRIQRELDRLPDGYDPDQHAALRTQAEQL |
| | | RPLYEESLQLAPIIQQRDALRARIEDAKTALQRVIAQCEHLETQIAQLG |
| | | YSEAAYQQAAEAYQQAEAQVNTLERSLAARQAEYASQTALRDQLRA |
| | | QLERLLELQRALREQEHQLRVHSLLRKAMQDFRADLNTRLRPTLAAL |
| | | ATEFLNALTNGRYSELDIDEEYRFTLIDEGHRKQVISGGEEDIVNLSLR |
| | | LALARLITERAGQPMSLLILDEVFASLDAERRHSVMELLNNLRSWFDQ |
| | | ILVISHFEEINESADRCLRVRRNPQTRASEIVEDALPDPATLATAALDD |
| | | ALAGDEETGLLPPP |
| SEQ ID | meta_gene_ | MAKKKKTPVAQIEPISLPDEDLAKARAWLEGLNADIAYSQAKRQLAE |
| NO: 19 | 15450 | ACGWERSKSNAVIVALHEEGFMAGEKNYFCNPNAPAEPGVVRGARE |
| | | VSNFTIMLQSDPEVSVPLPYAIHCLPGDVFMLRKTVTGNWRVSNFVA |
| | | RHQTRWVCKLRGRIRRGRRSGIAQVVPINGFAPVEMQMDLADVPAE |
| | | VDLEKAAFEVEFLPESMKPEPYVEIFVRFVKEIGNRFDPLGEIAIASAE |
| | | YDLPVEFSAAALDEAQALPDEVDPKNMGRRVDLRDIPFVTIDGEDAR |
| | | DFDDAVYCARVEDGRTRLLVAIADVSHYVKPGAPLDVDAQQRATSV |
| | | YFPASVVPMLPEKLSNGLCSLNPGVDRLTMVCDAVIDPEGRTEAYQF |
| | | YPAVIHSHARLTYTQVWGAMQGEEGGLAAVGDRLDDIRALYELFKT |
| | | LRKARDARHTLDLETKETMAVFDDKGVISEFKVREHNDAHRLIEECM |
| | | LVANVCAADFVIQKKRGALFRVHDAPSQERLETLRTVLKSFNEKLESP |
| | | TPEGFAELISRTKENEFLQTAILRSMSRACYSPDNVGHYGLQYEAYAH |
| | | FTSPIRRYPDLLLHRAIKGILSRRIYVPQVVFDDSSLMVSRQARGLGSR |
| | | PEAGDGDKPATQAEKRHSVWERLGILCSAAERRADDATRDVMNYLK |
| | | CDYMLRHGKGRHEAVVTGMIPAGVFVALKDIAVDGFIHISNLGWGY |
| | | YEFDEKNLTMTSREEMTQVRVGDRVIVRLEEVDLENRRMSFVLESNL |
| | | ERRLIKGGKGGSRRSSRRGSRLYGRQFDPFDIDDDDFDELFGQEGDDD |
| | | WDD |
| SEQ ID | meta_gene_ | MSVARKTGSQPRALHAADSHDLIRVQGARVNNLRDVSVVLPKRRLT |
| NO: 20 | 73412 | VFTGVSGSGKSSLVFGTIAAESQRMINETYSAFVQGFMPTLARPDVDV |
| | | LDGLTTAIIIDQERMGANARSTVGTATDANAMLRILFSRLGQPHIGSPQ |
| | | AYSFNVASISGAGAVSIERGGQTVKERRSFSITGGMCPRCEGRGAVND |
| | | IDLTALYDDSLSLNEGALTIPGYSMDGWFGRIFSGCGYFDPDKPIRKFT |
| | | KRELRDLLYREPTKIKVDGINLTYEGLIPKIQKSMLAKDIESLQPHIRSF |
| | | VERAVTFTTCPECHGTRLSEAARSSKIAGISIADTCAMQISDLAEWLG |
| | | GHYDPSVAPLLEALRHTVDSFVQIGLGYLSLERPSGTLSGGEAQRIKM |
| | | IRHLGSSLTDVTYVFDEPTIGLHPHDIARMNHLLLKLRDKGNTVLVVE |
| | | HKPEMIAIADHVVDLGPGAGIAGGEVVFEGTLDGLRASDTLTGRHLD |
| | | YRAAVKETVRTPTGALEVRGATANNLREVDVDIPLGVLCVITGVAGS |
| | | GKSSLVRGSIPAGADVVSVDQGAIKGSRRSNPATYTGLLDPIRKAFAK |
| | | ANGVKAALFSANSEGACPNCNGAGVIFTDLAMMAGVATSCEVCEGK |
| | | RFQASVLEYHLGGRDISEVLAMSVAGAEEFFGAGEAKTPAAHKILTH |
| | | LVDVGLGYLSLGQPLPTLSGGERQRLKLATHLGEKGGVYVLDEPTTG |
| | | LHLADVEQLLALLDRLVNSGKSIIVIEHHQAVMAHADWIIDLGPGAG |
| | | HEGGRVVFEGTPAELVAARCTLTGEHLAAYVGTGPRKVRTS |
| SEQ ID | gene_307407 | MQQTLGNEATTRALRRGKRPMAPRPPAIDERAEQGLVLPPYLMELEA |
| NO: 21 | | GGLSTAYGLTGQEFVSTAVAAVVGHGGGTVAGISAELAGRPESFFGR |
| | | GRAFAVEGAEGGDGFDVTVSIAPAPDDLPPTFHPAADLASAPPDPGG |
| | | APLAAVDDAEGKETKVDVQHNSGATASSTVGNSSSKGAGGTAFGLA |
| | | PVLPGLWLGAAATGSVQPWQSSRDSRSQRGVAEPRVLRSDKGSVEV |
| | | PRRVLYVVRVRPQAGGDEQVFRGSGGLTQRVPTEHLIPAGTEAPTLA |
| | | APASGAPGRSQQVDPDLARRVALADSLAPVGVSDTAGPHQGGGGLF |
| | | DAVASVLHPSLTAPGAPGRSRLYEATATPTVLEDLPRLLGGDGVTGD |
| | | DLYSKDGTSAGSYRMRAVVTGLTPAWGTGKTQLRTHQQAQHTATES |
| | | AGKGRSVAGGIGPAIGVGAAANAAVVRATAMPVAAARKARFSVNEQ |
| | | TVSSRQGAEVRGEKVLYLGTAQFTVEGTGPRSVRAILNPQARVATHA |
| | | MRVWIGLRADEARELGLPLPPGVTAGEFIKKPEPQQPAADADSDTDT |
| | | DTESESEGGGDARHLPFGAMGSSVTIGRLDTAPMVKAVREMFATDPR |
| | | LAGYLPAFGATPPPADLSREEDEAQRTNYRELMAALSEANLRVNKEQ |
| | | LLSTGIRVRLRRKTTMHSHDVQLRVHGTMGATRHLGEIDDWLVRAH |
| | | SGVAANAQSGRSSSRSIGGMVLAQARLIPGVLTGSARYERQSSGTRR |
| | | NQGGPTTRTDVLTNGSEKASAFGAALRLNVDVTMTSRQRKLARALT |
| | | PGGPGRDVPEAKLLTGLHMEEQDVRLLTPSEFTVGTDEKARLDAGAD |
| | | QAPGPARPVAGAAGIGDLAGLAPTPAAGQVVRDWQLVETLGDGQPV |
| | | RDLALALLSRAAARGEAGRQDTALATEGLAPRLAVEERFGPRAITAA |
| | | LRQAASSGWVVKNLRYPRRLAALNGAVGTRLALAAPQLVHEAAGPG |
| | | TETFVMGGHQAGGQQGEGTSSTVQVGVTGVQNGTEWRVGEGLSGY |
| | | RSTSRSDTESATVSGTVERNAHTPKKAPLYLVRCDLLVTMVAEVKVT |
| | | GGGPYVASAARTLPGAAAVWLTAEQLRAAGVDLPESARKALKVEDR |
| | | RPAAERTAGGSGGGERAEASTAAASTSTSVPAPSRARASASTATGGQ |
| | | AASPVRQGPALARELPLGFGMIEDLPDFVPLLDRLRGNLAITGQQDLA |
| | | DDILPRQQLRDRNDNVQRLLRVLDRDGSTGLLASAMDGGVTVELLD |
| | | GRNTPYWAVFKIVRSGDGVREGEADDGRDMEYITSAAAQQATSHGE |
| | | GETTGVEGILAGSGKPDAGAGQVKSAGAAAGLGVASGSGRRGGESA |
| | | RGQLGMKTVAEAKTAKSAKMRVPIVASLELHKGDRRLALAGSGRTS |
| | | LVHRILESDLTALHRVSAPRRAPRPAPGVPTSGAAGLGAWRAAGVPL |
| | | PMEAQANGFQGAAHVRELVNTAVRAAGGGDRFRQKGQAAAYTLGE |
| | | AVSTEWLIAALPLLTNAGAELPPVHASGAAGQDLQASVHARLRAGRI |
| | | LGAGDKMTFETAAQSSLGAPRPTQTEGQSQAEQSRQARGLFGAGVL |
| | | NADQFRLNQLMGNVDGAGSASGAAANGAGSMPLHKPKFTSVLVQF |
| | | TLDVRVVARVTNRVRTSRTEVAERDLTLPRPVVIRMPLPVAGRLLAA |
| | | HPTEITDQHDRLGLRAAAVPPPTGV |
| SEQ ID | gene_1432510 | MTTTQKNKPGSLDKKGMSDYTETQCSRQLYIKLGEHDPRWIQRDIQK |
| NO: 22 | | NTHFTGSALTLAASGKRYEQKVYTILRRLFRQQTHCTLKPPANKEVIE |
| | | TFLDPRLAKRLHQEVRGEAQLLLEYEWPLCDQFVRRVFGQQPDEEIA |
| | | TLGNQYGRVLRPDIMLLHPIPKGQKAPLKCLLPGGKAASFSPTALQGR |
| | | FGISILDIKYTPDERVGRRHFAELLFYIHALTEWLHETQLDEFFFVPCH |
| | | GHGILGFLEEDTLYDLTLDDLLWRSPDELSGKHTPKISPLLWEDTHQL |
| | | FTHAEKTVRTLWQLAKQRTPIEEIPLCVQPACGRCPFIDDCISTLKGTT |
| | | PTQSDSWDIRLIPYLKTAVAQQLNEHGIYTVGELLQGIEEIPLGNTPVP |
| | | LHAEIPALKLRAQALSTQRAVYPEGEHTSLSLPKYIDMALVFNLEVDH |
| | | TNELVFAFGFYLDTKQPSPKLQRLHNDWWRMWRSVLRGERELQDIS |
| | | SVLDLEALELGWHKGDDFSDKLSLLLQEMERLLRTLEADGVLILRAV |
| | | GESYQFGSQEYTTQKYPLVRCQYSYVSGGIEPEHEYMLLKNMIQQLH |
| | | RVMRMCSLTELLVTTKHETYDSLYHENFAGFYWSDEQVDHLRALVE |
| | | RHLPALQQDHALSKTFYELVDWMTPADSGVRHHALHKKMYDLREF |
| | | VGSSVGLPQIINYTWHQTRPLWKKDFEANPYFWTPHFNQMDFGIWHS |
| | | TIEEIDTNERSQKESDIRDQLVLKMRTLHEILRHFHKEASDVIPKESKT |
| | | MSSQDFQRDRRNRQYHQLGSLWQGYHQLNAAISALTNDAARLTWPE |
| | | QSIAKLQAGKLSGMTIKIDDRDGKDYEVVNFSLLGLSSHMKISVKDR |
| | | VLLLPRTMRDSHAFPFHNMGRLSKLIVEDLVWEPSEQGYCVTAVREL |
| | | KKRKEGDKETLHSFTELYALYDAEDWFVYPTDLDVWTGRLALNGDA |
| | | LLRRYQLGYSWLAERLMFLHGLGGEHLEAPKTLNVHAAELYTYAPQ |
| | | LLPQKRDCTGEDVLTPIRFRPDSSQQEGILHALSSSISCLQGPPGTGKSQ |
| | | TIIALIDEFIDRHKGPARILISAFSYSALQVVVQKLLDSRYGDGPAPDPT |
| | | QLSDASRLPIFYASSSESESFVHDPNQQDVMHLSLSSKGVHLDGERIDF |
| | | RRGSRKDKIFERMFAHKGLEGDGSFVLFANAHTLYHLGTLSKANKRR |
| | | LVHEDFGFDLIIIDEASQMPASYFTAIAQFVHPFEARLVLPKDEDALKR |
| | | EIRCGAPELSIEGVPSSDDLTHVVLVGDQEQLPPVQQIEPPRKLKPMLD |
| | | SVFRYFLEVHHVPKHQLSYNYRSHKDIVRCVRRLAIYDQLHAFHQDD |
| | | AYLSAIPDVLPDTIEAPWLRQLLGRRQVVSTLIHGRQWDTALSPFEAK |
| | | LTADVVLAFFAQMGVDSDERERQFWQEDVGVVSPHNAHGRLIVREI |
| | | AERLLSGVGARTYLPETELMECLSTTVYSVEKFQGSDRRLIVGSVGVS |
| | | SVDRLAAEEGFLYDMSRLNVLISRAKHKMLLICSQQYLDYVPRDRDV |
| | | MTVAARVREYAYDLCNESQVYDVPFGSGSEFIELRWMVSKDP |
| SEQ ID | gene_5570191 | MQSGSGVDLFRDFNEGEVSEVLRRCAGCSRFVLIGPPGSGKTFFKENY |
| NO: 23 | | LEGRLGTGVIVDEYTLGISTTAKIESEEARKGSGISKKAMKYLKRMIPL |
| | | IEKLRETAEVDDEELRKVLGDRAPKHIVEGARRSIGDSPHRAYYIPWK |
| | | CVDEPNACTFDANVSRALELIKKVFDDKKIRIRWFKAEYVPPGLVKD |
| | | VIDLIRVKGEDGAREELKGWVEAYSEADETLRKILGLSDDLLEWEESF |
| | | VEYLSNFVINYASYVISGLVVDPLIGASALALISVLTYMAFKREGEGYI |
| | | KGIIELKRGLERLRRSDGEFNELGKLLVYRVAYAMGMSYDEAKEAL |
| | | MDITGLSIDELKRRVNEIEWRIKELEKKIELFRLEVPAGIVTADVNEFA |
| | | KGRTYPNIKVENGELRIRVEDGYHSIVRAGKFNELVNEVRDGLLKQG |
| | | FVVVVGPKGIGKSTLAAAVIWELFMNSDIGLVARVDVLDLKNYSELA |
| | | TFVENYGEKFSEHFGKLLILYDPVSTKAYEKVGIDTEAPIQSNIERTIKN |
| | | LVNSKSSKASKPFTLIVLPSDVYNALSGEVKNALEGYRLDVSQVLINT |
| | | EFLAELIREYSKTKDKPNGCALSDDVLSKLAGELAKFDSGHALIARLI |
| | | GEELARSNCGVGKVEELINSAKGKAEAFIILHINGLFKVHENPDTAKA |
| | | LVEIFALRRPFISAVESDDSIPDTSKFLVKVYVLRSPFISAVKPGDPILTP |
| | | GIVELIGEAGGVKILYGAEGEELRSWLAIWLHDLIEEAIGKLLDCIEGK |
| | | GEGCKVLGDALKPWKTTGVIELLRKVSEKVNDVDSAVEYFASNYGE |
| | | RLTSALKVFSNECWKRAVYIIGHALAGDPLLPRRKYLSAFMSMNLSK |
| | | TGIESPSDALSRLGANGDKNPQRMSLAKYYASIVESLGDALKECGVD |
| | | NYLIVGDKIPSLMMGLIGNHACALAGVFIDKYNEAIAEIKRLLNIIKNR |
| | | GEFYYEEAYYGLGLATIIAKAAESGRPVGHSDADAALHIASFAMSHV |
| | | QSTLHIIRLLTALAPLRDKAPQRYLEVLVCALDKFTRLGTCHDWDTV |
| | | MNILNELDYILNKYGVEVKGHARTLVDVINTLTHSLYKCLERCVDYW |
| | | FEHRVASFRAKFERMISELADLLDKTNRWSPNLGIIAAYASLSALDSK |
| | | NKNKCVRMLIESELGIDVVNKTKEVAGELSELRGSVRELLRDEDLMG |
| | | FVRSRLAEADEKAAKRGILEVTSILKHTLAQYKFVNDELDEAGRLFNE |
| | | AAEESKVIGDYLNYLDNRDWALRVEAIKSPLAGDDLVKLVNGFRQL |
| | | YEEALNAERFMSASPDYGTLWKNILRDILGGYLVSLALTGGDEEIRRI |
| | | EELLKEQWQLKYEPRPILTRLTLNALLSPRVELSSELRDWLVVKPGELI |
| | | VAFGHGYLYIDYLPALKATYGTIKPGDGKRCSSVYLTFMLYALINGN |
| | | EKLAKAHALMGAMNHSGKLPARLFLEAYRACCDPNNEEFRRAIAKL |
| | | FFYTRALKSKTSGFWSASLSS |
| SEQ ID | gene_2435065 | MDRLKTDREKAVQHAEDLGYQVEVLRAKLHEARRALATRPHSYDT |
| NO: 24 | | ADLGYQAEQMLRNAQLQADQMRSDAERELREVRAQTQRILQEHAEQ |
| | | QARLQAELHTEAVNRRQQLDQELAERRATVESHVNENVAWAEQLR |
| | | ARSESQAQRLLDESRAQAEQSLASARAEAQRLTEEARRRLGEETENA |
| | | RTEAEALLRRARADAERMLNAASQQAQEATDHAEQLRTSTASEADQ |
| | | AHRRSAELTRAAEQRMSEADTALREATSRSEKLVAEAEATAAKRMA |
| | | AAEAAGEQRTRTAREQVARLVEEATKEAEAVRAEAEELRERAVAEA |
| | | EKARSEAAEKARAAAAEDSAAALAKAARTAEEVLQKASKDAEETRR |
| | | SASEEAERLRSEAEAEADRLRAEAHDLAEELKGAAKDDTKEYRAKT |
| | | VELQEEARRLRGEAEQLRAEAVAEGERIRSEARREAVQQIEESATTAE |
| | | ELLTKAREDAAEAREAGEADGERTRAESAERAAALRKQADDALERA |
| | | RTEAAKLGEEAEEAAARTREEAEQAARELREETEEGVRARREEAETE |
| | | LVRLREEAEQRVVAAEEALTEARAEAGRLRKEAAEEAERTRTEAAER |
| | | ARTLSDQAVEEAEALTATAAEEAAASRAEGEAVAVRLRADAAEEAE |
| | | RLKAEAQEAADRLRAEAASAAERTEAEATEALERAQEEADRRRRSAE |
| | | EALESARTEAGQERERAREQSEELLASARKRVEEAEAEAARLVEEAD |
| | | ARATELVSAAEATAQQVRDSVAGLQEQAQEEIAGLRSAAEHAAERTR |
| | | GEAQEEADRVRSDAHAERERASEDAARLRSEAAEELETARALAETAV |
| | | AEATAESERLRADAGSYAQRLRSEASDALASAEADASKARAEARQD |
| | | ANRMRTEAAEQADRLVSQAATEAESLGARSTEEAERLRAEARAEAE |
| | | RTVTEAAEEAERLRAEAARAVAEAEERAARAREEAERVESQALAAA |
| | | EELTSQARAEADRTLDEARADANKRRSEAAEQVDRLLSETAAEAEKL |
| | | TTEAQQAALKATTEAESRADSMVGAARAEAERLVAEATVEGNSLVE |
| | | RARADADELLVGARRDATAIRERAEELRERVTAEIEELHDRARRESSE |
| | | AMRNAGERCDALVKAAEEQEAKARADAKELLADASSEAGKVRIAA |
| | | VRKAEGLLKEAEQKKAELVREAEQIKREAEEEAERVVAEGQRELEVL |
| | | MRRRADINQEISRVQDVLEALEGFESQPAGKAAPGGSGTGVKAGASA |
| | | GSSRSGGKQNDN |
| SEQ ID | meta_gene_ | MENSGLSLDAEQKITVAEKVRKEPNKNYFISASAGTGKTYTLTNYYIG |
| NO: 25 | 343942 | ILEQHEKTGESDIVDRIVAVTFTNKAANEMKDRIVKEIQKKLESLSEN |
| | | DRAYKYWKDVYKNMSRAIISTIDSFCRRILIEQNIEAGVDPNFKIINEL |
| | | KQKKLIDKATQRAIQLAFDVYDAIESGENYTEKVTNYLYGLTTERTK |
| | | RIRELSDELAKSKEDIFRLFEIFGDISDVAEKIESVVTNWRLELNESKVS |
| | | ERLLEVFEEAGGALRAFRNISLIAAEFYESETLDNFEYDFKGVLEKTLK |
| | | VLENSVIREYYQKRFKYIIVDEFQDTNELQKKIFDLIHTNDNYIFYVGD |
| | | RKQSIYRFRGGDVSVFIKTMNEFEEKIKSGRTDYEMLSLNINYRSHPEL |
| | | IDYFNYISENTIFNNHVYEALSESPDTSKTTNNKSKSKKKDKNKSQAN |
| | | GEDIVLNEALQNVNDIFSTERDENIYIHEVFRLRYPELYQKLWFIKKDD |
| | | ESNAAFSPDSNEFLPGDLRRVNYITISKASLLENTQENDETAKEIGLDE |
| | | DNQSPGKMKKLKDMDERELEALHVAKVIKSLVGKEMTFYEKKDGKF |
| | | VPISRRITFKDFSILSYKLEGIEDVYREVFAREGIPLYIVKGRGFYRRPEI |
| | | KAVISALYAIQNPNSNYYFTQFFFTPFTDNLEQNPEVGVRNGKVKIFH |
| | | KIVMRYRESKGQGLKKSLFQCAKELAEENELPENVTKMIKLIAKYDE |
| | | LKYYLRPAETLKLFVKESGYLRKIPHYPNSSQRLRNVRKLLEQATEFD |
| | | DQAPTFFELTRLLERISEVQEVEASEISEEEDVVRMMTIHASKGLEFNI |
| | | VFLVNNDGVDKAEEKTFFPESEDGNGRYVYISQFLDKALKKFETSRV |
| | | TKELEKELKKLLEAEVIYDKTEILRKVYVAITRAKEMLFVVDLQRKNT |
| | | KGIPAIKYLTPKGFEERIKIISSLDEIDKLAGSGVESVSGKQEFAESIQSL |
| | | LDLENVVDKGLIFSDFTPKAYKRYISPTLLYGIKDEKSDLESVDESSED |
| | | FDSAETISITSTSNFEASKAKARLKVLNSLLEKATEITRGKQIHSMLASI |
| | | TKYEQLKLLVEKNALPEDILNVRVLESLFNESEKIFSEWRLAKSIEIYD |
| | | EKLKERKNYILFGVPDKVFLKDGEFYVVDFKSTDLYKEAEEIERYMF |
| | | QVKFYMMLLSDLGKVHCGYLVSVPRGQALRIDPPGEEFLDEIIYKIKQ |
| | | FEELMSI |
| SEQ ID | gene_1456430 | MLFGMTGCGTSSVTSSADAVTDTESVDDVKTESSGKTDEEKLSEKIG |
| NO: 26 | | ELTSAHSAGKGKDETVYVISSADGSKKSVIVSDHLKNGDGKDTLEDK |
| | | SELKDITNVNGYETFKKGSDGKLTWDAKGSDIYYQGTTDKELPVDVK |
| | | ITYLLDGKEVTPDEIAGKSGKVTIRFDYTNNTEKTVKIGGKDEKIKVPF |
| | | SVVSGVILPIENFDNVTVTNGRIISEGKNNIVVGLAFPGLKESIDLDDLK |
| | | NEAVSEDAKKEIDDIDIPDYVEITADAKNFKIDTTMTVAQSNLLSSVN |
| | | LTQDVDTKELTDKMDELQDGADKLQDGAGKLKDGTESLTDGTEKLK |
| | | DGSGDLKDGTKKLAGGTDDLKDGADKLKDGSADLKDGTKKLADGT |
| | | DDLSSGVSTLKDGSSKLAGGTDTLASGASQLKGGSSKLAGGTDDLSS |
| | | GVSKLKDGSSKLAGGTDTLASGASQLKDGTSQLSGGLKTLKAGTSQL |
| | | KAGTDQLSAAKPQLDQSLKDLQDMGTQLKEAENGSAKISDGIGKLG |
| | | DALTAKFAKTALNMKAMDEGVQKLSAGISQAANGIKELKTKFDNGV |
| | | VGIHGQVNQLIADLKDYSKDEASGIKGIGYRGIGKAAYNTGINQAQR |
| | | AAQSADENLQKAQEAVDEAQKAYDEALKAQQNSADAGNSLQQQND |
| | | DLAKENAKLQQKIDELQNSADQEKKTNNVASPADNGSASSGNASAE |
| | | KAGTQSTDSEGSKAAGTEPAETPAQNDAAADASSQSAAPADNTSSED |
| | | TNAGNSAADTTENVQSTQASLAGLAVSKLNEMKNALYESTVLVAKA |
| | | GESSETVAQAQQALEKAKESLQSAQQAKVAADATVSALKDMKSSVD |
| | | SAEKWKGTNDLKRVEKMTRIMGEAEAINSSLDILQQSVDAALDSLSS |
| | | GLDSAKTGLDKIHNGIDQSLNSDETKAEQQQLNESLTALKGGAGQLT |
| | | TGLDSGLQQLTDKSAATTKNIGDLKNGIDQLSRGANSLDDGAGKLAA |
| | | GAEQADNGAGSLAGGIQELGKGAHDLDNGIGTLKSGASDLKNGAHQ |
| | | LDDGIGTLKSGASDLQSGAHQLDDGVSKLQSGASDLQSGAHQLDNG |
| | | AGDLNDGIIKLDNGAGDLQKGAHDLDDGTQTLIDGINSLNDGAHDLD |
| | | DGMATLQDGVIKLNEEGIRKLTDLFGDNVQDVIDRINAVVDAGDDYT |
| | | SFAGTGDQENSAVKFIYKTDAIKAKED |
| SEQ ID | gene_317827 | VKKILFPKLDGPPSDDLENYMFLGTFEDENGSLTTAKFFVRSVSHVSP |
| NO: 27 | | GGCYEVEGDWKRTAKGEEFNSWCLIPSVPDTFALSCVYLNGLFPPEM |
| | | CGTSALSRRLSALTREYGPDVLVRALATPTILTRLSDQPEIFAANILRL |
| | | WEAATRESHMALMMHRAGFTTGDLDMVWRGCAFKVAERIGGDPY |
| | | QLVAIPGIDVAKADMLFRTLGGNPYDPRRIAGIIRRSLMASEGLSATN |
| | | DDGEKIGFTAHVEFPGSTAVDVTDILTSGKAEPLRDDLISGIDPKIGMR |
| | | LDVLRDFLSKPQEALKFGLRIRKTRDGRTLVARERVYQAEVRVARNI |
| | | ARLLQAPPLKDKATVQATCRNLFNQPDFQRFDAVQRTAVEMACYER |
| | | FCVITGGPGTGKSTILDAVIAARVAMGTEKRSFLLGAPTATAALRMTE |
| | | TTGLDAATIQSLLKCKGEKAGGEQWFDFNRNNPLPSGCTVYVDEGS |
| | | MVDIFLSDHLLDAIPTDASLLILGDDGQLMSVGPGAFLENLLNTRTMA |
| | | GDRVVPAICLQNTYRSNPKSNLAIQAKEIRYGGVPTINGDSSGGTSMQ |
| | | SVVPEKISNFIVYAMSNVMPALGIQNPLKDVAVLGPQNPGVGGLWEI |
| | | NSQMSRYFNPNGAKIPGLSAPRFAKEMPVPRVGDRVMRRKNVKGDK |
| | | LCVNGSRGFIEAYIPPSPADPDAKKGKIKIRFDNNEVRTEDVSWDWHK |
| | | KFELAYALTIHKSQGQQYQYVLMVITPEHANMLDNSLVYTGWTRAK |
| | | EGVAVVGSFDAFAGAVQRSRMNTRLTMLPDLLSEILVPGIADEFRSR |
| | | WYKKPPMDDLPRPGGREKWFQTKYGNASGHKIRTIEGIKVEAPANG |
| | | VQAGLRGGFPSPPSQPHSSGSGPTTPTASGSHQAPPVRYAVNQPTSSPP |
| | | RPMFTGGIGYRPNIPVSSALPNPPATPSYDKKGVINHVQENAPPRQPN |
| | | TSHQDATSPTHPKSNSALQPSQAVPLQAVGLQSPRRFGWSPTIRQPSA |
| | | APATSNAQPTARSAAPDHVPATSRPAQPHRPVRPTTPVESPSARPVPA |
| | | SRPSFGFIGWRPNIHPIKQTCHEPQPEMDSEMGMEDQHSSSYEDAPSP |
| SEQ ID | gene_4421494 | MTNKVESNVSDQTEKRLSPEVSEQFQQDTRVVAKQAAEFIEEIHPARL |
| NO: 28 | | LQTKQEIMDLSYAKSDELLDSFAFFRIVSCTTDEVDDMFDFLNEKMD |
| | | KFYTALYAVGKPVVYGIVSYGETTNLVVGLLDTEDNSDLLKSIMEGL |
| | | LDGIELLPYKTNFAARTACEKEVGLISAIPSVKIEEEKQIFSLAPLMKSL |
| | | NGQDYTVLFISRPLSQDIISKKRRALIQIKDQCFAVSKRNISRQQGISRS |
| | | KGNTEGRTDTITKSTSNTISESFGWALGFTFSESYSETTSESSSASENYS |
| | | QTITDAINQSEGISAEVQNGVALELMDYTDKAIERLRQGRSNGMWET |
| | | VISYSTDSKLAAGIIRACISGEFAKPNPVILPQVVHSFHLDKTEAEGKSL |
| | | LVPEILDAEPELSPLCTVVTSEELGFMCTLPDVPVPNFELKKGKTYPLI |
| | | TDNAVGVEVGHICEGRRILENMPFSLTHKDLARHTFVCGITGSGKTTT |
| | | VKGILKEADTPFLVIESAKKEYRNINLKDKKRPQIYTLGKPEINCLRFN |
| | | PFYIQCGVSPQMHIDFLKDLFNASFSFYGPMPYILEKCLQNVYKKKG |
| | | WNLTLGFHPYLVNTANSAKFFDADYMQKKYASAAHKYLFPTMQDL |
| | | KLEIERYIKTEMDYEGEVAGNIKTAIMARLESLCSGSKGYMFNTYEYA |
| | | DMNALLNHNTIFELEGLADDSDKAFCVGLLIIFINEYRQISQEMLDMN |
| | | RTLSHILVIEEAHRLLKNVSTEKSSEDLGNPKGKAVEHFANMLAEMRS |
| | | YGQGVIVAEQIPSKLAPDVIKNSSNKIIQRLVSADDQAVMANTIGLTG |
| | | EEGLDLGSLKTGTALCHKEGMSLPVRVQIAMVDDIKVTDDLLYGKDI |
| | | KKRLYQINVSLAKEVLADSLPLMGMKMLNTILVQDCNHVSHAVTVC |
| | | RQSFRSSLKKNNVTLVMCDNENEIYAELLYEGVLRYLLNGCYILKQM |
| | | IPDELCSDIYQLMLSPDNDKLVLVKEQLQAEYEENLEDQGCFIVAQLI |
| | | YKNAFERTDIVQTIKNYFFEISDEDILKIKAEWRGSD |
| SEQ ID | gene_3011455 | MSSWDPQTSGLTVRLRDNPGRVGHTTGRWKFAGSLTLVEVAFGPNE |
| NO: 29 | | KQFKNQELLEQVHSSEDPLDLLLGGKLGLPSDLRRVLAFEKVRGELT |
| | | NIFYSMESSNTDFYAHQFKPVLRFVESPLGRLLIADEVGLGKTIEAAYI |
| | | WKELQARYGARRLLIVCPAMLRDKWRRDLQAKFNIKAQVISASDLL |
| | | VKAREIVTDGALESFVAISSLEGLRPPADFEDDRKASRRAQFARLLDQ |
| | | NPTSADFALFDLVIFDEAHYLRNPSTANNRLGRLLREASRHLLLLTAT |
| | | PIQIGSQNLYQLLRLIDPDVYFNEAVFADVLTANAAIVSAQRALWANP |
| | | PKIREAEAAVRSARANSYFQGDPVLQRIEALLPEADTQTVMRIEALRL |
| | | LESRSLLAQHMTRSRKREVLKDRVRRASQVLAVEFSSLEKEVYDQVS |
| | | AAIRAKAKGESWAVVFSLICRQRQMASSIVGALESWKNTDFLEELVW |
| | | DDLGVLPQDLFGDRGDNQQEVAAPTINLTSDVDLARLEELDTKYRQL |
| | | IQFLKAELKRDPHEKFVLFAFFRGTLTYLHRRLQADGVQAIVLMGGA |
| | | DIDKDAVVETFSKTTGPTVLLSSEVGSEGIDLQFCRFVINYDLPWNPM |
| | | RVEQRIGRLDRLGQRAERISIISLAVSNTIEDRILMRLYERIAVFRESIGD |
| | | MEEILGDVTEKLIVQLFDPSLTEEEREQRAAQTELALENSRQQQGELE |
| | | QEAINLVGFSDFILDQINESRAQGRWLSGAELLALVDDFFARHFAGTR |
| | | IEPLDHEVTSASILLSEEAKLSLGQFIADTAPAVRTHLHQSLRPISCVFD |
| | | PRRVNRSVKGAEFIEPSHPLIQWVRQAYELEPAQIHRASALHLRSGET |
| | | DMPEGFYAYSIHRWSFQGIKRESVIAYAAQMLGQARPLTSIEAERLVG |
| | | LAASRGQPLANVFASGVDRHELSQAAQACEEQLGLEFEKRLVDFLVE |
| | | NTVRCDQQATSATKFAARRIAELQDRVERFQLEGNDRLVPMTEGLLK |
| | | KEESELKFKLQVVDKKRNVDPTMVHLGLGLIRVA |
| SEQ ID | gene_2590511 | MSNFNFLTDISPELAQFGKSAELYCHDDKQVALVKLRCFTEVVVGEIY |
| NO: 30 | | SRLSLTPPVRDDLYNRLRSYEFKDVVSDKGIWAKLDVLRHKGNKAA |
| | | HSSNGSDEISLNETLWLIKEAYLVARWYAQAILNKPITPPEFVDPVKPI |
| | | DHTSRLEAELERQRQELNKREAELKTQLADNSDKYQQQTSELIAQLD |
| | | EKNDTLSNVKKEQALLQIELEQKQKDLVASQQAFFDYRTREEFKQASI |
| | | SSASSFDLDMEVTRRNIDIFDCFEGVSLTKGQNQIVKQINEFLTDTKQN |
| | | VFLLNGYAGTGKTFITKGITQYLERIGREFAIMAPTGKAAKVISDKTM |
| | | QPASTIHRVIYNYDNVKEYKVDGVEGSETYRCYADLKVNVDTAEAV |
| | | YIIDEASMVSDRYSDGEFFRFGSGYLLKDLLKYINIDHNDHNKKVIFIG |
| | | DNAQLPPVGMNTSPALDASYLKENYQVAVASGYLTEVVRQKGDSGV |
| | | LNNAAMLRDGLEQNLFNKLKFEVNDHDVFNLSSENLLSTYLDSCDRK |
| | | VSRTGESIIIASSNRQVAEYNRLVREYFFTGQQQMVAGDKVISVANHY |
| | | RADACITNGEFGMIKEVLSPHSELISVDISVKGDTGDMVKRKVNLSFR |
| | | DVILGFRNDYGEPFFFEAKIVENLLYNDQPTLSSDEHKALYVHFLNRH |
| | | PELRRKGNEQKLRIALLQDPYFNAFKLKFGYSITGHKAQGSEWKTVF |
| | | LQCQTHQKALTKDYFRWLYTAITRTSGILYVMNPPQLRLGDGMKIAG |
| | | AYQPKAVNLDNSAPEGVEVVRPSTEATNSVATAKFDFQTDIPQLKKL |
| | | YQLVDACIEGTGITVVDVLHYNYQDRYILQRGNEQASISFNYKGNWK |
| | | VSGVKSITQDGFDVELMALLGQLEGTLLDVPEPSKDTQFHFSEPFLEE |
| | | FYLNVMDQINSVGADISKIESRSFCERYAFVKGNELAVIEFWYNKSSQ |
| | | FTKVQPMPQLSNSTRLIDEIICQIGVLL |
| SEQ ID | meta_gene_ | MVNNKKVMSDNTQPKASVAEAFGNAKKAKTINGIIKKIIFQNAESGFT |
| NO: 31 | 463174 | VLNVFSNDKFITASGTFFDKPLMDSKIKLKGEFTYHKKYGYQFNFTQY |
| | | EVSLSNTKTAIIEYLSSSIFKGIGKAIAREIYDKFKEKTLDVIDDEPEKLK |
| | | DVNGIGAIKLAVILEGLKESYGLRKTVMFFKPYQFSDYQIKAIYNRFK |
| | | DKSVTIAKENPYLFTDIKGIGFKKADIMSEKLGIKKDDPNRIKEAIKYV |
| | | VNQICESSGNCYIYYQDVKKGIGEIIEDLEETDLKKYLNDLIKERKLLL |
| | | DFKGIYGTDNYLSVVRDRYVSSKSIDLKGEEVLDFTSAKEGKRLGCA |
| | | RIYMPVYYHCELGAAKELKRIRESASPASDKIESLDDLDKFLELGNNH |
| | | VSLTNEQKTAVLNALKYKISIISGGPGTGKSTIIKTIVHLYSGEKIALTS |
| | | LAGKAAQRLADIVNSGQTLSSRNDHSQEKMGRLNISTIHRLLKAQYD |
| | | RQTGESYFTYNERNRLPHDLIVIDEMSMIDIIIFYKLLKAIKDDANIVFV |
| | | GDVNQIPAVSPGDVLRDLIYAGAGNMDGQDKTKPFFPSTFLTKVFRQ |
| | | NEGGLINLNAHNILNNKKFVTLRKDCKEKNISTAEKDDSFTIKYRKEY |
| | | DIAVGGKHELLIDFTRFIKRVVENRINKRDVGLKSANMSIPTMLFDDIQ |
| | | VLTPMRRGDLGYFNLNNILQDIFNPISPLHLSASVENIFICNGIQFRLYD |
| | | KVIQKRNNYDQDVFNGDTGYIVDVNHNEKYLTVDFSNYSDLSKKCN |
| | | EIGTGAESCANLTAQEGKMTNKAIKLVKYNFLDVYENISTAYALSIHK |
| | | AQGSEFNNVIVLFHQTHYMMLKKNLLYTAITRGKKNIVIFGTFKAIGI |
| | | AMGSKETVRNSGLKDRLSEEFLDAN |
| SEQ ID | gene_773846 | MIENLPPFSIILAPAYLHPILRADIMKQTSGCMGLQLLSPQTFFASFTQK |
| NO: 32 | | QARDHVEISFLYKQNIEKIISQLQTYQAIALTPSFLMECYDFIESMKFY |
| | | HISVDELPDKTQAQQEIKTILNNIFPIQTAQDIWNEAVLRVSDCSNVYI |
| | | YDAFYSLKDEKILNILTSKGAHTIPLPKPQQQKEFYHAINPRQEVEAIA |
| | | QYIIQHDLDADDIIITLASSTYKPLIEQIFKRYEIPYTLLQKNKASIVTQR |
| | | FVNLIAYALSFDQEDLFACMDAGVFQSEHLDELREYIEIFNCDIFQPFH |
| | | HLMNVQANGHILDEVEITKLKELEEIAESGRQELCETLSLFIEDDLHQL |
| | | VTHLLDILHNGMKEASMEDISVLSNIQDVVSSSWNYLNTKDDLAFLL |
| | | PFIEQISISKSVREIHGVIVGDLKQIIPNRTHHFLVGATQKNYPAFPSESG |
| | | IFDEIYLRDTTLPDMETRYQYYIAQCEKQLHTNSHLIVSFPLGTYEGKG |
| | | NEAALEIEEEMKCDPTAFPIMENYEKITQTYIIQPETAKALFVKGHHIK |
| | | GSISAIERYIHCPYSYFLRYGLSLREPMQHGFDNSYMGTMAHYALETL |
| | | VDELGKQYTKAAMERIEEIVNQEVEAIAAVFPNNADLMEVIKHRFLV |
| | | SFAQTLKRLDDFETHSSMGPYLQEYEFHEEFPITEDISFALKGFIDRIDA |
| | | SGNFHCILDYKSSAKSLSEDKVFAALQLQLLTYSIVAKKQLHKDILGA |
| | | YYISLKNQNIPYIAGKMKRRPVGFVETEKDDYEENILKAHRISGWTMR |
| | | KDIDMLDDNGSHIIGVSMNKDGIVKARKYYRYETIYEWFISLYRTIGN |
| | | RMLSGDIACSPDADACTYCAYYEICRFKGFASERKPLVDIDDSLYWE |
| | | GGVDDADME |
| SEQ ID | gene_1188229 | MKGSIKSHKSAIAVLLALALSGQSSWAAQNSAAVQGNDFLSSIQQIEV |
| NO: 33 | | KQIDFPAPTHRQQTPSASRAQINDLQQEIARLKKQLKAAEQEKKSLSA |
| | | PGDLQAQNTQLLKDNSALAKENDRLSRSLQNAQREQGAASTQQAAR |
| | | IEALEQKTAELQASLASKTEELAQLKKSSNSQAASESALQKQIARLET |
| | | EKAAIAERNTKDTARFNRDMQALRNELNKRADELVALKNAGDKRA |
| | | QSQTALQKQLAQLEKEKAALTAQSAQSIDVANKKVQALQAELDKRS |
| | | AELAALQKTGSEHEKSQSDLQKQLTQLEQEKAALTAQNAQSIDAANK |
| | | KAQALQAELDKRTAELTALQKAGSEHEKSQSALEKQLAQLEREKAA |
| | | LTAQNEKSIGALNKQLAQLEEEKASVTEQNSLLMKNSSLSKEEKAKL |
| | | QKAQAEQTALLEKNQAAEAALKAQIAALTEKLNASTTLAATSQEKV |
| | | AALASELASLKGSQSEKAQALQSQQQQAAQIAAAKEALTQQLATAQ |
| | | ADIATLKQSLAEKENRLQQSDKALLALKEEAQSAKALTTASATSQQK |
| | | TQAELDTLKRANEELNAKLASLSAENTAQKAQAEKEKAELLAQAEK |
| | | EKAELLAQAEKLKADAATQVQTVAATKAEPEVSAAALKDKANKQS |
| | | YANGVMFSRLVQKSMDQMADLGIKTNLPILLAGIKDGLAQKVAVEP |
| | | KTLLSLHESMLKELSSREEKKYQAGIDQLEKATAKKKLLKRNKSLFF |
| | | VQAKAGKKAIAPGETVNVTFKEATYEGRVINNNANVPVTYDENLPYI |
| | | FQQALELGKRGGVMEVYCFAGDLYNPDTMPPDLFNYSLMKLTVTIS |
| | | GGK |
| SEQ ID | gene_800233 | MDYDVSISIGTTANLGDLDKANKAVQDLGRSIDKLPPQLLPGGVGGT |
| NO: 34 | | GAGGSATPSYVGTPSTSGSMTWRLDGMTELTGALGQTEAAVKQVDK |
| | | SITITSQRLDKNSSWLSRSISTLASLPGKIQSWGSNTMQAWGQFNGPL |
| | | QNVKNMISVGKQAWDLGWSLGESLNEAFGVKTKQIDAKVAGIIQAA |
| | | QDKLARWQDSINSARAQHREDAFLKQEAAGVKQVNDAYAARLRTIE |
| | | AIDRKAMAGLELQQKLLQIENEKNRSIIRQRQIRGEISDAQARDELAKI |
| | | DAKDAGERMDIERKQAEQAAATSQAKAEAAEERYRKLMELSQSGM |
| | | ARQAVQDLKPMDILNKADSLKRAEEDLAKWRSIQQRQKEAQKEIQQ |
| | | AIKDQARASTMLPLVGAPIALARKQAEDQARQDYEAAVAAQHEFMH |
| | | DKGMSFNETDKGNEDALKKIVEQRRKALDSMLGKIDKTGLVGNMDG |
| | | MAEDQRLGEYLRILKLVQDAMAQDAAQLESIFLETEALKQQAAEDK |
| | | ERVQRVMQEHQSQQAANDAVTKETAATNARQDADKHADVMVGAQ |
| | | EERLRKEIETKQRQQEKQKEDLSKTNERLNANMERFQQYAESFEGND |
| | | ALSAKLKQFSDIFTRLKGRPRDTWNKKDLVDAKAAEKFAKELVEASK |
| | | NSTNQDKKGIAQAAMQAIKAWQESIKKERAIKKNDKALRELERTAQ |
| | | DVANLSGKLHDGQSKVLELDDWLAKMRRKVLGSSGEIANKAPIGAL |
| | | PQAEEVLKKVLSEQGDGGTAVTQGERKLLEHLKNKLKNDDRRLEAG |
| | | NEFDEMIGLIDQILTRYSSAQSTHSKLSGEVARLKARLDKIDSQGKFGP |
| | | HR |
| SEQ ID | gene_1538800 | MTDPTSSVQTKQGVRKIYAYTTPAEESVDWLNGKGNGRVKIGHTTRS |
| NO: 35 | | VAERIREQFGASSTDRTWYPRGEWDAQAEDGTWITDHMVHRYLSKR |
| | | YRRVPDTEWFEVDPEAVWEAVEVLKNDPKARPKGKDCYELRGEQR |
| | | AAIDAAMTYYEADPSNRWFLWNAKMRFGKTFTAFKLAERLRSKRIL |
| | | VLTYFPAVDDGWSEEIEDHVDFEEWQYAENGASYEDGDQVQVSFSSF |
| | | QMLEHKTLGKNRENANTKADALRREIAAVNWDLVIIDEYHHGAHHP |
| | | ARREFVSSLKTQRILALSGTPFRAIAKGDFATENKFDWTYLDERQALA |
| | | DWAKKETCEANPYEELPAIHFVGYRLPPHAALTGVDGDCDLTYSPTTI |
| | | FKADKDGFKNPEAVKDWLQSLSTLSGSARRAGGVPPPYHPDFTGDVL |
| | | SHVLWLLPSKHSCDAMKRLLEDGWFPGGEGEVIQVSGSEGETGKPAQ |
| | | ITKKVRDKIAAAKRSITLSVGKLTTGVTVPEWTAVFHLKAGSSLESYL |
| | | QASYRCQSSGSINLRNGDREVKTNCFVFDYDPDRMLVVMGDYIKSLK |
| | | GSGAPLDDRSNAAFPTVIFDDEKNGHIPLNVSDIENELNNYLLKRRPA |
| | | ELMSDSMRLLEDAISAGGLNKALCAQLVKAGKSKSPHHAMRDLDPS |
| | | LFKSKTPGTIIGDNGKQSAKSTEVADENPKNDEIRAIKEAMLVFIKSLG |
| | | HLAYIGDLREASVEDLFKVDDELFEKIMGNDKDQVREIIDGAGLDRV |
| | | QLNAMIQKILMWENFEFVVSGCRNRDELGECGKWYPSDEEATYAFSL |
| | | QPNR |
| SEQ ID | gene_5543656 | MQRTLGNAATARAVGRGKRPAFRPSPPAIDERAEQGLVLPPYLMDLE |
| NO: 36 | | AGGLSTAYGLTGHEFVRGAVAAVVGHGGGTVAGIAAELAGRPESFF |
| | | GRGRAFAVEAGPGGGSAGAQGGGGYDVTVSIAPAPDDRPPTFHPAA |
| | | GLGTAAPDPGGAPLAAVDDPEGKETKVDVQHNTGATASRSVGNSAS |
| | | KGVGGTAFGLAPVAPGLWLGGAATGNVQPWQSSRDSRSQRGVAEPR |
| | | VLRSDKGSVEVARRVVYVVRVRPQAGGDEQVFRGSGGLTQRVPTEH |
| | | LIPAGTGAPARPEPVDAGLARRVALADSLAPLGVFDEAGPHRGGGGL |
| | | FDAVASVLHPSLTAPGAPGRARLYEATATPTVLEDLPRLLGGDGVTG |
| | | DDLYAKGGSSAGSYRMRAAVTGLAPAWSTGKTQLRTHQQAQHTAT |
| | | ESAGKGRAVAGGIGPAAGVGAAANAAVVRATAMPVAAARKARFSV |
| | | NEQTVSSRQGAEVRGEKVLYTGTVRFTVEGTGPRSMRMIRHPEARVA |
| | | THAMRVWISLRADEAQELGLPLPPGVTAGHFIRPPRPGAAPTSAAGGE |
| | | GEASTPAAAGSERHLPFGAMGSSVTLGRLDTAPMMKAVRELFATDP |
| | | RLTGYLPAFGTTPPVAGLSQEEAAAQRANHRELTTALSEANLRVNKD |
| | | QLLSTGIRVRLRRKTAMHSHDVQLRVHGTMGEAGHLGDIDDWLVRA |
| | | HAGVASNAQSGRSSSRSIGGMVLAQARLIPGALTGSARYERTTSGTRR |
| | | NQAGPTTRTDVLTNGSEKAAAFGAALRLNVDVTMTSRPRKMTRALT |
| | | PGAPGRDVPEAKLLSGLHLEEQDVRLLTPTEFTVGAEEKRRLDAGAG |
| | | RAPGAESATTATGIGDLAGAAPTAPTGQHLLSDWQLVETVGDGRPIR |
| | | ELALSLLSRAAARGEAGRRDPALTTEGLAPRLAVEERFSPRAITASLR |
| | | QAASSGWVVKNLRYPRRLAALNGAVGTRLALSSPQLVHEAAGPGTE |
| | | TFVLGGHQAGGQQGGGTSTTVQAGATLVQNGADWRVGEGLSAYGS |
| | | TGTGDSEAATVAGTVERNAHTPKKAPLYLVRCDLLVTMVAEVKVTG |
| | | GGPYVASAARTLPGAAAVWLTAAQLRAAGVDLPRSARKELKADGTP |
| | | APTTTTSAAGVSGAGSGVRSAARSDHGPGTRSGAGSGPGEASGGGSR |
| | | PRPTLSRGLPLGFGMIEDVPDFVPLLSGLRTTLALTGHQDLADELLPR |
| | | QQLRDRNDNVQRLLRVLDRDGSTGLLASAMDGGVTVELLDGRRTPY |
| | | WAVFKVDRVGDGVWDGEADDGRDMEYITSAVAQQSTAHDEGESVG |
| | | VEGVLAASGRPDGGKGQVKSTGAAAGLGLAKGSGRRRGGATRGQL |
| | | GMKTVAEAKTAKAARMRVPVVPSLELHRGDRRLAVAGLGRTTLVH |
| | | RVLEADLKALSRVTTPRRPAAHPRPDAPQGSDAALGAWRASGVPLP |
| | | MEAQVNGFQGAPRVRDLVSRTVRAAGGNPRFREKGQAAAYTLGEA |
| | | VSTEWLIAALPLLTHAGAPLPPVHATGAKGQDLHASVHARLRAGRIL |
| | | GAGDKMTFETVAQSDLTAPRPTQTDAQSAAEKSRQARGLLGAGVLN |
| | | ADEFRLNQLMANGGGAGSATDASAGGAGSMPLHKPKFASVLVQFTL |
| | | DVRVVARVTDRVRSSRTAVAERELTLPQPVVVRMPLPVARRMLAAY |
| | | PEAVADSRGELGV |
| SEQ ID | gene_3943627 | MQQTLGNEATARAVRRGKRPANRPPAIDERAEQGLVLPPYLMELEA |
| NO: 37 | | GGLSTAYGLTGQEFVGSAVAAVVGHGGGTVAAISAELAGRPESFFGR |
| | | GRAFAVEGAEGGQGGRNGQGGNGFDVTVSIEPAPDDLPPTFHPAATL |
| | | ASAPPDPGGAPLAAVDDAEGKDTKVDVQHNSGTTASSTVGNSSSTG |
| | | AGGTAFGLAPVAPGLWLGAAATGSVQPWQSSRDSRSQRGVAEPRVL |
| | | RSDSGSVEVARRVVYVVRVRRQEGGDEQVFRGTGGLTQRVPTEHLIP |
| | | AGTEPLPSSGAGGQERPVDADLARRVALADSLAPLGVSDSAGPHQGG |
| | | GGLFDAVASVLHPSVTASGAPGRSRLYEATATPTVLEDLPRLLGGDG |
| | | VTGDDLYSKDGSSAGSYRMRAVVTGLTPAWGTGKTQLRTHQQAQH |
| | | TATESAGKGRSVAGGIGPAIGVGAAANAAVVRATAMPVAAARKARF |
| | | SVNEQTVSSRQGAEVRGEKVLYRGTVQFTVEGTGPRSVRAILRPEAR |
| | | VATHALRVWISLRADEARELGLPLPQGVEAGEFIKQPEAGAEERHLPF |
| | | GATGSSVTLGRLDTAPMMKAVRELFATDPRLTGYLPAFGATPPPADL |
| | | SREEEEAQRANDRELMAALSEANLRVNKDQLLSTGIRVRLRRKTAM |
| | | HAHDVQLRVHGTMGEAHHLGEIDDWLVRAHAGVAANAQTGRSSSR |
| | | SIGGMVLAQARLIPGVLTGSARYERQSSGTRRNQAGPTTRTDVLINGS |
| | | EKASAFGAALRLNVDVTMTSRQRKLARAVTPGGPGRDVPEAKLLSG |
| | | LHMEEQDVRLLTPSEFTVGPDEKARLDAGAGQAPGAERPVTGAAGIG |
| | | DLAGLAPTPTAGQLVRDWQLVETIGDGQPVRDLALALLSRAAARGE |
| | | AGRRDEALGTEGLAPRLAVEERFSPRAITASLRQAASSGWVVRNLRY |
| | | PRRMAALNGAVGTRLALSSPQLVHEAAGPGTETFILGGHQAGGQQGE |
| | | GTSTTVQAGATLVQNGPEWRVGEGLSASWSTSTGDTEAATVSGSVE |
| | | RNAHTPKKAPLYLVRCDLLVTMVAEVKVTGGGPYAAGSARTLPGAA |
| | | AVWLTAEQLRAAGVDLPESARKALKLERPRPENGPTTSRAEGSGGGT |
| | | QTPAREGVGATGGGPSRPGPGLSRDLPLGFGMIEDLPDFVPLLDGLRG |
| | | NLATTGRQDLADDLLPRQQLRDRNDNVQRLLRVLDRDGSAGLLASA |
| | | MDGGVTVELLDGRRTPYWAVFKVVRSGDGVREGEADDGRDMEYIT |
| | | SAAAQQATSHDEGESTGVEGVLAGSGKPDGGVGQLKSVGGAAGLGL |
| | | GSGSGRRRGGAARGQLGMKTVAEAKTAKSAKVRVPIVASLELHQGE |
| | | SRLAMAGSGRTSLVHRILESDLTALRRVTTPRRAPRPAPGAPTGGQAG |
| | | LGTWRAAGVPLPMEAQANGFQGAPRVRELVNATVRAAGGDDRFRE |
| | | KGQAAAYTLGEAVSTEWLIAALPLLTNAGAELPPVHASGAKGQDLN |
| | | ASVHARLRAGRVLGTGDKMTFETAAQSHLGAPRPTQTDGQSAAEQS |
| | | RQARGLLGAGVLNADEFRLNQLMGNTGGSGSATGAATNAAGSMPL |
| | | HKPKFGSVLIQFTLDLRVVACVTDRVRTSNTQVAERDLTLPTPVVIRM |
| | | PLPVAGRLLAAHPTEIADPHDRLGLRTGAVPPGP |
| SEQ ID | gene_5085315 | MKPLKSYLAWVAVTLAVAGATTACQDDIDDPIIDAPVAKDQPNTSIL |
| NO: 38 | | ELKTKYWNDATNYIDTIGTRDDGSHYVISGRVVSSDEAGNVFKSLVIQ |
| | | DGTAALSLSINSYNLYLKYRRGQEIVLDVTGMYIGKYNGLIQLGQPE |
| | | WYENGGAWEASFMSPEYFTAHAQLNGFPDTSKLDTLVVNSFSELPTD |
| | | PAGLIKWQSQLVRFNNVSFANGGKATFSEHKSNVNQSLVDAEGSSIN |
| | | VRTSGYSNFWNKTLPEGHGDVVAILSYYGTSGWQLILNDYEGCMNF |
| | | GNPTVPEGSQSKPWSVDKAIEIEKAGTEKSGWVSGYIVGAVGPEVTE |
| | | VKSNDDIEWKADPLLSNTLVIGQTADTKDIAHALVIELPDGSKLQTLG |
| | | NLVDNPGNYGKQIALHGTLAKAMGTFGITGNNGTTNEFSIEGLNPGG |
| | | EGIPEGTGVKESPYNCAQVIAGVSGNAWVKGYIVGSSAGKTAAEMTN |
| | | ATGAAASTSNIFIAAKADETDYSKCVPVQLPIGEIRTALNINANPGNLG |
| | | KVVAVKGSLEKYFGQPGVKTVTEFDLEGGVTPPTPPTTSGDGSENNP |
| | | YNPAEVIAFNPQSSQEAVKSGVWVTGYIVGWADVSAAPYAINAETAH |
| | | FDASATMATNILVASSADVKDVSKCIGVQLPTGEIRSALNLQANPGNL |
| | | GKSLQIKGDIMKYCGVPGIKNATAYKLEGGSTPTPTPTDPVASINENF |
| | | DASSSIPAGWTQKQVAGDKAWYVPSFNGNNYAAMTGFKGNGPFDQ |
| | | WLISPAIDMSKVSKKVLTFDTQVNGYGSTQSALKVFVLTAADPTTAK |
| | | TTQLNPTLATAPATGYSDWANSGELDLSAFSGIIYIGFEYTSPVADNY |
| | | ATWCVDNVKLNAEGGSTPDPTPTPTPSGDFKGDFNSFNNGQPLSKPY |
| | | GTYTNNTGWTATNAIILGGGETDANPIFTFIGAAGTLAPTLNGKTSAP |
| | | GSLVSPALTGSIKTLTFKYGFAFNESKCQFTVNVKDATGNVIKSEVVT |
| | | LDKIEKAKAYDFSLDVNYNGNFTIEIINNCYSQLDANKDRVSIWNLTW |
| | | TE |
| SEQ ID | gene_4028206 | MVGVNERARVPFALLGVVLLVGSASIAAGLGGTSPTREPATEAAIEQ |
| NO: 39 | | GRTSLGGTVHDATRTAARNVAASPVVAPANTTLGRVLAATGDPFRA |
| | | ALELRTYLAVRDRLSATTERGVTVDPSLPALRDSADIDAALSRTTVEP |
| | | VGANATAVRTTVANVTLTAMRDGRVIDRYAVSPTMTVQTPVFALHE |
| | | RTRTYQQRLDSGATEPGLARRATARLYGVAWARGLTQYGGGPIANV |
| | | VSNQHVAVATNHALLAQQRATFGATDDTGRRAVRVAAARAAGTDL |
| | | LAATGQSGKQIQELLAGVDAATPGSTLDPVAAANPPITPESALNVSVG |
| | | EQATTAFDRFVTTDLDAVLAAPYRVTVERRRAVTDSATTTAGRERPT |
| | | GDNWTLVGTEQTDETTVTDGDATVGSPVNPWHTLATTGRRVAETTR |
| | | TERRWRRNHTTHTTVETTTQTRRVSIRLVGRHDGGAAPPVGTSPIHER |
| | | GGAIDGPNLAAVERRAKTRLLGDEQDLDALAARTTSDGTTQTTIRGE |
| | | QPLELRDWVYRDLVRLRERVANVSVAVERGAVGTYQVNPSDELAGA |
| | | LRARRARLVDRPDEYDGVADRARVAARGAYLDAVITELERRADDRD |
| | | GVKERLAGLLAARGLSLGRLRSIMAARSQVTTPTSHSISGVGGSYSLD |
| | | VEGVPAYLTLASVNRTQTDSLREGSVRPLAARNTNIFTVPYGDAADGI |
| | | VGKLFGGDRVRLRSAARALAAGEELATHETLEADVETAVSRRRRGM |
| | | RRVLRRAGVGDSRSDRRRIVAAGLGAWETVAERAIAVTENRGPDAV |
| | | AAVALRRSPGSFDGPADRDDLRRSLRAVATDGRGVPESSVTPHVERA |
| | | RQMVGKLVKQSVGRAANQTTTAVRERLESKTGKLAAVPSGIPVTPVP |
| | | SQWYATANIWDIEARGGYDRFAVSVRNGGPGRRLTYVRDGSTVVID |
| | | WNGDGELERAGTATAVTFAYRTAVVVVVPPGGQGVGDVDGNADER |
| | | SAGWGER |
| SEQ ID | gene_277399 | MPTTFENIKLKEDGTEGQIITISTFYVWDCTNQRFSTSPPVVLRNTMLA |
| NO: 40 | | ALYPAKEFIIGEIPKTSTNPSLLDPFKVPAVSDPYFLDLASNSRTHGRFL |
| | | FTPKRTIGRDYFPKKDDWKRIIYGSILHTGCNRMFYREIKYIVVDDERR |
| | | NPSDSSPQDDGVNNTHWDTGDCHAKLSKSLLTLLESWETIGNEDNPT |
| | | TIQIRAAIFKEWTIKGTASHSYKFETDPRFAGVDLVIPLSCFKGNKPAP |
| | | GNYTGKVLIGVVHEAEERRAKPGWMLWQWFSFETLEEDGIISKLHEK |
| | | CQKLSTALDDIYKLADVLRIDLDEAEQELANLDDNPDAEVAYVDSVL |
| | | KIIKADKKGVLILHPYVLLKVKFRLREMWKNLAKSAGVRFYSVMCTP |
| | | DTSLEKYQKAYGNDFVFKPKVFCSPSFNEGQYIVFCNPMRHWGDVQ |
| | | LWENFHEGRFRNTRGVLAATRELLLSLGRDTDGDFIQLINSSRYPNLT |
| | | MALYDMDAPPKVKKFPKVALTGSLQQIAINSMNDITGVVASLLGRAR |
| | | AIGAELIVLDIPKEGEMRIIDFLSQELQIAVDSLKSAYPNNQDGLKVVK |
| | | EFLDKSGADIQWLADLKSDDCYFTRPCLVNNNLTDTVTRIVSLVNSY |
| | | YRQPNLKEDTIPMDYRFTLFSLVVSDAVQDAIALRERDAYRAEMGAA |
| | | LAHKAANDDDRLVKEVTAKFRASTEVIMRETLNPFRKPYPPKTWAAS |
| | | YWRVNHLAKSGTAGLVFLLFCDEIIEELKNLENKKVWLITIYAVQFTA |
| | | FARPQLNAWNGEELTVRSSFLNVNGKDKVSLEGKLDGQPGFINMGL |
| | | VNEKDIAQVPNGWTGRVKIYAKTYENDKYPRKMSANDVCTSLYCFS |
| | | VDMEQSDIDDFMNDHWSTNSRFNPI |
| SEQ ID | gene_1961732 | MNRSLVSAVVLTAVLFPNCVKSAPDLPTQPFAYHEDFETADPVQFWV |
| NO: 41 | | SNGEYEVNSKGLTEEKAFAGKKSFKLDVTLKTATYCYWSVPVKVAC |
| | | AGKLKFSGRISVSQASKARVGLGCNYVFPPTHHSGCGAFDTFDKATD |
| | | DWQLQEQNLVADGDERADGVLRQNTSDATGANVVTFTDRWGIFLY |
| | | GGEGSRVVVYVDEVRLDGEVPDAQVYAAEADQRFEPAREVFRKRLT |
| | | AWREELATARQGIDALGALPPVAQRMKEVALKAADSAEADLTKFAE |
| | | ASYASPTDITRLESSVRTVRYATPNLIDMSKPGVADRPFVTYIVKPITN |
| | | ARLLPTSFPIVGRIASELSVTGCAGEYEPASFAVSALKDVEKLVVTPTD |
| | | LNSGANLIPANAVDVSIVKCWYQAGVSISDTRHCLLTPELLLKDDALV |
| | | RVDTEKKENYLRSGEGEKYALISTKDSSTLTDIQPRDAKSLQPVDLAA |
| | | DTTRQFWVTVHIPDDATPGEYTGTLKLAAANAPAAELTLRLRVLPFK |
| | | LEPPALCYSVYYRGVLTPDGKGSISSEEKSPEQYAAEMRDLKAHGVD |
| | | HPTLYQSFNEPLLEQALDLRKQAGLPTDTLYTLGLGTGSPTNAADLD |
| | | KLRATATKWVEVAQRHGFGEVYGYGIDEATGDRLTAQRAAWQVLH |
| | | DAGAKVFVACYKGTFEVMGDLLDLAIYAGAPLADEAQKYHQAGQRI |
| | | FCYANPQVGVEEPETYRRNFGLLLWQAGYDGAMDYAYQHSFGHGW |
| | | NDFDSPQYRDHNFTYQTVDGVIDTIQWEGFREGVDDVRYVTTLVKA |
| | | MEAAREAKPALVKQAQTWLDGLDVKGDLDEVRGKTVEWILKLTK |
| SEQ ID | gene_2755817 | MLLVHIAGHADLGAPSPFEDPDKIGPLRAEELKNCMTPHEATRCLFDL |
| NO: 42 | | SFTQTPSHKYTDTAHSPHSGSALRKELTAVSQISAATSTDETTEVLIIG |
| | | VEGEDTPTDRLARALVDALRMASSEAADLAGTSEIIIRDACILPSLAVS |
| | | RESIELLERRIGAHDGHVLLAMAGGATTVLAEAAGVAAATHQDEWS |
| | | LMLVDRVEEGSDGQSLPLIPMSVDADPLRGWLMGLGLPTVLDDIYEQ |
| | | SDRIDTEVKKAADAVRRVMGELDSEPSAEDFAQVLQADVARGDLAA |
| | | GMTLRAWILAKYKHLRDAHSYTNDSCKQSNKQLRQELGRVIGRLRE |
| | | SAKSHALEEPESWLVAQGDLNDLGKYATHNLESPLRNLTSNNLQERI |
| | | KQAVGEPPEWLSMPSGDVCLLTAQGKAARNAPLTSGADAPDRKRRR |
| | | PIIVSLLTSEPSDSVRQACAVHGPLTLSPFIACSSSSLSEGRRVADEVKN |
| | | GEQPASHSPWTLDETSIKVHDYGESITRPGVSSETISSSMKGLSRAAEH |
| | | WLEERTSRPRAVVVTVLGEKAAAISLLHAAQIFGAKHGVPVFLLSMV |
| | | NSKDTETGESKESVQFHQLGLDRDVRQALLKATTYCLNRFDLLSASR |
| | | LLSLGDPAMEVLSNEANILADRLIESVNTNDLDGASSTVLSAMNAVA |
| | | DLVKIVPSDAQVRLTTIVGELLRTPDEKYRSPNFKAPVALACASPDFD |
| | | QGNDYKKKLKQLELEPSESLLRLLIRVRNKIPINHGRNTLDVATELSL |
| | | QNFPDGNRYTYPVLLQRAIAAVGSKHGARAGDWGHRFHSLRDQVEA |
| | | LGKTGYGEKP |
| SEQ ID | gene_2831443 | MTYHIRAGQLVLEINERGEARLQADKVGASEGLPMAMYPSPLLRLVQ |
| NO: 43 | | DGELQEPAGCEQEDRTGTLTLTYPNGTKIKVGVAVRDSYAALEVLTIE |
| | | SGSPDAVIWGPFRTRIGGSIGESVGVVHDGRFAIALQVLNAKTVGGWP |
| | | LELDRLAYMAPSYSEGDAPDPNGRRGSDNKFEYPVCTAWPTVDGGS |
| | | ALQAYARDRTKRSIRKAWNVPATEVRPFEGEDAVIVGSGIALFGCPVE |
| | | EVLETIEQIELGEGLPHPTIEGQWGKTSPAANQSYLITAFTEETIGEAVQ |
| | | YAKLAGLSYVYHPDPFEQWGHFKLKRGSFPSGDEGLRRCSEAARAE |
| | | GVSLGIHTLSNFTTLNDSYVTPVPDIRLQPLGAAVLAEEADERGDSLTI |
| | | DEPWPFTVALYRKTARIGSELVEYAAVSETKPWRLLGVKRGMHGTA |
| | | ASKHGKGETVARLWDHPYDVVFPDLELQDEYADRLAELMNGADIRQ |
| | | VSFDGLEGLYATGQDDYGVIRFVERQYRSWGREVINDASIVVPNYLW |
| | | HMATRFNWGEPWGAETREGQLEWRLSNQRYFERNFIPRMLGWFLVR |
| | | SASDRFESTALDEIEWVLSKAAGFGAGFALVADEEVLKRNGNIEALL |
| | | AAVREWETARRLGAFSAEQRERLVEPKGDWHLEPVGPQRWNLYPVQ |
| | | ATKPLVCTPAEQQPGQPGGSDWAMFNKYAEQPLRFTMRVRPSYGNE |
| | | DAAVQRPTFYTDGVYMTFDTEIAANQYLECDGTRTGRVYDANRNLL |
| | | RVVEASAEAPTVRHGGQTLSFSAKFIGDPKPDVAVKVWLYGDPETVS |
| | | ADE |
| SEQ ID | meta_gene_ | MPLSRLQNFLKSVRGNILYVNPNDLDATDSIENQGNSLTRPFKTIQRA |
| NO: 44 | 118560 | LVEASRFSYQTGLSNDRFAQTTVLLYPGEHVVDNRPGFIANDAGGGS |
| | | AEYTSRGGTTGLSISPFDLTSNFDLESSSNVLYKLNSIHGGVIVPRGTSI |
| | | VGYDLRKTKLRPKYVPDPENSNIENSAIFRVTGGCYFWQFSIFDASPS |
| | | GQGYKDYTDNTFLPNFSHHKLTCFEFADGVNNIAVKDSFLNVSKSFS |
| | | DLDNYYYKISDVYDNASGRAIAPDYPSGNVDIEPIIDETRIVGPKGGSV |
| | | GITSIRSGNGVTGNTTITVETSTALSGITVDMPLRIIGVTASGYDGQRTV |
| | | KSVGSGSTTFTYEVDTVPSTLFETPSNAKAELQVDTVSSASPYVENCS |
| | | LLSVYGMGGLHADGNKATGFKSMVAAQFTGISLQKDVKAFVKYNTS |
| | | SGVYDDSTTVDNIAADSLARYKPAYSNYHIRCSNDAVLQIVSCFGVG |
| | | FNGHFLAESGGDQSITNSNSNFGGAALVSDGYKEDAFSRDDVGYITHI |
| | | IPPKEITTSDSALEFVSLDVSKTLSVGNTSRLYLYDQTNADVKPETVIQ |
| | | GFRLGAKTDDKLKVLIPLSGTTTEYSARIIMHNTAYASDEPSSVKRFTL |
| | | NRSSVGINSITNSILTLTKVHNFLSGESVRVISESGHLPDGIDEKLTYNV |
| | | IDANIDSSLATNQIKLAQNETDALADNFATLNNKGGILTIESRVSDKLA |
| | | GDAGHPVQYDSGQNQWYVNVATAATENNIYSTVIGYSTAIGSNTPRT |
| | | YISRKSDDRSQQDTLFRARYVVPAGVSSARPPIDGYVMQESCGDIETT |
| | | ANIQLVTLTNSVQQRNQTFIADANYLAATGIATITTEKPHNLEVGAQV |
| | | QMLNVVSANNTTGIGTSGYNFKATVSGINSDRSFSVALDDDPGAFQN |
| | | DTSTRTVDLPYYKKKDYATNFYVYRSTEIKKHVKDQQDGVYHLTLL |
| | | NASNAPNITPFSGQNFSQNIIDLYPQTDRDNINSDPDSARSFATPDDIGE |
| | | VLTNDLKKSITKENIIRFGRDSKVGIGVTDICSDIVVGTSHTIYTDRDHG |
| | | LFGIKSVGLGSTGFGYGSGAAGTLYNATLTAVGSSTVGKSATAEITVD |
| | | GIGGITSVRITNPGSAFGIGNTLAVTGTATTTSHVQGWVTVLTTFDNT |
| | | NDSLSVLGVTSNTYSSRNTQYQVSGYEIGESKKIQVSTASSMTGIGAA |
| | | STMGIGATVCARAMVFNAGPGIGITYFSYDYLSGIATVGSGVTAHGLS |
| | | VGNVLSFVGSSNTAYNGDFRVTQVVGLTTFKVNAGVGTESPSESAGG |
| | | SFYALPRGYASNDGAISLENENLSSRMTPILSGISTTLNSAVTTKTATS |
| | | VEITNSFNSGLQKGNYIQIDEEIMRVATTPVGGSDAVTVLRGQLGTRR |
| | | ATHIDGSVIRVVSPIATEFRRNSILRASGHTFEYVGFGPGNYSTSLPEK |
| | | VDRVLTGKQELLAQSVKKGGGVNVYTGMNDKGNFYVGNKKVNSTT |
| | | GQEEVVDAPIATVTGEDLDIASGVAVGLDVITPLEVTVSRSLKVEGGT |
| | | DANIISEFDGPVLFNKKVTSLGAGGIEANTFFIQGNATVAREVSVGIST |
| | | PTVNGNPGDIKFFSDPKSGGSVGWVFTVENAWRRFGRISLYDFKDTNI |
| | | FDQVGIATTTPNNYELQIGAGSSIINASAGKLGVGVTTPVRKLDVYGD |
| | | VGATGFVTAGTYVYGDGSRLTNLPSDSQWTRTDAGINTISTNAGIGTT |
| | | NPAYSLDIRGGKSGNSGQLYVGGDSQFTGVATMANVQATTLSATDV |
| | | LIIDSDGQADVGIVTVRDYFNVGVGGTVIFTNSAGKVGINSATIDNQA |
| | | AVDIGGRVRLDDYYEKVTTVTSSSGVVTLDLAKSRTFNLTTSEAVTQ |
| | | FVLSNRLDSDDHTTFTLKINQGSSAYAVGINTFKQTSGGTAIPISWSGG |
| | | VVPSVVNVGLKTDIYSFQTFDGGASLYGIVVGQNFS |
| SEQ ID | meta_gene_ | MNTSTVTNNNAETTAIESLFAKKLLRSKGIAVIPPSTGSGKTREIARFA |
| NO: 45 | 324030 | SNPKEYIDNIKSNFSNGLCEIDESKKIKTIYISPQIKHCQDFISDIANDES |
| | | CKDFCYEKRACRILNIFEVAEKVVGAYEDTKEKLNKTGKKTPSLLNE |
| | | RLLYKDGKIGENGENKQIEQFIEILNGLDKSSNMSEQIKEELQSKAKSQ |
| | | FRDIKTMIAKNYLNLEENPDFKDIELETYLKEPSLNWFVLLFPAHFWD |
| | | EINTYSLTVKMSSFTIRDVIFSKDLSSLLKPEEEQSFVFIDEADTASEELI |
| | | DTESENATKNSSIDVIKLLITLSRILDFKDVFPNYSSQKEKKQFEKAIER |
| | | GRKKFIEHFGDVDSNSTLIPTKEVKKLANSKVLHNYILRDSVETRVIIK |
| | | QGESAKKYMKDYYLSFPKNSEESKETPAFLITKDQIEEFPSDKYKVFE |
| | | YKSFLKIASGLLNYFCEFVYPAIVELIKKNEEDDNEMRSLNGTLQTFK |
| | | ELYNVDEDFIKLLHDYKTQNYKKKITGASSGLLSYCDIGYEIFQVKVP |
| | | LGGRPAELSRLLVQGTPEQTVVELAENSRVVLVSATANVPSLKNFNL |
| | | DFLSNRFGDYFDNFTMEDKKDFEAKLNYSNHNKSIELISDVSYELKYY |
| | | EKDKEPDDTEETWLERKVEENFSYLSKKMKMYLTNELTKEGIHRAN |
| | | YYILLIKYYLAMKAAKTKANLMIFQPNLEKEVIETLLNIFDPKLNEEN |
| | | AIFCANTEKLKTDGFIEKVENAYLEGKTIFLITSLATMGKAVNFTFKAR |
| | | EDEKLIHITPNGWIDDATKPAKRTFDGIAIGDINFSFASKDNNESSNNE |
| | | SSALRLLIDKITEVERLYATNLISNQIKRRIIQEMIINYESLYSFRGEFSTI |
| | | RKLQGFYVYKEISQAIGRLYRTPNFSEKMLVLTTKNNHDNLSTIKDSIE |
| | | RKSFIETPLMTALMNEVQKEEITKKNSIEAKTMPLKNSGELFSRLLGTL |
| | | LSDALKFKDKTSIQILEEMRRICIKYGVFLTEETYNSITSEQKDVDITSI |
| | | KERLYQKVETSDFIKNGYKYKSHDDHSIIDFIDPKSSSEQGIPVSPTNCT |
| | | IQQFRNLEGFYDYKENCGYTYDKVFNGEYIYILNPTAYNNLFKGALG |
| | | EFVGKYIFEILFKLPLSRITDPEAYERADFFFAHDNSTAIDFKCYSNPKV |
| | | EKESLLEGIKNKAKALDIKEYHVINVFPYSTKGVPFTKETLLNEDGTA |
| | | LLNSNGEPVVVKIVQATARPTSNCIVTDEFHQYILDTFLNKKGN |
| SEQ ID | meta_gene_ | MENISFSREKALPFSLEKLETIFNNLIQRDTYSNKILKEPLSEFYRREIES |
| NO: 46 | 295919 | EGKYRDNFLQIVEYTLSSLETIVKNPKRELLKISELQSINEIRSTDYKTM |
| | | IWLGNKPGKTLAEKIGAKGKILAPKNKYSIDKKENRVVVYYFKEAYK |
| | | ILEERYKRYIENSVDIPENLKKIYERFYRIKREMINNELFFLDRPIDFSPN |
| | | NALIDHRDYSVVNRGLKHLKKYLEKLDYSENILLELAKKIVFLKLSYF |
| | | IARLENIDIFDEILDIEELLKTKKKIIKFYSSKLQYLIKVILNKSKSKIRIEF |
| | | QKIFFNRDTKEVEKRDKEILDIDIIDTYENSASYYKLKVKDVEYNEND |
| | | DDLKKILFENIKIDNLIKNKKESMNSERIINKYIYMNFNSQSLFIDNKAL |
| | | EIKSYNKKLDNFMDTKDYFLSHSEQQAHYHINEIVSSDETIDIFPKYLE |
| | | YIKEKRNIDKQNICIYSSLEALDSDSQKMLSSIYDSNFNKSYPIWRSILA |
| | | TYAIKNSSKKWLENKEKFFVLDLNSEIPTINTIEIEKNINRHHPVIILEES |
| | | ENEELKELSLQAYLKEYLEKYLNVYSIEMDEVEKTNLISSGKVYETIF |
| | | KRKRYLITNMNFYLEKDEDIIKNVGNKFYSNVQKFVSKFLIDKRKKLL |
| | | IISDYLGEKYSLNGVDVKVIKEKELSLGKDEIIEKIKNNKNLWNEYLPN |
| | | LTLETVKDGHFYNLDLIRENEDVEVIFGVEQKININENLVLPKGMDVI |
| | | KFPLYSQDSNNKKLYFLEIKSELFPLKENLVVNLELIYSYGSKEPYKIK |
| | | LKANGIDSSKFSTKWTENINKLKIVSLDYPEKNNKKNNYLGIKKILEKI |
| | | DLNNTNLKDYLKRNKNRFRNYIIEEIERGNLERIKEVLDRNSKILALLE |
| | | ILNKQEKEKGLLNEMIAVFLASFGVLIYDRIKVDILKFEYRKRSTLFLY |
| | | SLNNQLKLEDVLKYNKKDPEIIETVAEISWLDKVFINKLAEKEPELLEG |
| | | ALKFLKYTLKSLNQKFGEEYEKWSKENLLWMLANRFKNYLEFILAIL |
| | | TIKDKEKILKVLNKRDILKILYDIKAIDRKIQIDYPKLKEEFNKRIKLKF |
| | | DRVVEQKKEVGLEAMSDLAYTVYCYLSGNNGSEAIKIKEVLDDFND |
| SEQ ID | meta_gene_ | MYLHGHYYNEQNERIEVHIVTHGDKTDNQEISADTGDIQWTDDPVEI |
| NO: 47 | 237613 | ESQVSDTFDVLLPQQATIRLQVRNFVADLFCADLREAVVNIYREGECL |
| | | FAGFLEPQSYSQGYSEEFDEIELSCIDVLTALKSFKYGDVGSIGRLYHE |
| | | VKANARQRSFQEIITEMLTSLTSHIDILGGHSMSLYYDGSKAIDNQTDS |
| | | RYRIFSQLSINELLFLSDEEDNVWTQEEVLTELLKYLDVHVVQVGFTF |
| | | YIFSWESVKRAASITWQNLLTGQNSETPYRKMDIRTGDVIGDDTTMSI |
| | | GEVYNQLLLTCKVEKMEQLIESPLEDSALRSDFPAKQKYMNEFISWG |
| | | TGKRAIEGFRDLVFNSTTAYDAASIVDWYIWVKRHPHWTFPMHDNSL |
| | | QAGMSLSDYFGQTGRNQQAYLQWLGSHLGAALVAYGKVATEMAR |
| | | GDNSPIAKIDMDNYLVLSVNGNGQDDQAKTYPKETDLKAAIPYAVYE |
| | | GKKAGGVFSPADEQTTNYIVLSGKMILNPIMTQTATFRDLRTKPWTA |
| | | KNIFSGQPIEEGKACVYGNVVKDKNGSEKYYTCKYWKQTDSNPKLN |
| | | EEPQWDEQGDGGWYPFTGTAPESYEYNYSAVGDGTDKISKVGLVAC |
| | | MLIVGDKCVVEKGSGSQIEDFEWRKYKERSACSSDDEYYQQSFTIGF |
| | | DPKIGDKLIGREYSLQNNISWKRGIDTEGMAIPIRKRDHVSGAVRFVIL |
| | | GPVNVLWGDITRRHPTFFRHTKWTEHAVPLLAHVSSIQIKQFEVKLHS |
| | | DNGLIEHLGDEHDIIYMSDAKTSFCNKKDDLEFKITSALTYDESVQLGI |
| | | VNTPCLSTPVNMASGDGVLQVCNTLTGQQAKAEQLYVDAYYREYHE |
| | | PRVVLKQTFADRTNGIVDLFTHYRQAFMDKTFFVQAINRSLTEGSAEL |
| | | TLKEINND |
| SEQ ID | meta_gene_ | MPTNYKTIINFRDGIQVDANDLVSNNGLVGIGTTIPREELDIRGNLIVE |
| NO: 48 | 35066 | NQANFRDVNVVGQSTFYGDINIAVGNSVGIGTTVPEATFQVGVGTTG |
| | | FTVDSNGNVTALTFTGSGANLTNLPTAVWTNPYPGAGTTINAFRPVG |
| | | VSVTLPQADFAVGDLIKLDATSGVGTFEGLVAKNITAVNASGSGQGN |
| | | VNGEVGTFSTITATDTAVIDKLDGNLIGLSTIAGTASTANSVYVTDEST |
| | | DTLLFPLFVDGAVLSGQIVAGNKEVKAGTNLQFDSANGTLSATSLSA |
| | | AGGISIGPGGIMTATTFSGTATTALNASVAYAIAGQPDIQADKIDSLGI |
| | | NSIFIRNTGVSTFGGEVKVGNFLGVGATSSAIGKGMGVIGAADFSGAG |
| | | TFGGDLLVAGNLSVGGTFGGAVNITDVTAGEIIATGILSATTSSSCVLH |
| | | DTTITGNVVQSAGKNLTVGQNLSIGGTTTFGSQINFGDASTQVAAAGT |
| | | LFANLSGIITTGGINVGDLDISGTFSYTGGSIATFGSILLNSNTGFVSCSS |
| | | IEAGTGIISCTGLNARTGEITGGGLNLTGPTTSNNFFQSTSGVSTFFDIDI |
| | | TGGTNSNIQLTRLGFNTSLGALGITEGIALWDDAEIYVNDSPASGIGIG |
| | | TTSGKRDSNVALYVGYGRDGAGNFINGQSVFEGGVGIGTMMGNDDG |
| | | NMLEVYKETVFHSYHTGVGGTDAGPARVGFETNKPRTTLDLGFVTS |
| | | GFLRIPSYYNDDPNNTVPTNDTGSQGSLFFDTAINSISIKDMNDNWVGI |
| | | KTELSTGDDPAQYVQELGFIGGVTDQANRLSAEQGVANIIQPYDEVG |
| | | NQGIGWGTAHMWYNKTFNKHQYKTNQGIGVATHYRSYVSTGTSAID |
| | | IELDSSGTKVYITLPGIGSATFNLV |
| SEQ ID | meta_gene_ | MWWKFYLIPDYVSIRRDINGHPVFLLIKYAFNDQDRQENKNLPRGGG |
| NO: 49 | 524019 | FMVFDVELSVREADYPKIIAELQQSVNSQWQQLKALADAAGNDVRG |
| | | YSVNSWHYLNGNFQFSTLSVNDLQLGLHPERPEAPPGDAPPKVIISQP |
| | | TWKEGKFHVSAPQSTDLVAHRVSEGPVSLVGNNVVSANMDLTTGGA |
| | | TFMEKTLTNLDGSGATDLTPIQVVYELTFWARVPPVHLLVTVDSRSL |
| | | YEATKNIYHDYEGNGCDEDSINHSEQNLEMAVQSGLINIQIDTGTLSL |
| | | SDDFVQQLRSGALKFVQDQIKDNFFDKKQAPPPADDPTKDFVGSDKE |
| | | IYYLKSDIDFKSVSIGYNEQIDSIVEWKANPQGTLQTFLAGVSPSEMKR |
| | | YVRDVDLRDTFFMTLGLTTTVFADWEHEPIAFVECQISYTGRDENNQ |
| | | LIEKVQTFTFAKDHTAEFWDPSLIGSKREYEYRWRVGFFGHDAGEFTS |
| | | WLTETTPKLNISIADPGKITIKVLAGNIDFAQTTKQVQVDLKYGGPGL |
| | | EVPEEGTTLVLVNGQLEGNYERYIYSTWDHPVLYHARFYLKNEQVVE |
| | | SDWQETVSRQLLINQPFLDQLKVQLVPAGSWDGVVQTVVNLRYKDE |
| | | LHSYHSEEAYTIKSADEFKTWAIVLRDPNQRKFQYKILSTFKDGSTPA |
| | | QTDWIDADGDQAVLIRVQQHPELKVKLLAGQIDFKVTPVVECTLHYD |
| | | DLQGHIQKVDTFPFSKAEDAVWDFPLASDSRRTYRYQITYHTADGHTI |
| | | PMPEVSTDTTSVVIPPLEIPVISCTIFPKLVNFVQTPVVEVDFEYKDPDH |
| | | HIEFEDTAVFTDSNPQSFRVQVDKASPRNYNLAVTYYTADGKVIQRD |
| | | PVTLDKNKVVIPMYVATS |
| SEQ ID | meta_gene_ | MIYRDHQDKGLFYYIPERPRLARNDGVPEFIYLVYKRDITDNPAFDPE |
| NO: 50 | 523517 | TKASLGGGFLAFTVDLGVDDQQLAEMKQELARFSDGEEVKLTPVQF |
| | | HKGSVRLSISKDTADAPGTPPDQPKGLTFFEEVYGTTKPSLFGFNRAT |
| | | FSVVLSQEVAALFEAALQAGISPIGVIYDLEFLGLRPAFNVRITAEYKRI |
| | | YDHLEIEFGARGQIYAVALALDIDLAFQKLRDDGSIKVEVLSFTDDAN |
| | | LRKQADDAFNWFKTELLKDFFKSSLEPPSFMKQTNTTDLVGRLQSIFQ |
| | | GLNSAQTSPTLNPVRGEPTKEPLTPAAPPKKQEDGMKSTADMNRAAT |
| | | QSGSESSGGGSGADRGISPFQIGFTLKYYRQEELKTRTFEFSEQAAVAR |
| | | EAAPQGLFTTMVQGLDLSRAIQHVNLDSDFFKRLITTVSASDEFTIAGI |
| | | STLGVNLEYPGTRKPGEDPLFVDGFVYKSDDLKPRTFTTWLNDRKNL |
| | | TYRYQMDIHFTPDSPWVGKEGSVTSDWIITRSRQLTLDPMNEISLFDV |
| | | QLTLGNMISGQINQVEVELRYQDSANDFNTQKTFLLKPGDPVTHWKL |
| | | RLMDSEQKTYQYRITYFLQEGVRVQTDWVSSEDPTLVVAEPFKGTLN |
| | | IRMVPLLDPTTLLEADVELMYHEEDTGYTRRVEKVFSPSDLKGQQISI |
| | | PTLAENPTSYNYTINIIRTDGSTYTLPPTTATTPVLVVSDGAGVTHRILV |
| | | KLPSKDLSSFGLAALKVDLVGPGDDPDTASVLFTPSQTDDKMPALVQ |
| | | PGDGGTFTYSYKVTGYTTQGLPIEGDSGTSSGPTLIVKIPTR |

### Methods of Producing a CRISPR/Cas System

### Nucleic Acids and Methods of Introducing a Nucleic Acid in a Cell

Also provided herein are nucleic acids encoding any of the CRISPR-associated proteins or CRISPR-associated arrays as described herein.

Any of the isolated nucleic acids described herein can be introduced into any cell, e.g., a mammalian cell. Non-limiting examples of a mammalian cell include: a human cell, a rodent cell (e.g., a rat cell or a mouse cell), a rabbit cell, a dog cell, a cat cell, a porcine cell, or a non-human primate cell.

Methods of culturing cells are well known in the art. Cells can be maintained in vitro under conditions that favor cell proliferation, cell growth, and/or cell differentiation. For example, cells can be cultured by contacting a cell (e.g., any of the cells described herein) with a cell culture medium that includes supplemental growth factors to support cell viability and cell growth.

Methods of introducing nucleic acids (e.g., any of the exemplary nucleic acids described herein) and/or gene delivery vectors (e.g., any of the exemplary gene delivery vectors described herein (e.g., an AAV vector)) into cells (e.g., mammalian cells) are known in the art. Non-limiting examples of methods that can be used to introduce a nucleic acid (e.g., any of the exemplary nucleic acids described herein) and/or a gene delivery vector (e.g., any of the exemplary gene delivery vectors described herein (e.g., an AAV vector)) include: electroporation, lipofection, transfection, microinjection, calcium phosphate transfection, dendrimer-based transfection, anionic polymer transfection, cationic polymer transfection, transfection using highly branched organic compounds, cell-squeezing, sonoporation, optical transfection, magnetofection, particle-based transfection (e.g., nanoparticle transfection), transfection using liposomes (e.g., cationic liposomes), and viral transduction (e.g., lentiviral transduction, adenoviral transduction).

In some embodiments of any of the methods described herein, the method further includes formulating the CRISPR-associated protein, CRISPR-associated array, and/or guide RNA into a composition (e.g., a pharmaceutical composition).

Also provided herein are methods and compositions for specificity of transduction and/or infection, e.g., using any of the AAV capsid proteins or AAV virus serotypes. In some embodiments of any of the methods described herein, specificity of gene expression is determined, e.g., using any of the tissue-specific promoters and/or enhancers described herein.

### Promoters

In some embodiments, the gene delivery vector (e.g., any of the exemplary gene delivery vectors described herein) can include a promoter sequence. In some embodiments of any of the gene delivery vectors described herein, the promoter sequence is a tissue-specific promoter. In some embodiments, the promoter is an H1 promoter. In some embodiments, a promoter is a ubiquitous promoter. Non-limiting examples of ubiquitous promoters include CAG, EF1α, UBC, SV40, CMV, or PGK.

### Enhancers

In some embodiments, the gene delivery vector (e.g., any of the exemplary gene delivery vectors described herein) can include an enhancer sequence. In some embodiments, an enhancer sequence is a CMV enhancer, a CAG enhancer, or a cHS4 enhancer.

### Poly(A) Signal

In some embodiments, the gene delivery vector (e.g., any of the exemplary gene delivery vectors described herein) can include a polyadenylation (poly(A)) signal sequence. Poly(A) tails are added to most nascent eukaryotic messenger RNAs (mRNAs) at their 3′ end during a complex process that includes cleavage of the primary transcript and a coupled polyadenylation reaction driven by the poly(A) signal sequence. In some embodiments of any of the gene delivery vectors described herein, the gene delivery vector can include a poly(A) signal sequence at the 3′ end of the isolated nucleic acid encoding a fusion protein (e.g., any of the fusion proteins described herein).

The term “polyadenylation” refers to the covalent linkage of a polyadenylyl moiety, or its modified variant, to the 3′ end of an mRNA molecule. A poly(A) tail is a long sequence of adenine nucleotides (e.g., 40, 50, 100, 200, 500, 1000) added to the pre-mRNA by a polyadenylate polymerase.

The term “poly(A) signal sequence” or “poly(A) signal” is a sequence that triggers the endonuclease cleavage of a mRNA and the addition of a sequence of adenosine to the 3′end of the cleaved mRNA. Non-limiting examples of poly(A) signals include: bovine growth hormone (bGH) poly(A) signal, human growth hormone (hGH) poly(A) signal. In some embodiments of any of the AAV vectors described herein, the AAV vector can include a poly(A) signal sequence that includes the sequence AATAAA or variations thereof. Additional examples of poly(A) signal sequences are known in the art.

### Internal Ribosome Entry Site (IRES) and 2A-Self-Cleaving Peptide

In some embodiments, the gene delivery vector (e.g., any of the exemplary gene delivery vectors described herein) can include an internal ribosome entry site (IRES) sequence. An IRES sequence is used to produce more than one polypeptide from a single gene transcript, and forms a complex secondary structure that allows translation initiation to occur from any position with an mRNA immediately downstream from where the IRES is located. Non-limiting examples of IRES sequences include those from, e.g., hepatitis C virus (HCV), poliovirus (PV), hepatitis A virus (HAV), foot and mouth disease virus (FMDV).

In some embodiments, the gene delivery vector (e.g., any of the exemplary gene delivery vectors described herein) can include a sequence encoding a “self-cleaving” 2A peptide (e.g., T2A, P2A, E2A, or F2A). A self-cleaving 2A-peptide is used to produce more than one polypeptide from a single gene transcript by inducing ribosomal skipping during translation.

In some embodiments, the nucleic acid sequences are operably linked to a promoter or are operably linked to other nucleic acid sequences using a self-cleaving 2A peptide or an IRES sequence.

### Compositions and Kits

Also provided herein are compositions (e.g., pharmaceutical compositions) that include any of the delivery systems, CRISPR-associated proteins, CRISPR-associated arrays, and/or guide RNAs described herein. Any of the pharmaceutical compositions can include any of the delivery systems, CRISPR-associated proteins, CRISPR-associated arrays, and/or guide RNAs described herein and one or more (e.g., 1, 2, 3, 4, or 5) pharmaceutically or physiologically acceptable carriers, diluents, or excipients. In some embodiments, any of the pharmaceutical compositions described herein can include one or more buffers (e.g., a neutral-buffered saline, a phosphate-buffered saline (PBS)), one or more carbohydrates (e.g., glucose, mannose, sucrose, dextran, or mannitol), one or more proteins, polypeptides, or amino acids (e.g., glycine), one or more antioxidants, one or more chelating agents (e.g., glutathione or EDTA), one or more preservatives, and/or a pharmaceutically acceptable carrier (e.g., PBS, saline, or bacteriostatic water).

In some embodiments, any of the pharmaceutical compositions described herein can further include one or more (e.g., 1, 2, 3, 4, or 5) agents that promote the entry of any of the gene delivery vectors described herein into a cell (e.g., a mammalian cell) (e.g., a liposome or cationic lipid).

The pharmaceutical compositions provided herein can be, e.g., formulated to be compatible with their intended route of administration. In some embodiments, the compositions are formulated for subcutaneous, intramuscular, intravenous, or intrahepatic administration. In some examples, the compositions include a therapeutically effective amount of any of the gene delivery vectors described herein.

Also provided are kits that include any of the compositions (e.g., pharmaceutical compositions), isolated nucleic acids, gene delivery vectors, or fusion proteins described herein. In some embodiments, a kit can include a solid composition (e.g., a lyophilized composition including any of the gene delivery vectors described herein) and a liquid for solubilizing the lyophilized composition.

In some embodiments, a kit can include a pre-loaded syringe including any of the pharmaceutical compositions described herein.

In some embodiments, the kit includes a vial including any of the pharmaceutical compositions described herein (e.g., formulated as an aqueous pharmaceutical composition).

In some embodiments, the kit can include instructions for performing any of the methods described herein.

### Cells

Also provided herein is a mammalian cell (e.g., a peripheral mammalian cell, a mammalian neural cell, e.g., a human neural cell) that includes any of the gene delivery vectors, fusion proteins, or isolated nucleic acids described herein. Also provided is a mammalian cell (e.g., a mammalian neural cell, e.g. a human neural cell) that is transduced with any of the gene delivery vectors described herein, edited using lentiviral or CRISPR technologies, or otherwise engineered or modified to express any of the fusion proteins described herein. Skilled practitioners will appreciate that the gene delivery vectors described herein can be introduced into any mammalian cell (e.g., any neural cell), that a variety of technologies can be utilized for modifying the genome of mammalian cells, and that such modified human cells that secrete fusion proteins can be utilized as cell therapies. Non-limiting examples of gene delivery vectors and methods for introducing gene delivery vectors into mammalian cells (e.g., any neural cell, e.g., a human neural cell) are described herein.

In some embodiments, the mammalian cell is a human cell, a rodent cell (e.g., a rat cell or a mouse cell), a rabbit cell, a dog cell, a cat cell, a porcine cell, or a non-human primate cell. In some embodiments, the mammalian cell is present in a subject (e.g., a human subject). In some embodiments, the mammalian cell is an autologous cell obtained from a subject (e.g., a human subject) and cultured ex vivo. In some embodiments, the mammalian cell is in vitro.

### Methods of Identifying CRISPR-Associated Proteins

Provided herein are methods of identifying a Clustered Regularly Interspaced Short Palindromic Repeat (CRISPR)-associated protein including (a) obtaining a plurality of genomic sequences, wherein a genomic sequence of the plurality of genomic sequences comprises a CRISPR-associated array; (b) determining a subset of the plurality of genomic sequences comprising a plurality of coding sequences within a 20 kilobase (kb) sequence flanking region either at the 3′ or 5′ end of the CRISPR-associated array; and (c) analyzing a coding sequence of the plurality of coding sequences and thereby identifying the CRISPR-associated protein based on the coding sequence.

In some embodiments, the obtaining step comprises identifying, within the plurality of genomic sequences, a genomic sequence comprising a CRISPR-associated array.

Also provided herein are methods of identifying a CRISPR-associated proteins including (a) obtaining a plurality of genomic sequences; (b) selecting, within the plurality of genomic sequences, a genomic sequence comprising a CRISPR-associated array; (c) determining a subset of the plurality of genomic sequences comprising a plurality of coding sequences within a 20 kilobase (kb) sequence flanking region either at the 3′ or 5′ end of the CRISPR-associated array; and (d) analyzing a coding sequence of the plurality of coding sequences and thereby identifying the CRISPR-associated protein based on the coding sequence.

In some embodiments, the plurality of genomic sequences comprise one or more of genomes, wherein the one or more of genomes are selected from: a prokaryotic genome and metagenome. In some embodiments, the selecting step comprises using an algorithm selected from the group consisting of PILER-CR, and CRISPR Recognition Tool (CRT), and combinations thereof.

In some embodiments, the determining step includes filtering the genomic sequences according to the location of the genomic sequence relative to the 20 kb sequence flanking region. In some embodiments, the filtering can include selecting a genomic sequence that is located within the 20 kb flanking region. In some embodiments, the determining step also includes filtering the genomic sequences according to the size of the genomic sequence. In some embodiments, the filtering can include selecting a genomic sequence that is longer than 500 amino acids. In some embodiments, the determining step comprises using an algorithm selected from the group consisting of MetaGeneMark, and Prodigal, and combinations thereof.

As used herein, the term “analyzing” can refer to a process that includes filtering of a plurality of coding sequences based on the size of each coding sequence. In some embodiments, the filtering comprises selecting a coding sequence that comprises more than 500 amino acids (e.g., 550 amino acids, 600 amino acids, 650 amino acids, 700 amino acids, 750 amino acids, or 800 amino acids). In some embodiments, the filtering comprises selecting a coding sequence that comprises more than 800 amino acids (e.g., 850 amino acids, 900 amino acids, 950 amino acids, 1000 amino acids, 1100 amino acids, 1200 amino acids, 1300 amino acids, 1400 amino acids, or 1500 amino acids).

In some embodiments, the analyzing step further comprises classifying the CRISPR-associated arrays. In some embodiments, the classifying of the CRISPR-associated arrays comprises selecting a CRISPR-associated array comprising three or more coding sequences (e.g., 4 or more, 5 or more, 6 or more, 7 or more, 8 or more, 9 or more, 10 or more, 15 or more, 20 or more, 25 or more, 30 or more, 35 or more, 40 or more, 45 or more, or 50 or more coding sequences) present in the 20 kb flanking regions. In some embodiments, the classifying further comprises determining a relative position of the coding sequence in the 20 kb flanking region relative to the CRISPR-associated array. In some embodiments, the classifying comprises calculating the coding sequence position within the 20 kb flanking region adjacent to the CRISPR-associated array, wherein the coding sequence could be classified based on the position relative to the CRISPR-associated array.

In some embodiments, the analyzing of the coding sequences comprises removing known CRISPR-associated proteins from the identified CRISPR-associated proteins. In some embodiments, the analyzing of the coding sequence comprises using one or more algorithms selected from HHMSCAN and RPS-BLAST. In some embodiments, the analyzing of the coding sequence further comprises determining the presence of a structural domain. In some embodiments, the analyzing of the coding sequence further comprises determining the presence of a functional domain. In some embodiments, the functional domain comprises a functional domain selected from a DNA binding domain, a RNA binding domain, a nuclease, a helicase, a restriction domain, and or a structural maintenance of chromosomes (SMC) domain. In some embodiments, the analyzing of the coding sequence further comprises determining whether the coding sequence starts with a Methoinine

Also provided herein are computer implemented methods including (a) obtaining a plurality of genomic sequences; (b) selecting, within the plurality of genomic sequences, a genomic sequence comprising a CRISPR-associated array; (c) determining a subset of the plurality of genomic sequences comprising a plurality of coding sequences within a 20 kilobase (kb) sequence flanking region either at the 3′ or 5′ end of the CRISPR-associated array; and (d) analyzing a coding sequence of the plurality of coding sequences and thereby identifying a CRISPR-associated protein based on the coding sequence.

### Methods of Treatment

Also provided herein are methods for treating a condition or disease in a subject in need thereof, the method including administering to the subject any of the systems described herein, wherein the spacer sequence is substantially complementary to a target nucleic acid associated with the condition or disease; wherein the CRISPR-associated protein associates with the RNA guide to form a complex; wherein the complex binds to the target nucleic acid sequence; and wherein upon binding of the complex to the target nucleic acid sequence the CRISPR-associated protein cleaves the target nucleic acid, thereby treating the condition or disease in the subject.

In some embodiments of these methods, the method can result in at least a 2.0-fold (e.g., at least a 2.5-fold, at least a 3.0-fold, at least a 3.5-fold, at least a 4.0-fold, at least a 4.5-fold, at least a 5.0-fold, at least a 6.0-fold, at least a 7.0-fold, at least a 8.0-fold, at least a 9.0-fold, at least a 10-fold, at least a 15-fold, at least a 20-fold, at least a 30-fold, at least a 40-fold, at least a 50-fold, at least a 60-fold, at least a 80-fold, at least a 100-fold, at least a 120-fold, or at least a 150-fold) decrease in the level of one or more symptoms associated with the condition or disease as compared to the level of the one or more symptoms associated with the condition in the subject prior to the administering. In some examples of these methods, the method can result from about a 2-fold to about a 150-fold, about a 2-fold to about a 100-fold, about a 2-fold to about a 50-fold, about a 2-fold to about a 25-fold, about a 2-fold to about a 10-fold, about a 2-fold to about a 5-fold, about a 5-fold to about a 150-fold, about a 5-fold to about a 100-fold, about a 5-fold to about a 50-fold, about a 5-fold to about a 25-fold, about a 5-fold to about a 10-fold, about a 10-fold to about a 150-fold, a 10-fold to about a 100-fold, about a 10-fold to about a 50-fold, about a 10-fold to about a 25-fold, about a 25-fold to about a 150-fold, about a 25-fold to about a 100-fold, or about a 25-fold to about a 50-fold, decrease in the level of one or more symptoms associated with the condition or disease as compared to the level of the one or more symptoms associated with the condition in the subject prior to the administering.

In some embodiments, the condition or disease can include conditions such as cancers, neurodegeneration, cutaneous conditions, endocrine conditions, intestinal diseases, infectious conditions, neurological conditions, liver diseases, heart disorders, or autoimmune diseases. In some embodiments, the condition or disease can be a cancer. In some embodiments, the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer. In some embodiments, the cancer can be a B-cell acute lymphoblastic meukemia, lung cancer, esophageal cancer, multiple myeloma, or cervical cancer.

In some embodiments, the condition or disease can be a neurodegenerative disease. In some embodiments, the neurodegenerative disease can be Alzheimer's disease, Huntington's disease, Duchenne muscular dystrophy (DMD), frontotemporal dementia, ryanodine receptor type I (RYR1)-related myopathies, cystic fibrosis, or autosomal recessive juvenile parkinsonism.

In some embodiments, the condition or disease can be a blood disease or a hemoglobinopathies. In some embodiments, the blood disease can be sickle cell anemia or beta thalassemia. In some embodiments, the condition or disease can be an eye disease. In some embodiments, the eye disease can be retinitis pigmentosa, leber congenital amaurosis, specific retinal dystrophy, or autosomal dominant cone-rod dystrophy. In some embodiments, the condition or disease can be human immunodeficiency virus (HIV), diabetes, autism spectrum disorder, genetic liver disease, or congenital genetic lung disease.

### EXAMPLES

### Methods

### Identification/Prediction of Candidate CRISPR Associated Proteins

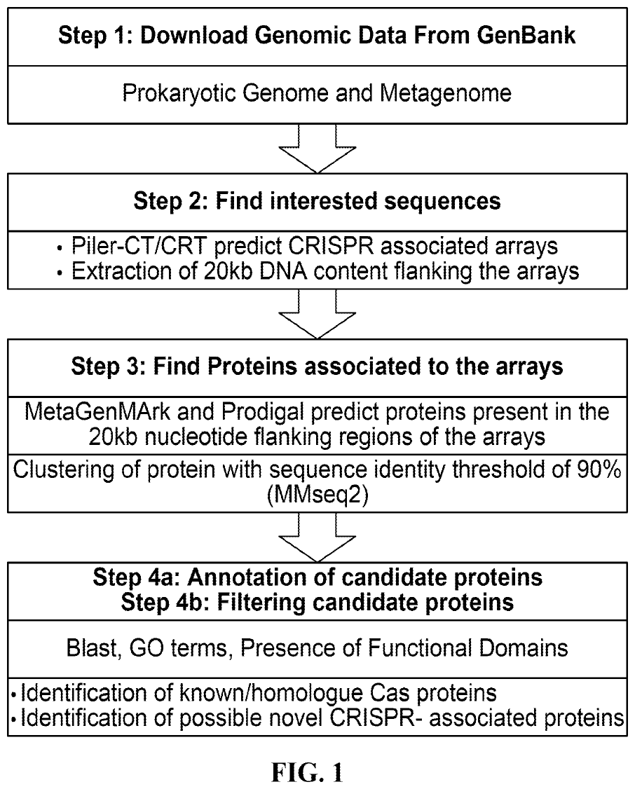

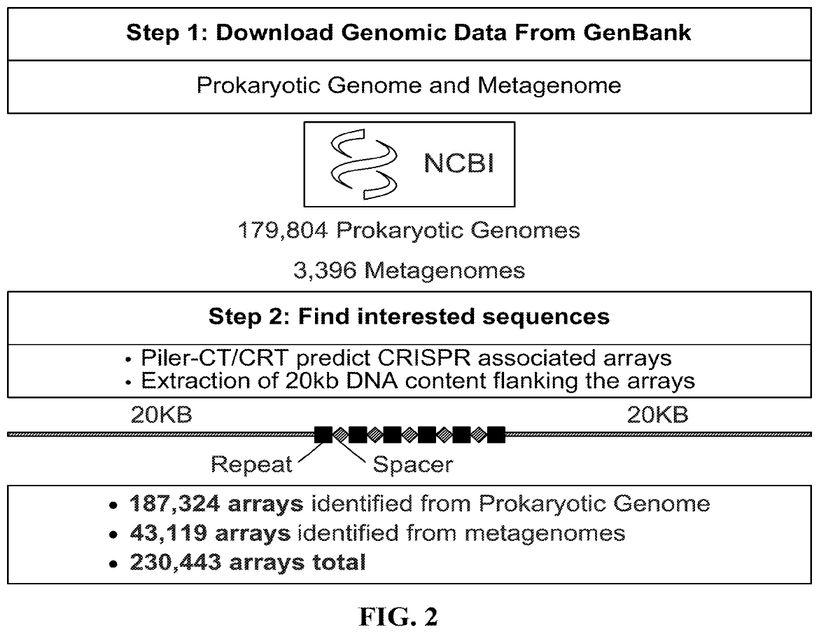

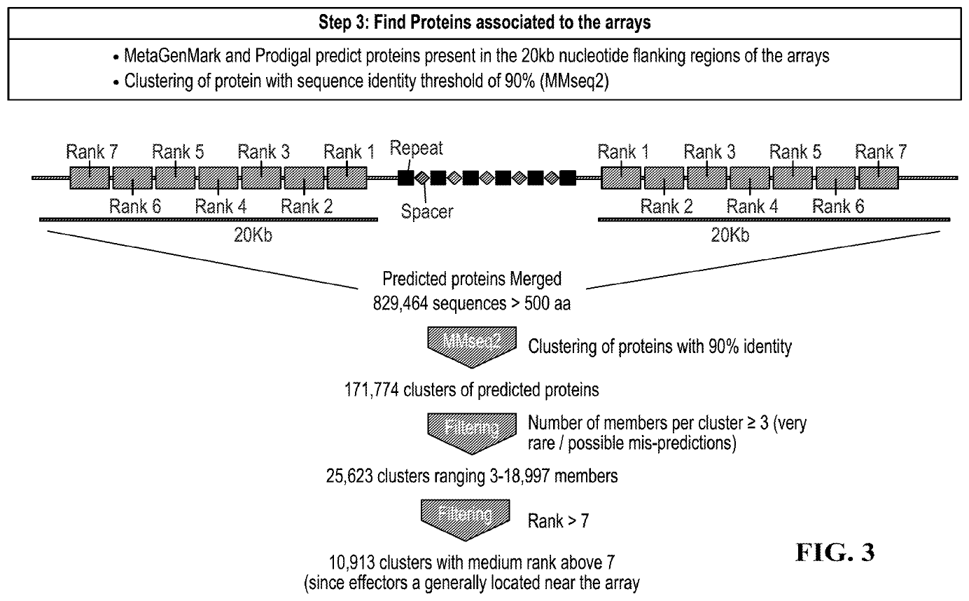

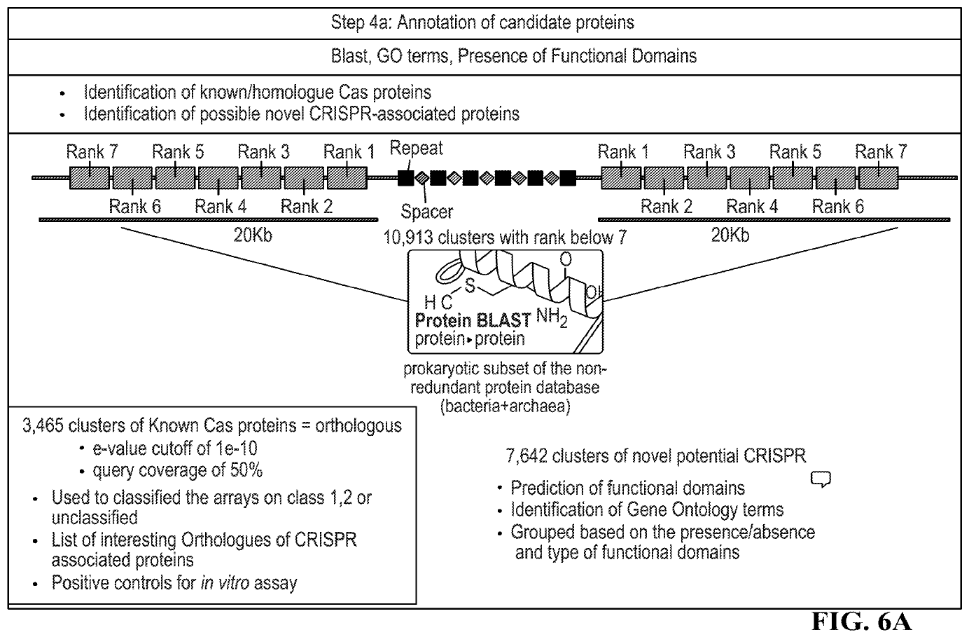

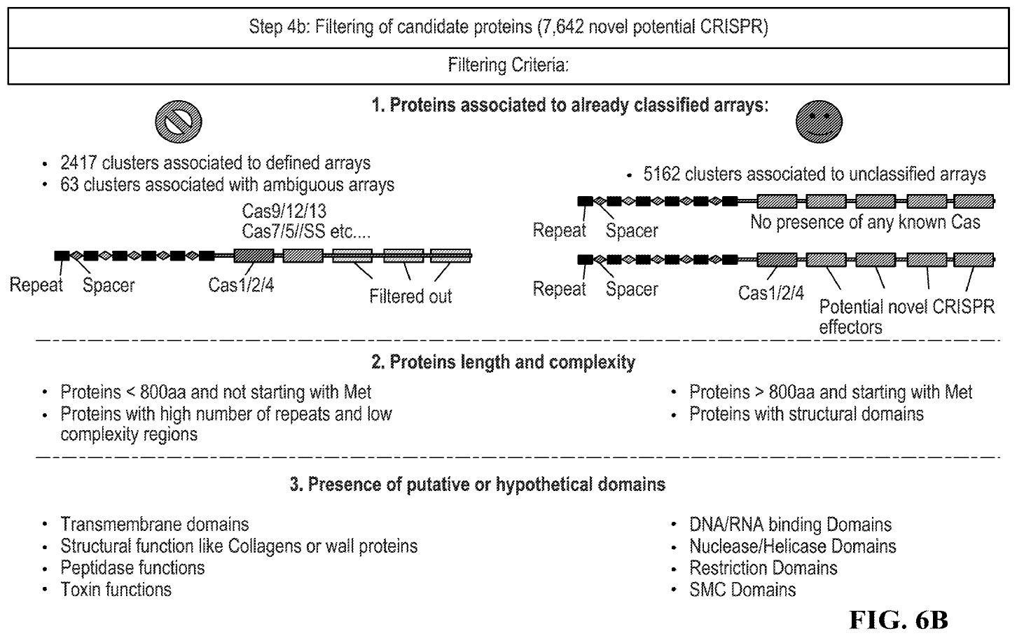

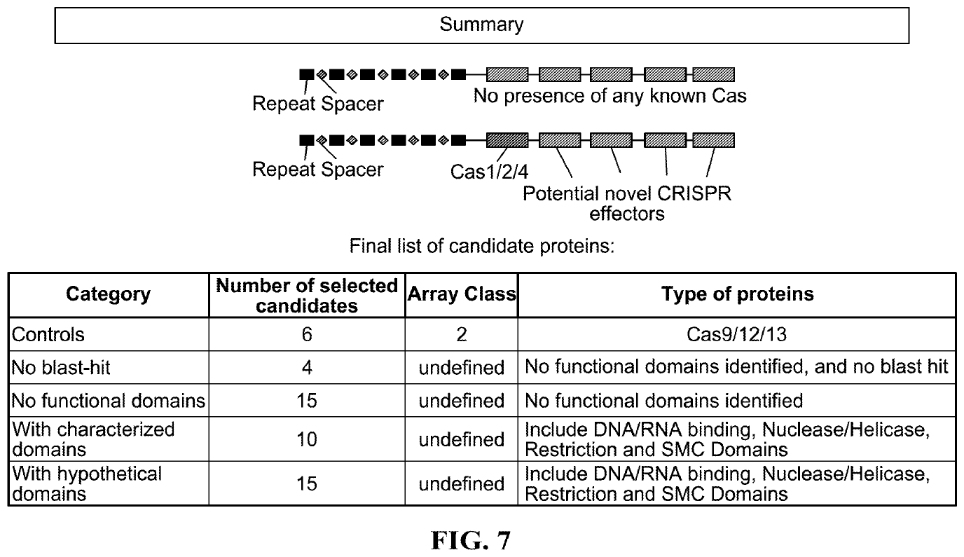

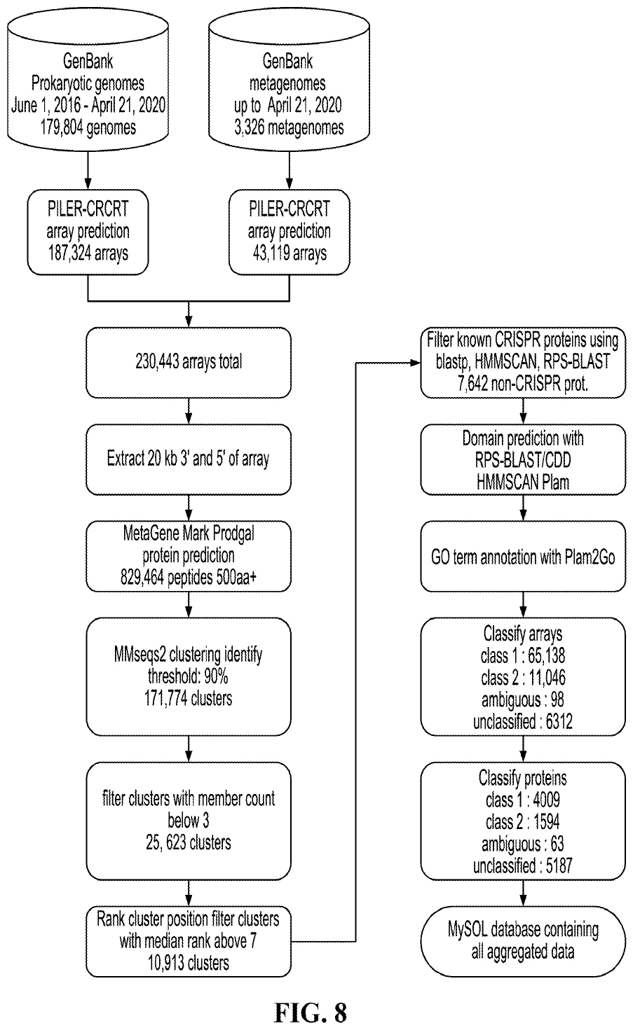

An exemplary method of identifying candidate CRISPR-association proteins is as described as shown in FIG. 1. In order to identify new candidate CRISPR associated proteins 179,804 prokaryotic genomes and 3,396 metagenomes deposited in Genbank from Jun. 1, 2016-Apr. 21, 2020 were downloaded and analyzed (FIG. 2). PILER-CR (see, e.g., Edgar et al., BMC Bioinformatics, 8, 18 (2007)) and CRT (CRISPR Recognition Tool) (see, e.g., Bland, C. et al., BMC Bioinformatics, 8, 209 (2007)) were used to identify CRISPR arrays (or “arrays”) (FIG. 2). Arrays located on sequence contigs shorter than 3 kilobases (kb) were filtered out and 20 kb flanking sequences on both sides of the arrays were extracted. As shown in FIG. 3, protein sequences were predicted from the 20 kb flanking sequences using MetaGeneMark (see, e.g., Zhu, et al., Nucleic Acids Research, 38 e132-e132 (2010); hereinafter “Zhu”) and Prodigal (see, e.g., Hyatt et al., BMC Bioinformatics, 11, 119 (2010); hereinafter “Hyatt”)). Proteins predicted from the two software were merged and sequences shorter than 500 amino acids were filtered out. Subsequently, protein sequences were clustered using MMseqs2 (see, e.g., Steinegger, Nat. Biotechnol. 35, 1026-1028 (2017)) with a sequence identity threshold of 90%. Clusters with less than 3 members were filtered out because they may represent very rare or mis-predicted sequences. For each cluster, the position of each gene (coding sequence) relative to the array was calculated. Ranks were assigned for each cluster, with rank 1 indicating the gene immediately adjacent to the array, rank 2 indicating the second gene adjacent to the array, rank 3 indicating the third gene adjacent to the array, rank 4 indicating the fourth gene adjacent to the array, rank 5 indicating the fifth gene adjacent to the array, rank 6 indicating the sixth gene adjacent to the array, and so forth. Clusters with a median rank above 7 were subsequently filtered out since known effectors are usually located in proximity to the array (FIG. 3). This analysis produced candidate clusters. FIGS. 6A and 6B shows further annotation and filtering done on the 10,913 candidate clusters. FIGS. 7 and 8 shows a summary of the method as described herein.

### Annotation/Classification of Predicted CRISPR Associated Proteins

In order to annotate and classify the 10,913 cluster sequences adjacent to the CRISPR arrays, from each cluster a representative sequence was searched against the prokaryotic subset of the non-redundant protein database (bacteria+archaea) using blastp in order to annotate protein sequences and identify known CRISPR genes. Protein sequences matching known CRISPR genes with e-value cutoff of 1e-10 and query coverage of 50% were considered orthologous to known CRISPR genes. Furthermore protein sequences were searched with HMMSCAN against known CRISPR-related profiles from (see, e.g., Burstein, D. et al., Nature 542, 237-241 (2017); hereinafter “Burstein”) and with RPS-BLAST against a collection of CRISPR profiles. These protein clusters represent orthologs and are considered known CRISPR associated proteins and thus filtered out or separated for further analysis. From the total 10,913 clusters, 3465 clusters were considered known CRISPR and 7,642 novel potential CRISPR associated candidates (FIG. 6A).

To further annotate the remaining 7,642 protein clusters, for each candidate protein, functional domains were predicted by running RPS-BLAST on CDD database and HMMSCAN against Pfam and associated GO (Gene Ontology) terms were added using Pfam2Go mapping software. Protein clusters were subsequently grouped in subsets based on the presence/absence of characterized and putative domains.

### Results

### Bioinformatic Search for Novel CRISPR Associated Proteins

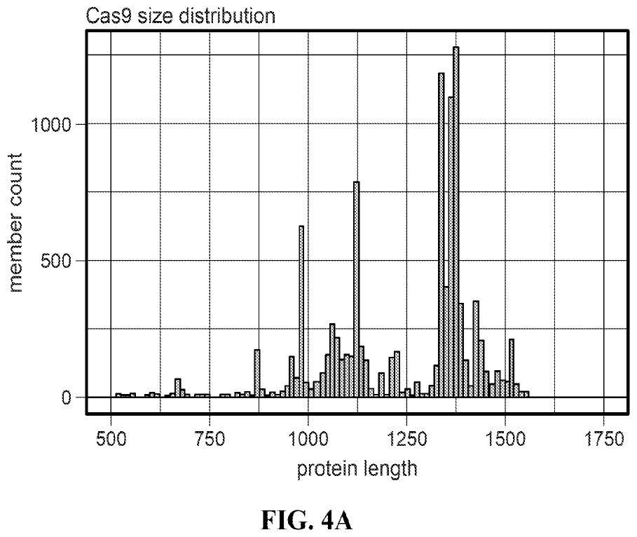

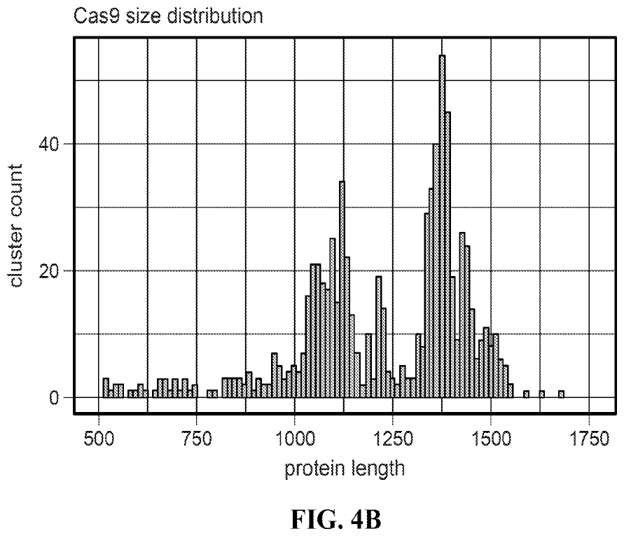

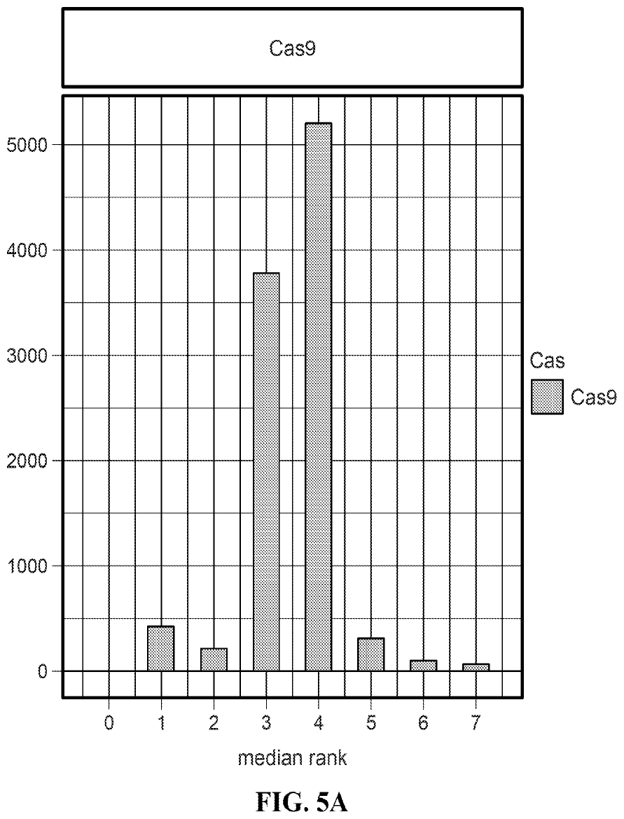

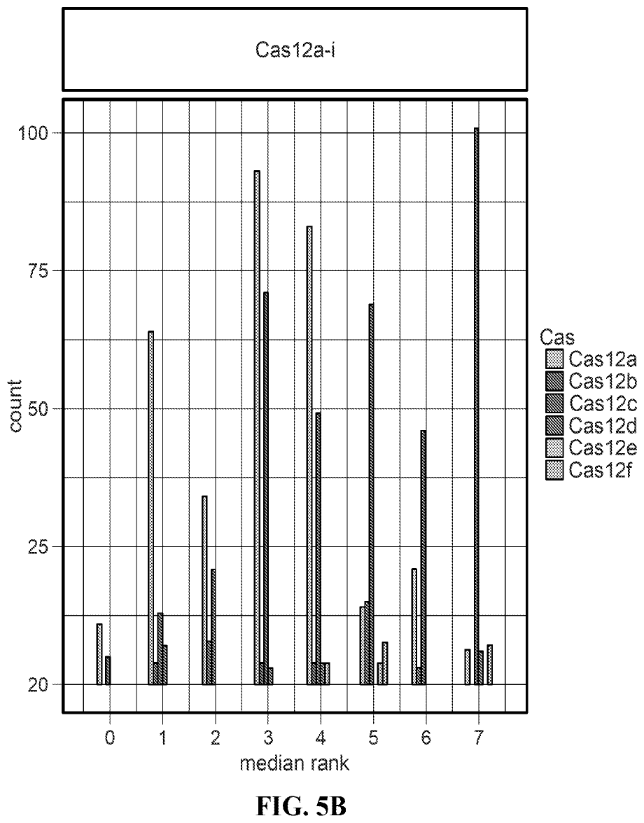

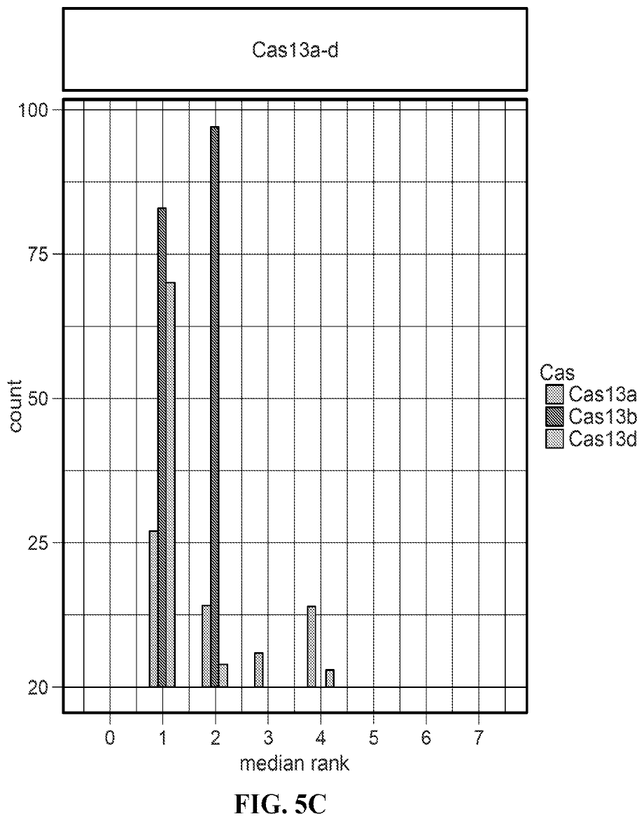

To identify novel CRISPR associated proteins, 179,804 prokaryotic genomes and 3,396 metagenomes deposited to Genbank from Jun. 1, 2016-Apr. 21, 2020 were downloaded and analyzed. Using PILER-CR and CRT (CRISPR Recognition Tool), 230,443 CRISPR arrays were identified with 187,324 derived from prokaryote genomes, and 43,119 from metagenomes. Given that most CRISPR class 2 effectors (i.e. single effector proteins like Cas9's, Cas12's, Cas13's) are located in close proximity to their arrays (Makarova, et al., Nat. Rev. Microbiology, 18: 67-83 (2020); hereinafter “Makarova”), the search for novel CRISPR associated proteins was limited to a 20 kb window flanking the arrays. Putative protein sequences within the flanking sequences were predicted using MetaGeneMark (Zhu) and Prodigial (Hyatt), filtering out sequences shorter than 500 amino acids as novel class 2 effectors are generally large multidomain proteins (Makarova). FIGS. 4A-4B show the Cas9 size distribution by member and cluster count. This prediction resulted in 829,464 total protein sequences located adjacent to the CRISPR arrays. Given that many of these are likely to be orthologous, protein sequences were clustered using MMseqs2 (Mirdita et al., Bioinformatics, 35: 2856-2858 (2019)) with sequence identity threshold set at 90% resulting in 171,774 unique clusters. Clusters with fewer than 3 members (very rare sequences or possible mis-predictions) were filtered out leaving 25,623 clusters. The number of sequences associated with each cluster ranged from 3 to 18,997 (FIG. 3). These 25,623 clusters were further analyzed to determine the position of each gene (coding sequence) relative to the array was calculated and assigned a rank within the cassette of genes based on the relative position to the array. As described above, rank 1 means that the gene is immediately adjacent to the array and rank 2 indicating the second gene adjacent to the array, and so forth. Known effectors are usually located close to the array. For instance, Cas9-type effectors are usually ranked 3-4, while Cas13-type effectors—are typically ranked 1-2, and Cas12-type effectors are more broadly distributed, but still close to the array (FIGS. 5A-5C). Filtering out all clusters with median rank above 7 reduced the cluster number to 10,913 (FIG. 3).

To annotate protein sequences and identify known CRISPR proteins, representative sequences for the 10,913 clusters were searched against the prokaryotic subset of the non-redundant protein database (bacteria+archaea) using blastp. Protein sequences matching known CRISPR genes with e-value cutoff of 1e-10 and query coverage of 50% were considered orthologous to known CRISPR genes. Additionally, protein sequences were searched with HMMSCAN against known CRISPR-related profiles (Burstein) and with RPS-BLAST against collection of CRISPR profiles. Hits for both of these searches mostly overlapped blastp-identified CRISPR sequences, with a few exceptions, which were also added to the CRISPR cluster ortholog set. Together, from the 10,913 clusters, 3465 clusters were considered orthologs to known CRISPR proteins leaving 7,642 potential cluster candidates to be further characterized. Given that many of the 10,913 clusters were generated with a stringent 90% identity using MMseqs2, these clusters were similar and therefore additional filtering was performed. To further reduce the number of sequences, 10,913 clusters can be further clustered with MMseqs2 using default settings, which requires the sequences to overlap by at least 80% (query coverage 0.8). MMseqs2 with default settings generated 4,205 “superclusters”. The supercluster classification reduced the number of known CRISPR-associated clusters to 343 and the number of unknown CRISPR superclusters to 3862. To narrow down the two lists (clusters and superclusters), proteins were further analyzed and protein domains were predicted by running RPS-BLAST on the CDD database and HMMSCAN against Pfam (FIGS. 6A-6B). Associated GO terms were added using Pfam2Go mapping.

For the 3465 clusters consisting of 51,094 orthologs of known CRIPSR proteins, and 343 superclusters consisting of 2614 clusters we found numerous class I systems which have effector modules composed of multiple Cas proteins (e.g. Cas1-4, 5-8, 10-11), and numerous class II systems which encompass a single multidomain crRNA-binding protein (e.g., Cas9, Cas12, Cas13 etc.).

### Predictions of TracR-RNAs

To annotate known candidates, the arrays were classified into class 1, 2, or unclassified based on the identified CRISPR-related proteins associated with each array. For each array with flanking regions length of at least 3 kb, all those CRISPR-related proteins were collected and if they consistently fell into class 1 or 2 that array was classified as such. If an array had no identifiable CRISPR proteins that could distinguish the class, like arrays flanked by Cas1/Cas2/Cas4 only or no Cas proteins, they were marked as unclassified. If an array had proteins from both classes, it was marked ambiguous. That is because if a cluster was classified as 2, that meant that the array already had an effector protein such as Cas9/Cas12/Cas13 since those are the only proteins that can distinguish class 2 reliably. Those arrays were unlikely to have yet another effector. If the array was classified as 1, which is the majority of classified arrays, naturally, it also could have been discarded since class 2 effector were of primary importance. As such, the aim was to narrow down the candidate CRISPR-associated proteins by further considering only unclassified or ambiguous arrays.

### Choosing the Top 50

Further filtering of the candidate clusters produced a list of 50 candidate proteins to be used for functional assay. Candidates were divided in four main categories: proteins with no blast hits, proteins with no predicted domains and blast hits against hypothetical and unknown proteins, proteins with predicted domains and blast hits against hypothetical and unknown proteins only and proteins with predicted domains and blast hits against characterized proteins. For each category protein shorter than 800 amino acids (aa) and proteins not starting with methionine (Met) were filtered out. The first category included 25 candidates, 6 are associated with classified arrays and thus not considered for further analysis. Since the majority of the proteins were filtered out because they had predicted domains with a structural potential function or were low complexity proteins including many SR repeats, the protein length threshold for this category was changed to 650 aa and four potential candidates were selected for functional analysis. The second category of proteins with no predicted domains and blast hits against hypothetical and unknown proteins contained 347 candidates of which 120 are associated with an already classified array and thus filtered out. From the remaining 227 proteins, 175 proteins were excluded for being shorter than 800 aa and 14 candidates were excluded for not starting with Met. In addition, proteins with high presence of low complexity/repeats regions were selected out and selected 15 candidates for further analysis. The third category included 1644 proteins with predicted domains and blast hits against hypothetical and unknown proteins of which only 552 candidates were longer of 800 aa. Exclusion of 152 proteins as already associated with classified arrays and proteins not starting with Met left 322 candidate proteins. From this shorter list, 15 were selected based on putative function of the hypothetical domains. Proteins with DNA/RNA binding domains, nucleases, helicases, restriction and SMC domains were included in the final list for further functional analysis. The most abundant category is represented by proteins with predicted domains and blast hits against characterized proteins with 5329 candidates of which 1442 were above 800 aa. After filtering out proteins associated with classified arrays and proteins not starting with Met, the candidate number decreased to 758. SEQ ID NOs: 1-50 represent proteins with DNA/RNA binding domains, nucleases, helicases, restriction and SMC domains that were selected for further analysis. The CRISPR arrays and spacer sequences corresponding to the CRISPR-associated proteins of SEQ ID NOs: 1-50 are listed in Tables 1-5.

TABLE 2
CRISPR arrays and spacer
sequences for candidate CRISPR-associated proteins

| | | | | | | | | CRISPR- |
|---|---|---|---|---|---|---|---|---|
| | | | | | | | | associated |
| | | | | | | | spacer sequence | protein |
| | other | | Domain | | | | (each row | Corre- |
| Protein | CAS | array | (y or | class | | | denotes a | sponding |
| ID | protein | name | n) | type | Notes | repeats | new spacer) | SEQ ID NO: |
| gene_ | cas1- | piler_ | cas9 | class 2 | Cas9- | GTTTTAG | AGAATACAACATTGTC | SEQ ID |
| 5155455\| | cas2- | crt_ | | | Streptococcus | AGCTGTG | TTAATAGGAGACAC | NO: 1 |
| GeneMark.hmm\| | cas4 | array_ | | | thermophilus | TTGTTTC | (SEQ ID NO: 101) | |
| 1389_aa\|+\| | | VBTK01000005.1_ | | | | GAATGGT | GAATCATGATTGGTTT | |
| 13650\|17819 | | 52517-54136: | | | | TCCAAAA | ATCTGTGGGCTTCA | |
| | | 41619 | | | | C | (SEQ ID NO: 102) | |
| | | | | | | (SEQ ID | AAAGAAATTAAAAAAA | |
| | | | | | | NO: 51) | CCTAGCGAAGCACT | |
| | | | | | | | (SEQ ID NO: 103) | |
| | | | | | | | TTCGCATAAGACTTCT | |
| | | | | | | | TCAAACCAAAACAT | |
| | | | | | | | (SEQ ID NO: 104) | |
| | | | | | | | GTCCATAGGTATTTCC | |
| | | | | | | | CTTTAATTAAAGT | |
| | | | | | | | (SEQ ID NO: 105) | |
| | | | | | | | TAGAGATGACGACGGA | |
| | | | | | | | CTACCTGGCAAGAA | |
| | | | | | | | (SEQ ID NO: 106) | |
| | | | | | | | TATCCCAGAGAATGGA | |
| | | | | | | | AGAACAATTATAGA | |
| | | | | | | | (SEQ ID NO: 107) | |
| | | | | | | | CTTCTTAAAATTGAAT | |
| | | | | | | | AATTCGAAGCACAT | |
| | | | | | | | (SEQ ID NO: 108) | |
| | | | | | | | AGGTAACATTGGTTCA | |
| | | | | | | | ACAGCAGTCTAATT | |
| | | | | | | | (SEQ ID NO: 109) | |
| | | | | | | | TCGTTACCTTGTCTTT | |
| | | | | | | | GCAAATCACGCAAA | |
| | | | | | | | (SEQ ID NO: 110) | |
| | | | | | | | AATGAAGAAGCCGATT | |
| | | | | | | | CAAGCTCAAGGGTC | |
| | | | | | | | (SEQ ID NO: 111) | |
| | | | | | | | TATTTCTGTCCGATAC | |
| | | | | | | | GAAGTATCAGGGAC | |
| | | | | | | | (SEQ ID NO: 112) | |
| | | | | | | | TACGCCCGTTTGGATT | |
| | | | | | | | GAACATGATAGAGC | |
| | | | | | | | (SEQ ID NO: 113) | |
| | | | | | | | GAGCCTACTAATGATT | |
| | | | | | | | ACATTTTGAGGACG | |
| | | | | | | | (SEQ ID NO: 114) | |
| | | | | | | | CCAAAGAATGGACCAC | |
| | | | | | | | CTTAATGAGAATAT | |
| | | | | | | | (SEQ ID NO: 115) | |
| | | | | | | | TTTCAAAATCTTCGAA | |
| | | | | | | | TAGGCAGTCGAGCA | |
| | | | | | | | (SEQ ID NO: 116) | |
| | | | | | | | AAAATGTACAAATTTT | |
| | | | | | | | CATGCTAGGGAATA | |
| | | | | | | | (SEQ ID NO: 117) | |
| | | | | | | | TACAGCTCTTGGTTTC | |
| | | | | | | | GTCTATCCTTATGT | |
| | | | | | | | (SEQ ID NO: 118) | |
| | | | | | | | CGCTAGGGTCTCTGGT | |
| | | | | | | | GACGCTGAGGTCTC | |
| | | | | | | | (SEQ ID NO: 119) | |
| | | | | | | | CCTGACGCATATGGAA | |
| | | | | | | | ATCCTAACGGTCAG | |
| | | | | | | | (SEQ ID NO: 120) | |
| | | | | | | | AAAATCATCTAAATAC | |
| | | | | | | | ATGTGTGTAACAAG | |
| | | | | | | | (SEQ ID NO: 121) | |
| | | | | | | | AAGCACTGGACGACAA | |
| | | | | | | | ATAAATAATTGAAG | |
| | | | | | | | (SEQ ID NO: 122) | |
| | | | | | | | GAACAAGAAACTTATG | |
| | | | | | | | AAGTCGAAAACCGA | |
| | | | | | | | (SEQ ID NO: 123) | |
| | | | | | | | TTCGCATAAGACTTCT | |
| | | | | | | | TCAAACCAAAACAT | |
| | | | | | | | (SEQ ID NO: 124) | |
| gene_ | cas1/ | piler_ | cas12b | class 2 | cas12b- | CTTTAAG | CAAACCGCCTGTTGCT | SEQ ID |
| 3815793\| | cas4- | crt_ | | | Laceyella | TGATTAG | CCCGCAACACGCATTC | NO: 2 |
| GeneMark.hmm\| | cas2 | array_ | | | sediminis | ATGAATT | GGTC | |
| 1090_aa\|+\| | | PVTZ01000002.1_ | | | | AAATGTG | (SEQ ID NO: 125) | |
| 14361\|17633 | | 339025-339866: | | | | ATTAGCA | GTGGAATCCTATTTGG | |
| | | 40841 | | | | C | CGCTTGAAGGGGACAA | |
| | | | | | | (SEQ ID | CCGC ((SEQ | |
| | | | | | | NO: 52) | ID NO: 126) | |
| | | | | | | | GCCGAAGATACCTGGT | |
| | | | | | | | GAGAAGTTTTCAGCAT | |
| | | | | | | | TCCAAATG | |
| | | | | | | | (SEQ ID NO: 127) | |
| | | | | | | | TTAACTCTATTTGATG | |
| | | | | | | | TTATTTTTAACTCTAT | |
| | | | | | | | TTGGAG | |
| | | | | | | | (SEQ ID NO: 128) | |
| | | | | | | | GGAATATCCCTTGATT | |
| | | | | | | | TCGTGGAATATTCCAC | |
| | | | | | | | GTTT | |
| | | | | | | | (SEQ ID NO: 129) | |
| | | | | | | | CCACTTTTTAAGAACA | |
| | | | | | | | TATACAAACGATCTCG | |
| | | | | | | | AAGCGG | |
| | | | | | | | (SEQ ID NO: 130) | |
| | | | | | | | GCTAACACAATCAACA | |
| | | | | | | | CGATTCCACCAACAAT | |
| | | | | | | | GGTTTTTCC | |
| | | | | | | | (SEQ ID NO: 131) | |
| | | | | | | | CCATTGATACAGGCAA | |
| | | | | | | | TCTCCATGTCTGATTT | |
| | | | | | | | GTTG | |
| | | | | | | | (SEQ ID NO: 132) | |
| | | | | | | | GGGAGATAAGGTAAAA | |
| | | | | | | | CATAGACTCCAAATAG | |
| | | | | | | | TGCT | |
| | | | | | | | (SEQ ID NO: 133) | |
| | | | | | | | TGAGTACATCGGGGGA | |
| | | | | | | | TAAAAAGCCGCATAGG | |
| | | | | | | | AATC | |
| | | | | | | | (SEQ ID NO: 134) | |
| | | | | | | | TTAACTGCCCAATTTC | |
| | | | | | | | CATTTTCCAGCTTAAC | |
| | | | | | | | GATC | |
| | | | | | | | (SEQ ID NO: 135) | |
| gene_ | cas1/ | piler_ | cas12a | class 2 | cas12 a- | GTTAAGT | ATGGCTGTCTGTATAA | SEQ ID |
| 2964877\| | cas4- | crt_ | | | Firmicutes | AACCTAA | GGTGTCTCTG | NO: 3 |
| GeneMark.hmm\| | cas2 | array_ | | | bacterium | ATAATTT | (SEQ ID NO: 136) | |
| 1305_aa\|+\| | | NALN01000012.1_ | | | | CTACTGT | TTAATTTTATTGTTGC | |
| 15109\|19026 | | 70224-71132: | | | | GTGTAGA | TGTTGTTTAGT | |
| | | 40908 | | | | T | (SEQ ID NO: 137) | |
| | | | | | | (SEQ ID | ATTTTACCGCTACAGG | |
| | | | | | | NO: 53) | AGAACACGAT | |
| | | | | | | | (SEQ ID NO: 138) | |
| | | | | | | | ATCGACAGGGATAACA | |
| | | | | | | | CAGGCATAGCT | |
| | | | | | | | (SEQ ID NO: 139) | |
| | | | | | | | CTATACGCCAGAGGGT | |
| | | | | | | | GAGCCTTGGAA | |
| | | | | | | | (SEQ ID NO: 140) | |
| | | | | | | | AAGTATTGAAAAATAT | |
| | | | | | | | CATATAGTAAT | |
| | | | | | | | (SEQ ID NO: 141) | |
| | | | | | | | CAAAATATCGATAAGG | |
| | | | | | | | CTCCAGAAGAA | |
| | | | | | | | (SEQ ID NO: 142) | |
| | | | | | | | CTATTGGGATACTCTC | |
| | | | | | | | ATTAAAAGT | |
| | | | | | | | (SEQ ID NO: 143) | |
| | | | | | | | CAAAATCTTATCTTTA | |
| | | | | | | | TCTTCTTGAG | |
| | | | | | | | (SEQ ID NO: 144) | |
| | | | | | | | TACTATGCCCGAATAT | |
| | | | | | | | TAAAAGCTGT | |
| | | | | | | | (SEQ ID NO: 145) | |
| | | | | | | | AAAATATGAAGCTCCC | |
| | | | | | | | TTACAATTTTC | |
| | | | | | | | (SEQ ID NO: 146) | |
| | | | | | | | ATAACAACCGCCTGTT | |
| | | | | | | | TAGTACTAGG | |
| | | | | | | | (SEQ ID NO: 147) | |
| | | | | | | | ATATCATTAATATGGG | |
| | | | | | | | CTGGGATACA | |
| | | | | | | | (SEQ ID NO: 148) | |
| gene_ | cas1/4 | piler_ | cas13a | class 2 | cas13a | AGTGAAA | TTTTGGAGGTCGCCTT | SEQ ID |
| 4147644\| | | crt_ | | | | GTAGCCC | TTGAAACCTTGAATCC | NO: 4 |
| GeneMark.hmm\| | | array_ | | | | GATATAG | TAAATTCCTA | |
| 1412_aa\|+\| | | QRUJ01000006.1_ | | | | AGGGCAA | (SEQ ID NO: 149) | |
| 20684\|24922 | | 107860-108175: | | | | TAAC | GTTTGGTACGGTTTTA | |
| | | 40315 | | | | (SEQ ID | TTTTCTTATAGTTTTT | |
| | | | | | | NO: 54) | ATATATATG | |
| | | | | | | | (SEQ ID NO: 150) | |
| | | | | | | | GTCATATTACAACATG | |
| | | | | | | | CTTCATACTGCTTGTC | |
| | | | | | | | ATCA | |
| | | | | | | | (SEQ ID NO: 151) | |
| | | | | | | | AAGCCAACCTAAATCA | |
| | | | | | | | ACACCATCATCATCAC | |
| | | | | | | | AAAC | |
| | | | | | | | (SEQ ID NO: 152) | |
| meta_gene_ | no | piler_ | cas13d | class 2 | CasRx | CTACTAC | TTGCAGTTTTCTTCAC | SEQ ID |
| 174274\| | | array_ | | | (From | ACTGGTG | GATACTTATCTAGCT | NO: 5 |
| GeneMark.hmm\| | | ODFV01004017.1_ | | | metsgenomes) | CGAATTT | (SEQ ID NO: 153) | |
| 921_aa\|−\| | | 2979-3331: | | | | GCACTAG | AGGTCAAGATCTGATT | |
| 66\|2831 | | 3577 | | | | TCTAAAA | TATGAATTTTGCCT | |
| | | | | | | C | (SEQ ID NO: 154) | |
| | | | | | | (SEQ ID | ATGGATTCCTCTACCT | |
| | | | | | | NO: 55) | CTTCATCTGTTACA | |
| | | | | | | | (SEQ ID NO: 155) | |
| | | | | | | | AATATTTCTTTTATAT | |
| | | | | | | | TCTTACACCCCTCGA | |
| | | | | | | | (SEQ ID NO: 156) | |
| gene_ | no | crt_ | cas13d | class 2 | * | CTACTAC | CTATGTAGCTTTTCTT | SEQ ID |
| 4200106\| | (addi- | array_ | | | | ACTGGTG | GTAAAACATATTT | NO: 6 |
| GeneMark.hmm\| | tional | QTXT01000036.1_ | | | | CGAATTT | (SEQ ID NO: 157) | |
| 568_aa\|+\| | cas13d) | 6154-6455: | | | | GCACTAG | CATCTGCCTTCTGCAT | |
| 6646\|8352 | | 16264 | | | | TCTAAAA | ATCGGACACTTGA | |
| | | | | | | CT | (SEQ ID NO: 158) | |
| | | | | | | (SEQ ID | TACACCTCCTTATGCG | |
| | | | | | | NO: 56) | ATTTTATCGTGCG | |
| | | | | | | | (SEQ ID NO: 159) | |
| | | | | | | | TAAAAATATCCTTTTT | |
| | | | | | | | GCTCATGTTCACGT | |
| | | | | | | | (SEQ ID NO: 160) | |
| meta_ | no | crt_ | n | unclas- | ** | | Not included | SEQ ID |
| gene_ | | array_ | | sified | | | | NO: 7 |
| 524079\| | | WNGK01002380.1_ | | | | | | |
| GeneMark.hmm\| | | 701-1392: | | | | | | |
| 759_aa\|+\| | | 21392 | | | | | | |
| 4762\|7041 | | | | | | | | |
| meta_ | | meta_ | n | unclas- | | GTCGCTA | GAAACTTGTGAGCTTC | SEQ ID |
| crt_ | | crt_ | | sified | | ATGGAGC | CATGAAACCGAATAAG | NO: 8 |
| array_ | | array_ | | | | GGCTTCT | TACTTA | |
| WNGG01011662.1 | | WNGG01011662.1_ | | | | CGGTTGA | (SEQ ID NO: 161) | |
| | | 9582-9832: | | | | GATT | GAAACATTCCCATCAC | |
| | | 9827 | | | | (SEQ ID | CCTCGATATCAAAGCC | |
| | | | | | | NO: 57) | ATAATCAT | |
| | | | | | | | (SEQ ID NO: 162) | |
| | | | | | | | GAAACCCGTTTAGCTT | |
| | | | | | | | GATACGAGAAGCCCCT | |
| | | | | | | | CGGCTTTA | |
| | | | | | | | (SEQ ID NO: 163) | |
| meta_ | no | piler_ | n | unclas- | | ATAAAGA | ACTCCAACATAACCTC | SEQ ID |
| gene_ | | crt_ | | sified | | ATTAACA | TTAAGTACTTAAAATC | NO: 9 |
| 336895\| | | array_ | | | | TAAGTTG | TTCTTT | |
| GeneMark.hmm\| | | OEIL01000106.1_ | | | | TTTTTAA | (SEQ ID NO: 164) | |
| 727_aa\|+\| | | 29209-29855: | | | | AT | TTCTTTTTGTCAATAT | |
| 10145\|12328 | | 40646 | | | | (SEQ ID | TTCTAAATTTATATTT | |
| | | | | | | NO: 58) | TCTT | |
| | | | | | | | (SEQ ID NO: 165) | |
| | | | | | | | AAAAGTGGATTATCTC | |
| | | | | | | | CACTGGAAGTGGTACT | |
| | | | | | | | CAA | |
| | | | | | | | (SEQ ID NO: 166) | |
| | | | | | | | GGTGTTCCTTTTTTGT | |
| | | | | | | | ATTGATTTCTTTTATT | |
| | | | | | | | TATT | |
| | | | | | | | (SEQ ID NO: 167) | |
| | | | | | | | AAAAGAAGAATTACAT | |
| | | | | | | | TTAAATTTTAAGA | |
| | | | | | | | (SEQ ID NO: 168) | |
| | | | | | | | ACTGTAACTCGATTTT | |
| | | | | | | | TTAAAAATATTTTTAC | |
| | | | | | | | TTC | |
| | | | | | | | (SEQ ID NO: 169) | |
| | | | | | | | AAAATGTGAGATAATT | |
| | | | | | | | TATACGAATTATTTT | |
| | | | | | | | (SEQ ID NO: 170) | |
| | | | | | | | ATTCCAGTTTTAAAAT | |
| | | | | | | | TCTTTCCTATTGGGAC | |
| | | | | | | | ACC | |
| | | | | | | | (SEQ ID NO: 171) | |
| | | | | | | | AGAGGTATTGGAAAAT | |
| | | | | | | | A | |
| | | | | | | | (SEQ ID NO: 172) | |
| meta_ | no | crt_ | n | unclas- | | | Not included | SEQ ID |
| gene_ | | array_ | | sified | | | | NO: 10 |
| 321445\| | | OEEO01000863.1_ | | | | | | |
| GeneMark.hmm\| | | 7543-7748: | | | | | | |
| 675_aa\|−\| | | 15683 | | | | | | |
| 5020\|7047 | | | | | | | | |

* short version casRx ([Ruminococcus sp.)
** crispr software failed to recognize array and spacer_only repeats not spacer

TABLE 3
CRISPR arrays and spacer sequences for candidate CRISPR-associated proteins

| | | | | | | | | | CRISPR- |
|---|---|---|---|---|---|---|---|---|---|
| | | | | | | | | | asso- |
| | | | | | | | | | ciated |
| | | | | | | | | | protein |
| | | | | | | | | spacer sequence | Corre- |
| | other | | | Domain | | | | (each row | sponding |
| Protein | CAS | tracr | array | (y or | class | | | denotes a | SEQ |
| ID | protein | RNA | name | n) | type | Notes | repeats | new spacer) | ID NO: |
| gene_ | cas2- | no | piler_ | y | unclas- | (Actino | GTCGGCCC | TGTTGAACGACCCTGA | SEQ ID |
| 3820393\| | cas3- | | crt_ | | sified | corallia | CGGGGATG | GGCCACGCAGCTGCAG | NO: 11 |
| GeneMark.hmm\| | cse1/ | | array_ | | | populi) | CGCACGCG | (SEQ ID NO: 173) | |
| 1351_aa\|+\| | CasA | | PVZV01000003.1_ | | | 3 arrays | TTCCG | ATCGACGCCAGCGACA | |
| 23286\|27341 | | | 163001-165104: | | | across | (SEQ ID | TCGGCTGGGTCCAGGC | |
| | | | 42103 | | | the | NO: 59) | (SEQ ID NO: 174) | |
| | | | | | | 40 Kb | | GTGAACATCGGCGGGA | |
| | | | | | | sequences | | TCACGATCAAGCGGGA | |
| | | | | | | | | (SEQ ID NO: 175) | |
| | | | | | | | | TGGCTGAGCGGACCGT | |
| | | | | | | | | CGAGGCCGGGGCGTCC | |
| | | | | | | | | (SEQ ID NO: 176) | |
| | | | | | | | | GGTTACGAGGTCGGGG | |
| | | | | | | | | GGGGGCCTTGAGCAG | |
| | | | | | | | | (SEQ ID NO: 177) | |
| | | | | | | | | TCCAGGCGACATTACG | |
| | | | | | | | | CCCGTTGCGGCCGATC | |
| | | | | | | | | (SEQ ID NO: 178) | |
| | | | | | | | | TCATGGGGCCAAGCCA | |
| | | | | | | | | AGAAAAGGGGCGATTA | |
| | | | | | | | | (SEQ ID NO: 179) | |
| | | | | | | | | TACCTGGGCGGGCGCG | |
| | | | | | | | | CGGCCCGAGCTGAGAA | |
| | | | | | | | | (SEQ ID NO: 180) | |
| | | | | | | | | CCCACGGGCGGACCCA | |
| | | | | | | | | TCGGAAGGCGCCTTCG | |
| | | | | | | | | (SEQ ID NO: 181) | |
| | | | | | | | | CGGCCAGCTCAGCCCC | |
| | | | | | | | | GGTGCCGCTGGTCTCC | |
| | | | | | | | | (SEQ ID NO: 182) | |
| | | | | | | | | TGCTCACCGCCTACGC | |
| | | | | | | | | GATGGATCCTGAACGC | |
| | | | | | | | | (SEQ ID NO: 183) | |
| | | | | | | | | AAGCCGGCGCCGAAGG | |
| | | | | | | | | TCGCGGGGATCGGCGC | |
| | | | | | | | | (SEQ ID NO: 184) | |
| | | | | | | | | AACTGCAGCGACTCAT | |
| | | | | | | | | CGACGAACAGGCAGGT | |
| | | | | | | | | (SEQ ID NO: 185) | |
| | | | | | | | | CGGTTCTCGTTCATCG | |
| | | | | | | | | TTCGGTCCTCTTCTTG | |
| | | | | | | | | (SEQ ID NO: 186) | |
| | | | | | | | | GGCGCACCGATGCCCC | |
| | | | | | | | | AGCAGCTCACCGACGA | |
| | | | | | | | | (SEQ ID NO: 187) | |
| | | | | | | | | GATTGTGTAGGCCCCC | |
| | | | | | | | | GGCACCTACAGAACCC | |
| | | | | | | | | (SEQ ID NO: 188) | |
| | | | | | | | | GTGTCTCCTACTGGTC | |
| | | | | | | | | CGGGTCGGGGAAGAGC | |
| | | | | | | | | G | |
| | | | | | | | | (SEQ ID NO: 189) | |
| | | | | | | | | CTGGAGGTCATCGCCG | |
| | | | | | | | | CCGAGGTCGCCGAGTT | |
| | | | | | | | | (SEQ ID NO: 190) | |
| | | | | | | | | CCGACCAGGCTGGCCA | |
| | | | | | | | | GGGCGCCGAGGGAGAC | |
| | | | | | | | | (SEQ ID NO: 191) | |
| | | | | | | | | GAGTTGTAGCTCTCGA | |
| | | | | | | | | TCTCGCCGAGCACGTT | |
| | | | | | | | | (SEQ ID NO: 192) | |
| | | | | | | | | CTGTTCGTGGAGCGCT | |
| | | | | | | | | CGAGCTGGGCGTGACC | |
| | | | | | | | | (SEQ ID NO: 193) | |
| | | | | | | | | AAGGCCGGGCTTCAGC | |
| | | | | | | | | GCTACGGCCGGTACCT | |
| | | | | | | | | (SEQ ID NO: 194) | |
| | | | | | | | | ATGATGGAGCTGGTCG | |
| | | | | | | | | CCCAGCTCTCCCCCGC | |
| | | | | | | | | (SEQ ID NO: 195) | |
| | | | | | | | | CACGCCCTCTGATCCC | |
| | | | | | | | | GACACCAAGGAGAGAC | |
| | | | | | | | | (SEQ ID NO: 196) | |
| | | | | | | | | TCATGGATGTCCGTCC | |
| | | | | | | | | GCTGGGTGGGGCCGCT | |
| | | | | | | | | (SEQ ID NO: 197) | |
| | | | | | | | | GCGGGCTACGAGATCG | |
| | | | | | | | | ACGGCGAGACCGTCGA | |
| | | | | | | | | (SEQ ID NO: 198) | |
| | | | | | | | | GGGCGCGCCAGTACGC | |
| | | | | | | | | GCGCGGCATCGTGGCG | |
| | | | | | | | | (SEQ ID NO: 199) | |
| | | | | | | | | CGTGCCGGGTGGTGGT | |
| | | | | | | | | GTCGACCGTGCCGTCG | |
| | | | | | | | | (SEQ ID NO: 200) | |
| | | | | | | | | ATCTTCGGGGCGGCGG | |
| | | | | | | | | GCGCCGAGGGCGGCGG | |
| | | | | | | | | (SEQ ID NO: 201) | |
| | | | | | | | | TCCCCGAACTCCAGCA | |
| | | | | | | | | GCCGGTGGATTCTGGC | |
| | | | | | | | | (SEQ ID NO: 202) | |
| | | | | | | | | GAGGCGCAGCTCGCCT | |
| | | | | | | | | ATGAGCAGGCGGTGCA | |
| | | | | | | | | (SEQ ID NO: 203) | |
| | | | | | | | | CGGAACTTCTTCCTCA | |
| | | | | | | | | ACAGCGCGGAGCCAGG | |
| | | | | | | | | (SEQ ID NO: 204) | |
| | | | | | | | | GTCGAGCTTGACAAGC | |
| | | | | | | | | AGAACCAGCCCCAGGG | |
| | | | | | | | | (SEQ ID NO: 205) | |
| | | | | | | | | CTGTCCAACGGCGAGT | |
| | | | | | | | | ACGTGCTGCCCGCCAA | |
| | | | | | | | | (SEQ ID NO: 206) | |
| meta_gene_ | | | piler_ | y | unclas- | | GTCGCTCC | ATCTACTGCAACGCTT | SEQ ID |
| 180752\| | | | crt_array | | sified | | CCTCGCGG | TTAACAAGATCGCTGA | NO: 12 |
| GeneMark.hmm\| | | | ODGV01001911.1_ | | | | GAGCGTGG | TT | |
| 827_aa\|−\| | | | 1-300:12864 | | | | ATTGAAAT | (SEQ ID NO: 207) | |
| 3170\|5653 | | | | | | | (SEQ ID | TTAGTTCTCTGTGAAC | |
| | | | | | | | NO: 60) | AACAAGTGTCATCTCA | |
| | | | | | | | | CTT | |
| | | | | | | | | (SEQ ID NO: 208) | |
| | | | | | | | | GATTATTGCTGATATA | |
| | | | | | | | | GTACAAGAAGCGTTTT | |
| | | | | | | | | GCA | |
| | | | | | | | | (SEQ ID NO: 209) | |
| | | | | | | | | CAAGCGTGGTACTTGG | |
| | | | | | | | | GAGATCGACAAAAAGA | |
| | | | | | | | | TCT | |
| | | | | | | | | (SEQ ID NO: 210) | |
| gene_ | no | no | piler_ | y | unclas- | | GTCACGCC | AACCCCGATGGGAAGG | SEQ ID |
| 771418\| | | | crt_array_ | | sified | | TTATGGAG | TCCTGCCGCTCTGGCT | NO: 13 |
| GeneMark.hmm\| | | | CABJCG010000021.1_ | | | | GCGTGTGG | GC | |
| 1452_ | | | 2381-2613: | | | | ATTGAAAT | (SEQ ID NO: 211) | |
| aa\|−\| | | | 22613 | | | | (SEQ ID | TTCCTGCGGTTCTGGC | |
| 2711\|7069 | | | | | | | NO: 61) | GGAGACCAGATCAAGT | |
| | | | | | | | | TCGT | |
| | | | | | | | | (SEQ ID NO: 212 | |
| | | | | | | | | GTAAGCTGTCAGGAGA | |
| | | | | | | | | TATGGTGCGAGTGTTT | |
| | | | | | | | | CGG | |
| | | | | | | | | (SEQ ID NO: 213) | |
| | | | | | | | | CGACAGCTGCGCCGCG | |
| | | | | | | | | GGCAAGTGCAAGGGCG | |
| | | | | | | | | GCAACGCGCTACT | |
| | | | | | | | | (SEQ ID NO: 214) | |
| gene_ | no | no | piler_ | y_ | unclas- | | GTCACAGT | GCTATAGTGTCCGGTT | SEQ ID |
| 1433645\| | | | crt_array_ | topoiso | sified | | GAGATCAG | TCCCGTTTTTTCCGAT | NO: 14 |
| GeneMark.hmm\| | | | DCOL01000139.1_ | meraes | | | CCGTTCAG | TT | |
| 1422_aa\|+\| | | | 10233-10618: | | | | GCTGTTGA | (SEQ ID NO: 215) | |
| 5489\|9757 | | | 10617 | | | | AAC | AACCATGCTACCGCAC | |
| | | | | | | | (SEQ ID | AGGGTGGATAATATTT | |
| | | | | | | | NO: 62) | TG | |
| | | | | | | | | (SEQ ID NO: 216) | |
| | | | | | | | | CTTTGTGGTTGCCAAG | |
| | | | | | | | | CTCACTACTTGCGCTG | |
| | | | | | | | | C | |
| | | | | | | | | (SEQ ID NO: 217) | |
| | | | | | | | | ACCACCGCGCTTGAAC | |
| | | | | | | | | GCGGGAAAATTCGTTC | |
| | | | | | | | | TGGCTAT | |
| | | | | | | | | (SEQ ID NO: 218) | |
| | | | | | | | | CACCATACGGTGCCAG | |
| | | | | | | | | AATCCGTATAGGACAC | |
| | | | | | | | | TGG | |
| | | | | | | | | (SEQ ID NO: 219) | |
| gene_ | WYL | | piler_ | y | unclas- | crispr | TAACTAAG | CCAGTGCTTCATGGTT | SEQ ID |
| 4426209\| | | | array_ | | sified | software | TTGGAAAC | AATGAAGGCAGCAGAT | NO: 15 |
| GeneMark.hmm\| | | | RQNV01000008.1_ | | | failed | T | TTGG | |
| 1255_aa\|+\| | | | 159035-159166: | | | to | (SEQ ID | (SEQ ID NO: 220) | |
| 28994\|32761 | | | 40131 | | | recognize | NO: 63) | | |
| | | | | | | array | | | |
| | | | | | | and | | | |
| | | | | | | spacer | | | |
| gene_ | | | | y | unclas- | | GACTAAAT | GCATCTGATTCATTCT | SEQ ID |
| 5411831\| | | | | | sified | | CCAAGTAG | CATATTTTGAACTTCT | NO: 16 |
| GeneMark.hmm\| | | | | | | | ATTGGAAT | AATTC | |
| 1213_aa\|+\| | | | | | | | TTTAAC | (SEQ ID NO: 221) | |
| 12801\|16442 | | | | | | | (SEQ ID | TGAAAAACTTCCAAAC | |
| | | | | | | | NO: 64) | ACGCTGACAAAGGAGC | |
| | | | | | | | | AACTA | |
| | | | | | | | | (SEQ ID NO: 222) | |
| | | | | | | | | ATCGAAAATTTTACGT | |
| | | | | | | | | TAAGAGAGCTTTCTGG | |
| | | | | | | | | AAAGA | |
| | | | | | | | | (SEQ ID NO: 223) | |
| | | | | | | | | AACTCAGGAAATCAAC | |
| | | | | | | | | GTCAGGAACTAAACGG | |
| | | | | | | | | AAAA | |
| | | | | | | | | (SEQ ID NO: 224) | |
| | | | | | | | | GCAACTCCTCTAACAT | |
| | | | | | | | | CGCCCCTAATTTCACA | |
| | | | | | | | | CGA | |
| | | | | | | | | (SEQ ID NO: 225) | |
| | | | | | | | | TCGGCAGTTCGGGACG | |
| | | | | | | | | CCTTAAAAGAAGCGGG | |
| | | | | | | | | AAAT | |
| | | | | | | | | (SEQ ID NO: 226) | |
| | | | | | | | | AATGTAGCCTTAATTC | |
| | | | | | | | | TCCATGATCGCCATAC | |
| | | | | | | | | TCTA | |
| | | | | | | | | (SEQ ID NO: 227) | |
| | | | | | | | | TTTTATCGATTCTCAT | |
| | | | | | | | | CACAATTTGAGCAACA | |
| | | | | | | | | TCTT | |
| | | | | | | | | (SEQ ID NO: 228) | |
| gene_ | | | | y | unclas- | | ATTTAAAT | TGGCCTAGCATGGCAG | SEQ ID |
| 941761\| | | | | | sified | | ACATCCTA | CTAGGAAAAATAAACT | NO: 17 |
| GeneMark.hmm\| | | | | | | | TGTTATGG | T | |
| 1123_aa\|−\| | | | | | | | TTCAATCA | (SEQ ID NO: 229) | |
| 22964\|26335 | | | | | | | (SEQ ID | CCTACAGATGTGCAAA | |
| | | | | | | | NO: 65) | ATGGTCTAAATAAAAT | |
| | | | | | | | | ATA | |
| | | | | | | | | (SEQ ID NO: 230) | |
| gene_ | | | | y | unclas- | | GTAGCATT | CTCCCCTGTGTCGGTT | SEQ ID |
| 1546948\| | | | | | sified | | CACCCCCA | CATCGCCCGTGGCGGG | NO: 18 |
| GeneMark.hmm\| | | | | | | | AGGGTGGG | AGTT | |
| 949_aa\|−\| | | | | | | | TGCCCGTT | (SEQ ID NO: 231) | |
| 10158\|13007 | | | | | | | GAAAC | GAAACTGCTATCGCTA | |
| | | | | | | | (SEQ ID | TTGCGTCGGTTTTTGT | |
| | | | | | | | NO: 66) | CATACGCTTA | |
| | | | | | | | | (SEQ ID NO: 232) | |
| | | | | | | | | CTCCCCTGTGTCGGTT | |
| | | | | | | | | CATCGCCCGTGGCGGG | |
| | | | | | | | | AGTT | |
| | | | | | | | | (SEQ ID NO: 233) | |
| meta_gene_ | | | | y | unclas- | | GTTTCAGA | CGTCAATTTCGGGCGT | SEQ ID |
| 15450\| | | | | | sified | | GCAGATGC | GAAGAATCGCGGGATA | NO: 19 |
| GeneMark.hmm\| | | | | | | | TGGCTTGA | TAGGC | |
| 803_aa\|+\| | | | | | | | GTTAAGAT | (SEQ ID NO: 234) | |
| 14847\|17258 | | | | | | | GTAAC | CGCGACGCGCAACATA | |
| | | | | | | | (SEQ ID | ACGCTCCAGTGCTTCG | |
| | | | | | | | NO: 67) | TTGT | |
| | | | | | | | | (SEQ ID NO: 235) | |
| | | | | | | | | GCGAGGGCCAGAAGGC | |
| | | | | | | | | CCAGAAAAACGAGAGT | |
| | | | | | | | | GCC | |
| | | | | | | | | (SEQ ID NO: 236) | |
| | | | | | | | | CCGGCGGCCACACGCT | |
| | | | | | | | | GGCGGATTTCTTCTAC | |
| | | | | | | | | CA | |
| | | | | | | | | (SEQ ID NO: 237) | |
| | | | | | | | | ACAAAGACTGGCTACG | |
| | | | | | | | | AGAAGGCGATTGAATG | |
| | | | | | | | | CGT | |
| | | | | | | | | (SEQ ID NO: 238) | |
| | | | | | | | | AGTACGACCCGCACGC | |
| | | | | | | | | TTGGAACAAATACCCC | |
| | | | | | | | | G | |
| | | | | | | | | (SEQ ID NO: 239) | |
| | | | | | | | | TGAAGGCTGTCCGCCT | |
| | | | | | | | | GCGCCCCATTCCCATG | |
| | | | | | | | | CA | |
| | | | | | | | | (SEQ ID NO: 240) | |
| | | | | | | | | CATCAAAAACTGGTCA | |
| | | | | | | | | TCCTGCACCGTTTCCT | |
| | | | | | | | | GAT | |
| | | | | | | | | (SEQ ID NO: 241) | |
| meta_gene_ | | | | y | unclas- | | GTGCTCCC | GGGCTTGGGGGCGTAG | SEQ ID |
| 73412\| | | | | | sified | | CGCTCAGG | AAGGGATCGCCGTGGC | NO: 20 |
| GeneMark.hmm\| | | | | | | | CGGGGGTG | (SEQ ID NO: 242) | |
| 804_aa\|−\| | | | | | | | ATCCC | TCCAGGCCTACGAGGC | |
| 16541\|18955 | | | | | | | (SEQ ID | TGAGGAGTCCGCGAAG | |
| | | | | | | | NO: 68) | (SEQ ID NO: 243) | |
| | | | | | | | | TGCCCGGCGTCCAACC | |
| | | | | | | | | GCGGCCCGTAGATCAC | |
| | | | | | | | | (SEQ ID NO: 244) | |
| | | | | | | | | GGCATGACGTACGAGG | |
| | | | | | | | | AGATCGGGCAAGAGGC | |
| | | | | | | | | (SEQ ID NO: 245) | |
| | | | | | | | | GGGCTGGCCCCACGCC | |
| | | | | | | | | ACCTCGTGCGTCACTG | |
| | | | | | | | | (SEQ ID NO: 246) | |

TABLE 4
CRISPR arrays and spacer sequences for candidate CRISPR-associated proteins

| | | | | | | | | | CRISPR- |
|---|---|---|---|---|---|---|---|---|---|
| | | | | | | | | | asso- |
| | | | | | | | | | ciated |
| | | | | | | | | | protein |
| | | | | | | | | spacer sequence | Corre- |
| | other | | | Domain | | | | (each row | sponding |
| Protein | CAS | tracr | array | (y or | class | | | denotes a | SEQ |
| ID | protein | RNA | name | n) | type | Notes | repeats | new spacer) | ID NO: |
| gene_307407\| | | | | Hipo- | unclas- | | GGGAA | CCGCACCCTGACCACC | SEQ ID |
| GeneMark.hmm\| | | | | thetical | sified | | CACCCC | GGGGCCGCCGGGCAGC | NO: 21 |
| 1697_aa\|+\| | | | | | | | CGCACG | (SEQ ID NO: 247) | |
| 14906\|19999 | | | | | | | CGCGGG | GACGAGGACCGGTATC | |
| | | | | | | | GACCAC | CCGCTGCCTGGGGAGT | |
| | | | | | | | (SEQ ID | (SEQ ID NO: 248) | |
| | | | | | | | NO: 69) | AACGGGTCGATCACGG | |
| | | | | | | | | ATGTGGCGACCCGGCC | |
| | | | | | | | | (SEQ ID NO: 249) | |
| | | | | | | | | GCGGTCCAGGTCGGGC | |
| | | | | | | | | GGCAGGTCGTACATGC | |
| | | | | | | | | (SEQ ID NO: 250) | |
| | | | | | | | | TATGGCGACATGTCTG | |
| | | | | | | | | CGTCGTTGGCGGCCGA | |
| | | | | | | | | (SEQ ID NO: 251) | |
| | | | | | | | | CCGCACTCCGACTACC | |
| | | | | | | | | CGACCGAGTGGCGCCA | |
| | | | | | | | | (SEQ ID NO: 252) | |
| | | | | | | | | GAGGCCCCCTCGGGCA | |
| | | | | | | | | GTGCCCCTCAGGCCAC | |
| | | | | | | | | (SEQ ID NO: 253) | |
| | | | | | | | | CAGCCCGGCCCGGGGG | |
| | | | | | | | | AGGAGGAGGCGCGGGC | |
| | | | | | | | | GC | |
| | | | | | | | | (SEQ ID NO: 254) | |
| | | | | | | | | GCCGCAGTCCAGCCCG | |
| | | | | | | | | GCCCCGACGGCGGATG | |
| | | | | | | | | (SEQ ID NO: 255) | |
| | | | | | | | | CAGGACACCACCTCGT | |
| | | | | | | | | CCTGCCGGGGCTTTCC | |
| | | | | | | | | (SEQ ID NO: 256) | |
| | | | | | | | | CAGCCGGGACAGCGGG | |
| | | | | | | | | GCCGGCCGGGCGCCCG | |
| | | | | | | | | (SEQ ID NO: 257) | |
| | | | | | | | | GGAGCACGCCCGATGA | |
| | | | | | | | | CCACCCCGCACGACCA | |
| | | | | | | | | (SEQ ID NO: 258) | |
| | | | | | | | | CCACCCCTCCACCGTG | |
| | | | | | | | | GCGCACCGGACAGCCC | |
| | | | | | | | | (SEQ ID NO: 259) | |
| | | | | | | | | GTCATCGTGCCCCTGC | |
| | | | | | | | | CCCCTGAGGGCCTCGC | |
| | | | | | | | | (SEQ ID NO: 260) | |
| | | | | | | | | GAGGTGGTCGCCCTCC | |
| | | | | | | | | GCGCCCAGCTCGCCCC | |
| | | | | | | | | (SEQ ID NO: 261) | |
| | | | | | | | | TGGGAGCTGATGCGGT | |
| | | | | | | | | CCCGGATGCCTGGCCG | |
| | | | | | | | | (SEQ ID NO: 262) | |
| gene_1432510\| | | | | Hipo- | unclas- | | CATAAG | TATTCACTTTTTGTGA | SEQ ID |
| GeneMark.hmm\| | | | | thetical | sified | | TCTTTT | TGATCTGCGGAGAGAT | NO: 22 |
| 1564_aa\|+\| | | | | | | | GTGGAT | GTTCTGGCGGT | |
| 27392\|32086 | | | | | | | GAGCTG | (SEQ ID NO: 263) | |
| | | | | | | | TGGAGG | TATTGTGGCAGACTGC | |
| | | | | | | | GACGCA | GAATGTTTTTGGAGGG | |
| | | | | | | | CTGGCA | GGAGGGGGT | |
| | | | | | | | GT | (SEQ ID NO: 264) | |
| | | | | | | | (SEQ ID | CTATGTGAGTGGCAAC | |
| | | | | | | | NO: 70) | AAGTATCTTGGTGCAG | |
| | | | | | | | | GGACGCAGAC | |
| | | | | | | | | (SEQ ID NO: 265) | |
| | | | | | | | | ACAACGAGGAACTTGA | |
| | | | | | | | | TCGTGGAGG | |
| | | | | | | | | (SEQ ID NO: 266) | |
| | | | | | | | | AAAATGAGAAGCTTGA | |
| | | | | | | | | TCGTGAAGG | |
| | | | | | | | | (SEQ ID NO: 267) | |
| | | | | | | | | ACAATGTGCCCAAATA | |
| | | | | | | | | AAATAACTGACGCAGA | |
| | | | | | | | | GTGTTCTGCGAAAT | |
| | | | | | | | | (SEQ ID NO: 268) | |
| | | | | | | | | GTTTTCTGTAGTAGGT | |
| | | | | | | | | TCCTTTCTATGACGAA | |
| | | | | | | | | ATAATGGTTTGGTGAG | |
| | | | | | | | | AG | |
| | | | | | | | | (SEQ ID NO: 269) | |
| | | | | | | | | ATCTCGTATCTAAAGC | |
| | | | | | | | | AAGACAGATCATGTGG | |
| | | | | | | | | AGTGTTTTGTGAGAT | |
| | | | | | | | | (SEQ ID NO: 270) | |
| | | | | | | | | TTCTTCTGTAGTGGGG | |
| | | | | | | | | GCCTTATTGTGACGAA | |
| | | | | | | | | AGAATTGTTCGGCTAG | |
| | | | | | | | | AG | |
| | | | | | | | | (SEQ ID NO: 271) | |
| | | | | | | | | TGTTATGGAGAGGAGC | |
| | | | | | | | | ATGGGG | |
| | | | | | | | | (SEQ ID NO: 272) | |
| gene_5570191\| | | | | Hipo- | unclas- | | AGCTCG | AGCTCGTGCACCGTCA | SEQ ID |
| GeneMark.hmm\| | | | | thetical | sified | | TGCACC | GCCGATAGAGCACCAG | NO: 23 |
| 1502_aa\|−\| | | | | | | | GTCAGC | GTCTTCCGGCCGA | |
| 1126\|5634 | | | | | | | CGATAG | (SEQ ID NO: 273) | |
| | | | | | | | AGCACC | GCGGGCTTGTCCAGGG | |
| | | | | | | | AGGTCT | ATATCCAGTTGCGGCG | |
| | | | | | | | TCCGGC | GTTCGGG | |
| | | | | | | | CGA | (SEQ ID NO: 274) | |
| | | | | | | | (SEQ ID | TCGGTTATTTCGCAGT | |
| | | | | | | | NO: 71) | CCGGCCGGGCGGCTTC | |
| | | | | | | | | CTGCACTGAA | |
| | | | | | | | | (SEQ ID NO: 275) | |
| | | | | | | | | AACATGCTTGAACCGT | |
| | | | | | | | | CTGGCATAGACCGCTA | |
| | | | | | | | | CAGGGGTCACC | |
| | | | | | | | | (SEQ ID NO: 276) | |
| | | | | | | | | ACCCTAAACCAGTAGC | |
| | | | | | | | | GCACTTCGGACGTCGT | |
| | | | | | | | | GTAGTGGATGC | |
| | | | | | | | | (SEQ ID NO: 277) | |
| gene_2435065\| | | | | Hipo- | unclas- | | TCTTTG | TCCTTGACGGCGAGGT | SEQ ID |
| GeneMark.hmm\| | | | | thetical | sified | | ACCGGC | CGGCACAGACCAGCAC | NO: 24 |
| 1265_aa\|+\| | | | | | | | AGGTCA | CCCTCGAT | |
| 13005\|16802 | | | | | | | CATCGG | (SEQ ID NO: 278) | |
| | | | | | | | ACGGCG | | |
| | | | | | | | CACAAC | | |
| | | | | | | | C | | |
| | | | | | | | (SEQ ID | | |
| | | | | | | | NO: 72) | | |
| meta_ | | | | Hipo- | unclas- | | | Not included | SEQ ID |
| gene_343942\| | | | | thetical | sified | | | | NO: 25 |
| GeneMark.hmm\| | | | | | | | | | |
| 1220_aa\|−\| | | | | | | | | | |
| 15010\|18672 | | | | | | | | | |
| gene_1456430\| | | | | Hipo- | unclas- | | GATTTA | GATCTTTCTTCCGGCG | SEQ ID |
| GeneMark.hmm\| | | | | thetical | sified | | AAGGA | TTTCAACGCTCAAGGA | NO: 26 |
| 1196_aa\|+\| | | | | | | | CGGCGC | CGGCTCT | |
| 19091\|22681 | | | | | | | GGACA | (SEQ ID NO: 279) | |
| | | | | | | | AATTAA | ACGCTTGCATCTGGCG | |
| | | | | | | | AAGAC | CATCACAGTTAAAGGG | |
| | | | | | | | GGCTCC | CGGTTCC | |
| | | | | | | | GCGGAC | (SEQ ID NO: 280) | |
| | | | | | | | CTCAAA | | |
| | | | | | | | GACGG | | |
| | | | | | | | GACG | | |
| | | | | | | | (SEQ ID | | |
| | | | | | | | NO: 73) | | |
| gene_317827\| | | | | Hipo- | unclas- | | CGATAA | CCTTCAGCAAAACGAA | SEQ ID |
| GeneMark.hmm\| | | | | thetical | sified | | GCATGT | TCATCTAAAAGTCGC | NO: 27 |
| 1089_aa\|−\| | | | | | | | GAGTGA | (SEQ ID NO: 281) | |
| 7063\|10332 | | | | | | | GACATC | CCTCATTTACCACTAT | |
| | | | | | | | CCGAAT | AACCGTACAAAATTA | |
| | | | | | | | A | (SEQ ID NO: 282) | |
| | | | | | | | (SEQ ID | CTCCATCTCTATCAAT | |
| | | | | | | | NO: 74) | AACAAATTTATTATA | |
| | | | | | | | | (SEQ ID NO: 283) | |
| | | | | | | | | CCGTGGCATTACCACT | |
| | | | | | | | | CGTACAGACTCTGAG | |
| | | | | | | | | (SEQ ID NO: 284) | |
| | | | | | | | | CGTTCATCGTTCAGAC | |
| | | | | | | | | AATCTGTCGATTGCT | |
| | | | | | | | | (SEQ ID NO: 285) | |
| | | | | | | | | ATGGCCGTGGCTTACA | |
| | | | | | | | | AGATTCTGCCGTGGC | |
| | | | | | | | | (SEQ ID NO: 286) | |
| | | | | | | | | TAAACTGGCACAAAAT | |
| | | | | | | | | GTAGTTATGTATTGA | |
| | | | | | | | | (SEQ ID NO: 287) | |
| | | | | | | | | TACAACGCCGCAATCG | |
| | | | | | | | | GACACACACATAGTG | |
| | | | | | | | | (SEQ ID NO: 288) | |
| | | | | | | | | ACCTGACCACAATCAA | |
| | | | | | | | | GAGTTATTGAGCTTG | |
| | | | | | | | | (SEQ ID NO: 289) | |
| | | | | | | | | GGTCATGAATGGATCG | |
| | | | | | | | | CAGTTCCTCAACCGC | |
| | | | | | | | | (SEQ ID NO: 290) | |
| | | | | | | | | TCGAATCCCACCCCAG | |
| | | | | | | | | CCGCCACACTCAGCA | |
| | | | | | | | | (SEQ ID NO: 291) | |
| gene_4421494\| | | | | Hipo- | unclas- | | GTTTAG | AATTAATACTTGTTCA | SEQ ID |
| GeneMark.hmm\| | | | | thetical | sified | | AACCTT | ACCATGTCAAACCGAA | NO: 28 |
| 1044_aa\|+\| | | | | | | | AATCCC | CTTCGTTGCT | |
| 24202\|27336 | | | | | | | CGTAAG | (SEQ ID NO: 292) | |
| | | | | | | | GGGAC | AGGGTAGTCTTTCCCT | |
| | | | | | | | GGAAA | CGATAGCAAAAAGTTC | |
| | | | | | | | C | CGA | |
| | | | | | | | (SEQ ID | (SEQ ID NO: 293) | |
| | | | | | | | NO: 75) | TTAATGTCGCTAAAAT | |
| | | | | | | | | TGGGCTCTTCGGCCTG | |
| | | | | | | | | A | |
| | | | | | | | | (SEQ ID NO: 294) | |
| gene_3011455\| | | | | Hipo- | unclas- | | AACCTA | Not included | SEQ ID |
| GeneMark.hmm\| | | | | thetical | sified | | CCGTCT | | NO: 29 |
| 1037_aa\|+\| | | | | | | | TGGCTA | | |
| 19556\|22669 | | | | | | | GCGGTT | | |
| | | | | | | | GCAGCG | | |
| | | | | | | | AAC | | |
| | | | | | | | (SEQ ID | | |
| | | | | | | | NO: 76) | | |
| gene_2590511\| | | | | Hipo- | unclas- | | CCGTCA | GGAACAATCTTGCAAA | SEQ ID |
| GeneMark.hmm\| | | | | thetical | sified | | AACAGC | GGCTGTGAAAGTTGG | NO: 30 |
| 979_aa\|−\| | | | | | | | AGTTTA | (SEQ ID NO: 295) | |
| 30548\|33487 | | | | | | | ATAATG | TTCACAGGTAACATAC | |
| | | | | | | | CGTGGA | TCCACCCACCA | |
| | | | | | | | AAGAA | (SEQ ID NO: 296) | |
| | | | | | | | AA | | |
| | | | | | | | (SEQ ID | | |
| | | | | | | | NO: 77) | | |
| meta_ | | | | Hipo- | unclas- | | ATGGAC | GGGTGATACCCTCAAA | SEQ ID |
| gene_463174\| | | | | thetical | sified | | ATCCAA | TTTGTCAGCTTGAAAG | NO: 31 |
| GeneMark.hmm\| | | | | | | | CAATAA | AGCTGG | |
| 896_aa\|+\| | | | | | | | AACCAC | (SEQ ID NO: 297) | |
| 10631\|13321 | | | | | | | AAGCCA | TGATGCTTAAAGCCTG | |
| | | | | | | | TTATA | CCATAATGCAGGTATT | |
| | | | | | | | (SEQ ID | CATACA | |
| | | | | | | | NO: 78) | (SEQ ID NO: 298) | |
| | | | | | | | | TATAATCTGGACATAC | |
| | | | | | | | | TTTGAAGATTTAGCCA | |
| | | | | | | | | TGCA | |
| | | | | | | | | (SEQ ID NO: 299) | |
| | | | | | | | | TAGGTGTAGCATTGGC | |
| | | | | | | | | GTCCTCTCACGCAAAA | |
| | | | | | | | | CAGCCGC | |
| | | | | | | | | (SEQ ID NO: 300) | |
| | | | | | | | | GTAGCAGTCAAATTTC | |
| | | | | | | | | CTTTAGGGGGTTCAAG | |
| | | | | | | | | ATAAG | |
| | | | | | | | | (SEQ ID NO: 301) | |
| | | | | | | | | CCTTGATGAGTTCACG | |
| | | | | | | | | TGGAAAACCCCAGCCG | |
| | | | | | | | | ATCTGCA | |
| | | | | | | | | (SEQ ID NO: 302) | |
| | | | | | | | | AATATAAGACATTCGT | |
| | | | | | | | | GATAACGTCTTATGGC | |
| | | | | | | | | GTTATC | |
| | | | | | | | | (SEQ ID NO: 303) | |
| | | | | | | | | AGGCGTCGAATATAAA | |
| | | | | | | | | ACTTTCGTGATAACGT | |
| | | | | | | | | CTTACG | |
| | | | | | | | | (SEQ ID NO: 304) | |
| gene_773846\| | | | | Hipo- | unclas- | | TCAGTT | GAACAAATAATATCAC | SEQ ID |
| GeneMark.hmm\| | | | | thetical | sified | | GTGCTG | TTTCATATAGTTTTCC | NO: 32 |
| 887_aa\|+\| | | | | | | | TGTCGG | ATT | |
| 3216\|5879 | | | | | | | TCATGC | (SEQ ID NO: 305) | |
| | | | | | | | GGCACC | TGATTTACAGCCATTC | |
| | | | | | | | GC | TTTGATAAAGCAATAG | |
| | | | | | | | (SEQ ID | AA | |
| | | | | | | | NO: 79) | (SEQ ID NO: 306) | |
| | | | | | | | | AAAGAAGTACGAAAAT | |
| | | | | | | | | CTGTTATGAAATTAAA | |
| | | | | | | | | TT | |
| | | | | | | | | (SEQ ID NO: 307) | |
| | | | | | | | | AAACTAGCAGATGTCT | |
| | | | | | | | | TTGGTGTAACTACTGA | |
| | | | | | | | | T | |
| | | | | | | | | (SEQ ID NO: 308) | |
| | | | | | | | | ATTTTTGCTGTATAAT | |
| | | | | | | | | ATAAGTGAAGTGAGGT | |
| | | | | | | | | GA | |
| | | | | | | | | (SEQ ID NO: 309) | |
| | | | | | | | | AGGTCAAGGGATTTAT | |
| | | | | | | | | GAGAGGAAAAGGCAAT | |
| | | | | | | | | AT | |
| | | | | | | | | (SEQ ID NO: 310) | |
| | | | | | | | | ATTGTCTAACATCTTA | |
| | | | | | | | | CCAACGTCTGCTCCGT | |
| | | | | | | | | T | |
| | | | | | | | | (SEQ ID NO: 311) | |
| | | | | | | | | TTTCAATACTAAAATT | |
| | | | | | | | | TCGGGTATTTCCATCA | |
| | | | | | | | | A | |
| | | | | | | | | (SEQ ID NO: 312) | |
| | | | | | | | | GGAGATAGTAAGGAAG | |
| | | | | | | | | TTGCACAGGCATTAGA | |
| | | | | | | | | A | |
| | | | | | | | | (SEQ ID NO: 313) | |
| gene_1188229\| | | | | Hipo- | unclas- | | | TGAATGCGCCAGCCGC | SEQ ID |
| GeneMark.hmm\| | | | | thetical | sified | | | TGCCGCCGGATGCACC | NO: 33 |
| 840_aa\|+\| | | | | | | | | (SEQ ID NO: 314) | |
| 13070\|15592 | | | | | | | | TCGATAACGCCCGGTA | |
| | | | | | | | | AATACGTGTCAACTAA | |
| | | | | | | | | (SEQ ID NO: 315) | |
| | | | | | | | | GCGCTTCCCATCGCAC | |
| | | | | | | | | AGCGCACGGCGCTTCC | |
| | | | | | | | | (SEQ ID NO: 316) | |
| | | | | | | | | GTGACACGCTGTGACA | |
| | | | | | | | | ACCCCACTTTCCCAGC | |
| | | | | | | | | (SEQ ID NO: 317) | |
| | | | | | | | | CAGCACAATAAATCCC | |
| | | | | | | | | CTTGACAGCCCCCTCG | |
| | | | | | | | | (SEQ ID NO: 318) | |
| | | | | | | | | TTTGCGGTATACGACG | |
| | | | | | | | | CCGCGACCGGCGGAAA | |
| | | | | | | | | (SEQ ID NO: 319) | |
| | | | | | | | | GGTGATTTTATTCAAA | |
| | | | | | | | | AAAAAGAGAGAGGTGA | |
| | | | | | | | | (SEQ ID NO: 320) | |
| | | | | | | | | CGCGACCGCGCCATCA | |
| | | | | | | | | ATTTTGTTCTCGTTGC | |
| | | | | | | | | (SEQ ID NO: 321) | |
| | | | | | | | | GGTTCGGGGGGTTCGT | |
| | | | | | | | | GGTGGAGTGCAACCGC | |
| | | | | | | | | (SEQ ID NO: 322) | |
| | | | | | | | | TTATCGGAGAGCAGCA | |
| | | | | | | | | AGAGTTTGTCGATGAT | |
| | | | | | | | | (SEQ ID NO: 323) | |
| | | | | | | | | ATTTCTGGCGTCGGGC | |
| | | | | | | | | TCTGCTCTCAAGTGGA | |
| | | | | | | | | (SEQ ID NO: 324) | |
| | | | | | | | | GCCGCTACGGCAATTA | |
| | | | | | | | | AAAAGGTTTTCACCAC | |
| | | | | | | | | (SEQ ID NO: 325) | |
| | | | | | | | | AGCCCCAATTTTTTTA | |
| | | | | | | | | GTGACGCAAAGCCTCG | |
| | | | | | | | | (SEQ ID NO: 326) | |
| | | | | | | | | GCCTTTAACCGTTACG | |
| | | | | | | | | ATCCCGGCCGGTCGTG | |
| | | | | | | | | (SEQ ID NO: 327) | |
| | | | | | | | | TTGAAAATATTGTTGC | |
| | | | | | | | | TGCGTGTTTTTGTGTG | |
| | | | | | | | | (SEQ ID NO: 328) | |
| gene_800233\| | | | | Hipo- | unclas- | UPI000C9AE9FB | GTTTCA | CCCCATCGCCTGAAGC | SEQ ID |
| GeneMark.hmm\| | | | | thetical | sified | | ATCCAC | ACGGGCCCTACCATCT | NO: 34 |
| 838_aa\|−\| | | | | | | | GCACTC | C | |
| 23798\|26314 | | | | | | | GTGAGA | (SEQ ID NO: 329) | |
| | | | | | | | GTGCGA | GGCATCAAGGCTTCCG | |
| | | | | | | | C | GTGCGTCCTCCTGGTG | |
| | | | | | | | (SEQ ID | GA | |
| | | | | | | | NO: 80) | (SEQ ID NO: 330) | |
| | | | | | | | | GAGGCTGGGGGGACAA | |
| | | | | | | | | CTCCGAGTTTTGCGGC | |
| | | | | | | | | CA | |
| | | | | | | | | (SEQ ID NO: 331) | |
| | | | | | | | | TCTAACCTGCTGGCAA | |
| | | | | | | | | TCAAAGACGCCTTGCG | |
| | | | | | | | | CG | |
| | | | | | | | | (SEQ ID NO: 332) | |
| | | | | | | | | GCACGATCTCGGAGAA | |
| | | | | | | | | TGGGATAGCGAAAAGA | |
| | | | | | | | | A | |
| | | | | | | | | (SEQ ID NO: 333) | |
| | | | | | | | | GGGTGAAACATCCGGG | |
| | | | | | | | | ATTTATCGCTTATTGG | |
| | | | | | | | | ACG | |
| | | | | | | | | (SEQ ID NO: 334) | |
| | | | | | | | | TGACGCCAAGGGCCGC | |
| | | | | | | | | CCGCAGTGCAAATTAG | |
| | | | | | | | | TG | |
| | | | | | | | | (SEQ ID NO: 335 | |
| | | | | | | | | AGAAAAGAGGGAATGG | |
| | | | | | | | | TTCAGCCCGAAAGATG | |
| | | | | | | | | TT | |
| | | | | | | | | (SEQ ID NO: 336) | |
| | | | | | | | | TTTGATTTCCAAGGCG | |
| | | | | | | | | CGAAGGTAGCCGGATT | |
| | | | | | | | | CC | |
| | | | | | | | | (SEQ ID NO: 337) | |
| | | | | | | | | CTGGCAAACGGCCAGG | |
| | | | | | | | | TGGCCCAGGCGGCGGA | |
| | | | | | | | | CG | |
| | | | | | | | | (SEQ ID NO: 338) | |

TABLE 5
CRISPR arrays and spacer sequences for candidate CRISPR-associated proteins

| | | | | | | | | | CRISPR- |
|---|---|---|---|---|---|---|---|---|---|
| | | | | | | | | | asso- |
| | | | | | | | | | ciated |
| | | | | | | | | | protein |
| | | | | | | | | spacer sequence | Corre- |
| | other | | | Domain | | | | (each row | sponding |
| Protein | CAS | tracr | array | (y or | class | | | denotes a | SEQ |
| ID | protein | RNA | name | n) | type | Notes | repeats | new spacer) | ID NO: |
| gene_5543656\| | | | | n | unclas- | | 7 | CCGCCCGCCGATCTGG | SEQ ID |
| GeneMark.hmm\| | | | | | sified | | GTGGTCCC | AAACGGCCGGGCAGCA | NO: 36 |
| 1679_aa\|−\| | | | | | | | CGCGCGTG | (SEQ ID NO: 339) | |
| 20468\|25507 | | | | | | | CGGGGGTG | AGTTGCTGCAGGACCC | |
| | | | | | | | GTCCC | GCATGAACATCGCCGC | |
| | | | | | | | (SEQ ID | (SEQ ID NO: 340) | |
| | | | | | | | NO: 81) | CATGACGGGGTCGGTC | |
| | | | | | | | | CGGACGATCATGACGG | |
| | | | | | | | | (SEQ ID NO: 341) | |
| | | | | | | | | GGGTGGCCCTCGCTTC | |
| | | | | | | | | GTTGTGCGGACCATAC | |
| | | | | | | | | (SEQ ID NO: 342) | |
| | | | | | | | | CGTGCCGGGTCAGCTC | |
| | | | | | | | | GCCTCGGTGCACCCAG | |
| | | | | | | | | (SEQ ID NO: 343) | |
| | | | | | | | | TTCATCGCGGGCGGCG | |
| | | | | | | | | CGATCCGGACGAGCAT | |
| | | | | | | | | (SEQ ID NO: 344) | |
| gene_3943627\| | | | | n | unclas- | | 4 | CCGAGCCGACGTCGCG | SEQ ID |
| GeneMark.hmm\| | | | | | sified | | GTGGTCCC | GCGATGCTCCGCGCAG | NO: 37 |
| 1660_aa\|−\| | | | | | | | CGCGCGTG | (SEQ ID NO: 345) | |
| 25075\|30057 | | | | | | | CGGGGGTG | CCGGGTCGTCGACAAG | |
| | | | | | | | TTCCC | CCAGCCGACGAGCAGG | |
| | | | | | | | (SEQ ID | (SEQ ID NO: 346) | |
| | | | | | | | NO: 82) | GCGGAGCAGTGCGGGC | |
| | | | | | | | | TCGGCGGCATGATCAT | |
| | | | | | | | | (SEQ ID NO: 347) | |
| gene_5085315\| | | | | n | unclas- | | 4 | GATTCCCACTTTTGTC | SEQ ID |
| GeneMark.hmm\| | | | | | sified | | CTCCGAGA | TTTCCACATATAGCCT | NO: 38 |
| 1043_aa\|+\| | | | | | | | CCATCCTCC | GTG | |
| 31940\|35071 | | | | | | | ACTAAAAC | (SEQ ID NO: 348) | |
| | | | | | | | AAGGATTA | GTTTCGATTGTGAACT | |
| | | | | | | | AGAC | CGATACGCGGATTTTC | |
| | | | | | | | (SEQ ID | CTTGTC | |
| | | | | | | | NO: 83) | (SEQ ID NO: 349) | |
| | | | | | | | | CCCCCTCTATAATTAC | |
| | | | | | | | | TATAGATTTGGATGGG | |
| | | | | | | | | GCGAT | |
| | | | | | | | | (SEQ ID NO: 350) | |
| gene_4028206\| | | | | n | unclas- | | 3 reverse | TAACATGAGTGACTAT | SEQ ID |
| GeneMark.hmm\| | | | | | sified | | GGTACAGA | GGCGCTGACTTTCTGA | NO: 39 |
| 986_aa\|+\| | | | | | | | CGAACCCT | CGG | |
| 15028\|17988 | | | | | | | TGTGGGAT | (SEQ ID NO: 351) | |
| | | | | | | | TGAAGC | CTCGAAGGCGCGCCGA | |
| | | | | | | | (SEQ ID | TCGACGACGGCGAAGG | |
| | | | | | | | NO: 84) | GGCG | |
| | | | | | | | | (SEQ ID NO: 352) | |
| gene_1961732\| | | | | n | unclas- | | 4 | CTGATCGCCGTAGGTG | SEQ ID |
| GeneMark.hmm\| | | | | | sified | | GTCACCGA | AGCAGCTTCAGCGTAT | NO: 41 |
| 838_aa\|−\| | | | | | | | CCACGATC | CCTCG | |
| 1836\|4352 | | | | | | | CACCAGAA | (SEQ ID NO: 353) | |
| | | | | | | | CAAGGATT | CGGAGTTCAATGTGTG | |
| | | | | | | | GAAAC | GGCGGTCCTTGAACTT | |
| | | | | | | | (SEQ ID | CCAC | |
| | | | | | | | NO: 85) | (SEQ ID NO: 354) | |
| | | | | | | | | CAATTCTGTTCGCCCA | |
| | | | | | | | | ATCCGGCGAACTGTAC | |
| | | | | | | | | CAAAC | |
| | | | | | | | | (SEQ ID NO: 355) | |
| gene_2755817\| | | | | n | unclas- | | 4 | GTACGACCGGGAATTC | SEQ ID |
| GeneMark.hmm\| | | | | | sified | | GTCAGAAA | GACAGCTGAGGCACGG | NO: 42 |
| 816_aa\|+\| | | | | | | | GCACCCAG | CCA | |
| 11462\|13912 | | | | | | | CACCAGAA | (SEQ ID NO: 356) | |
| | | | | | | | GGTGCATT | GTGTTCTCCTGGGCGG | |
| | | | | | | | AAGAC | AGAGCACCGATAGCAG | |
| | | | | | | | (SEQ ID | TGTCG | |
| | | | | | | | NO: 86) | (SEQ ID NO: 357) | |
| | | | | | | | | TTCCAGATTTAAATGC | |
| | | | | | | | | ACGCATCAACCTACGA | |
| | | | | | | | | TA | |
| | | | | | | | | (SEQ ID NO: 358) | |
| gene_2831443\| | | | | n | unclas- | | 8 reverse | AATAAAGATATCCGCA | SEQ ID |
| GeneMark.hmm\| | | | | | sified | | GTCGCTCCT | AATCTGTCGGCCTTAA | NO: 43 |
| 802_aa\|+\| | | | | | | | TGTACGGG | G | |
| 17489\|19897 | | | | | | | AGCGTGGA | (SEQ ID NO: 359) | |
| | | | | | | | TTGAAAC | GGTACTGGTGGAGGTT | |
| | | | | | | | (SEQ ID | TATTACTAGGAAGCGC | |
| | | | | | | | NO: 87) | AAG | |
| | | | | | | | | (SEQ ID NO: 360) | |
| | | | | | | | | CGTTCGGATCGATGGT | |
| | | | | | | | | AAAGACCTGAGTTCGG | |
| | | | | | | | | CC | |
| | | | | | | | | (SEQ ID NO: 361) | |
| | | | | | | | | TAAGGAGGTAACGGAC | |
| | | | | | | | | TAATGCCTTTCATCGA | |
| | | | | | | | | CA | |
| | | | | | | | | (SEQ ID NO: 362) | |
| | | | | | | | | TAGATCCAAAATATTA | |
| | | | | | | | | CACGACACGATTCGAC | |
| | | | | | | | | A | |
| | | | | | | | | (SEQ ID NO: 363) | |
| | | | | | | | | GACTGTACAAGGAATT | |
| | | | | | | | | AGGTAATGCTTTTGAA | |
| | | | | | | | | G | |
| | | | | | | | | (SEQ ID NO: 364) | |
| | | | | | | | | TATATTATCCCTAATC | |
| | | | | | | | | AAGAAGCTAAAGCTGC | |
| | | | | | | | | C | |
| | | | | | | | | (SEQ ID NO: 365) | |
| meta_ | | | | n | unclas- | | 4 | CCGAGCCGACGTCGCG | SEQ ID |
| gene_118560\| | | | | | sified | | GTGGTCCC | GCGATGCTCCGCGCAG | NO: 44 |
| GeneMark.hmm\| | | | | | | | CGCGCGTG | (SEQ ID NO: 366) | |
| 1958_aa\|+\| | | | | | | | CGGGGGTG | CCGGGTCGTCGACAAG | |
| 6937\|12813 | | | | | | | TTCCC | CCAGCCGACGAGCAGG | |
| | | | | | | | (SEQ ID | (SEQ ID NO: 367) | |
| | | | | | | | NO: 88) | GCGGAGCAGTGCGGGC | |
| | | | | | | | | TCGGCGGCATGATCAT | |
| | | | | | | | | (SEQ ID NO: 368) | |
| meta_ | | | | n | unclas- | | 3 | GGTACCAAAGGCGTTA | SEQ ID |
| gene_324030\| | | | | | sified | | GTTTTGGA | TGATACGTAGCCATGG | NO: 45 |
| GeneMark.hmm\| | | | | | | | ACCATTCT | CTGAAACAA | |
| 1264_aa\|−\| | | | | | | | GTTTAGCA | (SEQ ID NO: 369) | |
| 24458\|28252 | | | | | | | TGGTACCA | GGTACCAAAGGAGTAG | |
| | | | | | | | AAGG | CTATAAATTAAGCGAA | |
| | | | | | | | (SEQ ID | ATCGATAGA | |
| | | | | | | | NO: 89) | (SEQ ID NO: 370) | |
| meta_ | | | | n | unclas- | | 4 | CAGCTAAAGTTAGAAG | SEQ ID |
| gene_295919\| | | | | | sified | | TTAGAAAA | ATGCTACTAAAGATCT | NO: 46 |
| GeneMark.hmm\| | | | | | | | AGAAATTA | AAGAGAT | |
| 1129_aa\|+\| | | | | | | | AAGAAAAA | (SEQ ID NO: 371) | |
| 18998\|22387 | | | | | | | (SEQ ID | AATAAAATTCAAGAAG | |
| | | | | | | | NO: 90) | ATTTAAAAAAGAGAAA | |
| | | | | | | | | GG | |
| | | | | | | | | (SEQ ID NO: 372) | |
| | | | | | | | | CAACAAGAATTAAAAA | |
| | | | | | | | | ATGCTACTAAAGATCT | |
| | | | | | | | | AGGAGAT | |
| | | | | | | | | (SEQ ID NO: 373) | |
| meta_ | | | | n | unclas- | | 4 | TTACGAGTTCGTTGAT | SEQ ID |
| gene_237613\| | | | | | sified | | GTTGTGATT | TTTCGCCGTCA | NO: 47 |
| GeneMark.hmm\| | | | | | | | TGCTTAAA | (SEQ ID NO: 374) | |
| 908_aa\|−\| | | | | | | | AATATCTA | TGAACGATGCCTTTGA | |
| 25932\|28658 | | | | | | | TCTTTGTGG | CCCTGTCCGCCG | |
| | | | | | | | TAGCAACA | (SEQ ID NO: 375) | |
| | | | | | | | ACAACCT | GCGCAACGCAGACCTG | |
| | | | | | | | (SEQ ID | AACGCTTTTAAG | |
| | | | | | | | NO: 91) | (SEQ ID NO: 376) | |
| meta_ | | | | n | unclas- | crispr | 4 | Not included | SEQ ID |
| gene_35066\| | | | | | sified | software | GGAACACC | | NO: 48 |
| GeneMark.hmm\| | | | | | | failed | TGGTACAC | | |
| 890_aa\|+\| | | | | | | to | CTGGTGG | | |
| 10428\|13100 | | | | | | recognize | (SEQ ID | | |
| | | | | | | array | NO: 92) | | |
| | | | | | | and | | | |
| | | | | | | spacer | | | |
| meta_ | | | | n | unclas- | | 3 | TTCAAGTATTGGCACA | SEQ ID |
| gene_524019\| | | | | | sified | | CACTTGCA | TGCTGGGGGAAGAGCG | NO: 49 |
| GeneMark.hmm\| | | | | | | | GTCCCCTA | TG | |
| 872_aa\|−\| | | | | | | | AATCGGGG | (SEQ ID NO: 377) | |
| 8834\|11452 | | | | | | | TGAGACCA | CGCGCTGCTTTCCACG | |
| | | | | | | | TTGCAAC | GCGGAGATGGCCCTCG | |
| | | | | | | | (SEQ ID | C | |
| | | | | | | | NO: 93) | (SEQ ID NO: 378) | |
| meta_ | | | | n | unclas- | | 3 | TTCAAGTATTGGCACA | SEQ ID |
| gene_523517\| | | | | | sified | | CACTTGCA | TGCTGGGGGAAGAGCG | NO: 50 |
| GeneMark.hmm\| | | | | | | | GTCCCCTA | TG | |
| 809_aa\|−\| | | | | | | | AATCGGGG | (SEQ ID NO: 379) | |
| 1421\|3850 | | | | | | | TGAGACCA | CGCGCTGCTTTCCACG | |
| | | | | | | | TTGCAAC | GCGGAGATGGCCCTCG | |
| | | | | | | | (SEQ ID | C | |
| | | | | | | | NO: 94) | (SEQ ID NO: 380) | |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of identifying a Clustered Regularly Interspaced Short Palindromic Repeat (CRISPR)-associated protein comprising:
(a) obtaining a plurality of genomic sequences, wherein a genomic sequence of the plurality of genomic sequences comprises a CRISPR-associated array;
(b) determining a subset of the plurality of genomic sequences comprising a plurality of coding sequences within a 20 kilobase (kb) sequence flanking region either at the 3′ or 5′ end of the CRISPR-associated array; and
(c) analyzing a coding sequence of the plurality of coding sequences and thereby identifying the CRISPR-associated protein based on the coding sequence.

2. The method of claim 1, wherein the obtaining step comprises selecting, within the plurality of genomic sequences, a genomic sequence comprising a CRISPR-associated array.

3. A method of identifying a CRISPR-associated protein comprising:
(a) obtaining a plurality of genomic sequences;
(b) selecting, within the plurality of genomic sequences, a genomic sequence comprising a CRISPR-associated array;
(c) determining a subset of the plurality of genomic sequences comprising a plurality of coding sequences within a 20 kilobase (kb) sequence flanking region either at the 3′ or 5′ end of the CRISPR-associated array; and
(d) analyzing a coding sequence of the plurality of coding sequences and thereby identifying the CRISPR-associated protein based on the coding sequence.

4. The method of any one of the preceding claims, wherein the plurality of genomic sequences comprise one or more of genomes, wherein the one or more of genomes are selected from: a prokaryotic genome and metagenome.

5. The method of any one of claims 2-4, wherein the selecting step comprises using an algorithm selected from the group consisting of PILER-CR, CRISPR Recognition Tool (CRT), and combinations thereof.

6. The method of any one of the preceding claims, wherein the determining step comprises using an algorithm selected from the group consisting of MetaGeneMark, Prodigal, and combinations thereof.

7. The method of any one of the preceding claims, wherein the analyzing step comprises filtering the coding sequence that comprises more than 500 amino acids.

8. The method of any one of the preceding claims, wherein the analyzing step comprises filtering a coding sequence that comprises more than 800 amino acids.

9. The method of any one of the preceding claims, wherein the analyzing step further comprises classifying the CRISPR-associated array based on having three or more coding sequences present in the 20 kb flanking region.

10. The method of any one of the preceding claims, wherein the analyzing step further comprises determining a relative position of the coding sequence in the 20 kb flanking region relative to the CRISPR-associated array.

11. The method of any one of the preceding claims, wherein the analyzing of the coding sequence further comprises removing known CRISPR-associated proteins from the identified CRISPR-associated proteins.

12. The method of any one of the preceding claims, wherein the analyzing of the coding sequence comprises using an algorithm selected from the group consisting of HHMSCAN and RPS-BLAST.

13. The method of any one of the preceding claims, wherein the analyzing of the coding sequence further comprises determining the presence of a structural domain.

14. The method of any one of the preceding claims, wherein the analyzing of the coding sequence comprises determining the presence of a functional domain.

15. The method of claim 14, wherein the functional domain comprises a DNA binding domain, a RNA binding domain, a nuclease, a helicase, a restriction domain, or a structural maintenance of chromosomes (SMC) domain.

16. A computer implemented method comprising:
(a) obtaining a plurality of genomic sequences;
(b) selecting, within the plurality of genomic sequences, a genomic sequence comprising a CRISPR-associated array;
(c) determining a subset of the plurality of genomic sequences comprising a plurality of coding sequences within a 20 kilobase (kb) sequence flanking region either at the 3′ or 5′ end of the CRISPR-associated array; and
(d) analyzing a coding sequence of the plurality of coding sequences and thereby identifying a CRISPR-associated protein based on the coding sequence.

17. The method of claim 16, wherein the plurality of genomic sequences comprises one or more of genomes, wherein the one or more of genomes are selected from: a prokaryotic genome and metagenome.

18. The method of claim 16 or 17, wherein the selecting step comprises using an algorithm selected from the group consisting of PILER-CR, CRISPR Recognition Tool (CRT), and combinations thereof.

19. The method of any one of claims 16-18, wherein the determining step comprises using an algorithm selected from the group consisting of MetaGeneMark, Prodigal, and combinations thereof.

20. The method of any one of claims 16-19, wherein the analyzing step comprises filtering the coding sequence that comprises more than 500 amino acids.

21. The method of any one of claims 16-20, wherein the analyzing step comprises filtering a coding sequence that comprises more than 800 amino acids.

22. The method of any one of claims 16-21, wherein the analyzing step further comprises classifying the CRISPR-associated array based on having three or more coding sequences present in the 20 kb flanking region.

23. The method of any one of claims 16-22, wherein the analyzing step further comprises determining a relative position of the coding sequence in the 20 kb flanking region relative to the CRISPR-associated array.

24. The method of any one of claims 16-23, wherein the analyzing of the coding sequence further comprises removing known CRISPR-associated proteins from the identified CRISPR-associated proteins.

25. The method of any one of claims 16-24, wherein the analyzing of the coding sequence comprises using an algorithm selected from the group consisting of HHMSCAN and RPS-BLAST.

26. The method of any one of claims 16-25, wherein the analyzing of the coding sequence further comprises determining the presence of a structural domain.

27. The method of any one of claims 16-26, wherein the analyzing of the coding sequence comprises determining the presence of a functional domain.

28. The method of claim 27, wherein the functional domain comprises a DNA binding domain, a RNA binding domain, a nuclease, a helicase, a restriction domain, or a structural maintenance of chromosomes (SMC) domain.

29. A non-naturally occurring CRISPR/Cas system comprising:
(a) a guide RNA, wherein the guide RNA comprises a repeat sequence and a spacer sequence capable of hybridizing to a target nucleic acid; and
(b) a CRISPR-associated protein or a nucleic acid encoding the CRISPR-associated protein, wherein the CRISPR-associated protein comprises an amino acid sequence that is at least 80% identical to a sequence selected from SEQ ID NOs: 1-50.

30. The system of claim 29, wherein the CRISPR-associated protein is capable of binding to the guide RNA.

31. The system of claim 29 or 30, wherein the CRISPR-associated protein comprises an amino acid sequence that is at least 85% identical to a sequence selected from SEQ ID NOs: 1-50.

32. The system of any one of claims 29-31, wherein the CRISPR-associated protein comprises an amino acid sequence that is at least 90% identical to a sequence selected from SEQ ID NOs: 1-50.

33. The system of any one of claims 29-32, wherein the CRISPR-associated protein comprises an amino acid sequence that is at least 95% identical to a sequence selected from SEQ ID NOs: 1-50.

34. The system of any one of claims 29-33, wherein the CRISPR-associated protein comprises an amino acid sequence selected from SEQ ID NO: 1-50.

35. The system of any one of claims 29-34, wherein the target nucleic acid is an RNA or DNA.

36. The system of any one of claims 29-35, wherein the targeting of the target nucleic acid results in a modification of the target nucleic acid.

37. The system of claim 36, wherein the modification of the target nucleic acid is a cleavage event.

38. The system of any one of claims 29-37, wherein the guide RNA further comprises a trans-activating CRISPR RNA (tracrRNA).

39. The system of any one of claims 29-38, wherein the system is present in a delivery system.

40. The system of claim 39, wherein the delivery system comprises a delivery vehicle selected from the group consisting of an adeno-associated virus, a nanoparticle, and a liposome.

41. A method of treating a condition or disease in a subject in need thereof, the method comprising administering to the subject a system of any one of claims 29-40,
wherein the spacer sequence is substantially complementary to a target nucleic acid associated with the condition or disease;
wherein the CRISPR-associated protein associates with the guide RNA to form a complex;
wherein the complex binds to the target nucleic acid sequence; and
wherein upon binding of the complex to the target nucleic acid sequence the CRISPR-associated protein cleaves the target nucleic acid, thereby treating the condition or disease in the subject.


SEQUENCE LISTING (379 sequences)

SEQ ID NO: 1 - protein, 1389 aa, Artificial
  gene_5155455; CRISPR-associated protein
MTKPYSIGLDIGTNSVGWAVITDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNSNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 2 - protein, 1090 aa, Artificial
  gene_3815793; CRISPR-associated protein
MSIRSFKLKIKTKSGVNAEELRRGLWRTHQLINDGIAYYMNWLVLLRQEDLFIRNEETNEIEKRSKEEIQGELLERVHKQQQRNQWSGEVDDQTLLQTLRHLYEEIVPSVIGKSGNASLKARFFLGPLVDPNNKTTKDVSKSGPTPKWKKMKDAGDPNWVQEYEKYMAERQTLVRLEEMGLIPLFPMYTDEVGDIHWLPQASGYTRTWDRDMFQQAIERLLSWESWNRRVRERRAQFEKKTHDFASRFSESDVQWMNKLREYEAQQEKSLEENAFAPNEPYALTKKALRGWERVYHSWMRLDSAASEEAYWQEVATCQTAMRGEFGDPAIYQFLAQKENHDIWRGYPERVIDFAELNHLQRELRRAKEDATFTLPDSVDHPLWVRYEAPGGTNIHGYDLVQDTKRNLTLILDKFILPDENGSWHEVKKVPFSLAKSKQFHRQVWLQEEQKQKKREVVFYDYSTNLPHLGTLAGAKLQWDRNFLNKRTQQQIEETGEIGKVFFNISVDVRPAVEVKNGRLQNGLGKALTVLTHPDGTKIVTGWKAEQLEKWVGESGRVSSLGLDSLSEGLRVMSIDLGQRTSATVSVFEITKEAPDNPYKFFYQLEGTELFAVHQRSFLLALPGENPPQKIKQMREIRWKERNRIKQQVDQLSAILRLHKKVNEDERIQAIDKLLQKVASWQLNEEIATAWNQALSQLYSKAKENDLQWNQAIKNAHHQLEPVVGKQISLWRKDLSTGRQGIAGLSLWSIEELEATKKLLTRWSKRSREPGVVKRIERFETFAKQIQHHINQVKENRLKQLANLIVMTALGYKYDQEQKKWIEVYPACQVVLFENLRSYRFSYERSRRENKKLMEWSHRSIPKLVQMQGELFGLQVADVYAAYSSRYHGRTGAPGIRCHALTEADLRNETNIIHELIEAGFIKEEHRPYLQQGDLVPWSGGELFATLQKPYDNPRILTLHADINAAQNIQKRFWHPSMWFRVNCESVMEGEIVTYVPKNKTVHKKQGKTFRFVKVEGSDVYEWAKWSKNRNKNTFSSITERKPPSSMILFRDPSGTFFKEQEWVEQKTFWGKVQSMIQAYMKKTIVQRMEE

SEQ ID NO: 3 - protein, 1305 aa, Artificial
  gene_2964877; CRISPR-associated protein
MNKAADNYTGGNYDEFIALSKVQKTLRNELKPTPFTAEHIKQRGIISEDEYRAQQSLELKKIADEYYRNYITHKLNDINNLDFYNLFDAIEEKYKKNDKDNRDKLDLVEKSKRGEIAKMLSADDNFKSMFEAKLITKLLPDYVERNYTGEDKEKALETLALFKGFTTYFKGYFKTRKNMFSGEGGASSICHRIVNVNASIFYDNLKTFMRIQEKAGDEIALIEEELTEKLDGWRLEHIFSRDYYNEVLAQKGIDYYNQICGDINKHMNLYCQQNKFKANIFKMMKLQKQIMGISEKVFEIPPMYQNDEEVYASFNEFISRLEEVKLTDRLRNILQNINIYNTAKIYINARYYTNVSTYVYGGWGVIESAIERYLCNTIAGKGQSKVKKIENAKKDNKFMSVKELDSIVAEYEPDYFNAPYIDDDDNAVKVFGGQGVLGYFNKMSELLADVSLYTIDYNSDDSLIENKESALRIKKQLDDIMSLYHWLQTFIIDEVVEKDNAFYAELEDICCELENVVTLYDRIRNYVTKKPYSTQKFKLNFASPTLAAGWSRSKEFDNNAIILLRNNKYYIAIFNVNNKPDKQIIKGSEEQRLSTDYKKMVYNLLPGPNKMLPKVFIKSDTGKRDYNPSSYILEGYEKNRHIKSSGNFDINYCHDLIDYYKACINKHPEWKNYGFKFEETTQYNDIGQFYKDVEKQGYSISWVYISEADINRLDEEGKIYLFEIYNKDLSSHSTGKDNLHTMYLKNIFSEDNLKNICIELNGNAELFYRKSSMKRNITHKKDTVLVNKTYINEAGVRVSLTDEDYIKVYNYYNNDYVIDVEKDKKLVEILERIGHRKNPIDIIKDKRYTEDKYFLHLPITINYGVDDENINAKMIEYIAKHNNMNVIGIDRGERNLIYISVINNKGNIIEQKSFNLVNNYDYKNKLKNMEKTRDNARKNWQEIGKIKDVKSGYLSGVISEIARMVIDYNAIIVMEDLNKGFKRGRFKVERQVYQKFENMLISKLNYLVFKERKADENGGILRGYQLTYIPKSIKNVGKQCGCIFYVPAAYTSKIDPATGFINIFDFKKYSGSGINAKVKDKKEFLMSMNSIRYINEGSEEYEKIGHRELFAFSFDYNNFKTYNVSSPVNEWTAYTYGERIKKLYKDGRWLRSEVLNLTENLIKLMEQYNIEYKDGHDIREDISHMDETRNADFICSLFEELKYTVQLRNSKSEAEDENYDRLVSPILNSSNGFYDSSDYMENENNTTHIMPKDADANGAYCIALKGLYEINKIKQNWSDDKKFKENELYINVVEWLDYIQNRRFE

SEQ ID NO: 4 - protein, 1412 aa, Artificial
  gene_4147644; CRISPR-associated protein
MKLSKEKHTRSAVANNGDIKSAEVNNGNTKSEEVNNGDIRSAVANEEQNIGGILYRFPGKSIDGVKDQMLRRDKEVKKLYNVFNQIQVGTKPKKWNNDEKLSPEENERRAQQKNIKMKNYKWREACSKYVESSQRIINDVIFYSYRKAENKLRYMRKNEDILKKMQEAEKLSKFSGGKLEDFVAYTLRKSLVVSKYDTQEFDSVAAMVVFLECIGKNNISDHEREIVCKLLELIRKDFSKLDPNVKGSQGANIVRSVRNQNMIVQPQGDRFLFPQVYAKENETVTNKNVEKEGLNEFLLNYANLDDEKRAESLRKLRRILDVYFSAPNHYEKDMDITLSDNIEKEKFNVWEKHECGKKETGLFVDIPDVLMEAEAENIKLDAVVEKRERKVLNDRVRKQNIICYRYTRAVVEKYNSNEPLFFENNAINQYWIHHIENAVERILKNCKAGKLFKLRKGYLAEKVWKDAINLISIKYIALGKAVYNFALDDIWKDKKNKELGIVDERIRNGITSFDYEMIKAHENLQRELAVDIAFSVNNLARAVCDMSNLGNKESDFLLWKRNDIADKLKNKDDMASVSAVLQFFGGKSSWDINIFKEAYKGKKKYNYEVRFIDDLRKAIYCARNENFHFKTALVNDEKWNTELFGKIFERETEFCLNVEKDRFYSNNLYMFYQVSELRNMLDHLYSRSVSRAAQVPSYNSVIVRTAFPEYITNVLGYQKPGYDADTLGKWYSACYYLLKEIYYNSFLQSDRALQLFEKSVKTLSWDDKKQQRAVDNFKDHFSDIKSACTSLAQVCQIYMTEYNQQNNQIKKVRSSNDSIFDQPVYQHYKVLLKKAIANAFADYLKNNKDLFGFIGKPFKANEIREIDKEQFLPDWTSRKYEALCIEVSGSQELQKWYIVGKFLNAMSLNLMVGSMRSYIQYVTDIKRRAASIGNELHVSVQDVEKVEKWVQVIEVCSLLASRTSNQFEDYFNDKDDYARYLKSYVDFSNVDMPSEYSALVDFSNEEQSDLYVDPKNPKVNRNIVHSKLFAADHILRDIVEPVSKDNIEEFYSQKAEIAYCKIKGKEITAEEQKAVLKYQKLKNRVELRDIVEYGEIINELLGQLINWSFMRERDLLYFQLGFHYDCLRNDSKKPEGYKNIKVDENSIKDAILYQIIGMYVNGVTVYAPEKDGDKLKEQCVKGGVGVKVSAFHRYSKYLGLNEKTLYNAGLEIFEVVAEHEDIINLRNGIDHFKYYLGDYRSMLSIYSEVFDRFFTYDIKYQKNVLNLLQNILLRHNVIVEPILESGFKTIGEQTKPGAKLSIRSIKSDTFQYKVKGGTLITDAKDERYLETIRKILYYAENEEDNLKKSVVVTNADKYEKNKESDDQNKQKEKKNKDNKGKKNEETKSDAEKNNNERLSYNPFANFDFKLLN

SEQ ID NO: 5 - protein, 921 aa, Artificial
  meta_gene_174274; CRISPR-associated protein
MAKKNKMKPRELREAQKKARQLKAAEINNNAAPAIAAMPVAEAAAPAAEKKKSSVKAAGMKSILVSENKMYITSFGKGNSAVLEYEVDNNDYNKTQLSSKDNSNIELGDVNEVNITFSSKHGFESGVEINTSNPTHRSGESSPVRGDMLGLKSELEKRFFGKTFDDNIHIQLIYNILDIEKILAVYVTNIVYALNNMLGEGDESNYDFMGYLSTFNTYKVFTNPNGSTLSDDKKENIRKSLSKFNALLKTKRLGYFGLEEPKTKDTRVLEAYKKRVYYMLAIVGQIRQCVFHDLSEHSEYDLYSFIDNSKKVYRECRETLDYLVDERFDSINKGFIQGNKVNISLLIDMMKGYEPDDIIRLYYDFIVLKSQKNLGFSIKKLREKMLDEYGFRFKDKQYDSVRSKMYKLMDFLLFCNYYRNDVAAGEALVRKLRFSMTDDEKEGIYADEAAKLWGKFRNDFENIADHMNGDVIKELGKADMNFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDKITDDRISEILKLKEKGKGIHGLRNFITNNVIESSRFVYLIKYANAQKIREVAKNEKVVMFVLGGIPDTQIERYYKSCVEFPDMNSSLEAKRSELARMIKNISFDDFKNVKQQAKGRENVAKERAKAVIGLYLTVMYLLVKNLVNVNARYVIAIHCLERDFGLYKEIIPELASKNLKNDYRILSQTLCELCDKSPNLFLKKNERLRKCVEVDINNADSSMTRKYRNRIAHLTVVRELKEYIGDIRTVDSYFSIYHYVMQRCITKREDDTKQGEKIKYEDDLLKNHGYTKDFVKALNSPFGYNIPRFKNLSIEQLFDRNEYLTEK

SEQ ID NO: 6 - protein, 568 aa, Artificial
  gene_4200106; CRISPR-associated protein
MPAAEVIAPAAEKKKSSVKAAGMKSILVSENKMYITSFGKGNSAVLEYEVDNNDYNQTQLSSEDSSNIELCGVTKVNITFSSKHGLESGVEINTSNPTHRSGESSPVRWDMLGLKSELEKRFFGKTFDDNIHIQLIYNILDIEKILAVYVTNIVYALNNMLGIKKSESYDDFMGYLSARNTYEVFTHPDKSNLSDKAKGNIKKSFSTFNDLLKTKRLGYFGLEEPKTKDTRVSQAYKKRVYHMLAIVGQIRQCVFHDKSGAKKFDLYSFINNIDSEYRETLDYLVDERFDSINKGFIQGNKVNISLLIDMMKGYKADDIIRLYYDFIVLKSQKNLGFSIKKLREKMLDEYGFRFKDKQYDSVRSKMYKLMDFLLFCNYYRNDVIAGEDLVRKLRFSMTDDEKEGIYADEAEKLWGKFRNDFENIADHMNGDVIKELGQADMDFDEKILDSEKKNASDLLYFSKMIYMLTYFLDGKEINDLLTTLISKFDNIKEFLKIMKSSAVDVECELTAGYKLFNDSQRITNELFIVKNIASMRKPAASAKLTMFRDALTILGIDDKITDDRISEI

SEQ ID NO: 7 - protein, 759 aa, Artificial
  meta_gene_524079; CRISPR-associated protein
MLQQPYTIDYGSRKTGSKAAAVGDNYYPTFFLDLLIIIGRPLQPITQSNDRFFDNVTVTFKNWRASYSSKHVHGLPFDLDHRTFRLATAATREAWYIVMHPTASTITDLPSSRRERRKRLEKSSQSSALQLHHAHFLAGYIKWVFLIDDLLGEGVEPSWTINGPHLTKITFNKWTAFQNRFMEEWDSYVQEYSCDNFWMENQPAFHAYDYGANIEIEIREESELSKQLKSLPKETRLRRNNEESESEEEDTNILEDGTQLMSSRSNSREVSEAPEEINYQSLYTEGLRQLRTELERKYILNNISSISYALAVDIGCQDSNSPDPEDKQVYCLLADRNKVLGDFRGPRDFTFYPLAFHPAYGNFSSPGPPSFLIDNVLAVMRDNMSYQNDGADTLSYGYFQAYSNIKRSIRHKPEDLLATKGIATAALALPESEANASSHIKAKRQRLLQRLQGQATPEDPDSSKPFERERQLIEAAIVAEKFDFRMEQVLTIQVSRLIDSRRNFSTVLNPIFQLVRFYLMESHRYTHLLRWFPPSVFPGILGSFARIFGLAIDEIYARFKAGGSKGLSIALAEGVSALDRLGSYCFTGFPKSLMGSVLSPLGTIDGIEQGAWPYINPRMLDLQDGGGSLCLSQWPRGENKRPLLMHVASIGFYYGPEVAASRHSNVWFKEFGGMSIKGPSGAAKFLEDLFQDLWIPQTVAFVDHQLNRGLRQGSGSADKTKEELLLLEHQQALIRQWLQSEHPFSWAYVNDRRAVKCCS

SEQ ID NO: 8 - protein, 769 aa, Artificial
  meta_crt_array_WNGG01011662.1; CRISPR- associated protein
  misc_feature (769)..(769): Xaa can be any naturally occurring amino acid
MRIPVTQARNNLLGGERSWRDMVSPAQRFLQPRSARAPRSLAGSKMPNSPRETRLTHNNFRGSRLLAEHRHDCAGAGMARIRGIARVGLDDYDVKIIPGDDGPGLRDLARHDHGHVGRDSGRRWRAVARVCGPVHATGGGSRTVLEALERGWSRTRAQRVVLRPLRAPEVHQLPDRSRDGANRLVSNDKGAARAEGEEQDAARGVSATAAHVTDVDFDGVGAKRRVRVPAAHGEPAAAGGNGSRRGGTAVTPVDGRCVVGHRAVRVSIGEAGHHRIGRDGVGAAQGLAGCRQGGIGDGRRARSGGAVADVVDVGDRRCDREGPLLSVGVRATHREGSTGRTGDGARGGNAAVAPVDRRRVFARRCLGVGICDGGDCAANGYSLSCRDRGSRGLDGRVCGGHPRLGSGGLLQCSSVINKGRRDGICPHSATVGVGERDLAVDVRRARDRAAGAHGAHVGPAVDAEGDGLPRLRAATLEDGRGHGVAAADWVSGRCRSQADVGLDSDAAAAEQDVWRARDGSPSVDVRRRVGLGHGRVCSQCQHCPVTREVVEEGMHRAAPVGQVGVVEPGRRPGRGDDGDAAGTGGAPRCELMRSVGAQSVHDWHRGSRWSSAIPGVRGAELATRVDVGAGRACHCTREGNAFSLEEIASPGAARARVVEGRVGADQNLVASTGHHHRLPDGGVGLSGGVGLVALAGRIADDERLIGGELACRHSVPLGVSRQGDGEAVGLSLLLLQALPAAIGSGARGYAASQDEHLGGGSMRVSDGAX

SEQ ID NO: 9 - protein, 727 aa, Artificial
  meta_gene_336895; CRISPR-associated protein
MEIIDKVNANSFYKMRDKFLYSSDVRENIALRDNVFAPIFILCEVNEIRNFDGERNDKLSYFEMKLNYETKEIDLGSNYRSLVDRIKVIKKEMKIFYEEMLKNERDVDVSFINSNKEIKEKLIDFIKEKKEFFEMSDDDFIDLYKRFYRLLYCINDEQSKLIIGENIKREINFYRNIVNTKDKTRLLEKKYNVEDDPYLVLFSVFYNGISDYEYSIFNIGMLKREVNKLFMLNKLNNEFYNLLIDMHVMSMEEILFSENNLGIKYSELETMVKYSLHDRIGIVENADRLLIIKEDEKTKEKNTETNIYKNFNLEYYDKVKTLDLINIEFKISEDIDENIKKLLDIKTQENLKFFVVTYLIEHGYINYYKGNKLKNITISTKQEKNEEERTINRGFIYLKLSEKDFQFEGTLELEFEIRLGLKIIQKREKLSFTEEDIKSDKVESVSKILLNSNDSNKNYFDGNTTFGLPRNQVDFNVFFRKFKEYFSSNRSRKFTGYDIKINRDRTREKNIPITLKRINKSAYCEKIRVNPLTGEIISNNKYNKSLEDVMLHTYLYIYMQSLFLMVKTRLKNNGEFKTLDLFNFESFMGLINNINVPDTRHGLFRYINYYYFEKYEPFIENKIKYKPITKDGKIIKNEIENIINNMEDYILLAKISEFIYLQILHNFSLENIIDIEIATNEEIKSILNLNYNDVELKKESKYIKTIMNYYAEFLETFNNEIKIKEEN

SEQ ID NO: 10 - protein, 675 aa, Artificial
  meta_gene_321445; CRISPR-associated protein
MVGEYLGLTTFEKAIEQKPITLTKVDIDKSIINDYKEINDFILAKKSTVSLLNEDNKKMVEYAKIHGIDTKEILKEIKSLHKAENKELEKDMKSSELDKNYAWYLENKENKAIKDVLETKKNTFLSEWSKEIGNLESDEKLAYLKGTNDVNFKNEIYNFSDEKIKKVLDIYTNFKEEYTANQKEYFNNNGVEIEKEEEKEKNFHSINEINQNYLSDKEKINDILSVLKITVEDIEKTEEQIKKRNPDLNDKTIADMITNHIYDNMIKENVALIDFSKKEFFNFGNENITKEMELERYFNLKEKNSIESFDEKDLVSFDKVDYLIKDRENIINNERQYLLSNELKEHQDINYQAKKEIALILTNTNALDREIKAEMLEFKDDKLIVITPEYNAEIKTEKLTENNIDKIRNIILNGIENKTSLNDMEKLNKNNEILFNLGQRDKLREFGNSFYKDMIFDKENEIIKEIKVEIVKEKYLELSDREKVLERAKELGITLEKEPVFTTKSITKDMEDAEPNIDKDNEVTINYFENRNDIYQFFENSLYLKNALRIYEDTLDFDYKEIYPYNNLEENAKFIVANILELIPEIKNNKEEFGIDSINLHEYLKEMSFDDLELWINNIDETIKEVIENKVEKDDDYVPEVTDKTEDISNNNEDENKRIENDKEKNKEKDDEYNF

SEQ ID NO: 11 - protein, 1351 aa, Artificial
  gene_3820393; CRISPR-associated protein
MIEAPGDPVERQFDEWLTRWSRWAEPEAARRSRETLRRELAAAARQLDLHSDTQELILGVGLLCWRSPRGDEVFRHLLTAPVQIVVDKQTGRVGVHLRDEGELALEDQYFLTEQDGYVASRVEPLRGALSEVSDPLDDQAKALLHKWASHGLETPCKFEPVWSTPETGGPHALVSLSPALVLRHRSSNRLAEFYQGIHASLSDPEGVAPLGWAQLMFPMEPEERLAWHRATRGTAGTSRLLSEEPLFPLAMNDEQRLAFDKLSKDTVLVIEGPPGTGKTHTIANLMSALLAEGKRVLVTSARDKALNVLFDDGMLPKPLQRLCVRLDDQRGNRGKELTRSVTALSDASAERSKEEILERARMLTDRRSELKREISLVHRQLWELIEAETTDLGEVAPGYRGRRADIAERVADTASTHSWIGIMPDSAAPVPLNSQEAQELAQLLRTPAQNDQPLPTLRAGNPPTPDEFTALVSAAHQTLPASGVGARLAERLSTLDEGAFRTVSAFWELACNALQGLRLPGDTASWSSIDWQGTAALSILQGGDVSAWKHLWEATRTAAPHAQELARLTGRYLQIPALHGAGAAEAASAAEAYSRFLKAGGRPGKIKKSPEQRMAERSLAECFVDGRRPSTVADFDMLTTALRAVAVLSGLSNRWRRSGVKTNTPDNVSQNLEALVGREADLAHLIRFAEALESLHQHLPDRSAIHASGSWDWPALVEGFTAAPAHMKSARARRNLDSLRARIADADHPLFREMTTAVERRDLAAYTTAFEIWKTQAHSQRLAERRSELVDRVAAVHPALAHRLATATMDDDWTSRLETLDEAWAWSAAAAAVSSRSVESTAELQRELDRLEDALMKTTAELASEQAWWHCLQRMSVREASALRSFAREMKRVGRGKGRYAGRHRQGAREAMRLARDAVPAWVMPVRQVAETIDPRPDAFDVIIIDEASQLPVESAFLLWLAPQVIVVGDDKQCSPPMRVSGELEPIYERIEEYLPDVPRAFRHDLTPKSNLYELMNVRFPGGQRLTDHHRSMPEIIAWSSRMFYDGSLTPLRQYGTDRLPPLRVVDVPDGYREGRDQNVRNPPEAEKLVTELKAMIEDPAYSGKTFGIISLQGGERSGHIRLIEQLLDEHLPDQALRERLKIRVGTPPDFQGDQRDVILLSMVATGTPRIQGGADFEQQRWNVAATRARDQMVLFASTTLTQLKSDDLRASLLKHMLDTPMRETTPQHLLHVEPQTKHPEFDSLFEQKVFLKIRERGYEVVPQYPAGRNMRIDLVIVGEKGRLAVECDGRYWHSGAKQVQDDLLRERILRRAGWTFWRLRESDFLLDPDVSLRPLWALLDRIGIHPAKGQ

SEQ ID NO: 12 - protein, 827 aa, Artificial
  meta_gene_180752; CRISPR-associated protein
MAQFNFTKKLDIDETQIEQTDVMTGDNNRNRYLYYQLKLSMLHAKKIDIIVSFLMESGVRLILNDLKTALDRGVQIRILTGNYLGITQPSALYLLKNELGNRVDMRFYNDKHRSFHPKAYIFHYENYEDIYIGSSNISRSALTSGIEWNYRLNSQDNHKDFVLFYDTFQDLFENHSIIIDDNELKRYSKNWHKPAVSKDLARYDAVEDNSDTPVRKLFQPRGPQIEALYALADSRSEGATKGLVHTATGIGKTYLAAFDSAKYQKVLFVAHREEILKQAAISFRNVRQSNDYGFFYGKQKDKDKSVIFASVATLGRSEYLTENYFAPDYFDYLIIDEFHHAVNDQYQRIINYFKPKFLLGLTATPERLDGKDIYEICDYNVPYEISLKEAINKGVLVPFHYYGIYDTVDYSSIHLVRGHYDEKQLDKAYIGNKDRYDLIYKYYKKYPSKRALGFCCSRKHANEMAKEFCARGIDAVAVYSNTNGEPSEERNIAIQKLKSQEIKVIFSVDMFNEGVDIPDLDMVMFLRPTESPVVFLQQLGRGLRISKGKTYLNVLDFIGNYEKAGRVPLLLTGGGDSNKNAPTDLSSIEYPDDCIVDFDMRLIDLFKKLDQKSLTAKERITHEFYRVKEKLDGKIPTRMQLFTYMDDDVYRYCITHAKENPFRHYLEFLEKLHELSETEETLCSGLGKDFLTLIETTDMQKVYKMPILYSFFNHGNVRLAVKDDEVLAAWKDFFNTGKNWKDFAADITYDEYKSITDKQHLRKAKSMPIKYLKASGKGFFVEKDGFALAIRDDLKDIVKNDAFIKHMHDILEYRTMEYYRRRYLEKI

SEQ ID NO: 13 - protein, 1452 aa, Artificial
  gene_771418; CRISPR-associated protein
MRRNPEFTFFSHKNVPEVSGYEGGLVNSTIMNSLHTSPTLGIDIGSTTVKVALLDAEHNILFSDYERHYANIQETLAELLRKAREKAGPMEVVSVITGSGGLALSHHLQVPFVQEVVAVASALQDYAPKTDVAIELGGEDAKIIYFSGGIDQRMNGICAGGTGSFIDQMASLLQTDAAGLNDYARHYKAIYPIAARCGVFAKSDIQPLINEGATREDLSASIFQAVVNQTISGLACGKPIRGNVAFLGGPLHFLPELRNAFIRTLHLTGSQIIAPDNSHLFAAIGAALNPQEGQTSSSLLSMIERLSSGIKMDFEVKRMEPLFRDQADYDEFDRRHAGHQVKTGDLARYSGNCYLGIDAGSTTTKVALVGEGGELLYRFYDNNNGSPLATAIRAMSEIREILPPTAHIAWSCSTGYGEALLKSALMLDEGEVETISHYYAAAFFEPDVDCILDIGGQDMKCIKIKDGTVDSVQLNEACSSGCGSFIETFAKSLNYSVEDFAKEALFAENPTDLGTRCTVFMNSNVKQAQKEGATVADISAGLAYSVIKNALFKVIKITRPSDLGRHVVVQGGTFYNDAVLRSFEKISGCEAVRPDIAGIMGAFGAALIARERWHMQPADSGRETSMLPLDKITSLKYTTSMTRCKGCNNHCVLTINQFGSGRRFISGNRCERGLGIEKSKKEIPNLFDYKYHRMFGYTPLPLDKAHRGVVGIPRVLNMYENFPFWAVFFERLGYHVTLSPQSTRQLYELGIESIPSESECYPAKLVHGHISWLIKQGVKFIFYPCIPYERNETPDAGNHYNCPMVTSYAENIKNNVEELAEEHVNFMNPFMAFTNEEILTKALVAEFANAFDIPAAEVRMAAHAGWEELLQSRRDMEAKGEEVLDWLKQTGKRGIVLAGRPYHVDPEIHHGIPELITSYGFAVLTEDSVSHLGKVERPLVVTDQWMYHSRLYAAASFVKTQENLDLIQLNSFGCGLDAVTTDQVSDILTRSGKIYTVLKIDEVNNLGAARIRIRSLIAALRVRDQRNFERKVVSSAYHRAVFTKEMKKDYTLLCPQMSPIHFDLIEPAIRSFGYKIEVLQNHNRSAVDVGLQYVNNDACYPSLIVIGQIMDALLSGRYDLNHTAVFMSQTGGGCRASNYIGFIRRALEKAGMPQIPVISVNANGMETNPGFTITLPLLTKAMQGVVYGDIFMRVLYATRPYEAEPGSANALHEKWKKRCVASLSKRSSSMMEFGRNIRGIIRDFDALPLRDVRKPRVGIVGEILVKFSPLANNHIVELLESEGAEAVMPDLMDFLLYCFYNSNFKSKHLGTKKSTTYLCNAGIALLEYFRRTARKELEASKHFTPPAAIDELARMAQGFVSLGNQTGEGWFLTGEMLELIHSGVENIICTQPFGCLPNHIVGKGVIKELRRHYPQSNIIAVDYDPGASEVNQLNRIKLMLATAQKNLKKGTN

SEQ ID NO: 14 - protein, 1422 aa, Artificial
  gene_1433645; CRISPR-associated protein
MGSGSEYGIKSLKNLDGIEHIRLRPRMYTDIGSEIGCHHIAQEVLDNCGDEAIGGFCSRITVEIESDHVICISDNGRGIPVETDEASGMSGVEMVLTQDKAGGKFDHDSYQVSGGLHGVGVTVTNALSSFLEATVKRDGGEWFMRLEKGRVIEKLRRVADCGPRTRGTSIRFSPDPEIYEQAKFRVQQIRQQAMDKAILIPGLEVIFKAPGLEAERFCFKRGLAEYMEANMADSPVFEFSGALGDVEKVHWFFAAFDEPVDSFIRSYANTVPTPRGGTHEKGFADGMLKAVREYLDLRPELKKTLGKNTRIAPSDVMANSQMGLSVYIKDISFEGQTKQKLGSREATKFVGGVIHDAASLLLHRDVELSDAWVKMVIDRASARTALENGKKKKVERKSYTGRTPLPGKLQDCRFNGIEGTEIYIVEGDSAGGSAKQACNRDTQAVIPIKGKILNCEGINQEDAIASEAVADLVTAVGSGVGDVCDPANRRYGKVIIMTDADVDGLHIQNLLGTFFYRLMKPLIDAGCVYIVQPPLYGVTIGKQKHYAQDQEELDGLKAMALAEKKKISYTRYKGLGEMDPPELAETCMDAENRVLVKVLPRSDKRMDALMTKLMGDDADQRKNLLMGVEIEDAVHLEPVEEPCDVTEDVKELCQPNSYDSGNNKVAPFETVFREMYRGYGLQVVGGRAIPDVRDGLKPVHRRILYAMEMLKLRSDGPTKKAARVVGDVIGKYHPHGDSSVYDAMVRMSQPWKMRYPYIHPQGNWGSIDGDSAAAMRYTEARLTPIAEAMLSTDLKEGISEYQPNYDDEDIEPLLLPAPFPSVLMNGTTGNPGVGFKSEIPPHNLTELMGACIALADKRIRTGEAESPQDFASVRKHITAPDFPGGGIIAGSHDDLEKMYASGRGKMLLRSKWHVEKLERGAWQIVITEIPYGIEKSPLLISMGQCISDPTLPERKRLPMLEDIRDESEGTDIRIILYPKSKGLDPHDIMLHLFSVTNLQVTIEYASYALEDWVLAPNGDRYRLPRLFALDQMIRSFLNNREQIVTARSTVRLAEIEKRLHILDGLLLAYPNIRDIVEIILENDEPKPIIMKKYALSDPQVVAILAIRLSQLRKLEEMKLQGEHNQLSAEAVELRQTIDDYTHRWKKIKKELQHVRKTFGDERRTEVDPDAARARIMSKEQLVAREPVTAVLSKAGWLKGMRGSNIDVENVKFREGDTILDHAAGHTTSRVVLIGRTGRAFNMLAADLPSGRGNGEPISKNFIFSIDEAPTRLFMINPDAEYMVVTTLGHAFRAKGEDMLTANKKGKAFINFPTGSKLLCIREIDPGHDAIAFITDDGCLGIVKLDEFPLLAKGKGLTAVTMKKGVKLLRDAAPVNTSAAVRVGTEKRSTAFEPDEQAETYIIERGRPARPLPKACVNGMLII

SEQ ID NO: 15 - protein, 1255 aa, Artificial
  gene_4426209; CRISPR-associated protein
MKEMNKSETKSSKLLGIVLFHSFIPGKLFKVKAIGHSNNTGDNGAGKSTLLSLLPAFYGADPSKLVDRQADKVSFVDYYLPTPKSVIVFEYEKLGERKCSVMYRNGSSVAYRFLTGTAEQLFSQHLYDELIKQGSETRTWLKNLVSQSMSVSSQIETSVDYRSVILNNKKRLAQRRSTGKNLVAIAHEYSLCSASHNMNHIDILTATMMRHKKMLSRFKTMIVDCFLNNTSMDDVPYKKEYSELINSLDVFVQLETKKSKFDEALANKDSLEEYIKQLNSYRAQIASYLHQLALSDTQLSDKIRSQKEQHEILVNERKGKLHTFNSELNNQRIEFERKSKIIDAIYNKRDKYENEDDILGKITLYNSLSDMLREVESARKHYDNLLEDVRTEETELKSQVQKLELECSDFRFRKQQEINSVLKAKEEIVEQKSERLEAMQSDLNFEKKKLQDAFDEQSERIKQEQLRLATLEGQSLDFTSEQKAELRILENDLDKKRREFNASQNTVIYLNEQLRQATKTHEGSLSAYHACRDELKEISDEIISVSRALLPTKGSLNEFLEQKVPGWRCNIGKVIDPNLLNSKNLKPFFDLDTTESMFGLHLDLDSISLPDFCLSEEKLSERLSTLKIKELETETREEKAKSRAKSDEQETEKLQKEVKIQSQRSKVLEDELSKLNLLKDQKTAQFESDAESRTYEVKKQKSVLESEFFAIKSELKAKLENEEQRHQQERVQVKANFDYRLSEEDAKKSAIEALIKDKEKVTSDRISDCKLAFNQALMNKGVDPVSIESAKLKWELLERQCEEIKAFQALIIDYHTWLEAEWKYIDTYNSEKLDLERQIARGVAKRDDYEKSVGRKIDDVATSIKLDEQELITVKEAIGQLTTCTNNLEKAVDESDLASLEDVSVDLEFHSVEHAVSLVTDKITAINTLKKEIVSKVKDVSNTILGLDDNNEIKMMWEQMRSATMTKLSDKYDYAINYDSPQFSLACLGDLEGLVLNVIPDVRDVKIETLRSISTQISNYHQTLKQVNSKVDSVSSTLDKSIETGNPFPAIDSIHIKLSSKIHTFDLWKDLNLFSVELDRWSGETSRGLPSKAFLASFKQLLASFKEAQISKNLESLVEMEITIVENGRPAVVRNDEDLEKVGSEGISKLAIIVVFCGMTRFLCQDEDVAIHWPLDELGKISISNLAILFDMMAQKGICLFTAQPDLHPATYKYFATKNHIVKNVGVKSFIGGRRSRVNPLLSESKLNQSTEVVE

SEQ ID NO: 16 - protein, 1213 aa, Artificial
  gene_5411831; CRISPR-associated protein
MNKSNLKKFAIEARQELREKTKAQLKRLGIEEKKIEEGKDMGSQVEIYGKLYSKSSYQHLLVKYHSLGYEELVEESAYLWFNRLTALAYMELHDCFTEHMIFSKGNKGEPDILDEYFQADFFQKMPLEKQEELHQLRDKNTSDSLETLYSILMEEKCEELSKIMPFLFSKKGKYADILFPSGLLMQDSVLKKLQVILLEIQEEDQSIPVEILGWLYQYYNSERREVVYDGSMKKSKIKREFIPAATQLFTPDWIVRYIVDNTVGRLAEEQFSISKDIIKKWQYYIAPEIVSKNEKMQIESLKILDPAMGSGHMLTYAFDILFDVYQELGWSKKESVLSILQNNLYGLEIDDRAGQLAAFALLMKGKEKFPRLFQVLEREENFEMPVISLQESNAISKRMYTMLEECPTLQDLLKGFEDTKEYGSILKIDSFEESILQEEYHKLQEKIQNQGQFSLLNNNEFLEGDLEEDLERLEHIIRQYKIMIQKYDVVITNPPYMGNARMNPKLKTYIEKYYPNVKTDLFSVFFIKCCEMTTEKGYLGFMSPFVWMFIKSYEELRTLFIHSKTIISLVQLEYSGFEDATVPICTFILQNTVIKKIGEYIKLSDFKGVKNQPIKTLEAIQNENCTWRYQANQKDFTKIPGSPIAYWVSDRIREIFEKEKKLGEVGDAKVGLQTGDNNKFVRLWHEINFNKIGFGMQNSEEALKSKKKWFPYNKGGEKRKWYGNQEYVVNWERDGYEIKHFCDTNGKLRSRPQNTEYYFKKSISWGLITSSGSSFRFYPEGFIYDVSGMSYFIEDKFLTYLGILNTKIYSKLTKLINPTINLQIGDILNLPVANIQNPLFEQLVSLILWISFEEWASRETSWDFERLTLLNGENLSKAYKKYCTYWESKFFSVHSSEEDLNRILLESYSLQEEMDEKVDFSDITLLKKEASIVENTDSAASCGYLENRGVRLEFHSLELVKQFLSYAIGCIMGRYSLDKPGLIMANSDDVLTMSSNKITVSGVNGAIRHEILNPSFFPEEFGILSVTTEERFENDVVSRVIAFISAAYGKEHLAENLEFITEVLGKKAGESHEEVLRNYFIKDFYTDHCQRYQKRPIYWMLHSGKKNGFSALIYLHRYEKDTIARMRSDYLLPYQEFMEQQEAHYSKIASDEISTPKEKKDAQKKVKELHDILKELKDYANKVKHIAEQRISLDLDDGVKVNYEKLGSILKKI

SEQ ID NO: 17 - protein, 1123 aa, Artificial
  gene_941761; CRISPR-associated protein
MALKGDKLLCTNFEFLKVKKEFTSFSDACIEAEKSILVSPATTAILSRRALELAVKWVYSFDEELGIPYRDNISSLIHNGSFIELIDSEMLPLLKFVINLGNVAVHTNKTVTREEAILSLHNLYQFINWIDYCYGDDYKEKKFNENSLLQGEEKRVRPEELKDLYDKLSSKDKKLEEIIKENEELRKVITQKRKENIENYDFNIEEISEFDTRKIYIDVELKLAGWDFNKDIGEEIELFGMPNNAEKGYADYVLYGDNGKPLAVVEAKRTSRDAKAGQQQAKLYADCLEKQYNVRPVIFFTNGLETYIWDDYNGYSERRIYGFFKKDELQLMIDRRTQKKTLRNIDIKDEISNRYYQKEAITACCEELERRKRKLLLVMATGTGKTRTAISLVDVLTRHTWVKNILFLADRTALVKQAKKNFSNLLPDLSLCNLLDSKDNPEESRMIFSTYPTMMNAIDDTKAKDGKKLFTCGHFDLIIVDESHRSIYKKYKAVFDYFDAYLIGLTATPKDEVDKNTYGIFDMENGVPTYAYEFDKAVEDEFLVEYETIEVKSKIMEDGIKYDELSDEDKEEYEEKFDKDENIGEEIQSSAINQWLFNANTIDLVLNKLMEKGLRIEGNEKLGKTIIFAKNHKHAEAIKERFDILYPELGSNYAKVIDNQINYVDSLIDDFSGKDKLPQIAISVDMLDTGIDIPEILNLVFFKKIRSKTKFWQMIGRGTRLCEDLLGIGQHKDKFLIFDFCNNFEFFRMNPKGFKGNLGQTLSERIFNLKLDLVKELQDLRYSDEEYVSHRNELLKYLIEDVNNLNEDSFMVKMNLKYVQKYKNKNEWQSLGAVNAKDIKEHIAPLISKLNDDEFAKRFDILMYTIELANLQGNNATRPIKSVIETAESLSKLGTIPQIQQQKYIIDKVRTTEFWEDVDLFELDEVRSALRELLKYLGKTTQKTYYTHFEDMIINEESHGAMYNVNDLKNYRKKVEYYLKEHENELAIYKLKNNKQLTKQDLETLESIMWQELGTKADYEKEFGDMPVNKLVRKMVGLNRNTTNELFSEFLNNENLNIKQIHFVKLIIDYVVKNGFIDDNRILMEDPFRTVGNLSVLFKDNMKEAKSIMGKISQIKENAEKIV

SEQ ID NO: 18 - protein, 949 aa, Artificial
  gene_1546948; CRISPR-associated protein
MRLIALELENFRQYAHAQVAFESGVTAIVGANGAGKTTLLEAILWALYGARVLRDDTHTLRFLWSQGGAKVRVLLEFALGSRRYRVRRTPTDAELAQLNPDGAWLSLARGANAVNRLVEQLLGMNHLQFQTSFCARQKELEFLGYTPQKRREEISRMLGYERVGAAVEAIGRAERELKASVEGLRQGVGDPRALEAQLDAVEQALQATETALHAEQVALQRAVAARDAARAHYDAQAALREQYLQLHQQRTLLQNDRQHAERRIDELRAQWEQLKAACDRYKVIKPDAERYRQLARELEAMEQLAQAAQQRAQLQARLDALGERRAQLHAERDALLQKQAHLDALQPQRARAEQLARELQTLRHIARQAAQRAQLEAQLQAIAEQRQRLHALATERDALAQQAQRAEADLHARHTACAQTEAELQQTLQAWSQQRADLDAQLRAVQTTLQQQRARVQQLEALGESSECPTCGQPLGDAYQRVLTAAQQEAQATERELRALRQQRRALEQEPDAIRTLRQQLAQQQQARDDAQRQLAELQARLRQLDAELRQTAALERQQRDLEQRLAQIPPYDPEAEQRAQAELDALQPALQQAHALEGELRRLPAIERELSQTEREAQRIQRELDRLPDGYDPDQHAALRTQAEQLRPLYEESLQLAPIIQQRDALRARIEDAKTALQRVIAQCEHLETQIAQLGYSEAAYQQAAEAYQQAEAQVNTLERSLAARQAEYASQTALRDQLRAQLERLLELQRALREQEHQLRVHSLLRKAMQDFRADLNTRLRPTLAALATEFLNALTNGRYSELDIDEEYRFTLIDEGHRKQVISGGEEDIVNLSLRLALARLITERAGQPMSLLILDEVFASLDAERRHSVMELLNNLRSWFDQILVISHFEEINESADRCLRVRRNPQTRASEIVEDALPDPATLATAALDDALAGDEETGLLPPP

SEQ ID NO: 19 - protein, 803 aa, Artificial
  meta_gene_15450; CRISPR-associated protein
MAKKKKTPVAQIEPISLPDEDLAKARAWLEGLNADIAYSQAKRQLAEACGWERSKSNAVIVALHEEGFMAGEKNYFCNPNAPAEPGVVRGAREVSNFTIMLQSDPEVSVPLPYAIHCLPGDVFMLRKTVTGNWRVSNFVARHQTRWVCKLRGRIRRGRRSGIAQVVPINGFAPVEMQMDLADVPAEVDLEKAAFEVEFLPESMKPEPYVEIFVRFVKEIGNRFDPLGEIAIASAEYDLPVEFSAAALDEAQALPDEVDPKNMGRRVDLRDIPFVTIDGEDARDFDDAVYCARVEDGRTRLLVAIADVSHYVKPGAPLDVDAQQRATSVYFPASVVPMLPEKLSNGLCSLNPGVDRLTMVCDAVIDPEGRTEAYQFYPAVIHSHARLTYTQVWGAMQGEEGGLAAVGDRLDDIRALYELFKTLRKARDARHTLDLETKETMAVFDDKGVISEFKVREHNDAHRLIEECMLVANVCAADFVIQKKRGALFRVHDAPSQERLETLRTVLKSFNEKLESPTPEGFAELISRTKENEFLQTAILRSMSRACYSPDNVGHYGLQYEAYAHFTSPIRRYPDLLLHRAIKGILSRRIYVPQVVFDDSSLMVSRQARGLGSRPEAGDGDKPATQAEKRHSVWERLGILCSAAERRADDATRDVMNYLKCDYMLRHGKGRHEAVVTGMIPAGVFVALKDIAVDGFIHISNLGWGYYEFDEKNLTMTSREEMTQVRVGDRVIVRLEEVDLENRRMSFVLESNLERRLIKGGKGGSRRSSRRGSRLYGRQFDPFDIDDDDFDELFGQEGDDDWDD

SEQ ID NO: 20 - protein, 804 aa, Artificial
  meta_gene_73412; CRISPR-associated protein
MSVARKTGSQPRALHAADSHDLIRVQGARVNNLRDVSVVLPKRRLTVFTGVSGSGKSSLVFGTIAAESQRMINETYSAFVQGFMPTLARPDVDVLDGLTTAIIIDQERMGANARSTVGTATDANAMLRILFSRLGQPHIGSPQAYSFNVASISGAGAVSIERGGQTVKERRSFSITGGMCPRCEGRGAVNDIDLTALYDDSLSLNEGALTIPGYSMDGWFGRIFSGCGYFDPDKPIRKFTKRELRDLLYREPTKIKVDGINLTYEGLIPKIQKSMLAKDIESLQPHIRSFVERAVTFTTCPECHGTRLSEAARSSKIAGISIADTCAMQISDLAEWLGGHYDPSVAPLLEALRHTVDSFVQIGLGYLSLERPSGTLSGGEAQRIKMIRHLGSSLTDVTYVFDEPTIGLHPHDIARMNHLLLKLRDKGNTVLVVEHKPEMIAIADHVVDLGPGAGIAGGEVVFEGTLDGLRASDTLTGRHLDYRAAVKETVRTPTGALEVRGATANNLREVDVDIPLGVLCVITGVAGSGKSSLVRGSIPAGADVVSVDQGAIKGSRRSNPATYTGLLDPIRKAFAKANGVKAALFSANSEGACPNCNGAGVIFTDLAMMAGVATSCEVCEGKRFQASVLEYHLGGRDISEVLAMSVAGAEEFFGAGEAKTPAAHKILTHLVDVGLGYLSLGQPLPTLSGGERQRLKLATHLGEKGGVYVLDEPTTGLHLADVEQLLALLDRLVNSGKSIIVIEHHQAVMAHADWIIDLGPGAGHEGGRVVFEGTPAELVAARCTLTGEHLAAYVGTGPRKVRTS

SEQ ID NO: 21 - protein, 1697 aa, Artificial
  gene_307407; CRISPR-associated protein
MQQTLGNEATTRALRRGKRPMAPRPPAIDERAEQGLVLPPYLMELEAGGLSTAYGLTGQEFVSTAVAAVVGHGGGTVAGISAELAGRPESFFGRGRAFAVEGAEGGDGFDVTVSIAPAPDDLPPTFHPAADLASAPPDPGGAPLAAVDDAEGKETKVDVQHNSGATASSTVGNSSSKGAGGTAFGLAPVLPGLWLGAAATGSVQPWQSSRDSRSQRGVAEPRVLRSDKGSVEVPRRVLYVVRVRPQAGGDEQVFRGSGGLTQRVPTEHLIPAGTEAPTLAAPASGAPGRSQQVDPDLARRVALADSLAPVGVSDTAGPHQGGGGLFDAVASVLHPSLTAPGAPGRSRLYEATATPTVLEDLPRLLGGDGVTGDDLYSKDGTSAGSYRMRAVVTGLTPAWGTGKTQLRTHQQAQHTATESAGKGRSVAGGIGPAIGVGAAANAAVVRATAMPVAAARKARFSVNEQTVSSRQGAEVRGEKVLYLGTAQFTVEGTGPRSVRAILNPQARVATHAMRVWIGLRADEARELGLPLPPGVTAGEFIKKPEPQQPAADADSDTDTDTESESEGGGDARHLPFGAMGSSVTIGRLDTAPMVKAVREMFATDPRLAGYLPAFGATPPPADLSREEDEAQRTNYRELMAALSEANLRVNKEQLLSTGIRVRLRRKTTMHSHDVQLRVHGTMGATRHLGEIDDWLVRAHSGVAANAQSGRSSSRSIGGMVLAQARLIPGVLTGSARYERQSSGTRRNQGGPTTRTDVLTNGSEKASAFGAALRLNVDVTMTSRQRKLARALTPGGPGRDVPEAKLLTGLHMEEQDVRLLTPSEFTVGTDEKARLDAGADQAPGPARPVAGAAGIGDLAGLAPTPAAGQVVRDWQLVETLGDGQPVRDLALALLSRAAARGEAGRQDTALATEGLAPRLAVEERFGPRAITAALRQAASSGWVVKNLRYPRRLAALNGAVGTRLALAAPQLVHEAAGPGTETFVMGGHQAGGQQGEGTSSTVQVGVTGVQNGTEWRVGEGLSGYRSTSRSDTESATVSGTVERNAHTPKKAPLYLVRCDLLVTMVAEVKVTGGGPYVASAARTLPGAAAVWLTAEQLRAAGVDLPESARKALKVEDRRPAAERTAGGSGGGERAEASTAAASTSTSVPAPSRARASASTATGGQAASPVRQGPALARELPLGFGMIEDLPDFVPLLDRLRGNLAITGQQDLADDILPRQQLRDRNDNVQRLLRVLDRDGSTGLLASAMDGGVTVELLDGRNTPYWAVFKIVRSGDGVREGEADDGRDMEYITSAAAQQATSHGEGETTGVEGILAGSGKPDAGAGQVKSAGAAAGLGVASGSGRRGGESARGQLGMKTVAEAKTAKSAKMRVPIVASLELHKGDRRLALAGSGRTSLVHRILESDLTALHRVSAPRRAPRPAPGVPTSGAAGLGAWRAAGVPLPMEAQANGFQGAAHVRELVNTAVRAAGGGDRFRQKGQAAAYTLGEAVSTEWLIAALPLLTNAGAELPPVHASGAAGQDLQASVHARLRAGRILGAGDKMTFETAAQSSLGAPRPTQTEGQSQAEQSRQARGLFGAGVLNADQFRLNQLMGNVDGAGSASGAAANGAGSMPLHKPKFTSVLVQFTLDVRVVARVTNRVRTSRTEVAERDLTLPRPVVIRMPLPVAGRLLAAHPTEITDQHDRLGLRAAAVPPPTGV

SEQ ID NO: 22 - protein, 1564 aa, Artificial
  gene_1432510; CRISPR-associated protein
MTTTQKNKPGSLDKKGMSDYTETQCSRQLYIKLGEHDPRWIQRDIQKNTHFTGSALTLAASGKRYEQKVYTILRRLFRQQTHCTLKPPANKEVIETFLDPRLAKRLHQEVRGEAQLLLEYEWPLCDQFVRRVFGQQPDEEIATLGNQYGRVLRPDIMLLHPIPKGQKAPLKCLLPGGKAASFSPTALQGRFGISILDIKYTPDERVGRRHFAELLFYIHALTEWLHETQLDEFFFVPCHGHGILGFLEEDTLYDLTLDDLLWRSPDELSGKHTPKISPLLWEDTHQLFTHAEKTVRTLWQLAKQRTPIEEIPLCVQPACGRCPFIDDCISTLKGTTPTQSDSWDIRLIPYLKTAVAQQLNEHGIYTVGELLQGIEEIPLGNTPVPLHAEIPALKLRAQALSTQRAVYPEGEHTSLSLPKYIDMALVFNLEVDHTNELVFAFGFYLDTKQPSPKLQRLHNDWWRMWRSVLRGERELQDISSVLDLEALELGWHKGDDFSDKLSLLLQEMERLLRTLEADGVLILRAVGESYQFGSQEYTTQKYPLVRCQYSYVSGGIEPEHEYMLLKNMIQQLHRVMRMCSLTELLVTTKHETYDSLYHENFAGFYWSDEQVDHLRALVERHLPALQQDHALSKTFYELVDWMTPADSGVRHHALHKKMYDLREFVGSSVGLPQIINYTWHQTRPLWKKDFEANPYFWTPHFNQMDFGIWHSTIEEIDTNERSQKESDIRDQLVLKMRTLHEILRHFHKEASDVIPKESKTMSSQDFQRDRRNRQYHQLGSLWQGYHQLNAAISALTNDAARLTWPEQSIAKLQAGKLSGMTIKIDDRDGKDYEVVNFSLLGLSSHMKISVKDRVLLLPRTMRDSHAFPFHNMGRLSKLIVEDLVWEPSEQGYCVTAVRELKKRKEGDKETLHSFTELYALYDAEDWFVYPTDLDVWTGRLALNGDALLRRYQLGYSWLAERLMFLHGLGGEHLEAPKTLNVHAAELYTYAPQLLPQKRDCTGEDVLTPIRFRPDSSQQEGILHALSSSISCLQGPPGTGKSQTIIALIDEFIDRHKGPARILISAFSYSALQVVVQKLLDSRYGDGPAPDPTQLSDASRLPIFYASSSESESFVHDPNQQDVMHLSLSSKGVHLDGERIDFRRGSRKDKIFERMFAHKGLEGDGSFVLFANAHTLYHLGTLSKANKRRLVHEDFGFDLIIIDEASQMPASYFTAIAQFVHPFEARLVLPKDEDALKREIRCGAPELSIEGVPSSDDLTHVVLVGDQEQLPPVQQIEPPRKLKPMLDSVFRYFLEVHHVPKHQLSYNYRSHKDIVRCVRRLAIYDQLHAFHQDDAYLSAIPDVLPDTIEAPWLRQLLGRRQVVSTLIHGRQWDTALSPFEAKLTADVVLAFFAQMGVDSDERERQFWQEDVGVVSPHNAHGRLIVREIAERLLSGVGARTYLPETELMECLSTTVYSVEKFQGSDRRLIVGSVGVSSVDRLAAEEGFLYDMSRLNVLISRAKHKMLLICSQQYLDYVPRDRDVMTVAARVREYAYDLCNESQVYDVPFGSGSEFIELRWMVSKDP

SEQ ID NO: 23 - protein, 1502 aa, Artificial
  gene_5570191; CRISPR-associated protein
MQSGSGVDLFRDFNEGEVSEVLRRCAGCSRFVLIGPPGSGKTFFKENYLEGRLGTGVIVDEYTLGISTTAKIESEEARKGSGISKKAMKYLKRMIPLIEKLRETAEVDDEELRKVLGDRAPKHIVEGARRSIGDSPHRAYYIPWKCVDEPNACTFDANVSRALELIKKVFDDKKIRIRWFKAEYVPPGLVKDVIDLIRVKGEDGAREELKGWVEAYSEADETLRKILGLSDDLLEWEESFVEYLSNFVINYASYVISGLVVDPLIGASALALISVLTYMAFKREGEGYIKGIIELKRGLERLRRSDGEFNELGKLLVYRVAYAMGMSYDEAKEALMDITGLSIDELKRRVNEIEWRIKELEKKIELFRLEVPAGIVTADVNEFAKGRTYPNIKVENGELRIRVEDGYHSIVRAGKFNELVNEVRDGLLKQGFVVVVGPKGIGKSTLAAAVIWELFMNSDIGLVARVDVLDLKNYSELATFVENYGEKFSEHFGKLLILYDPVSTKAYEKVGIDTEAPIQSNIERTIKNLVNSKSSKASKPFTLIVLPSDVYNALSGEVKNALEGYRLDVSQVLINTEFLAELIREYSKTKDKPNGCALSDDVLSKLAGELAKFDSGHALIARLIGEELARSNCGVGKVEELINSAKGKAEAFIILHINGLFKVHENPDTAKALVEIFALRRPFISAVESDDSIPDTSKFLVKVYVLRSPFISAVKPGDPILTPGIVELIGEAGGVKILYGAEGEELRSWLAIWLHDLIEEAIGKLLDCIEGKGEGCKVLGDALKPWKTTGVIELLRKVSEKVNDVDSAVEYFASNYGERLTSALKVFSNECWKRAVYIIGHALAGDPLLPRRKYLSAFMSMNLSKTGIESPSDALSRLGANGDKNPQRMSLAKYYASIVESLGDALKECGVDNYLIVGDKIPSLMMGLIGNHACALAGVFIDKYNEAIAEIKRLLNIIKNRGEFYYEEAYYGLGLATIIAKAAESGRPVGHSDADAALHIASFAMSHVQSTLHIIRLLTALAPLRDKAPQRYLEVLVCALDKFTRLGTCHDWDTVMNILNELDYILNKYGVEVKGHARTLVDVINTLTHSLYKCLERCVDYWFEHRVASFRAKFERMISELADLLDKTNRWSPNLGIIAAYASLSALDSKNKNKCVRMLIESELGIDVVNKTKEVAGELSELRGSVRELLRDEDLMGFVRSRLAEADEKAAKRGILEVTSILKHTLAQYKFVNDELDEAGRLFNEAAEESKVIGDYLNYLDNRDWALRVEAIKSPLAGDDLVKLVNGFRQLYEEALNAERFMSASPDYGTLWKNILRDILGGYLVSLALTGGDEEIRRIEELLKEQWQLKYEPRPILTRLTLNALLSPRVELSSELRDWLVVKPGELIVAFGHGYLYIDYLPALKATYGTIKPGDGKRCSSVYLTFMLYALINGNEKLAKAHALMGAMNHSGKLPARLFLEAYRACCDPNNEEFRRAIAKLFFYTRALKSKTSGFWSASLSS

SEQ ID NO: 24 - protein, 1265 aa, Artificial
  gene_2435065; CRISPR-associated protein
MDRLKTDREKAVQHAEDLGYQVEVLRAKLHEARRALATRPHSYDTADLGYQAEQMLRNAQLQADQMRSDAERELREVRAQTQRILQEHAEQQARLQAELHTEAVNRRQQLDQELAERRATVESHVNENVAWAEQLRARSESQAQRLLDESRAQAEQSLASARAEAQRLTEEARRRLGEETENARTEAEALLRRARADAERMLNAASQQAQEATDHAEQLRTSTASEADQAHRRSAELTRAAEQRMSEADTALREATSRSEKLVAEAEATAAKRMAAAEAAGEQRTRTAREQVARLVEEATKEAEAVRAEAEELRERAVAEAEKARSEAAEKARAAAAEDSAAALAKAARTAEEVLQKASKDAEETRRSASEEAERLRSEAEAEADRLRAEAHDLAEELKGAAKDDTKEYRAKTVELQEEARRLRGEAEQLRAEAVAEGERIRSEARREAVQQIEESATTAEELLTKAREDAAEAREAGEADGERTRAESAERAAALRKQADDALERARTEAAKLGEEAEEAAARTREEAEQAARELREETEEGVRARREEAETELVRLREEAEQRVVAAEEALTEARAEAGRLRKEAAEEAERTRTEAAERARTLSDQAVEEAEALTATAAEEAAASRAEGEAVAVRLRADAAEEAERLKAEAQEAADRLRAEAASAAERTEAEATEALERAQEEADRRRRSAEEALESARTEAGQERERAREQSEELLASARKRVEEAEAEAARLVEEADARATELVSAAEATAQQVRDSVAGLQEQAQEEIAGLRSAAEHAAERTRGEAQEEADRVRSDAHAERERASEDAARLRSEAAEELETARALAETAVAEATAESERLRADAGSYAQRLRSEASDALASAEADASKARAEARQDANRMRTEAAEQADRLVSQAATEAESLGARSTEEAERLRAEARAEAERTVTEAAEEAERLRAEAARAVAEAEERAARAREEAERVESQALAAAEELTSQARAEADRTLDEARADANKRRSEAAEQVDRLLSETAAEAEKLTTEAQQAALKATTEAESRADSMVGAARAEAERLVAEATVEGNSLVERARADADELLVGARRDATAIRERAEELRERVTAEIEELHDRARRESSEAMRNAGERCDALVKAAEEQEAKARADAKELLADASSEAGKVRIAAVRKAEGLLKEAEQKKAELVREAEQIKREAEEEAERVVAEGQRELEVLMRRRADINQEISRVQDVLEALEGFESQPAGKAAPGGSGTGVKAGASAGSSRSGGKQNDN

SEQ ID NO: 25 - protein, 1220 aa, Artificial
  meta_gene_343942; CRISPR-associated protein
MENSGLSLDAEQKITVAEKVRKEPNKNYFISASAGTGKTYTLTNYYIGILEQHEKTGESDIVDRIVAVTFTNKAANEMKDRIVKEIQKKLESLSENDRAYKYWKDVYKNMSRAIISTIDSFCRRILIEQNIEAGVDPNFKIINELKQKKLIDKATQRAIQLAFDVYDAIESGENYTEKVTNYLYGLTTERTKRIRELSDELAKSKEDIFRLFEIFGDISDVAEKIESVVTNWRLELNESKVSERLLEVFEEAGGALRAFRNISLIAAEFYESETLDNFEYDFKGVLEKTLKVLENSVIREYYQKRFKYIIVDEFQDTNELQKKIFDLIHTNDNYIFYVGDRKQSIYRFRGGDVSVFIKTMNEFEEKIKSGRTDYEMLSLNINYRSHPELIDYFNYISENTIFNNHVYEALSESPDTSKTTNNKSKSKKKDKNKSQANGEDIVLNEALQNVNDIFSTERDENIYIHEVFRLRYPELYQKLWFIKKDDESNAAFSPDSNEFLPGDLRRVNYITISKASLLENTQENDETAKEIGLDEDNQSPGKMKKLKDMDERELEALHVAKVIKSLVGKEMTFYEKKDGKFVPISRRITFKDFSILSYKLEGIEDVYREVFAREGIPLYIVKGRGFYRRPEIKAVISALYAIQNPNSNYYFTQFFFTPFTDNLEQNPEVGVRNGKVKIFHKIVMRYRESKGQGLKKSLFQCAKELAEENELPENVTKMIKLIAKYDELKYYLRPAETLKLFVKESGYLRKIPHYPNSSQRLRNVRKLLEQATEFDDQAPTFFELTRLLERISEVQEVEASEISEEEDVVRMMTIHASKGLEFNIVFLVNNDGVDKAEEKTFFPESEDGNGRYVYISQFLDKALKKFETSRVTKELEKELKKLLEAEVIYDKTEILRKVYVAITRAKEMLFVVDLQRKNTKGIPAIKYLTPKGFEERIKIISSLDEIDKLAGSGVESVSGKQEFAESIQSLLDLENVVDKGLIFSDFTPKAYKRYISPTLLYGIKDEKSDLESVDESSEDFDSAETISITSTSNFEASKAKARLKVLNSLLEKATEITRGKQIHSMLASITKYEQLKLLVEKNALPEDILNVRVLESLFNESEKIFSEWRLAKSIEIYDEKLKERKNYILFGVPDKVFLKDGEFYVVDFKSTDLYKEAEEIERYMFQVKFYMMLLSDLGKVHCGYLVSVPRGQALRIDPPGEEFLDEIIYKIKQFEELMSI

SEQ ID NO: 26 - protein, 1196 aa, Artificial
  gene_1456430; CRISPR-associated protein
MLFGMTGCGTSSVTSSADAVTDTESVDDVKTESSGKTDEEKLSEKIGELTSAHSAGKGKDETVYVISSADGSKKSVIVSDHLKNGDGKDTLEDKSELKDITNVNGYETFKKGSDGKLTWDAKGSDIYYQGTTDKELPVDVKITYLLDGKEVTPDEIAGKSGKVTIRFDYTNNTEKTVKIGGKDEKIKVPFSVVSGVILPIENFDNVTVTNGRIISEGKNNIVVGLAFPGLKESIDLDDLKNEAVSEDAKKEIDDIDIPDYVEITADAKNFKIDTTMTVAQSNLLSSVNLTQDVDTKELTDKMDELQDGADKLQDGAGKLKDGTESLTDGTEKLKDGSGDLKDGTKKLAGGTDDLKDGADKLKDGSADLKDGTKKLADGTDDLSSGVSTLKDGSSKLAGGTDTLASGASQLKGGSSKLAGGTDDLSSGVSKLKDGSSKLAGGTDTLASGASQLKDGTSQLSGGLKTLKAGTSQLKAGTDQLSAAKPQLDQSLKDLQDMGTQLKEAENGSAKISDGIGKLGDALTAKFAKTALNMKAMDEGVQKLSAGISQAANGIKELKTKFDNGVVGIHGQVNQLIADLKDYSKDEASGIKGIGYRGIGKAAYNTGINQAQRAAQSADENLQKAQEAVDEAQKAYDEALKAQQNSADAGNSLQQQNDDLAKENAKLQQKIDELQNSADQEKKTNNVASPADNGSASSGNASAEKAGTQSTDSEGSKAAGTEPAETPAQNDAAADASSQSAAPADNTSSEDTNAGNSAADTTENVQSTQASLAGLAVSKLNEMKNALYESTVLVAKAGESSETVAQAQQALEKAKESLQSAQQAKVAADATVSALKDMKSSVDSAEKWKGTNDLKRVEKMTRIMGEAEAINSSLDILQQSVDAALDSLSSGLDSAKTGLDKIHNGIDQSLNSDETKAEQQQLNESLTALKGGAGQLTTGLDSGLQQLTDKSAATTKNIGDLKNGIDQLSRGANSLDDGAGKLAAGAEQADNGAGSLAGGIQELGKGAHDLDNGIGTLKSGASDLKNGAHQLDDGIGTLKSGASDLQSGAHQLDDGVSKLQSGASDLQSGAHQLDNGAGDLNDGIIKLDNGAGDLQKGAHDLDDGTQTLIDGINSLNDGAHDLDDGMATLQDGVIKLNEEGIRKLTDLFGDNVQDVIDRINAVVDAGDDYTSFAGTGDQENSAVKFIYKTDAIKAKED

SEQ ID NO: 27 - protein, 1089 aa, Artificial
  gene_317827; CRISPR-associated protein
VKKILFPKLDGPPSDDLENYMFLGTFEDENGSLTTAKFFVRSVSHVSPGGCYEVEGDWKRTAKGEEFNSWCLIPSVPDTFALSCVYLNGLFPPEMCGTSALSRRLSALTREYGPDVLVRALATPTILTRLSDQPEIFAANILRLWEAATRESHMALMMHRAGFTTGDLDMVWRGCAFKVAERIGGDPYQLVAIPGIDVAKADMLFRTLGGNPYDPRRIAGIIRRSLMASEGLSATNDDGEKIGFTAHVEFPGSTAVDVTDILTSGKAEPLRDDLISGIDPKIGMRLDVLRDFLSKPQEALKFGLRIRKTRDGRTLVARERVYQAEVRVARNIARLLQAPPLKDKATVQATCRNLFNQPDFQRFDAVQRTAVEMACYERFCVITGGPGTGKSTILDAVIAARVAMGTEKRSFLLGAPTATAALRMTETTGLDAATIQSLLKCKGEKAGGEQWFDFNRNNPLPSGCTVYVDEGSMVDIFLSDHLLDAIPTDASLLILGDDGQLMSVGPGAFLENLLNTRTMAGDRVVPAICLQNTYRSNPKSNLAIQAKEIRYGGVPTINGDSSGGTSMQSVVPEKISNFIVYAMSNVMPALGIQNPLKDVAVLGPQNPGVGGLWEINSQMSRYFNPNGAKIPGLSAPRFAKEMPVPRVGDRVMRRKNVKGDKLCVNGSRGFIEAYIPPSPADPDAKKGKIKIRFDNNEVRTEDVSWDWHKKFELAYALTIHKSQGQQYQYVLMVITPEHANMLDNSLVYTGWTRAKEGVAVVGSFDAFAGAVQRSRMNTRLTMLPDLLSEILVPGIADEFRSRWYKKPPMDDLPRPGGREKWFQTKYGNASGHKIRTIEGIKVEAPANGVQAGLRGGFPSPPSQPHSSGSGPTTPTASGSHQAPPVRYAVNQPTSSPPRPMFTGGIGYRPNIPVSSALPNPPATPSYDKKGVINHVQENAPPRQPNTSHQDATSPTHPKSNSALQPSQAVPLQAVGLQSPRRFGWSPTIRQPSAAPATSNAQPTARSAAPDHVPATSRPAQPHRPVRPTTPVESPSARPVPASRPSFGFIGWRPNIHPIKQTCHEPQPEMDSEMGMEDQHSSSYEDAPSP

SEQ ID NO: 28 - protein, 1044 aa, Artificial
  gene_4421494; CRISPR-associated protein
MTNKVESNVSDQTEKRLSPEVSEQFQQDTRVVAKQAAEFIEEIHPARLLQTKQEIMDLSYAKSDELLDSFAFFRIVSCTTDEVDDMFDFLNEKMDKFYTALYAVGKPVVYGIVSYGETTNLVVGLLDTEDNSDLLKSIMEGLLDGIELLPYKTNFAARTACEKEVGLISAIPSVKIEEEKQIFSLAPLMKSLNGQDYTVLFISRPLSQDIISKKRRALIQIKDQCFAVSKRNISRQQGISRSKGNTEGRTDTITKSTSNTISESFGWALGFTFSESYSETTSESSSASENYSQTITDAINQSEGISAEVQNGVALELMDYTDKAIERLRQGRSNGMWETVISYSTDSKLAAGIIRACISGEFAKPNPVILPQVVHSFHLDKTEAEGKSLLVPEILDAEPELSPLCTVVTSEELGFMCTLPDVPVPNFELKKGKTYPLITDNAVGVEVGHICEGRRILENMPFSLTHKDLARHTFVCGITGSGKTTTVKGILKEADTPFLVIESAKKEYRNINLKDKKRPQIYTLGKPEINCLRFNPFYIQCGVSPQMHIDFLKDLFNASFSFYGPMPYILEKCLQNVYKKKGWNLTLGFHPYLVNTANSAKFFDADYMQKKYASAAHKYLFPTMQDLKLEIERYIKTEMDYEGEVAGNIKTAIMARLESLCSGSKGYMFNTYEYADMNALLNHNTIFELEGLADDSDKAFCVGLLIIFINEYRQISQEMLDMNRTLSHILVIEEAHRLLKNVSTEKSSEDLGNPKGKAVEHFANMLAEMRSYGQGVIVAEQIPSKLAPDVIKNSSNKIIQRLVSADDQAVMANTIGLTGEEGLDLGSLKTGTALCHKEGMSLPVRVQIAMVDDIKVTDDLLYGKDIKKRLYQINVSLAKEVLADSLPLMGMKMLNTILVQDCNHVSHAVTVCRQSFRSSLKKNNVTLVMCDNENEIYAELLYEGVLRYLLNGCYILKQMIPDELCSDIYQLMLSPDNDKLVLVKEQLQAEYEENLEDQGCFIVAQLIYKNAFERTDIVQTIKNYFFEISDEDILKIKAEWRGSD

SEQ ID NO: 29 - protein, 1037 aa, Artificial
  gene_3011455; CRISPR-associated protein
MSSWDPQTSGLTVRLRDNPGRVGHTTGRWKFAGSLTLVEVAFGPNEKQFKNQELLEQVHSSEDPLDLLLGGKLGLPSDLRRVLAFEKVRGELTNIFYSMESSNTDFYAHQFKPVLRFVESPLGRLLIADEVGLGKTIEAAYIWKELQARYGARRLLIVCPAMLRDKWRRDLQAKFNIKAQVISASDLLVKAREIVTDGALESFVAISSLEGLRPPADFEDDRKASRRAQFARLLDQNPTSADFALFDLVIFDEAHYLRNPSTANNRLGRLLREASRHLLLLTATPIQIGSQNLYQLLRLIDPDVYFNEAVFADVLTANAAIVSAQRALWANPPKIREAEAAVRSARANSYFQGDPVLQRIEALLPEADTQTVMRIEALRLLESRSLLAQHMTRSRKREVLKDRVRRASQVLAVEFSSLEKEVYDQVSAAIRAKAKGESWAVVFSLICRQRQMASSIVGALESWKNTDFLEELVWDDLGVLPQDLFGDRGDNQQEVAAPTINLTSDVDLARLEELDTKYRQLIQFLKAELKRDPHEKFVLFAFFRGTLTYLHRRLQADGVQAIVLMGGADIDKDAVVETFSKTTGPTVLLSSEVGSEGIDLQFCRFVINYDLPWNPMRVEQRIGRLDRLGQRAERISIISLAVSNTIEDRILMRLYERIAVFRESIGDMEEILGDVTEKLIVQLFDPSLTEEEREQRAAQTELALENSRQQQGELEQEAINLVGFSDFILDQINESRAQGRWLSGAELLALVDDFFARHFAGTRIEPLDHEVTSASILLSEEAKLSLGQFIADTAPAVRTHLHQSLRPISCVFDPRRVNRSVKGAEFIEPSHPLIQWVRQAYELEPAQIHRASALHLRSGETDMPEGFYAYSIHRWSFQGIKRESVIAYAAQMLGQARPLTSIEAERLVGLAASRGQPLANVFASGVDRHELSQAAQACEEQLGLEFEKRLVDFLVENTVRCDQQATSATKFAARRIAELQDRVERFQLEGNDRLVPMTEGLLKKEESELKFKLQVVDKKRNVDPTMVHLGLGLIRVA

SEQ ID NO: 30 - protein, 979 aa, Artificial
  gene_2590511; CRISPR-associated protein
MSNFNFLTDISPELAQFGKSAELYCHDDKQVALVKLRCFTEVVVGEIYSRLSLTPPVRDDLYNRLRSYEFKDVVSDKGIWAKLDVLRHKGNKAAHSSNGSDEISLNETLWLIKEAYLVARWYAQAILNKPITPPEFVDPVKPIDHTSRLEAELERQRQELNKREAELKTQLADNSDKYQQQTSELIAQLDEKNDTLSNVKKEQALLQIELEQKQKDLVASQQAFFDYRTREEFKQASISSASSFDLDMEVTRRNIDIFDCFEGVSLTKGQNQIVKQINEFLTDTKQNVFLLNGYAGTGKTFITKGITQYLERIGREFAIMAPTGKAAKVISDKTMQPASTIHRVIYNYDNVKEYKVDGVEGSETYRCYADLKVNVDTAEAVYIIDEASMVSDRYSDGEFFRFGSGYLLKDLLKYINIDHNDHNKKVIFIGDNAQLPPVGMNTSPALDASYLKENYQVAVASGYLTEVVRQKGDSGVLNNAAMLRDGLEQNLFNKLKFEVNDHDVFNLSSENLLSTYLDSCDRKVSRTGESIIIASSNRQVAEYNRLVREYFFTGQQQMVAGDKVISVANHYRADACITNGEFGMIKEVLSPHSELISVDISVKGDTGDMVKRKVNLSFRDVILGFRNDYGEPFFFEAKIVENLLYNDQPTLSSDEHKALYVHFLNRHPELRRKGNEQKLRIALLQDPYFNAFKLKFGYSITGHKAQGSEWKTVFLQCQTHQKALTKDYFRWLYTAITRTSGILYVMNPPQLRLGDGMKIAGAYQPKAVNLDNSAPEGVEVVRPSTEATNSVATAKFDFQTDIPQLKKLYQLVDACIEGTGITVVDVLHYNYQDRYILQRGNEQASISFNYKGNWKVSGVKSITQDGFDVELMALLGQLEGTLLDVPEPSKDTQFHFSEPFLEEFYLNVMDQINSVGADISKIESRSFCERYAFVKGNELAVIEFWYNKSSQFTKVQPMPQLSNSTRLIDEIICQIGVLL

SEQ ID NO: 31 - protein, 896 aa, Artificial
  meta_gene_463174; CRISPR-associated protein
MVNNKKVMSDNTQPKASVAEAFGNAKKAKTINGIIKKIIFQNAESGFTVLNVFSNDKFITASGTFFDKPLMDSKIKLKGEFTYHKKYGYQFNFTQYEVSLSNTKTAIIEYLSSSIFKGIGKAIAREIYDKFKEKTLDVIDDEPEKLKDVNGIGAIKLAVILEGLKESYGLRKTVMFFKPYQFSDYQIKAIYNRFKDKSVTIAKENPYLFTDIKGIGFKKADIMSEKLGIKKDDPNRIKEAIKYVVNQICESSGNCYIYYQDVKKGIGEIIEDLEETDLKKYLNDLIKERKLLLDFKGIYGTDNYLSVVRDRYVSSKSIDLKGEEVLDFTSAKEGKRLGCARIYMPVYYHCELGAAKELKRIRESASPASDKIESLDDLDKFLELGNNHVSLTNEQKTAVLNALKYKISIISGGPGTGKSTIIKTIVHLYSGEKIALTSLAGKAAQRLADIVNSGQTLSSRNDHSQEKMGRLNISTIHRLLKAQYDRQTGESYFTYNERNRLPHDLIVIDEMSMIDIIIFYKLLKAIKDDANIVFVGDVNQIPAVSPGDVLRDLIYAGAGNMDGQDKTKPFFPSTFLTKVFRQNEGGLINLNAHNILNNKKFVTLRKDCKEKNISTAEKDDSFTIKYRKEYDIAVGGKHELLIDFTRFIKRVVENRINKRDVGLKSANMSIPTMLFDDIQVLTPMRRGDLGYFNLNNILQDIFNPISPLHLSASVENIFICNGIQFRLYDKVIQKRNNYDQDVFNGDTGYIVDVNHNEKYLTVDFSNYSDLSKKCNEIGTGAESCANLTAQEGKMTNKAIKLVKYNFLDVYENISTAYALSIHKAQGSEFNNVIVLFHQTHYMMLKKNLLYTAITRGKKNIVIFGTFKAIGIAMGSKETVRNSGLKDRLSEEFLDAN

SEQ ID NO: 32 - protein, 887 aa, Artificial
  gene_773846; CRISPR-associated protein
MIENLPPFSIILAPAYLHPILRADIMKQTSGCMGLQLLSPQTFFASFTQKQARDHVEISFLYKQNIEKIISQLQTYQAIALTPSFLMECYDFIESMKFYHISVDELPDKTQAQQEIKTILNNIFPIQTAQDIWNEAVLRVSDCSNVYIYDAFYSLKDEKILNILTSKGAHTIPLPKPQQQKEFYHAINPRQEVEAIAQYIIQHDLDADDIIITLASSTYKPLIEQIFKRYEIPYTLLQKNKASIVTQRFVNLIAYALSFDQEDLFACMDAGVFQSEHLDELREYIEIFNCDIFQPFHHLMNVQANGHILDEVEITKLKELEEIAESGRQELCETLSLFIEDDLHQLVTHLLDILHNGMKEASMEDISVLSNIQDVVSSSWNYLNTKDDLAFLLPFIEQISISKSVREIHGVIVGDLKQIIPNRTHHFLVGATQKNYPAFPSESGIFDEIYLRDTTLPDMETRYQYYIAQCEKQLHTNSHLIVSFPLGTYEGKGNEAALEIEEEMKCDPTAFPIMENYEKITQTYIIQPETAKALFVKGHHIKGSISAIERYIHCPYSYFLRYGLSLREPMQHGFDNSYMGTMAHYALETLVDELGKQYTKAAMERIEEIVNQEVEAIAAVFPNNADLMEVIKHRFLVSFAQTLKRLDDFETHSSMGPYLQEYEFHEEFPITEDISFALKGFIDRIDASGNFHCILDYKSSAKSLSEDKVFAALQLQLLTYSIVAKKQLHKDILGAYYISLKNQNIPYIAGKMKRRPVGFVETEKDDYEENILKAHRISGWTMRKDIDMLDDNGSHIIGVSMNKDGIVKARKYYRYETIYEWFISLYRTIGNRMLSGDIACSPDADACTYCAYYEICRFKGFASERKPLVDIDDSLYWEGGVDDADME

SEQ ID NO: 33 - protein, 840 aa, Artificial
  gene_1188229; CRISPR-associated protein
MKGSIKSHKSAIAVLLALALSGQSSWAAQNSAAVQGNDFLSSIQQIEVKQIDFPAPTHRQQTPSASRAQINDLQQEIARLKKQLKAAEQEKKSLSAPGDLQAQNTQLLKDNSALAKENDRLSRSLQNAQREQGAASTQQAARIEALEQKTAELQASLASKTEELAQLKKSSNSQAASESALQKQIARLETEKAAIAERNTKDTARFNRDMQALRNELNKRADELVALKNAGDKRAQSQTALQKQLAQLEKEKAALTAQSAQSIDVANKKVQALQAELDKRSAELAALQKTGSEHEKSQSDLQKQLTQLEQEKAALTAQNAQSIDAANKKAQALQAELDKRTAELTALQKAGSEHEKSQSALEKQLAQLEREKAALTAQNEKSIGALNKQLAQLEEEKASVTEQNSLLMKNSSLSKEEKAKLQKAQAEQTALLEKNQAAEAALKAQIAALTEKLNASTTLAATSQEKVAALASELASLKGSQSEKAQALQSQQQQAAQIAAAKEALTQQLATAQADIATLKQSLAEKENRLQQSDKALLALKEEAQSAKALTTASATSQQKTQAELDTLKRANEELNAKLASLSAENTAQKAQAEKEKAELLAQAEKEKAELLAQAEKLKADAATQVQTVAATKAEPEVSAAALKDKANKQSYANGVMFSRLVQKSMDQMADLGIKTNLPILLAGIKDGLAQKVAVEPKTLLSLHESMLKELSSREEKKYQAGIDQLEKATAKKKLLKRNKSLFFVQAKAGKKAIAPGETVNVTFKEATYEGRVINNNANVPVTYDENLPYIFQQALELGKRGGVMEVYCFAGDLYNPDTMPPDLFNYSLMKLTVTISGGK

SEQ ID NO: 34 - protein, 838 aa, Artificial
  gene_800233; CRISPR-associated protein
MDYDVSISIGTTANLGDLDKANKAVQDLGRSIDKLPPQLLPGGVGGTGAGGSATPSYVGTPSTSGSMTWRLDGMTELTGALGQTEAAVKQVDKSITITSQRLDKNSSWLSRSISTLASLPGKIQSWGSNTMQAWGQFNGPLQNVKNMISVGKQAWDLGWSLGESLNEAFGVKTKQIDAKVAGIIQAAQDKLARWQDSINSARAQHREDAFLKQEAAGVKQVNDAYAARLRTIEAIDRKAMAGLELQQKLLQIENEKNRSIIRQRQIRGEISDAQARDELAKIDAKDAGERMDIERKQAEQAAATSQAKAEAAEERYRKLMELSQSGMARQAVQDLKPMDILNKADSLKRAEEDLAKWRSIQQRQKEAQKEIQQAIKDQARASTMLPLVGAPIALARKQAEDQARQDYEAAVAAQHEFMHDKGMSFNETDKGNEDALKKIVEQRRKALDSMLGKIDKTGLVGNMDGMAEDQRLGEYLRILKLVQDAMAQDAAQLESIFLETEALKQQAAEDKERVQRVMQEHQSQQAANDAVTKETAATNARQDADKHADVMVGAQEERLRKEIETKQRQQEKQKEDLSKTNERLNANMERFQQYAESFEGNDALSAKLKQFSDIFTRLKGRPRDTWNKKDLVDAKAAEKFAKELVEASKNSTNQDKKGIAQAAMQAIKAWQESIKKERAIKKNDKALRELERTAQDVANLSGKLHDGQSKVLELDDWLAKMRRKVLGSSGEIANKAPIGALPQAEEVLKKVLSEQGDGGTAVTQGERKLLEHLKNKLKNDDRRLEAGNEFDEMIGLIDQILTRYSSAQSTHSKLSGEVARLKARLDKIDSQGKFGPHR

SEQ ID NO: 35 - protein, 803 aa, Artificial
  gene_1538800; CRISPR-associated protein
MTDPTSSVQTKQGVRKIYAYTTPAEESVDWLNGKGNGRVKIGHTTRSVAERIREQFGASSTDRTWYPRGEWDAQAEDGTWITDHMVHRYLSKRYRRVPDTEWFEVDPEAVWEAVEVLKNDPKARPKGKDCYELRGEQRAAIDAAMTYYEADPSNRWFLWNAKMRFGKTFTAFKLAERLRSKRILVLTYFPAVDDGWSEEIEDHVDFEEWQYAENGASYEDGDQVQVSFSSFQMLEHKTLGKNRENANTKADALRREIAAVNWDLVIIDEYHHGAHHPARREFVSSLKTQRILALSGTPFRAIAKGDFATENKFDWTYLDERQALADWAKKETCEANPYEELPAIHFVGYRLPPHAALTGVDGDCDLTYSPTTIFKADKDGFKNPEAVKDWLQSLSTLSGSARRAGGVPPPYHPDFTGDVLSHVLWLLPSKHSCDAMKRLLEDGWFPGGEGEVIQVSGSEGETGKPAQITKKVRDKIAAAKRSITLSVGKLTTGVTVPEWTAVFHLKAGSSLESYLQASYRCQSSGSINLRNGDREVKTNCFVFDYDPDRMLVVMGDYIKSLKGSGAPLDDRSNAAFPTVIFDDEKNGHIPLNVSDIENELNNYLLKRRPAELMSDSMRLLEDAISAGGLNKALCAQLVKAGKSKSPHHAMRDLDPSLFKSKTPGTIIGDNGKQSAKSTEVADENPKNDEIRAIKEAMLVFIKSLGHLAYIGDLREASVEDLFKVDDELFEKIMGNDKDQVREIIDGAGLDRVQLNAMIQKILMWENFEFVVSGCRNRDELGECGKWYPSDEEATYAFSLQPNR

SEQ ID NO: 36 - protein, 1679 aa, Artificial
  gene_5543656; CRISPR-associated protein
MQRTLGNAATARAVGRGKRPAFRPSPPAIDERAEQGLVLPPYLMDLEAGGLSTAYGLTGHEFVRGAVAAVVGHGGGTVAGIAAELAGRPESFFGRGRAFAVEAGPGGGSAGAQGGGGYDVTVSIAPAPDDRPPTFHPAAGLGTAAPDPGGAPLAAVDDPEGKETKVDVQHNTGATASRSVGNSASKGVGGTAFGLAPVAPGLWLGGAATGNVQPWQSSRDSRSQRGVAEPRVLRSDKGSVEVARRVVYVVRVRPQAGGDEQVFRGSGGLTQRVPTEHLIPAGTGAPARPEPVDAGLARRVALADSLAPLGVFDEAGPHRGGGGLFDAVASVLHPSLTAPGAPGRARLYEATATPTVLEDLPRLLGGDGVTGDDLYAKGGSSAGSYRMRAAVTGLAPAWSTGKTQLRTHQQAQHTATESAGKGRAVAGGIGPAAGVGAAANAAVVRATAMPVAAARKARFSVNEQTVSSRQGAEVRGEKVLYTGTVRFTVEGTGPRSMRMIRHPEARVATHAMRVWISLRADEAQELGLPLPPGVTAGHFIRPPRPGAAPTSAAGGEGEASTPAAAGSERHLPFGAMGSSVTLGRLDTAPMMKAVRELFATDPRLTGYLPAFGTTPPVAGLSQEEAAAQRANHRELTTALSEANLRVNKDQLLSTGIRVRLRRKTAMHSHDVQLRVHGTMGEAGHLGDIDDWLVRAHAGVASNAQSGRSSSRSIGGMVLAQARLIPGALTGSARYERTTSGTRRNQAGPTTRTDVLTNGSEKAAAFGAALRLNVDVTMTSRPRKMTRALTPGAPGRDVPEAKLLSGLHLEEQDVRLLTPTEFTVGAEEKRRLDAGAGRAPGAESATTATGIGDLAGAAPTAPTGQHLLSDWQLVETVGDGRPIRELALSLLSRAAARGEAGRRDPALTTEGLAPRLAVEERFSPRAITASLRQAASSGWVVKNLRYPRRLAALNGAVGTRLALSSPQLVHEAAGPGTETFVLGGHQAGGQQGGGTSTTVQAGATLVQNGADWRVGEGLSAYGSTGTGDSEAATVAGTVERNAHTPKKAPLYLVRCDLLVTMVAEVKVTGGGPYVASAARTLPGAAAVWLTAAQLRAAGVDLPRSARKELKADGTPAPTTTTSAAGVSGAGSGVRSAARSDHGPGTRSGAGSGPGEASGGGSRPRPTLSRGLPLGFGMIEDVPDFVPLLSGLRTTLALTGHQDLADELLPRQQLRDRNDNVQRLLRVLDRDGSTGLLASAMDGGVTVELLDGRRTPYWAVFKVDRVGDGVWDGEADDGRDMEYITSAVAQQSTAHDEGESVGVEGVLAASGRPDGGKGQVKSTGAAAGLGLAKGSGRRRGGATRGQLGMKTVAEAKTAKAARMRVPVVPSLELHRGDRRLAVAGLGRTTLVHRVLEADLKALSRVTTPRRPAAHPRPDAPQGSDAALGAWRASGVPLPMEAQVNGFQGAPRVRDLVSRTVRAAGGNPRFREKGQAAAYTLGEAVSTEWLIAALPLLTHAGAPLPPVHATGAKGQDLHASVHARLRAGRILGAGDKMTFETVAQSDLTAPRPTQTDAQSAAEKSRQARGLLGAGVLNADEFRLNQLMANGGGAGSATDASAGGAGSMPLHKPKFASVLVQFTLDVRVVARVTDRVRSSRTAVAERELTLPQPVVVRMPLPVARRMLAAYPEAVADSRGELGV

SEQ ID NO: 37 - protein, 1660 aa, Artificial
  gene_3943627; CRISPR-associated protein
MQQTLGNEATARAVRRGKRPANRPPAIDERAEQGLVLPPYLMELEAGGLSTAYGLTGQEFVGSAVAAVVGHGGGTVAAISAELAGRPESFFGRGRAFAVEGAEGGQGGRNGQGGNGFDVTVSIEPAPDDLPPTFHPAATLASAPPDPGGAPLAAVDDAEGKDTKVDVQHNSGTTASSTVGNSSSTGAGGTAFGLAPVAPGLWLGAAATGSVQPWQSSRDSRSQRGVAEPRVLRSDSGSVEVARRVVYVVRVRRQEGGDEQVFRGTGGLTQRVPTEHLIPAGTEPLPSSGAGGQERPVDADLARRVALADSLAPLGVSDSAGPHQGGGGLFDAVASVLHPSVTASGAPGRSRLYEATATPTVLEDLPRLLGGDGVTGDDLYSKDGSSAGSYRMRAVVTGLTPAWGTGKTQLRTHQQAQHTATESAGKGRSVAGGIGPAIGVGAAANAAVVRATAMPVAAARKARFSVNEQTVSSRQGAEVRGEKVLYRGTVQFTVEGTGPRSVRAILRPEARVATHALRVWISLRADEARELGLPLPQGVEAGEFIKQPEAGAEERHLPFGATGSSVTLGRLDTAPMMKAVRELFATDPRLTGYLPAFGATPPPADLSREEEEAQRANDRELMAALSEANLRVNKDQLLSTGIRVRLRRKTAMHAHDVQLRVHGTMGEAHHLGEIDDWLVRAHAGVAANAQTGRSSSRSIGGMVLAQARLIPGVLTGSARYERQSSGTRRNQAGPTTRTDVLTNGSEKASAFGAALRLNVDVTMTSRQRKLARAVTPGGPGRDVPEAKLLSGLHMEEQDVRLLTPSEFTVGPDEKARLDAGAGQAPGAERPVTGAAGIGDLAGLAPTPTAGQLVRDWQLVETIGDGQPVRDLALALLSRAAARGEAGRRDEALGTEGLAPRLAVEERFSPRAITASLRQAASSGWVVRNLRYPRRMAALNGAVGTRLALSSPQLVHEAAGPGTETFILGGHQAGGQQGEGTSTTVQAGATLVQNGPEWRVGEGLSASWSTSTGDTEAATVSGSVERNAHTPKKAPLYLVRCDLLVTMVAEVKVTGGGPYAAGSARTLPGAAAVWLTAEQLRAAGVDLPESARKALKLERPRPENGPTTSRAEGSGGGTQTPAREGVGATGGGPSRPGPGLSRDLPLGFGMIEDLPDFVPLLDGLRGNLATTGRQDLADDLLPRQQLRDRNDNVQRLLRVLDRDGSAGLLASAMDGGVTVELLDGRRTPYWAVFKVVRSGDGVREGEADDGRDMEYITSAAAQQATSHDEGESTGVEGVLAGSGKPDGGVGQLKSVGGAAGLGLGSGSGRRRGGAARGQLGMKTVAEAKTAKSAKVRVPIVASLELHQGESRLAMAGSGRTSLVHRILESDLTALRRVTTPRRAPRPAPGAPTGGQAGLGTWRAAGVPLPMEAQANGFQGAPRVRELVNATVRAAGGDDRFREKGQAAAYTLGEAVSTEWLIAALPLLTNAGAELPPVHASGAKGQDLNASVHARLRAGRVLGTGDKMTFETAAQSHLGAPRPTQTDGQSAAEQSRQARGLLGAGVLNADEFRLNQLMGNTGGSGSATGAATNAAGSMPLHKPKFGSVLIQFTLDLRVVACVTDRVRTSNTQVAERDLTLPTPVVIRMPLPVAGRLLAAHPTEIADPHDRLGLRTGAVPPGP

SEQ ID NO: 38 - protein, 1043 aa, Artificial
  gene_5085315; CRISPR-associated protein
MKPLKSYLAWVAVTLAVAGATTACQDDIDDPIIDAPVAKDQPNTSILELKTKYWNDATNYIDTIGTRDDGSHYVISGRVVSSDEAGNVFKSLVIQDGTAALSLSINSYNLYLKYRRGQEIVLDVTGMYIGKYNGLIQLGQPEWYENGGAWEASFMSPEYFTAHAQLNGFPDTSKLDTLVVNSFSELPTDPAGLIKWQSQLVRFNNVSFANGGKATFSEHKSNVNQSLVDAEGSSINVRTSGYSNFWNKTLPEGHGDVVAILSYYGTSGWQLILNDYEGCMNFGNPTVPEGSQSKPWSVDKAIEIEKAGTEKSGWVSGYIVGAVGPEVTEVKSNDDIEWKADPLLSNTLVIGQTADTKDIAHALVIELPDGSKLQTLGNLVDNPGNYGKQIALHGTLAKAMGTFGITGNNGTTNEFSIEGLNPGGEGIPEGTGVKESPYNCAQVIAGVSGNAWVKGYIVGSSAGKTAAEMTNATGAAASTSNIFIAAKADETDYSKCVPVQLPIGEIRTALNINANPGNLGKVVAVKGSLEKYFGQPGVKTVTEFDLEGGVTPPTPPTTSGDGSENNPYNPAEVIAFNPQSSQEAVKSGVWVTGYIVGWADVSAAPYAINAETAHFDASATMATNILVASSADVKDVSKCIGVQLPTGEIRSALNLQANPGNLGKSLQIKGDIMKYCGVPGIKNATAYKLEGGSTPTPTPTDPVASINENFDASSSIPAGWTQKQVAGDKAWYVPSFNGNNYAAMTGFKGNGPFDQWLISPAIDMSKVSKKVLTFDTQVNGYGSTQSALKVFVLTAADPTTAKTTQLNPTLATAPATGYSDWANSGELDLSAFSGIIYIGFEYTSPVADNYATWCVDNVKLNAEGGSTPDPTPTPTPSGDFKGDFNSFNNGQPLSKPYGTYTNNTGWTATNAIILGGGETDANPIFTFIGAAGTLAPTLNGKTSAPGSLVSPALTGSIKTLTFKYGFAFNESKCQFTVNVKDATGNVIKSEVVTLDKIEKAKAYDFSLDVNYNGNFTIEIINNCYSQLDANKDRVSIWNLTWTE

SEQ ID NO: 39 - protein, 986 aa, Artificial
  gene_4028206; CRISPR-associated protein
MVGVNERARVPFALLGVVLLVGSASIAAGLGGTSPTREPATEAAIEQGRTSLGGTVHDATRTAARNVAASPVVAPANTTLGRVLAATGDPFRAALELRTYLAVRDRLSATTERGVTVDPSLPALRDSADIDAALSRTTVEPVGANATAVRTTVANVTLTAMRDGRVIDRYAVSPTMTVQTPVFALHERTRTYQQRLDSGATEPGLARRATARLYGVAWARGLTQYGGGPIANVVSNQHVAVATNHALLAQQRATFGATDDTGRRAVRVAAARAAGTDLLAATGQSGKQIQELLAGVDAATPGSTLDPVAAANPPITPESALNVSVGEQATTAFDRFVTTDLDAVLAAPYRVTVERRRAVTDSATTTAGRERPTGDNWTLVGTEQTDETTVTDGDATVGSPVNPWHTLATTGRRVAETTRTERRWRRNHTTHTTVETTTQTRRVSIRLVGRHDGGAAPPVGTSPIHERGGAIDGPNLAAVERRAKTRLLGDEQDLDALAARTTSDGTTQTTIRGEQPLELRDWVYRDLVRLRERVANVSVAVERGAVGTYQVNPSDELAGALRARRARLVDRPDEYDGVADRARVAARGAYLDAVITELERRADDRDGVKERLAGLLAARGLSLGRLRSIMAARSQVTTPTSHSISGVGGSYSLDVEGVPAYLTLASVNRTQTDSLREGSVRPLAARNTNIFTVPYGDAADGIVGKLFGGDRVRLRSAARALAAGEELATHETLEADVETAVSRRRRGMRRVLRRAGVGDSRSDRRRIVAAGLGAWETVAERAIAVTENRGPDAVAAVALRRSPGSFDGPADRDDLRRSLRAVATDGRGVPESSVTPHVERARQMVGKLVKQSVGRAANQTTTAVRERLESKTGKLAAVPSGIPVTPVPSQWYATANIWDIEARGGYDRFAVSVRNGGPGRRLTYVRDGSTVVIDWNGDGELERAGTATAVTFAYRTAVVVVVPPGGQGVGDVDGNADERSAGWGER

SEQ ID NO: 40 - protein, 881 aa, Artificial
  gene_277399; CRISPR-associated protein
MPTTFENIKLKEDGTEGQIITISTFYVWDCTNQRFSTSPPVVLRNTMLAALYPAKEFIIGEIPKTSTNPSLLDPFKVPAVSDPYFLDLASNSRTHGRFLFTPKRTIGRDYFPKKDDWKRIIYGSILHTGCNRMFYREIKYIVVDDERRNPSDSSPQDDGVNNTHWDTGDCHAKLSKSLLTLLESWETIGNEDNPTTIQIRAAIFKEWTIKGTASHSYKFETDPRFAGVDLVIPLSCFKGNKPAPGNYTGKVLIGVVHEAEERRAKPGWMLWQWFSFETLEEDGIISKLHEKCQKLSTALDDIYKLADVLRIDLDEAEQELANLDDNPDAEVAYVDSVLKIIKADKKGVLILHPYVLLKVKFRLREMWKNLAKSAGVRFYSVMCTPDTSLEKYQKAYGNDFVFKPKVFCSPSFNEGQYIVFCNPMRHWGDVQLWENFHEGRFRNTRGVLAATRELLLSLGRDTDGDFIQLINSSRYPNLTMALYDMDAPPKVKKFPKVALTGSLQQIAINSMNDITGVVASLLGRARAIGAELIVLDIPKEGEMRIIDFLSQELQIAVDSLKSAYPNNQDGLKVVKEFLDKSGADIQWLADLKSDDCYFTRPCLVNNNLTDTVTRIVSLVNSYYRQPNLKEDTIPMDYRFTLFSLVVSDAVQDAIALRERDAYRAEMGAALAHKAANDDDRLVKEVTAKFRASTEVIMRETLNPFRKPYPPKTWAASYWRVNHLAKSGTAGLVFLLFCDEIIEELKNLENKKVWLITIYAVQFTAFARPQLNAWNGEELTVRSSFLNVNGKDKVSLEGKLDGQPGFINMGLVNEKDIAQVPNGWTGRVKIYAKTYENDKYPRKMSANDVCTSLYCFSVDMEQSDIDDFMNDHWSTNSRFNPI

SEQ ID NO: 41 - protein, 838 aa, Artificial
  gene_1961732; CRISPR-associated protein
MNRSLVSAVVLTAVLFPNCVKSAPDLPTQPFAYHEDFETADPVQFWVSNGEYEVNSKGLTEEKAFAGKKSFKLDVTLKTATYCYWSVPVKVACAGKLKFSGRISVSQASKARVGLGCNYVFPPTHHSGCGAFDTFDKATDDWQLQEQNLVADGDERADGVLRQNTSDATGANVVTFTDRWGIFLYGGEGSRVVVYVDEVRLDGEVPDAQVYAAEADQRFEPAREVFRKRLTAWREELATARQGIDALGALPPVAQRMKEVALKAADSAEADLTKFAEASYASPTDITRLESSVRTVRYATPNLIDMSKPGVADRPFVTYIVKPITNARLLPTSFPIVGRIASELSVTGCAGEYEPASFAVSALKDVEKLVVTPTDLNSGANLIPANAVDVSIVKCWYQAGVSISDTRHCLLTPELLLKDDALVRVDTEKKENYLRSGEGEKYALISTKDSSTLTDIQPRDAKSLQPVDLAADTTRQFWVTVHIPDDATPGEYTGTLKLAAANAPAAELTLRLRVLPFKLEPPALCYSVYYRGVLTPDGKGSISSEEKSPEQYAAEMRDLKAHGVDHPTLYQSFNEPLLEQALDLRKQAGLPTDTLYTLGLGTGSPTNAADLDKLRATATKWVEVAQRHGFGEVYGYGIDEATGDRLTAQRAAWQVLHDAGAKVFVACYKGTFEVMGDLLDLAIYAGAPLADEAQKYHQAGQRIFCYANPQVGVEEPETYRRNFGLLLWQAGYDGAMDYAYQHSFGHGWNDFDSPQYRDHNFTYQTVDGVIDTIQWEGFREGVDDVRYVTTLVKAMEAAREAKPALVKQAQTWLDGLDVKGDLDEVRGKTVEWILKLTK

SEQ ID NO: 42 - protein, 816 aa, Artificial
  gene_2755817; CRISPR-associated protein
MLLVHIAGHADLGAPSPFEDPDKIGPLRAEELKNCMTPHEATRCLFDLSFTQTPSHKYTDTAHSPHSGSALRKELTAVSQISAATSTDETTEVLIIGVEGEDTPTDRLARALVDALRMASSEAADLAGTSEIIIRDACILPSLAVSRESIELLERRIGAHDGHVLLAMAGGATTVLAEAAGVAAATHQDEWSLMLVDRVEEGSDGQSLPLIPMSVDADPLRGWLMGLGLPTVLDDIYEQSDRIDTEVKKAADAVRRVMGELDSEPSAEDFAQVLQADVARGDLAAGMTLRAWILAKYKHLRDAHSYTNDSCKQSNKQLRQELGRVIGRLRESAKSHALEEPESWLVAQGDLNDLGKYATHNLESPLRNLTSNNLQERIKQAVGEPPEWLSMPSGDVCLLTAQGKAARNAPLTSGADAPDRKRRRPIIVSLLTSEPSDSVRQACAVHGPLTLSPFIACSSSSLSEGRRVADEVKNGEQPASHSPWTLDETSIKVHDYGESITRPGVSSETISSSMKGLSRAAEHWLEERTSRPRAVVVTVLGEKAAAISLLHAAQIFGAKHGVPVFLLSMVNSKDTETGESKESVQFHQLGLDRDVRQALLKATTYCLNRFDLLSASRLLSLGDPAMEVLSNEANILADRLIESVNTNDLDGASSTVLSAMNAVADLVKIVPSDAQVRLTTIVGELLRTPDEKYRSPNFKAPVALACASPDFDQGNDYKKKLKQLELEPSESLLRLLIRVRNKIPINHGRNTLDVATELSLQNFPDGNRYTYPVLLQRAIAAVGSKHGARAGDWGHRFHSLRDQVEALGKTGYGEKP

SEQ ID NO: 43 - protein, 802 aa, Artificial
  gene_2831443; CRISPR-associated protein
MTYHIRAGQLVLEINERGEARLQADKVGASEGLPMAMYPSPLLRLVQDGELQEPAGCEQEDRTGTLTLTYPNGTKIKVGVAVRDSYAALEVLTIESGSPDAVIWGPFRTRIGGSIGESVGVVHDGRFAIALQVLNAKTVGGWPLELDRLAYMAPSYSEGDAPDPNGRRGSDNKFEYPVCTAWPTVDGGSALQAYARDRTKRSIRKAWNVPATEVRPFEGEDAVIVGSGIALFGCPVEEVLETIEQIELGEGLPHPTIEGQWGKTSPAANQSYLITAFTEETIGEAVQYAKLAGLSYVYHPDPFEQWGHFKLKRGSFPSGDEGLRRCSEAARAEGVSLGIHTLSNFTTLNDSYVTPVPDIRLQPLGAAVLAEEADERGDSLTIDEPWPFTVALYRKTARIGSELVEYAAVSETKPWRLLGVKRGMHGTAASKHGKGETVARLWDHPYDVVFPDLELQDEYADRLAELMNGADIRQVSFDGLEGLYATGQDDYGVIRFVERQYRSWGREVINDASIVVPNYLWHMATRFNWGEPWGAETREGQLEWRLSNQRYFERNFIPRMLGWFLVRSASDRFESTALDEIEWVLSKAAGFGAGFALVADEEVLKRNGNIEALLAAVREWETARRLGAFSAEQRERLVEPKGDWHLEPVGPQRWNLYPVQATKPLVCTPAEQQPGQPGGSDWAMFNKYAEQPLRFTMRVRPSYGNEDAAVQRPTFYTDGVYMTFDTEIAANQYLECDGTRTGRVYDANRNLLRVVEASAEAPTVRHGGQTLSFSAKFIGDPKPDVAVKVWLYGDPETVSADE

SEQ ID NO: 44 - protein, 1958 aa, Artificial
  meta_gene_118560; CRISPR-associated protein
MPLSRLQNFLKSVRGNILYVNPNDLDATDSIENQGNSLTRPFKTIQRALVEASRFSYQTGLSNDRFAQTTVLLYPGEHVVDNRPGFIANDAGGGSAEYTSRGGTTGLSISPFDLTSNFDLESSSNVLYKLNSIHGGVIVPRGTSIVGYDLRKTKLRPKYVPDPENSNIENSAIFRVTGGCYFWQFSIFDASPSGQGYKDYTDNTFLPNFSHHKLTCFEFADGVNNIAVKDSFLNVSKSFSDLDNYYYKISDVYDNASGRAIAPDYPSGNVDIEPIIDETRIVGPKGGSVGITSIRSGNGVTGNTTITVETSTALSGITVDMPLRIIGVTASGYDGQRTVKSVGSGSTTFTYEVDTVPSTLFETPSNAKAELQVDTVSSASPYVFNCSLLSVYGMGGLHADGNKATGFKSMVAAQFTGISLQKDVKAFVKYNTSSGVYDDSTTVDNIAADSLARYKPAYSNYHIRCSNDAVLQIVSCFGVGFNGHFLAESGGDQSITNSNSNFGGAALVSDGYKEDAFSRDDVGYITHIIPPKEITTSDSALEFVSLDVSKTLSVGNTSRLYLYDQTNADVKPETVIQGFRLGAKTDDKLKVLIPLSGTTTEYSARIIMHNTAYASDEPSSVKRFTLNRSSVGINSITNSILTLTKVHNFLSGESVRVISESGHLPDGIDEKLTYNVIDANIDSSLATNQIKLAQNETDALADNFATLNNKGGILTIESRVSDKLAGDAGHPVQYDSGQNQWYVNVATAATENNIYSTVIGYSTAIGSNTPRTYISRKSDDRSQQDTLFRARYVVPAGVSSARPPIDGYVMQESCGDIETTANIQLVTLTNSVQQRNQTFIADANYLAATGIATITTEKPHNLEVGAQVQMLNVVSANNTTGIGTSGYNFKATVSGINSDRSFSVALDDDPGAFQNDTSTRTVDLPYYKKKDYATNFYVYRSTEIKKHVKDQQDGVYHLTLLNASNAPNITPFSGQNFSQNIIDLYPQTDRDNINSDPDSARSFATPDDIGEVLTNDLKKSITKENIIRFGRDSKVGIGVTDICSDIVVGTSHTIYTDRDHGLFGIKSVGLGSTGFGYGSGAAGTLYNATLTAVGSSTVGKSATAEITVDGIGGITSVRITNPGSAFGIGNTLAVTGTATTTSHVQGWVTVLTTFDNTNDSLSVLGVTSNTYSSRNTQYQVSGYEIGESKKIQVSTASSMTGIGAASTMGIGATVCARAMVFNAGPGIGITYFSYDYLSGIATVGSGVTAHGLSVGNVLSFVGSSNTAYNGDFRVTQVVGLTTFKVNAGVGTESPSESAGGSFYALPRGYASNDGAISLENENLSSRMTPILSGISTTLNSAVTTKTATSVEITNSFNSGLQKGNYIQIDEEIMRVATTPVGGSDAVTVLRGQLGTRRATHIDGSVIRVVSPIATEFRRNSILRASGHTFEYVGFGPGNYSTSLPEKVDRVLTGKQELLAQSVKKGGGVNVYTGMNDKGNFYVGNKKVNSTTGQEEVVDAPIATVTGEDLDIASGVAVGLDVITPLEVTVSRSLKVEGGTDANIISEFDGPVLFNKKVTSLGAGGIEANTFFIQGNATVAREVSVGISTPTVNGNPGDIKFFSDPKSGGSVGWVFTVENAWRRFGRISLYDFKDTNIFDQVGIATTTPNNYELQIGAGSSIINASAGKLGVGVTTPVRKLDVYGDVGATGFVTAGTYVYGDGSRLTNLPSDSQWTRTDAGINTISTNAGIGTTNPAYSLDIRGGKSGNSGQLYVGGDSQFTGVATMANVQATTLSATDVLIIDSDGQADVGIVTVRDYFNVGVGGTVIFTNSAGKVGINSATIDNQAAVDIGGRVRLDDYYEKVTTVTSSSGVVTLDLAKSRTFNLTTSEAVTQFVLSNRLDSDDHTTFTLKINQGSSAYAVGINTFKQTSGGTAIPISWSGGVVPSVVNVGLKTDIYSFQTFDGGASLYGIVVGQNFS

SEQ ID NO: 45 - protein, 1264 aa, Artificial
  meta_gene_324030; CRISPR-associated protein
MNTSTVTNNNAETTAIESLFAKKLLRSKGIAVIPPSTGSGKTREIARFASNPKEYIDNIKSNFSNGLCEIDESKKIKTIYISPQIKHCQDFISDIANDESCKDFCYEKRACRILNIFEVAEKVVGAYEDTKEKLNKTGKKTPSLLNERLLYKDGKIGENGENKQIEQFIEILNGLDKSSNMSEQIKEELQSKAKSQFRDIKTMIAKNYLNLEENPDFKDIELETYLKEPSLNWFVLLFPAHFWDEINTYSLTVKMSSFTIRDVIFSKDLSSLLKPEEEQSFVFIDEADTASEELIDTESENATKNSSIDVIKLLITLSRILDFKDVFPNYSSQKEKKQFEKAIERGRKKFIEHFGDVDSNSTLIPTKEVKKLANSKVLHNYILRDSVETRVIIKQGESAKKYMKDYYLSFPKNSEESKETPAFLITKDQIEEFPSDKYKVFEYKSFLKIASGLLNYFCEFVYPAIVELIKKNEEDDNEMRSLNGTLQTFKELYNVDEDFIKLLHDYKTQNYKKKITGASSGLLSYCDIGYEIFQVKVPLGGRPAELSRLLVQGTPEQTVVELAENSRVVLVSATANVPSLKNFNLDFLSNRFGDYFDNFTMEDKKDFEAKLNYSNHNKSIELISDVSYELKYYEKDKEPDDTEETWLERKVEENFSYLSKKMKMYLTNELTKEGIHRANYYILLIKYYLAMKAAKTKANLMIFQPNLEKEVIETLLNIFDPKLNEENAIFCANTEKLKTDGFIEKVENAYLEGKTIFLITSLATMGKAVNFTFKAREDEKLIHITPNGWIDDATKPAKRTFDGIAIGDINFSFASKDNNESSNNESSALRLLIDKITEVERLYATNLISNQIKRRIIQEMIINYESLYSFRGEFSTIRKLQGFYVYKEISQAIGRLYRTPNFSEKMLVLTTKNNHDNLSTIKDSIERKSFIETPLMTALMNEVQKEEITKKNSIEAKTMPLKNSGELFSRLLGTLLSDALKFKDKTSIQILEEMRRICIKYGVFLTEETYNSITSEQKDVDITSIKERLYQKVETSDFIKNGYKYKSHDDHSIIDFIDPKSSSEQGIPVSPTNCTIQQFRNLEGFYDYKENCGYTYDKVFNGEYIYILNPTAYNNLFKGALGEFVGKYIFEILFKLPLSRITDPEAYERADFFFAHDNSTAIDFKCYSNPKVEKESLLEGIKNKAKALDIKEYHVINVFPYSTKGVPFTKETLLNEDGTALLNSNGEPVVVKIVQATARPTSNCIVTDEFHQYILDTFLNKKGN

SEQ ID NO: 46 - protein, 1129 aa, Artificial
  meta_gene_295919; CRISPR-associated protein
MENISFSREKALPFSLEKLETIFNNLIQRDTYSNKILKEPLSEFYRREIESEGKYRDNFLQIVEYTLSSLETIVKNPKRELLKISELQSINEIRSTDYKTMIWLGNKPGKTLAEKIGAKGKILAPKNKYSIDKKENRVVVYYFKEAYKILEERYKRYIENSVDIPENLKKIYERFYRIKREMINNELFFLDRPIDFSPNNALIDHRDYSVVNRGLKHLKKYLEKLDYSENILLELAKKIVFLKLSYFIARLENIDIFDEILDIEELLKTKKKIIKFYSSKLQYLIKVILNKSKSKIRIEFQKIFFNRDTKEVEKRDKEILDIDIIDTYENSASYYKLKVKDVEYNFNDDDLKKILFENIKIDNLIKNKKESMNSERIINKYIYMNFNSQSLFIDNKALEIKSYNKKLDNFMDTKDYFLSHSEQQAHYHINEIVSSDETIDIFPKYLEYIKEKRNIDKQNICIYSSLEALDSDSQKMLSSIYDSNFNKSYPIWRSILATYAIKNSSKKWLENKEKFFVLDLNSEIPTINTIEIEKNINRHHPVIILEESENEELKELSLQAYLKEYLEKYLNVYSIEMDEVEKTNLISSGKVYETIFKRKRYLITNMNFYLEKDEDIIKNVGNKFYSNVQKFVSKFLIDKRKKLLIISDYLGEKYSLNGVDVKVIKEKELSLGKDEIIEKIKNNKNLWNEYLPNLTLETVKDGHFYNLDLIRENEDVEVIFGVEQKININENLVLPKGMDVIKFPLYSQDSNNKKLYFLEIKSELFPLKENLVVNLELIYSYGSKEPYKIKLKANGIDSSKFSTKWTENINKLKIVSLDYPEKNNKKNNYLGIKKILEKIDLNNTNLKDYLKRNKNRFRNYIIEEIERGNLERIKEVLDRNSKILALLEILNKQEKEKGLLNEMIAVFLASFGVLIYDRIKVDILKFEYRKRSTLFLYSLNNQLKLEDVLKYNKKDPEIIETVAEISWLDKVFINKLAEKEPELLEGALKFLKYTLKSLNQKFGEEYEKWSKENLLWMLANRFKNYLEFILAILTIKDKEKILKVLNKRDILKILYDIKAIDRKIQIDYPKLKEEFNKRIKLKFDRVVEQKKEVGLEAMSDLAYTVYCYLSGNNGSEAIKIKEVLDDFND

SEQ ID NO: 47 - protein, 908 aa, Artificial
  meta_gene_237613; CRISPR-associated protein
MYLHGHYYNEQNERIEVHIVTHGDKTDNQEISADTGDIQWTDDPVEIESQVSDTFDVLLPQQATIRLQVRNFVADLFCADLREAVVNIYREGECLFAGFLEPQSYSQGYSEEFDEIELSCIDVLTALKSFKYGDVGSIGRLYHEVKANARQRSFQEIITEMLTSLTSHIDILGGHSMSLYYDGSKAIDNQTDSRYRIFSQLSINELLFLSDEEDNVWTQEEVLTELLKYLDVHVVQVGFTFYIFSWESVKRAASITWQNLLTGQNSETPYRKMDIRTGDVIGDDTTMSIGEVYNQLLLTCKVEKMEQLIESPLEDSALRSDFPAKQKYMNEFISWGTGKRAIEGFRDLVFNSTTAYDAASIVDWYIWVKRHPHWTFPMHDNSLQAGMSLSDYFGQTGRNQQAYLQWLGSHLGAALVAYGKVATEMARGDNSPIAKIDMDNYLVLSVNGNGQDDQAKTYPKETDLKAAIPYAVYEGKKAGGVFSPADEQTTNYIVLSGKMILNPIMTQTATFRDLRTKPWTAKNIFSGQPIEEGKACVYGNVVKDKNGSEKYYTCKYWKQTDSNPKLNEEPQWDEQGDGGWYPFTGTAPESYEYNYSAVGDGTDKISKVGLVACMLIVGDKCVVEKGSGSQIEDFEWRKYKERSACSSDDEYYQQSFTIGFDPKIGDKLIGREYSLQNNISWKRGIDTEGMAIPIRKRDHVSGAVRFVILGPVNVLWGDITRRHPTFFRHTKWTEHAVPLLAHVSSIQIKQFEVKLHSDNGLIEHLGDEHDIIYMSDAKTSFCNKKDDLEFKITSALTYDESVQLGIVNTPCLSTPVNMASGDGVLQVCNTLTGQQAKAEQLYVDAYYREYHEPRVVLKQTFADRTNGIVDLFTHYRQAFMDKTFFVQAINRSLTEGSAELTLKEINND

SEQ ID NO: 48 - protein, 890 aa, Artificial
  meta_gene_35066; CRISPR-associated protein
MPTNYKTIINFRDGIQVDANDLVSNNGLVGIGTTIPREELDIRGNLIVENQANFRDVNVVGQSTFYGDINIAVGNSVGIGTTVPEATFQVGVGTTGFTVDSNGNVTALTFTGSGANLTNLPTAVWTNPYPGAGTTINAFRPVGVSVTLPQADFAVGDLIKLDATSGVGTFEGLVAKNITAVNASGSGQGNVNGEVGTFSTITATDTAVIDKLDGNLIGLSTIAGTASTANSVYVTDESTDTLLFPLFVDGAVLSGQIVAGNKEVKAGTNLQFDSANGTLSATSLSAAGGISIGPGGIMTATTFSGTATTALNASVAYAIAGQPDIQADKIDSLGINSIFIRNTGVSTFGGEVKVGNFLGVGATSSAIGKGMGVIGAADFSGAGTFGGDLLVAGNLSVGGTFGGAVNITDVTAGEIIATGILSATTSSSCVLHDTTITGNVVQSAGKNLTVGQNLSIGGTTTFGSQINFGDASTQVAAAGTLFANLSGIITTGGINVGDLDISGTFSYTGGSIATFGSILLNSNTGFVSCSSIEAGTGIISCTGLNARTGEITGGGLNLTGPTTSNNFFQSTSGVSTFFDIDITGGTNSNIQLTRLGFNTSLGALGITEGIALWDDAEIYVNDSPASGIGIGTTSGKRDSNVALYVGYGRDGAGNFINGQSVFEGGVGIGTMMGNDDGNMLEVYKETVFHSYHTGVGGTDAGPARVGFETNKPRTTLDLGFVTSGFLRIPSYYNDDPNNTVPTNDTGSQGSLFFDTAINSISIKDMNDNWVGIKTELSTGDDPAQYVQELGFIGGVTDQANRLSAEQGVANIIQPYDEVGNQGIGWGTAHMWYNKTFNKHQYKTNQGIGVATHYRSYVSTGTSAIDIELDSSGTKVYITLPGIGSATFNLV

SEQ ID NO: 49 - protein, 872 aa, Artificial
  meta_gene_524019; CRISPR-associated protein
MWWKFYLIPDYVSIRRDINGHPVFLLIKYAFNDQDRQENKNLPRGGGFMVFDVELSVREADYPKIIAELQQSVNSQWQQLKALADAAGNDVRGYSVNSWHYLNGNFQFSTLSVNDLQLGLHPERPEAPPGDAPPKVIISQPTWKEGKFHVSAPQSTDLVAHRVSEGPVSLVGNNVVSANMDLTTGGATFMEKTLTNLDGSGATDLTPIQVVYELTFWARVPPVHLLVTVDSRSLYEATKNIYHDYEGNGCDEDSINHSEQNLEMAVQSGLINIQIDTGTLSLSDDFVQQLRSGALKFVQDQIKDNFFDKKQAPPPADDPTKDFVGSDKEIYYLKSDIDFKSVSIGYNEQIDSIVEWKANPQGTLQTFLAGVSPSEMKRYVRDVDLRDTFFMTLGLTTTVFADWEHEPIAFVECQISYTGRDENNQLIEKVQTFTFAKDHTAEFWDPSLIGSKREYEYRWRVGFFGHDAGEFTSWLTETTPKLNISIADPGKITIKVLAGNIDFAQTTKQVQVDLKYGGPGLEVPEEGTTLVLVNGQLEGNYERYIYSTWDHPVLYHARFYLKNEQVVESDWQETVSRQLLINQPFLDQLKVQLVPAGSWDGVVQTVVNLRYKDELHSYHSEEAYTIKSADEFKTWAIVLRDPNQRKFQYKILSTFKDGSTPAQTDWIDADGDQAVLIRVQQHPELKVKLLAGQIDFKVTPVVECTLHYDDLQGHIQKVDTFPFSKAEDAVWDFPLASDSRRTYRYQITYHTADGHTIPMPEVSTDTTSVVIPPLEIPVISCTIFPKLVNFVQTPVVEVDFEYKDPDHHIEFEDTAVFTDSNPQSFRVQVDKASPRNYNLAVTYYTADGKVIQRDPVTLDKNKVVIPMYVATS

SEQ ID NO: 50 - protein, 809 aa, Artificial
  meta_gene_523517; CRISPR-associated protein
MIYRDHQDKGLFYYIPERPRLARNDGVPEFIYLVYKRDITDNPAFDPETKASLGGGFLAFTVDLGVDDQQLAEMKQELARFSDGEEVKLTPVQFHKGSVRLSISKDTADAPGTPPDQPKGLTFFEEVYGTTKPSLFGFNRATFSVVLSQEVAALFEAALQAGISPIGVIYDLEFLGLRPAFNVRITAEYKRIYDHLEIEFGARGQIYAVALALDIDLAFQKLRDDGSIKVEVLSFTDDANLRKQADDAFNWFKTELLKDFFKSSLEPPSFMKQTNTTDLVGRLQSIFQGLNSAQTSPTLNPVRGEPTKEPLTPAAPPKKQEDGMKSTADMNRAATQSGSESSGGGSGADRGISPFQIGFTLKYYRQEELKTRTFEFSEQAAVAREAAPQGLFTTMVQGLDLSRAIQHVNLDSDFFKRLITTVSASDEFTIAGISTLGVNLEYPGTRKPGEDPLFVDGFVYKSDDLKPRTFTTWLNDRKNLTYRYQMDIHFTPDSPWVGKEGSVTSDWIITRSRQLTLDPMNEISLFDVQLTLGNMISGQINQVEVELRYQDSANDFNTQKTFLLKPGDPVTHWKLRLMDSEQKTYQYRITYFLQEGVRVQTDWVSSEDPTLVVAEPFKGTLNIRMVPLLDPTTLLEADVELMYHEEDTGYTRRVEKVFSPSDLKGQQISIPTLAENPTSYNYTINIIRTDGSTYTLPPTTATTPVLVVSDGAGVTHRILVKLPSKDLSSFGLAALKVDLVGPGDDPDTASVLFTPSQTDDKMPALVQPGDGGTFTYSYKVTGYTTQGLPIEGDSGTSSGPTLIVKIPTR

SEQ ID NO: 51 - DNA, 36 bp, Artificial
  CRISPR repeat sequence
gttttagagctgtgttgtttcgaatggttccaaaac

SEQ ID NO: 52 - DNA, 36 bp, Artificial
  CRISPR repeat sequence
ctttaagtgattagatgaattaaatgtgattagcac

SEQ ID NO: 53 - DNA, 36 bp, Artificial
  CRISPR repeat sequence
gttaagtaacctaaataatttctactgtgtgtagat

SEQ ID NO: 54 - DNA, 32 bp, Artificial
  CRISPR repeat sequence
agtgaaagtagcccgatatagagggcaataac

SEQ ID NO: 55 - DNA, 36 bp, Artificial
  CRISPR repeat sequence
ctactacactggtgcgaatttgcactagtctaaaac

SEQ ID NO: 56 - DNA, 37 bp, Artificial
  CRISPR repeat sequence
ctactacactggtgcgaatttgcactagtctaaaact

SEQ ID NO: 57 - DNA, 32 bp, Artificial
  CRISPR repeat sequence
gtcgctaatggagcggcttctcggttgagatt

SEQ ID NO: 58 - DNA, 30 bp, Artificial
  CRISPR repeat sequence
ataaagaattaacataagttgtttttaaat

SEQ ID NO: 59 - DNA, 29 bp, Artificial
  CRISPR repeat sequence
gtcggccccgcacgcgcggggatgttccg

SEQ ID NO: 60 - DNA, 32 bp, Artificial
  CRISPR repeat sequence
gtcgctcccctcgcgggagcgtggattgaaat

SEQ ID NO: 61 - DNA, 32 bp, Artificial
  CRISPR repeat sequence
gtcacgccttatggaggcgtgtggattgaaat

SEQ ID NO: 62 - DNA, 35 bp, Artificial
  CRISPR repeat sequence
gtcacagtgagatcagccgttcaggctgttgaaac

SEQ ID NO: 63 - DNA, 17 bp, Artificial
  CRISPR repeat sequence
taactaagttggaaact

SEQ ID NO: 64 - DNA, 30 bp, Artificial
  CRISPR repeat sequence
gactaaatccaagtagattggaattttaac

SEQ ID NO: 65 - DNA, 32 bp, Artificial
  CRISPR repeat sequence
atttaaatacatcctatgttatggttcaatca

SEQ ID NO: 66 - DNA, 37 bp, Artificial
  CRISPR repeat sequence
gtagcattcacccccaagggtgggtgcccgttgaaac

SEQ ID NO: 67 - DNA, 37 bp, Artificial
  CRISPR repeat sequence
gtttcagagcagatgctggcttgagttaagatgtaac

SEQ ID NO: 68 - DNA, 29 bp, Artificial
  CRISPR repeat sequence
gtgctccccgctcaggcgggggtgatccc

SEQ ID NO: 69 - DNA, 29 bp, Artificial
  CRISPR repeat sequence
gggaacacccccgcacgcgcggggaccac

SEQ ID NO: 70 - DNA, 44 bp, Artificial
  CRISPR repeat sequence
cataagtcttttgtggatgagctgtggagggacgcactggcagt

SEQ ID NO: 71 - DNA, 45 bp, Artificial
  CRISPR repeat sequence
agctcgtgcaccgtcagccgatagagcaccaggtcttccggccga

SEQ ID NO: 72 - DNA, 37 bp, Artificial
  CRISPR repeat sequence
tctttgaccggcaggtcacatcggacggcgcacaacc

SEQ ID NO: 73 - DNA, 60 bp, Artificial
  CRISPR repeat sequence
gatttaaaggacggcgcggacaaattaaaagacggctccgcggacctcaaagacgggacg

SEQ ID NO: 74 - DNA, 31 bp, Artificial
  CRISPR repeat sequence
cgataagcatgtgagtgagacatcccgaata

SEQ ID NO: 75 - DNA, 35 bp, Artificial
  CRISPR repeat sequence
gtttagaaccttaatccccgtaaggggacggaaac

SEQ ID NO: 76 - DNA, 33 bp, Artificial
  CRISPR repeat sequence
aacctaccgtcttggctagcggttgcagcgaac

SEQ ID NO: 77 - DNA, 37 bp, Artificial
  CRISPR repeat sequence
ccgtcaaacagcagtttaataatgcgtggaaagaaaa

SEQ ID NO: 78 - DNA, 35 bp, Artificial
  CRISPR repeat sequence
atggacatccaacaataaaaccacaagccattata

SEQ ID NO: 79 - DNA, 32 bp, Artificial
  CRISPR repeat sequence
tcagttgtgctgtgtcggtcatgcggcaccgc

SEQ ID NO: 80 - DNA, 31 bp, Artificial
  CRISPR repeat sequence
gtttcaatccacgcactcgtgagagtgcgac

SEQ ID NO: 81 - DNA, 29 bp, Artificial
  CRISPR repeat sequence
gtggtccccgcgcgtgcgggggtggtccc

SEQ ID NO: 82 - DNA, 29 bp, Artificial
  CRISPR repeat sequence
gtggtccccgcgcgtgcgggggtgttccc

SEQ ID NO: 83 - DNA, 37 bp, Artificial
  CRISPR repeat sequence
ctccgagaccatcctccactaaaacaaggattaagac

SEQ ID NO: 84 - DNA, 30 bp, Artificial
  CRISPR repeat sequence
ggtacagacgaacccttgtgggattgaagc

SEQ ID NO: 85 - DNA, 37 bp, Artificial
  CRISPR repeat sequence
gtcaccgaccacgatccaccagaacaaggattgaaac

SEQ ID NO: 86 - DNA, 37 bp, Artificial
  CRISPR repeat sequence
gtcagaaagcacccagcaccagaaggtgcattaagac

SEQ ID NO: 87 - DNA, 32 bp, Artificial
  CRISPR repeat sequence
gtcgctccttgtacgggagcgtggattgaaac

SEQ ID NO: 88 - DNA, 29 bp, Artificial
  CRISPR repeat sequence
gtggtccccgcgcgtgcgggggtgttccc

SEQ ID NO: 89 - DNA, 36 bp, Artificial
  CRISPR repeat sequence
gttttggaaccattctgtttagcatggtaccaaagg

SEQ ID NO: 90 - DNA, 24 bp, Artificial
  CRISPR repeat sequence
ttagaaaaagaaattaaagaaaaa

SEQ ID NO: 91 - DNA, 49 bp, Artificial
  CRISPR repeat sequence
gttgtgatttgcttaaaaatatctatctttgtggtagcaacaacaacct

SEQ ID NO: 92 - DNA, 23 bp, Artificial
  CRISPR repeat sequence
ggaacacctggtacacctggtgg

SEQ ID NO: 93 - DNA, 39 bp, Artificial
  CRISPR repeat sequence
cacttgcagtcccctaaatcggggtgagaccattgcaac

SEQ ID NO: 94 - DNA, 39 bp, Artificial; identical to SEQ ID NO: 93
  CRISPR repeat sequence

SEQ ID NO: 95-99 - skipped (5 entries)

SEQ ID NO: 100 - DNA, 30 bp, Artificial
  Spacer sequence
agaatacaacattgtcttaataggagacac

SEQ ID NO: 101 - DNA, 30 bp, Artificial
  Spacer sequence
gaatcatgattggtttatctgtgggcttca

SEQ ID NO: 102 - DNA, 30 bp, Artificial
  Spacer sequence
aaagaaattaaaaaaacctagcgaagcact

SEQ ID NO: 103 - DNA, 30 bp, Artificial
  Spacer sequence
ttcgcataagacttcttcaaaccaaaacat

SEQ ID NO: 104 - DNA, 29 bp, Artificial
  Spacer sequence
gtccataggtatttccctttaattaaagt

SEQ ID NO: 105 - DNA, 30 bp, Artificial
  Spacer sequence
tagagatgacgacggactacctggcaagaa

SEQ ID NO: 106 - DNA, 30 bp, Artificial
  Spacer sequence
tatcccagagaatggaagaacaattataga

SEQ ID NO: 107 - DNA, 30 bp, Artificial
  Spacer sequence
cttcttaaaattgaataattcgaagcacat

SEQ ID NO: 108 - DNA, 30 bp, Artificial
  Spacer sequence
aggtaacattggttcaacagcagtctaatt

SEQ ID NO: 109 - DNA, 30 bp, Artificial
  Spacer sequence
tcgttaccttgtctttgcaaatcacgcaaa

SEQ ID NO: 110 - DNA, 30 bp, Artificial
  Spacer sequence
aatgaagaagccgattcaagctcaagggtc

SEQ ID NO: 111 - DNA, 30 bp, Artificial
  Spacer sequence
tatttctgtccgatacgaagtatcagggac

SEQ ID NO: 112 - DNA, 30 bp, Artificial
  Spacer sequence
tacgcccgtttggattgaacatgatagagc

SEQ ID NO: 113 - DNA, 30 bp, Artificial
  Spacer sequence
gagcctactaatgattacattttgaggacg

SEQ ID NO: 114 - DNA, 30 bp, Artificial
  Spacer sequence
ccaaagaatggaccaccttaatgagaatat

SEQ ID NO: 115 - DNA, 30 bp, Artificial
  Spacer sequence
tttcaaaatcttcgaataggcagtcgagca

SEQ ID NO: 116 - DNA, 30 bp, Artificial
  Spacer sequence
aaaatgtacaaattttcatgctagggaata

SEQ ID NO: 117 - DNA, 30 bp, Artificial
  Spacer sequence
tacagctcttggtttcgtctatccttatgt

SEQ ID NO: 118 - DNA, 30 bp, Artificial
  Spacer sequence
cgctagggtctctggtgacgctgaggtctc

SEQ ID NO: 119 - DNA, 30 bp, Artificial
  Spacer sequence
cctgacgcatatggaaatcctaacggtcag

SEQ ID NO: 120 - DNA, 30 bp, Artificial
  Spacer sequence
aaaatcatctaaatacatgtgtgtaacaag

SEQ ID NO: 121 - DNA, 30 bp, Artificial
  Spacer sequence
aagcactggacgacaaataaataattgaag

SEQ ID NO: 122 - DNA, 30 bp, Artificial
  Spacer sequence
gaacaagaaacttatgaagtcgaaaaccga

SEQ ID NO: 123 - DNA, 30 bp, Artificial; identical to SEQ ID NO: 103
  Spacer sequence

SEQ ID NO: 124 - DNA, 36 bp, Artificial
  Spacer sequence
caaaccgcctgttgctcccgcaacacgcattcggtc

SEQ ID NO: 125 - DNA, 36 bp, Artificial
  Spacer sequence
gtggaatcctatttggcgcttgaaggggacaaccgc

SEQ ID NO: 126 - DNA, 40 bp, Artificial
  Spacer sequence
gccgaagatacctggtgagaagttttcagcattccaaatg

SEQ ID NO: 127 - DNA, 38 bp, Artificial
  Spacer sequence
ttaactctatttgatgttatttttaactctatttggag

SEQ ID NO: 128 - DNA, 36 bp, Artificial
  Spacer sequence
ggaatatcccttgatttcgtggaatattccacgttt

SEQ ID NO: 129 - DNA, 38 bp, Artificial
  Spacer sequence
ccactttttaagaacatatacaaacgatctcgaagcgg

SEQ ID NO: 130 - DNA, 41 bp, Artificial
  Spacer sequence
gctaacacaatcaacacgattccaccaacaatggtttttcc

SEQ ID NO: 131 - DNA, 36 bp, Artificial
  Spacer sequence
ccattgatacaggcaatctccatgtctgatttgttg

SEQ ID NO: 132 - DNA, 36 bp, Artificial
  Spacer sequence
gggagataaggtaaaacatagactccaaatagtgct

SEQ ID NO: 133 - DNA, 36 bp, Artificial
  Spacer sequence
tgagtacatcgggggataaaaagccgcataggaatc

SEQ ID NO: 134 - DNA, 36 bp, Artificial
  Spacer sequence
ttaactgcccaatttccattttccagcttaacgatc

SEQ ID NO: 135 - DNA, 26 bp, Artificial
  Spacer sequence
atggctgtctgtataaggtgtctctg

SEQ ID NO: 136 - DNA, 27 bp, Artificial
  Spacer sequence
ttaattttattgttgctgttgtttagt

SEQ ID NO: 137 - DNA, 26 bp, Artificial
  Spacer sequence
attttaccgctacaggagaacacgat

SEQ ID NO: 138 - DNA, 27 bp, Artificial
  Spacer sequence
atcgacagggataacacaggcatagct

SEQ ID NO: 139 - DNA, 27 bp, Artificial
  Spacer sequence
ctatacgccagagggtgagccttggaa

SEQ ID NO: 140 - DNA, 27 bp, Artificial
  Spacer sequence
aagtattgaaaaatatcatatagtaat

SEQ ID NO: 141 - DNA, 27 bp, Artificial
  Spacer sequence
caaaatatcgataaggctccagaagaa

SEQ ID NO: 142 - DNA, 25 bp, Artificial
  Spacer sequence
ctattgggatactctcattaaaagt

SEQ ID NO: 143 - DNA, 26 bp, Artificial
  Spacer sequence
caaaatcttatctttatcttcttgag

SEQ ID NO: 144 - DNA, 26 bp, Artificial
  Spacer sequence
tactatgcccgaatattaaaagctgt

SEQ ID NO: 145 - DNA, 27 bp, Artificial
  Spacer sequence
aaaatatgaagctcccttacaattttc

SEQ ID NO: 146 - DNA, 26 bp, Artificial
  Spacer sequence
ataacaaccgcctgtttagtactagg

SEQ ID NO: 147 - DNA, 26 bp, Artificial
  Spacer sequence
atatcattaatatgggctgggataca

SEQ ID NO: 148 - DNA, 42 bp, Artificial
  Spacer sequence
ttttggaggtcgccttttgaaaccttgaatcctaaattccta

SEQ ID NO: 149 - DNA, 41 bp, Artificial
  Spacer sequence
gtttggtacggttttattttcttatagtttttatatatatg

SEQ ID NO: 150 - DNA, 36 bp, Artificial
  Spacer sequence
gtcatattacaacatgcttcatactgcttgtcatca

SEQ ID NO: 151 - DNA, 36 bp, Artificial
  Spacer sequence
aagccaacctaaatcaacaccatcatcatcacaaac

SEQ ID NO: 152 - DNA, 31 bp, Artificial
  Spacer sequence
ttgcagttttcttcacgatacttatctagct

SEQ ID NO: 153 - DNA, 30 bp, Artificial
  Spacer sequence
aggtcaagatctgatttatgaattttgcct

SEQ ID NO: 154 - DNA, 30 bp, Artificial
  Spacer sequence
atggattcctctacctcttcatctgttaca

SEQ ID NO: 155 - DNA, 31 bp, Artificial
  Spacer sequence
aatatttcttttatattcttacacccctcga

SEQ ID NO: 156 - DNA, 29 bp, Artificial
  Spacer sequence
ctatgtagcttttcttgtaaaacatattt

SEQ ID NO: 157 - DNA, 29 bp, Artificial
  Spacer sequence
catctgccttctgcatatcggacacttga

SEQ ID NO: 158 - DNA, 29 bp, Artificial
  Spacer sequence
tacacctccttatgcgattttatcgtgcg

SEQ ID NO: 159 - DNA, 30 bp, Artificial
  Spacer sequence
taaaaatatcctttttgctcatgttcacgt

SEQ ID NO: 160 - DNA, 38 bp, Artificial
  Spacer sequence
gaaacttgtgagcttccatgaaaccgaataagtactta

SEQ ID NO: 161 - DNA, 40 bp, Artificial
  Spacer sequence
gaaacattcccatcaccctcgatatcaaagccataatcat

SEQ ID NO: 162 - DNA, 40 bp, Artificial
  Spacer sequence
gaaacccgtttagcttgatacgagaagcccctcggcttta

SEQ ID NO: 163 - DNA, 38 bp, Artificial
  Spacer sequence
actccaacataacctcttaagtacttaaaatcttcttt

SEQ ID NO: 164 - DNA, 36 bp, Artificial
  Spacer sequence
ttctttttgtcaatatttctaaatttatattttctt

SEQ ID NO: 165 - DNA, 35 bp, Artificial
  Spacer sequence
aaaagtggattatctccactggaagtggtactcaa

SEQ ID NO: 166 - DNA, 36 bp, Artificial
  Spacer sequence
ggtgttccttttttgtattgatttcttttatttatt

SEQ ID NO: 167 - DNA, 29 bp, Artificial
  Spacer sequence
aaaagaagaattacatttaaattttaaga

SEQ ID NO: 168 - DNA, 35 bp, Artificial
  Spacer sequence
actgtaactcgattttttaaaaatatttttacttc

SEQ ID NO: 169 - DNA, 31 bp, Artificial
  Spacer sequence
aaaatgtgagataatttatacgaattatttt

SEQ ID NO: 170 - DNA, 35 bp, Artificial
  Spacer sequence
attccagttttaaaattctttcctattgggacacc

SEQ ID NO: 171 - DNA, 17 bp, Artificial
  Spacer sequence
agaggtattggaaaata

SEQ ID NO: 172 - DNA, 32 bp, Artificial
  Spacer sequence
tgttgaacgaccctgaggccacgcagctgcag

SEQ ID NO: 173 - DNA, 32 bp, Artificial
  Spacer sequence
atcgacgccagcgacatcggctgggtccaggc

SEQ ID NO: 174 - DNA, 32 bp, Artificial
  Spacer sequence
gtgaacatcggcgggatcacgatcaagcggga

SEQ ID NO: 175 - DNA, 32 bp, Artificial
  Spacer sequence
tggctgagcggaccgtcgaggccggggcgtcc

SEQ ID NO: 176 - DNA, 32 bp, Artificial
  Spacer sequence
ggttacgaggtcgggggcggggccttgagcag

SEQ ID NO: 177 - DNA, 32 bp, Artificial
  Spacer sequence
tccaggcgacattacgcccgttgcggccgatc

SEQ ID NO: 178 - DNA, 32 bp, Artificial
  Spacer sequence
tcatggggccaagccaagaaaaggggcgatta

SEQ ID NO: 179 - DNA, 32 bp, Artificial
  Spacer sequence
tacctgggcgggcgcgcggcccgagctgagaa

SEQ ID NO: 180 - DNA, 32 bp, Artificial
  Spacer sequence
cccacgggcggacccatcggaaggcgccttcg

SEQ ID NO: 181 - DNA, 32 bp, Artificial
  Spacer sequence
cggccagctcagccccggtgccgctggtctcc

SEQ ID NO: 182 - DNA, 32 bp, Artificial
  Spacer sequence
tgctcaccgcctacgcgatggatcctgaacgc

SEQ ID NO: 183 - DNA, 32 bp, Artificial
  Spacer sequence
aagccggcgccgaaggtcgcggggatcggcgc

SEQ ID NO: 184 - DNA, 32 bp, Artificial
  Spacer sequence
aactgcagcgactcatcgacgaacaggcaggt

SEQ ID NO: 185 - DNA, 32 bp, Artificial
  Spacer sequence
cggttctcgttcatcgttcggtcctcttcttg

SEQ ID NO: 186 - DNA, 32 bp, Artificial
  Spacer sequence
ggcgcaccgatgccccagcagctcaccgacga

SEQ ID NO: 187 - DNA, 32 bp, Artificial
  Spacer sequence
gattgtgtaggcccccggcacctacagaaccc

SEQ ID NO: 188 - DNA, 33 bp, Artificial
  Spacer sequence
gtgtctcctactggtccgggtcggggaagagcg

SEQ ID NO: 189 - DNA, 32 bp, Artificial
  Spacer sequence
ctggaggtcatcgccgccgaggtcgccgagtt

SEQ ID NO: 190 - DNA, 32 bp, Artificial
  Spacer sequence
ccgaccaggctggccagggcgccgagggagac

SEQ ID NO: 191 - DNA, 32 bp, Artificial
  Spacer sequence
gagttgtagctctcgatctcgccgagcacgtt

SEQ ID NO: 192 - DNA, 32 bp, Artificial
  Spacer sequence
ctgttcgtggagcgctcgagctgggcgtgacc

SEQ ID NO: 193 - DNA, 32 bp, Artificial
  Spacer sequence
aaggccgggcttcagcgctacggccggtacct

SEQ ID NO: 194 - DNA, 32 bp, Artificial
  Spacer sequence
atgatggagctggtcgcccagctctcccccgc

SEQ ID NO: 195 - DNA, 32 bp, Artificial
  Spacer sequence
cacgccctctgatcccgacaccaaggagagac

SEQ ID NO: 196 - DNA, 32 bp, Artificial
  Spacer sequence
tcatggatgtccgtccgctgggtggggccgct

SEQ ID NO: 197 - DNA, 32 bp, Artificial
  Spacer sequence
gcgggctacgagatcgacggcgagaccgtcga

SEQ ID NO: 198 - DNA, 32 bp, Artificial
  Spacer sequence
gggcgcgccagtacgcgcgcggcatcgtggcg

SEQ ID NO: 199 - DNA, 32 bp, Artificial
  Spacer sequence
cgtgccgggtggtggtgtcgaccgtgccgtcg

SEQ ID NO: 200 - DNA, 32 bp, Artificial
  Spacer sequence
atcttcggggcggcgggcgccgagggcggcgg

SEQ ID NO: 201 - DNA, 32 bp, Artificial
  Spacer sequence
tccccgaactccagcagccggtggattctggc

SEQ ID NO: 202 - DNA, 32 bp, Artificial
  Spacer sequence
gaggcgcagctcgcctatgagcaggcggtgca

SEQ ID NO: 203 - DNA, 32 bp, Artificial
  Spacer sequence
cggaacttcttcctcaacagcgcggagccagg

SEQ ID NO: 204 - DNA, 32 bp, Artificial
  Spacer sequence
gtcgagcttgacaagcagaaccagccccaggg

SEQ ID NO: 205 - DNA, 32 bp, Artificial
  Spacer sequence
ctgtccaacggcgagtacgtgctgcccgccaa

SEQ ID NO: 206 - DNA, 34 bp, Artificial
  Spacer sequence
atctactgcaacgcttttaacaagatcgctgatt

SEQ ID NO: 207 - DNA, 35 bp, Artificial
  Spacer sequence
ttagttctctgtgaacaacaagtgtcatctcactt

SEQ ID NO: 208 - DNA, 35 bp, Artificial
  Spacer sequence
gattattgctgatatagtacaagaagcgttttgca

SEQ ID NO: 209 - DNA, 35 bp, Artificial
  Spacer sequence
caagcgtggtacttgggagatcgacaaaaagatct

SEQ ID NO: 210 - DNA, 34 bp, Artificial
  Spacer sequence
aaccccgatgggaaggtcctgccgctctggctgc

SEQ ID NO: 211 - DNA, 36 bp, Artificial
  Spacer sequence
ttcctgcggttctggcggagaccagatcaagttcgt

SEQ ID NO: 212 - DNA, 35 bp, Artificial
  Spacer sequence
gtaagctgtcaggagatatggtgcgagtgtttcgg

SEQ ID NO: 213 - DNA, 45 bp, Artificial
  Spacer sequence
cgacagctgcgccgcgggcaagtgcaagggcggcaacgcgctact

SEQ ID NO: 214 - DNA, 34 bp, Artificial
  Spacer sequence
gctatagtgtccggtttcccgttttttccgattt

SEQ ID NO: 215 - DNA, 34 bp, Artificial
  Spacer sequence
aaccatgctaccgcacagggtggataatattttg

SEQ ID NO: 216 - DNA, 33 bp, Artificial
  Spacer sequence
ctttgtggttgccaagctcactacttgcgctgc

SEQ ID NO: 217 - DNA, 39 bp, Artificial
  Spacer sequence
accaccgcgcttgaacgcgggaaaattcgttctggctat

SEQ ID NO: 218 - DNA, 35 bp, Artificial
  Spacer sequence
caccatacggtgccagaatccgtataggacactgg

SEQ ID NO: 219 - DNA, 36 bp, Artificial
  Spacer sequence
ccagtgcttcatggttaatgaaggcagcagatttgg

SEQ ID NO: 220 - DNA, 37 bp, Artificial
  Spacer sequence
gcatctgattcattctcatattttgaacttctaattc

SEQ ID NO: 221 - DNA, 37 bp, Artificial
  Spacer sequence
tgaaaaacttccaaacacgctgacaaaggagcaacta

SEQ ID NO: 222 - DNA, 37 bp, Artificial
  Spacer sequence
atcgaaaattttacgttaagagagctttctggaaaga

SEQ ID NO: 223 - DNA, 36 bp, Artificial
  Spacer sequence
aactcaggaaatcaacgtcaggaactaaacggaaaa

SEQ ID NO: 224 - DNA, 35 bp, Artificial
  Spacer sequence
gcaactcctctaacatcgcccctaatttcacacga

SEQ ID NO: 225 - DNA, 36 bp, Artificial
  Spacer sequence
tcggcagttcgggacgccttaaaagaagcgggaaat

SEQ ID NO: 226 - DNA, 36 bp, Artificial
  Spacer sequence
aatgtagccttaattctccatgatcgccatactcta

SEQ ID NO: 227 - DNA, 36 bp, Artificial
  Spacer sequence
ttttatcgattctcatcacaatttgagcaacatctt

SEQ ID NO: 228 - DNA, 33 bp, Artificial
  Spacer sequence
tggcctagcatggcagctaggaaaaataaactt

SEQ ID NO: 229 - DNA, 35 bp, Artificial
  Spacer sequence
cctacagatgtgcaaaatggtctaaataaaatata

SEQ ID NO: 230 - DNA, 36 bp, Artificial
  Spacer sequence
ctcccctgtgtcggttcatcgcccgtggcgggagtt

SEQ ID NO: 231 - DNA, 42 bp, Artificial
  Spacer sequence
gaaactgctatcgctattgcgtcggtttttgtcatacgctta

SEQ ID NO: 232 - DNA, 36 bp, Artificial; identical to SEQ ID NO: 230
  Spacer sequence

SEQ ID NO: 233 - DNA, 37 bp, Artificial
  Spacer sequence
cgtcaatttcgggcgtgaagaatcgcgggatataggc

SEQ ID NO: 234 - DNA, 36 bp, Artificial
  Spacer sequence
cgcgacgcgcaacataacgctccagtgcttcgttgt

SEQ ID NO: 235 - DNA, 35 bp, Artificial
  Spacer sequence
gcgagggccagaaggcccagaaaaacgagagtgcc

SEQ ID NO: 236 - DNA, 34 bp, Artificial
  Spacer sequence
ccggcggccacacgctggcggatttcttctacca

SEQ ID NO: 237 - DNA, 35 bp, Artificial
  Spacer sequence
acaaagactggctacgagaaggcgattgaatgcgt

SEQ ID NO: 238 - DNA, 33 bp, Artificial
  Spacer sequence
agtacgacccgcacgcttggaacaaataccccg

SEQ ID NO: 239 - DNA, 34 bp, Artificial
  Spacer sequence
tgaaggctgtccgcctgcgccccattcccatgca

SEQ ID NO: 240 - DNA, 35 bp, Artificial
  Spacer sequence
catcaaaaactggtcatcctgcaccgtttcctgat

SEQ ID NO: 241 - DNA, 32 bp, Artificial
  Spacer sequence
gggcttgggggcgtagaagggatcgccgtggc

SEQ ID NO: 242 - DNA, 32 bp, Artificial
  Spacer sequence
tccaggcctacgaggctgaggagtccgcgaag

SEQ ID NO: 243 - DNA, 32 bp, Artificial
  Spacer sequence
tgcccggcgtccaaccgcggcccgtagatcac

SEQ ID NO: 244 - DNA, 32 bp, Artificial
  Spacer sequence
ggcatgacgtacgaggagatcgggcaagaggc

SEQ ID NO: 245 - DNA, 32 bp, Artificial
  Spacer sequence
gggctggccccacgccacctcgtgcgtcactg

SEQ ID NO: 246 - DNA, 32 bp, Artificial
  Spacer sequence
ccgcaccctgaccaccggggccgccgggcagc

SEQ ID NO: 247 - DNA, 32 bp, Artificial
  Spacer sequence
gacgaggaccggtatcccgctgcctggggagt

SEQ ID NO: 248 - DNA, 32 bp, Artificial
  Spacer sequence
aacgggtcgatcacggatgtggcgacccggcc

SEQ ID NO: 249 - DNA, 32 bp, Artificial
  Spacer sequence
gcggtccaggtcgggcggcaggtcgtacatgc

SEQ ID NO: 250 - DNA, 32 bp, Artificial
  Spacer sequence
tatggcgacatgtctgcgtcgttggcggccga

SEQ ID NO: 251 - DNA, 32 bp, Artificial
  Spacer sequence
ccgcactccgactacccgaccgagtggcgcca

SEQ ID NO: 252 - DNA, 32 bp, Artificial
  Spacer sequence
gaggccccctcgggcagtgcccctcaggccac

SEQ ID NO: 253 - DNA, 34 bp, Artificial
  Spacer sequence
cagcccggcccgggggaggaggaggcgcgggcgc

SEQ ID NO: 254 - DNA, 32 bp, Artificial
  Spacer sequence
gccgcagtccagcccggccccgacggcggatg

SEQ ID NO: 255 - DNA, 32 bp, Artificial
  Spacer sequence
caggacaccacctcgtcctgccggggctttcc

SEQ ID NO: 256 - DNA, 32 bp, Artificial
  Spacer sequence
cagccgggacagcggggccggccgggcgcccg

SEQ ID NO: 257 - DNA, 32 bp, Artificial
  Spacer sequence
ggagcacgcccgatgaccaccccgcacgacca

SEQ ID NO: 258 - DNA, 32 bp, Artificial
  Spacer sequence
ccacccctccaccgtggcgcaccggacagccc

SEQ ID NO: 259 - DNA, 32 bp, Artificial
  Spacer sequence
gtcatcgtgcccctgccccctgagggcctcgc

SEQ ID NO: 260 - DNA, 32 bp, Artificial
  Spacer sequence
gaggtggtcgccctccgcgcccagctcgcccc

SEQ ID NO: 261 - DNA, 32 bp, Artificial
  Spacer sequence
tgggagctgatgcggtcccggatgcctggccg

SEQ ID NO: 262 - DNA, 43 bp, Artificial
  Spacer sequence
tattcactttttgtgatgatctgcggagagatgttctggcggt

SEQ ID NO: 263 - DNA, 41 bp, Artificial
  Spacer sequence
tattgtggcagactgcgaatgtttttggagggggagggggt

SEQ ID NO: 264 - DNA, 42 bp, Artificial
  Spacer sequence
ctatgtgagtggcaacaagtatcttggtgcagggacgcagac

SEQ ID NO: 265 - DNA, 25 bp, Artificial
  Spacer sequence
acaacgaggaacttgatcgtggagg

SEQ ID NO: 266 - DNA, 25 bp, Artificial
  Spacer sequence
aaaatgagaagcttgatcgtgaagg

SEQ ID NO: 267 - DNA, 46 bp, Artificial
  Spacer sequence
acaatgtgcccaaataaaataactgacgcagagtgttctgcgaaat

SEQ ID NO: 268 - DNA, 50 bp, Artificial
  Spacer sequence
gttttctgtagtaggttcctttctatgacgaaataatggtttggtgagag

SEQ ID NO: 269 - DNA, 47 bp, Artificial
  Spacer sequence
atctcgtatctaaagcaagacagatcatgtggagtgttttgtgagat

SEQ ID NO: 270 - DNA, 50 bp, Artificial
  Spacer sequence
ttcttctgtagtgggggccttattgtgacgaaagaattgttcggctagag

SEQ ID NO: 271 - DNA, 22 bp, Artificial
  Spacer sequence
tgttatggagaggagcatgggg

SEQ ID NO: 272 - DNA, 45 bp, Artificial; identical to SEQ ID NO: 71
  Spacer sequence

SEQ ID NO: 273 - DNA, 39 bp, Artificial
  Spacer sequence
gcgggcttgtccagggatatccagttgcggcggttcggg

SEQ ID NO: 274 - DNA, 42 bp, Artificial
  Spacer sequence
tcggttatttcgcagtccggccgggcggcttcctgcactgaa

SEQ ID NO: 275 - DNA, 43 bp, Artificial
  Spacer sequence
aacatgcttgaaccgtctggcatagaccgctacaggggtcacc

SEQ ID NO: 276 - DNA, 43 bp, Artificial
  Spacer sequence
accctaaaccagtagcgcacttcggacgtcgtgtagtggatgc

SEQ ID NO: 277 - DNA, 40 bp, Artificial
  Spacer sequence
tccttgacggcgaggtcggcacagaccagcacccctcgat

SEQ ID NO: 278 - DNA, 39 bp, Artificial
  Spacer sequence
gatctttcttccggcgtttcaacgctcaaggacggctct

SEQ ID NO: 279 - DNA, 39 bp, Artificial
  Spacer sequence
acgcttgcatctggcgcatcacagttaaagggcggttcc

SEQ ID NO: 280 - DNA, 31 bp, Artificial
  Spacer sequence
ccttcagcaaaacgaatcatctaaaagtcgc

SEQ ID NO: 281 - DNA, 31 bp, Artificial
  Spacer sequence
cctcatttaccactataaccgtacaaaatta

SEQ ID NO: 282 - DNA, 31 bp, Artificial
  Spacer sequence
ctccatctctatcaataacaaatttattata

SEQ ID NO: 283 - DNA, 31 bp, Artificial
  Spacer sequence
ccgtggcattaccactcgtacagactctgag

SEQ ID NO: 284 - DNA, 31 bp, Artificial
  Spacer sequence
cgttcatcgttcagacaatctgtcgattgct

SEQ ID NO: 285 - DNA, 31 bp, Artificial
  Spacer sequence
atggccgtggcttacaagattctgccgtggc

SEQ ID NO: 286 - DNA, 31 bp, Artificial
  Spacer sequence
taaactggcacaaaatgtagttatgtattga

SEQ ID NO: 287 - DNA, 31 bp, Artificial
  Spacer sequence
tacaacgccgcaatcggacacacacatagtg

SEQ ID NO: 288 - DNA, 31 bp, Artificial
  Spacer sequence
acctgaccacaatcaagagttattgagcttg

SEQ ID NO: 289 - DNA, 31 bp, Artificial
  Spacer sequence
ggtcatgaatggatcgcagttcctcaaccgc

SEQ ID NO: 290 - DNA, 31 bp, Artificial
  Spacer sequence
tcgaatcccaccccagccgccacactcagca

SEQ ID NO: 291 - DNA, 42 bp, Artificial
  Spacer sequence
aattaatacttgttcaaccatgtcaaaccgaacttcgttgct

SEQ ID NO: 292 - DNA, 35 bp, Artificial
  Spacer sequence
agggtagtctttccctcgatagcaaaaagttccga

SEQ ID NO: 293 - DNA, 33 bp, Artificial
  Spacer sequence
ttaatgtcgctaaaattgggctcttcggcctga

SEQ ID NO: 294 - DNA, 31 bp, Artificial
  Spacer sequence
ggaacaatcttgcaaaggctgtgaaagttgg

SEQ ID NO: 295 - DNA, 27 bp, Artificial
  Spacer sequence
ttcacaggtaacatactccacccacca

SEQ ID NO: 296 - DNA, 38 bp, Artificial
  Spacer sequence
gggtgataccctcaaatttgtcagcttgaaagagctgg

SEQ ID NO: 297 - DNA, 38 bp, Artificial
  Spacer sequence
tgatgcttaaagcctgccataatgcaggtattcataca

SEQ ID NO: 298 - DNA, 36 bp, Artificial
  Spacer sequence
tataatctggacatactttgaagatttagccatgca

SEQ ID NO: 299 - DNA, 39 bp, Artificial
  Spacer sequence
taggtgtagcattggcgtcctctcacgcaaaacagccgc

SEQ ID NO: 300 - DNA, 37 bp, Artificial
  Spacer sequence
gtagcagtcaaatttcctttagggggttcaagataag

SEQ ID NO: 301 - DNA, 39 bp, Artificial
  Spacer sequence
ccttgatgagttcacgtggaaaaccccagccgatctgca

SEQ ID NO: 302 - DNA, 38 bp, Artificial
  Spacer sequence
aatataagacattcgtgataacgtcttatggcgttatc

SEQ ID NO: 303 - DNA, 38 bp, Artificial
  Spacer sequence
aggcgtcgaatataaaactttcgtgataacgtcttacg

SEQ ID NO: 304 - DNA, 35 bp, Artificial
  Spacer sequence
gaacaaataatatcactttcatatagttttccatt

SEQ ID NO: 305 - DNA, 34 bp, Artificial
  Spacer sequence
tgatttacagccattctttgataaagcaatagaa

SEQ ID NO: 306 - DNA, 34 bp, Artificial
  Spacer sequence
aaagaagtacgaaaatctgttatgaaattaaatt

SEQ ID NO: 307 - DNA, 33 bp, Artificial
  Spacer sequence
aaactagcagatgtctttggtgtaactactgat

SEQ ID NO: 308 - DNA, 34 bp, Artificial
  Spacer sequence
atttttgctgtataatataagtgaagtgaggtga

SEQ ID NO: 309 - DNA, 34 bp, Artificial
  Spacer sequence
aggtcaagggatttatgagaggaaaaggcaatat

SEQ ID NO: 310 - DNA, 33 bp, Artificial
  Spacer sequence
attgtctaacatcttaccaacgtctgctccgtt

SEQ ID NO: 311 - DNA, 33 bp, Artificial
  Spacer sequence
tttcaatactaaaatttcgggtatttccatcaa

SEQ ID NO: 312 - DNA, 33 bp, Artificial
  Spacer sequence
ggagatagtaaggaagttgcacaggcattagaa

SEQ ID NO: 313 - DNA, 32 bp, Artificial
  Spacer sequence
tgaatgcgccagccgctgccgccggatgcacc

SEQ ID NO: 314 - DNA, 32 bp, Artificial
  Spacer sequence
tcgataacgcccggtaaatacgtgtcaactaa

SEQ ID NO: 315 - DNA, 32 bp, Artificial
  Spacer sequence
gcgcttcccatcgcacagcgcacggcgcttcc

SEQ ID NO: 316 - DNA, 32 bp, Artificial
  Spacer sequence
gtgacacgctgtgacaaccccactttcccagc

SEQ ID NO: 317 - DNA, 32 bp, Artificial
  Spacer sequence
cagcacaataaatccccttgacagccccctcg

SEQ ID NO: 318 - DNA, 32 bp, Artificial
  Spacer sequence
tttgcggtatacgacgccgcgaccggcggaaa

SEQ ID NO: 319 - DNA, 32 bp, Artificial
  Spacer sequence
ggtgattttattcaaaaaaaagagagaggtga

SEQ ID NO: 320 - DNA, 32 bp, Artificial
  Spacer sequence
cgcgaccgcgccatcaattttgttctcgttgc

SEQ ID NO: 321 - DNA, 32 bp, Artificial
  Spacer sequence
ggttcggggggttcgtggtggagtgcaaccgc

SEQ ID NO: 322 - DNA, 32 bp, Artificial
  Spacer sequence
ttatcggagagcagcaagagtttgtcgatgat

SEQ ID NO: 323 - DNA, 32 bp, Artificial
  Spacer sequence
atttctggcgtcgggctctgctctcaagtgga

SEQ ID NO: 324 - DNA, 32 bp, Artificial
  Spacer sequence
gccgctacggcaattaaaaaggttttcaccac

SEQ ID NO: 325 - DNA, 32 bp, Artificial
  Spacer sequence
agccccaatttttttagtgacgcaaagcctcg

SEQ ID NO: 326 - DNA, 32 bp, Artificial
  Spacer sequence
gcctttaaccgttacgatcccggccggtcgtg

SEQ ID NO: 327 - DNA, 32 bp, Artificial
  Spacer sequence
ttgaaaatattgttgctgcgtgtttttgtgtg

SEQ ID NO: 328 - DNA, 33 bp, Artificial
  Spacer sequence
ccccatcgcctgaagcacgggccctaccatctc

SEQ ID NO: 329 - DNA, 34 bp, Artificial
  Spacer sequence
ggcatcaaggcttccggtgcgtcctcctggtgga

SEQ ID NO: 330 - DNA, 34 bp, Artificial
  Spacer sequence
gaggctggggggacaactccgagttttgcggcca

SEQ ID NO: 331 - DNA, 34 bp, Artificial
  Spacer sequence
tctaacctgctggcaatcaaagacgccttgcgcg

SEQ ID NO: 332 - DNA, 33 bp, Artificial
  Spacer sequence
gcacgatctcggagaatgggatagcgaaaagaa

SEQ ID NO: 333 - DNA, 35 bp, Artificial
  Spacer sequence
gggtgaaacatccgggatttatcgcttattggacg

SEQ ID NO: 334 - DNA, 34 bp, Artificial
  Spacer sequence
tgacgccaagggccgcccgcagtgcaaattagtg

SEQ ID NO: 335 - DNA, 34 bp, Artificial
  Spacer sequence
agaaaagagggaatggttcagcccgaaagatgtt

SEQ ID NO: 336 - DNA, 34 bp, Artificial
  Spacer sequence
tttgatttccaaggcgcgaaggtagccggattcc

SEQ ID NO: 337 - DNA, 34 bp, Artificial
  Spacer sequence
ctggcaaacggccaggtggcccaggcggcggacg

SEQ ID NO: 338 - DNA, 32 bp, Artificial
  Spacer sequence
ccgcccgccgatctggaaacggccgggcagca

SEQ ID NO: 339 - DNA, 32 bp, Artificial
  Spacer sequence
agttgctgcaggacccgcatgaacatcgccgc

SEQ ID NO: 340 - DNA, 32 bp, Artificial
  Spacer sequence
catgacggggtcggtccggacgatcatgacgg

SEQ ID NO: 341 - DNA, 32 bp, Artificial
  Spacer sequence
gggtggccctcgcttcgttgtgcggaccatac

SEQ ID NO: 342 - DNA, 32 bp, Artificial
  Spacer sequence
cgtgccgggtcagctcgcctcggtgcacccag

SEQ ID NO: 343 - DNA, 32 bp, Artificial
  Spacer sequence
ttcatcgcgggcggcgcgatccggacgagcat

SEQ ID NO: 344 - DNA, 32 bp, Artificial
  Spacer sequence
ccgagccgacgtcgcggcgatgctccgcgcag

SEQ ID NO: 345 - DNA, 32 bp, Artificial
  Spacer sequence
ccgggtcgtcgacaagccagccgacgagcagg

SEQ ID NO: 346 - DNA, 32 bp, Artificial
  Spacer sequence
gcggagcagtgcgggctcggcggcatgatcat

SEQ ID NO: 347 - DNA, 35 bp, Artificial
  Spacer sequence
gattcccacttttgtctttccacatatagcctgtg

SEQ ID NO: 348 - DNA, 38 bp, Artificial
  Spacer sequence
gtttcgattgtgaactcgatacgcggattttccttgtc

SEQ ID NO: 349 - DNA, 37 bp, Artificial
  Spacer sequence
ccccctctataattactatagatttggatggggcgat

SEQ ID NO: 350 - DNA, 35 bp, Artificial
  Spacer sequence
taacatgagtgactatggcgctgactttctgacgg

SEQ ID NO: 351 - DNA, 36 bp, Artificial
  Spacer sequence
ctcgaaggcgcgccgatcgacgacggcgaaggggcg

SEQ ID NO: 352 - DNA, 37 bp, Artificial
  Spacer sequence
ctgatcgccgtaggtgagcagcttcagcgtatcctcg

SEQ ID NO: 353 - DNA, 36 bp, Artificial
  Spacer sequence
cggagttcaatgtgtgggcggtccttgaacttccac

SEQ ID NO: 354 - DNA, 37 bp, Artificial
  Spacer sequence
caattctgttcgcccaatccggcgaactgtaccaaac

SEQ ID NO: 355 - DNA, 35 bp, Artificial
  Spacer sequence
gtacgaccgggaattcgacagctgaggcacggcca

SEQ ID NO: 356 - DNA, 37 bp, Artificial
  Spacer sequence
gtgttctcctgggcggagagcaccgatagcagtgtcg

SEQ ID NO: 357 - DNA, 34 bp, Artificial
  Spacer sequence
ttccagatttaaatgcacgcatcaacctacgata

SEQ ID NO: 358 - DNA, 33 bp, Artificial
  Spacer sequence
aataaagatatccgcaaatctgtcggccttaag

SEQ ID NO: 359 - DNA, 35 bp, Artificial
  Spacer sequence
ggtactggtggaggtttattactaggaagcgcaag

SEQ ID NO: 360 - DNA, 34 bp, Artificial
  Spacer sequence
cgttcggatcgatggtaaagacctgagttcggcc

SEQ ID NO: 361 - DNA, 34 bp, Artificial
  Spacer sequence
taaggaggtaacggactaatgcctttcatcgaca

SEQ ID NO: 362 - DNA, 33 bp, Artificial
  Spacer sequence
tagatccaaaatattacacgacacgattcgaca

SEQ ID NO: 363 - DNA, 33 bp, Artificial
  Spacer sequence
gactgtacaaggaattaggtaatgcttttgaag

SEQ ID NO: 364 - DNA, 33 bp, Artificial
  Spacer sequence
tatattatccctaatcaagaagctaaagctgcc

SEQ ID NO: 365 - DNA, 32 bp, Artificial; identical to SEQ ID NO: 344
  Spacer sequence

SEQ ID NO: 366 - DNA, 32 bp, Artificial; identical to SEQ ID NO: 345
  Spacer sequence

SEQ ID NO: 367 - DNA, 32 bp, Artificial; identical to SEQ ID NO: 346
  Spacer sequence

SEQ ID NO: 368 - DNA, 41 bp, Artificial
  Spacer sequence
ggtaccaaaggcgttatgatacgtagccatggctgaaacaa

SEQ ID NO: 369 - DNA, 41 bp, Artificial
  Spacer sequence
ggtaccaaaggagtagctataaattaagcgaaatcgataga

SEQ ID NO: 370 - DNA, 39 bp, Artificial
  Spacer sequence
cagctaaagttagaagatgctactaaagatctaagagat

SEQ ID NO: 371 - DNA, 34 bp, Artificial
  Spacer sequence
aataaaattcaagaagatttaaaaaagagaaagg

SEQ ID NO: 372 - DNA, 39 bp, Artificial
  Spacer sequence
caacaagaattaaaaaatgctactaaagatctaggagat

SEQ ID NO: 373 - DNA, 27 bp, Artificial
  Spacer sequence
ttacgagttcgttgattttcgccgtca

SEQ ID NO: 374 - DNA, 28 bp, Artificial
  Spacer sequence
tgaacgatgcctttgaccctgtccgccg

SEQ ID NO: 375 - DNA, 28 bp, Artificial
  Spacer sequence
gcgcaacgcagacctgaacgcttttaag

SEQ ID NO: 376 - DNA, 34 bp, Artificial
  Spacer sequence
ttcaagtattggcacatgctgggggaagagcgtg

SEQ ID NO: 377 - DNA, 33 bp, Artificial
  Spacer sequence
cgcgctgctttccacggcggagatggccctcgc

SEQ ID NO: 378 - DNA, 34 bp, Artificial; identical to SEQ ID NO: 376
  Spacer sequence

SEQ ID NO: 379 - DNA, 33 bp, Artificial; identical to SEQ ID NO: 377
  Spacer sequence